(19) United States
(12) Patent Application Publication
(10) Pub. No.: US 2022/0234007 A1
(43) Pub. Date: Jul. 28, 2022

(54) FUNCTIONAL COMPOSITE MEMBRANES FOR CHROMATOGRAPHY AND CATALYSIS

(71) Applicant: California Institute of Technology, Pasadena, CA (US)

(72) Inventors: Julia A. KORNFIELD, Pasadena, CA (US); Katherine T. FABER, Pasadena, CA (US); Mamadou DIALLO, Pasadena, CA (US); Orland BATEMAN, Pasadena, CA (US); Noriaki ARAI, Pasadena, CA (US)

(21) Appl. No.: 17/558,186

(22) Filed: Dec. 21, 2021

Related U.S. Application Data
(60) Provisional application No. 63/129,105, filed on Dec. 22, 2020.

(51) Int. Cl. B01D 69/12; C08L 27/16; B01J 31/16; B01J 31/06; B01J 37/00; C07K 1/22; B01D 71/34; B01D 71/60; B01D 15/36; B01D 15/38

(52) U.S. Cl. CPC B01D 69/12; C08L 27/16; B01J 31/1691; B01J 31/06; B01D 15/3809; C07K 1/22; B01D 71/34; B01D 71/60; B01D 15/361; B01J 37/009

(57) ABSTRACT

A composite, method of making the composite, and method of using the composite are disclosed. The composite comprises a macroporous scaffold comprising pores; and a polymer matrix positioned within the pores; wherein the polymer matrix comprises: a functional polymer particle; and a structural polymer. The method of using can comprise applications such as chromatography, catalysis, and sensing, among others.

### CROSS-REFERENCE TO RELATED APPLICATIONS

This application claims the benefit of priority to U.S. Provisional Patent Application No. 63/129,105, filed Dec. 22, 2020, which is hereby incorporated by reference in its entirety for all purposes.

### STATEMENT REGARDING FEDERALLY SPONSORED RESEARCH OR DEVELOPMENT

This invention was made with government support under Grant No. CBET1911972 awarded by the National Science Foundation. The government has certain rights in the invention.

### BACKGROUND

The field of membrane chromatography has expanded rapidly as an alternative to the conventional packed bed chromatography in pharmaceutical separations. The shift in technology has been motivated by a need to reduce downstream bioprocessing costs associated with long processing times, high operating pressures, and volumetric binding capacity dependent on flow rate. Membrane chromatography reduces the processing time by utilizing convective, as opposed to diffusive, mechanisms to transport molecules of interest to the associated binding sites. The change in transport mechanism enables the system to operate at faster flow rates while maintaining a low operating pressure. In addition, the use of convective transport allows for processing to be operated at a wide range of flow rates with minimal impact on the binding capacity of the membrane. These flow properties are amenable to the scale up of the separation processes, further reducing downstream costs. The adoption of membrane chromatography has also benefited from drawing on the experience of related fields in membrane science, i.e. identification of porous polymeric membranes with good chemical and physical stability to act as supports. As a result, many membrane adsorbers are derivatives of membranes used in other separation processes which are already produced on industrial scale, keeping down the cost of membrane adsorber modules.

In order to capitalize on the advantages outlined above, recent work has focused on addressing the key drawbacks of membrane chromatography. Two such drawbacks, relative to resin counterparts, are the low volumetric binding capacity of membrane adsorbers and loss of binding capacity when salt concentration is increased. While resins have a high binding surface area per volume ratio due to the tortuosity of the resin beads, membrane adsorbers initially relied solely on the pore surface area as the active binding area resulting in low volumetric binding capacity. One promising method to overcome this barrier is to use various polymerization techniques to graft polyelectrolyte chains or polymer brushes with appropriate functionalities onto the porous membrane supports. The resulting membranes benefit from the porosity of the support while increasing the available binding surface area to improve volumetric binding capacity. However, the improvement in volumetric binding capacity has only been demonstrated for solutions with low salt concentrations. Operating pharmaceutical separations in solutions with low ionic strength requires a buffer exchange step which increases capital and processing costs. Efforts to reduce the volume of buffer needed to dilute the salt and reduce the cost and time required to re-concentrate the antibody product have had limited success. In one approach, the widely used quaternary ammonium ligand is replaced with the less ion-sensitive primary amine ligand. Membranes using this approach retain their binding capacity even at salt concentrations of 150 mM; unfortunately, their binding capacity is very low. An alternative method fills the pores of the porous membrane supports with a functional hydrogel, the resulting composites have been utilized as membrane adsorbers for antibody purification. The functional hydrogel may bring a host of beneficial properties to the composite including responsiveness to environmental stimuli, hydrophilicity and unique binding chemistry. However, many of these functional hydrogels lack the mechanical properties required to be useful in separations or similar processes. Placing the functional hydrogels within an appropriate porous membrane support provides the necessary robustness, reduces swelling, and preserves the useful properties of the hydrogel. More recent efforts employing a pore-filling method with both polymeric and ceramic porous supports have focused predominately on using in-situ polymerization to synthesize these functional composites.

Thus, there is a need in the art for improved membranes for use in chromatography or catalysis applications. The invention is directed to these, as well as other, important goals.

### SUMMARY

In some aspects, disclosed is a composite comprising:
a macroporous scaffold comprising pores; and
a polymer matrix positioned within the pores;
wherein the polymer matrix comprises:
   a functional gel; and
   a structural polymer
wherein each of the macroporous scaffold, polymer matrix, pores, functional gel, and structural polymer are as defined elsewhere herein.

In some aspects, disclosed is a composite, comprising:
a macroporous scaffold comprising pores; and
a polymer matrix positioned within the pores;
wherein the polymer matrix comprises:
   a functional polymer particle; and
   a structural polymer.

In some aspects, disclosed is a method for making the composite of any preceding claim, the method comprising: infiltrating the pores with a liquid comprising the polymer matrix or a precursor thereof; and performing nonsolvent induced phase separation (NIPS) on the macroporous scaffold infiltrated with the liquid.

In some aspects, disclosed is a method for separating a component in a mixture and/or catalyzing a mixture, the method comprising passing the mixture through a composite.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A is a schematic diagram of a macroporous scaffold with pores containing a polymer matrix comprising a functional polymer particle (e.g., functional gel) and a structural polymer.

FIG. 1B: Static measurements of the BSA volumetric binding capacity of polymeric membranes at different salt concentrations utilizing the indicated crosslinker. Each membrane had a normalized crosslink density of 0.5. Each condition (crosslinker type and salt concentration) was repeated 6 times and the standard deviation was used for the error bars.

FIG. 2A: Schematic of the dope solution synthesis procedure using PVDF as structural polymer, PEI as functional precursor, and BCAH as crosslinker. FIG. 2B: alternative crosslinkers which have been tested in the polymeric and ceramic-polymer composite membranes.

FIGS. 3A-3C: Schematic of the freeze-casting process used to produce macroporous ceramic scaffold with a plurality of directionally aligned pores with (FIG. 3A) freeze casting apparatus, (FIG. 3B) image demonstrating freeze casting, (FIG. 3C) schematic detailing steps to achieve macroporous ceramic with cellular pores.

FIGS. 4A-4F: SEM micrographs showing the surface and cross section of neat ceramic (FIGS. 4A-4B), composite without functional polymer particle (e.g., functional gel) layer (FIGS. 4C-4D), composite with functional polymer particle (e.g., functional gel) layer (FIGS. 4E-4F).

FIGS. 5A-5D: SEM micrographs of composite cross-sections with NCD of (FIG. 5A) 0.5 (composition A), (FIG. 5B) 0.25 (composition B), (FIG. 5C) 0.125 (composition C), (FIG. 5D) 0.0625 (composition D)

FIG. 6: Static measurements of the BSA volumetric binding capacity of polymer and polymer-ceramic composite membranes at different crosslinker concentrations. BCAH was used as the crosslinker for these experiments. Experiments are currently being replicated.

FIG. 7: Static BSA binding measurements of polymer-ceramic composite B with NCD of 0.25 at different salt concentrations. Each experiment has been performed once.

FIG. 8: Illustration of different membrane regimes and corresponding transport (solution-diffusion and pore-flow).

FIGS. 9A-9B: Routes to form mixed-matrix membranes using (FIG. 9A) preformed particles and (FIG. 9B) in situ generated functional polymer particles.

FIG. 10: Ternary phase diagram representing the states in during nonsolvent induced phase separation for a Polymer/Solvent/Nonsolvent system.

FIGS. 11A-11E: The (FIG. 11A) ternary phase diagram for DMAc/PVDF/nonsolvent with corresponding cross-sectional SEM micrographs for nonsolvent of (FIG. 11B) water, (FIG. 11C) methanol, (FIG. 11D) ethanol, and (FIG. 11E) isopropanol.

FIGS. 12A-12D: SEM micrographs of the cross-sections of membranes cast from a polymer dope solution containing 15 wt. % PVDF in (FIG. 12A) TEP, (FIG. 12B) NMP, (FIG. 12C) DMF, (FIG. 12D) DMAc. See ref. 29 of Example 7.

FIGS. 13A-13D: Cross-sectional SEM micrographs of PVDF membranes prepared using the indicated nonsolvent. Bottom row micrographs are higher magnification images of top row.

FIG. 14: Illustration comparing mass transport mechanisms between packed beds (resins) and membrane chromatography.

FIG. 15: Illustration depicting the rejection capabilities of microfiltration (MF), ultrafiltration (UF), nanofiltration (NF), and reverse osmosis (RO).

FIG. 16: Flow regime map for different flow patterns based on Re and microcavity size.

FIG. 17: Fluorescent microscopic images using dilute fluorescent particles (d=1 μm) to illustrate growth of the microvortices with increasing Re.

FIGS. 18A-18I: Cross-sectional SEM micrographs for membranes prepared using nonsolvent and particle loading (FIG. 18A) IPA & 6 wt. %, (FIG. 18B) IPA & 38 wt. %, (FIG. 18C) IPA & 54 wt. %, (FIG. 18D) H₂O & 6 wt. %, (FIG. 18E) H₂O & 38 wt. %, (FIG. 18F) H₂O & 54 wt. %, (FIG. 18G) NMP:H₂O & 6 wt. %, (FIG. 18H) NMP:H₂O & 38 wt. %, and (FIG. 18I) NMP:H₂O & 54 wt. %.

FIGS. 19A-19I: Surface SEM micrographs for membranes prepared using nonsolvent and particle loading of (FIG. 19A) IPA & 6 wt. %, (FIG. 19B) IPA & 38 wt. %, (FIG. 19C) IPA & 54 wt. %, (FIG. 19D) H₂O & 6 wt. %, (FIG. 19E) H₂O & 38 wt. %, (FIG. 19F) H₂O & 54 wt. %, (FIG. 19G) NMP:H₂O & 6 wt. %, (FIG. 19H) NMP:H₂O & 38 wt. %, and (FIG. 19I) NMP:H₂O & 54 wt. %.

FIGS. 20A-20F: Schematics showing the casting solution-nonsolvent interface at t=0 (FIGS. 20A, 20C and 20E) and t=td, the characteristic length scale for mutual diffusion (FIGS. 20B, 20D and 20F). With gray circle—TEP, green circle—IPA, red circle—NMP, blue circle—H2O, blue line—PVDF, brown cluster—PEI.

FIGS. 21A-21B: Plots showing (FIG. 21A) background corrected x-ray scattering scans for samples cast in IPA, and (FIG. 21B) Intensity obtained from subtracting off the neat IPA scan signal from the indicated sample signal.

FIGS. 22A-22B: Plots showing (FIG. 22A) background corrected x-ray scattering scans for samples cast in H₂O, and (FIG. 22B) Signal obtained from subtracting IPA scan from H₂O scan at the indicated particle loading.

FIGS. 23A-23B: Plots showing (FIG. 23A) background corrected x-ray scattering scans for samples cast in NMP:H₂O, and (FIG. 23B) Signal obtained from subtracting IPA scan from NMP:H₂O scan at the indicated particle loading.

FIGS. 24A-24C: Plots of water flux as a function of time and particle loading for membranes prepared using (FIG. 24A) IPA, (FIG. 24B) H₂O, and (FIG. 24C) NMP:H₂O.

FIGS. 25A-25C: SEM micrographs of membrane cross-sections showing change in microgel shape and distribution using NCD of 0.5 and crosslinker chemistry of (FIG. 25A) ECH, (FIG. 25B) EGA, (FIG. 25C) BCAH.

FIGS. 26A-26C: SEM micrographs of membrane cross-sections showing changes in microgel distribution and structural polymer morphology with changing crosslink density (FIG. 26A) NCD—0.25, (FIG. 26B) NCD—0.5, (FIG. 26C) NCD—1.0.

FIG. 27: Static binding measurements depicting differences in binding capacity as a function of crosslinker chemistry and crosslink density in H₂O.

FIG. 28: Static binding capacity of BSA dissolved in water and TRIS/PBS buffers with varying conductivities.

FIG. 29: Breakthrough curves for membrane 54H in 50 mM TRIS at various flowrates to demonstrate regime of flowrate dependence at low volumetric flows.

FIG. 30: Breakthrough curves for membrane 54H at 0.6 mL/min (4 MV/min) in TRIS buffer with varying amounts of added salt demonstrating salt tolerance under flow.

FIG. 31: Dynamic binding capacities for membrane 54H at three different flowrates and 5 different buffer conditions, highlighting trends in salt tolerance behavior.

FIGS. 32A-32D: Composite membranes consisting of a porous support and a hydrogel comprised of (FIG. 32A) linear or (FIG. 32B) crosslinked polymer chains, (FIG. 32C) pore-filling polymer network, and (FIG. 32D) microgels supported by structural polymer.

FIGS. 33A-33B: Reaction schemes for (FIG. 33A) initial functionalization of amine surface terminating with aminosilane linker, (FIG. 33B) further surface functionalization using (1) ECH in IPA and (2) ECH+PEI in IPA.

FIG. 34: A visual depiction of the phase inversion micromolding process.

FIGS. 35A-35H: SEM micrographs showing the following: neat ceramic (FIG. 35A) cross-section & (FIG. 35B) surface, composite without surface functionality (FIG. 35C) cross-section & (FIG. 35D) surface, composite with ECH functionality (FIG. 35E) cross-section & (FIG. 35F) surface, and composite with PEI gel layer (FIG. 35G) cross-section & (FIG. 35H) surface.

FIGS. 36A-36B: Plots of static binding capacities for (FIG. 36A) both composite (CH) and polymeric membranes with 54% PEI loading using 2 mg/mL BSA in H₂O and (FIG. 36B) composite membranes using 2 mg/mL BSA in 50 mM TRIS at pH 7.4.

FIGS. 37A-37C: Depiction of composite membranes with PEI microgels in (FIG. 37A) an unswollen state, (FIG. 37B) a semi-swollen state physically restricted by the pore walls and other microgels, (FIG. 37C) fully swollen state under with no external restrictions.

FIG. 38: BSA binding breakthrough curves of an SiOC scaffold, an 54 wt. % PEI with NCD 0.25 composite membrane, and an 38 wt. % PEI with NCD 0.4 composite membrane.

FIGS. 39A-39F: Surface SEM micrographs, (FIG. 39A) formulation 1, IPA; (FIG. 39B) formulation 3, IPA; (FIG. 39C) formulation 5, IPA; (FIG. 39D) formulation 1, H2O; (FIG. 39E) formulation 3, H2O; (FIG. 39F) formulation 5, H2O.

FIGS. 40A-40F: SEM micrographs of the following cross-sections, (FIG. 40A) formulation 1, IPA; (FIG. 40B) formulation 3, IPA; (FIG. 40C) formulation 5, IPA; (FIG. 40D) formulation 1, H2O; (FIG. 40E) formulation 3, H2O; (FIG. 40F) formulation 5, H2O.

FIGS. 41A-41C: Static protein binding experiments performed using BSA in the following solvents: (FIG. 41A) H2O, pH 6.5; (FIG. 41B) 50 mM TRIS Buffer, pH 7.5; (FIG. 41C) 1×PBS, pH 7.5.

FIG. 42: BSA static binding demonstrating high volumetric binding capacity and improved salt tolerance.

FIG. 43: Measurements demonstrating the influence of flowrate on dynamic binding capacity. Measurement solution was 2 mg/mL BSA in 50 mM Tris buffer.

FIG. 44: Dynamic binding salt tolerance measurements with an operating flowrate of 600 μL/min (4 MV/min).

FIGS. 45A-45B: Scanning electron microscopy (SEM) micrographs of IPA-induced PVDF solidification showing loose spherulitic PVDF structures and PEI particles located on the edges of the PVDF structure.

FIG. 46: A graph showing number of particles versus diameter. See Example 14.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “composite” and “composite membrane” are used interchangeably herein and refer to a combination of at least a macroporous scaffold and a functional polymer particle (e.g., functional gel) as those terms are defined herein. Generally, the functional polymer particle (e.g., functional gel) is disposed within the pores of the scaffold. In some aspects, the functional polymer particle (e.g., functional gel) is part of a polymer matrix that includes a structural polymer, as those terms are defined herein.

The terms “macroporous scaffold” and “scaffold” are used interchangeably herein and refer to a porous material that provides structural support for the functional polymer particle (e.g., functional gel). In some aspects, the porous material comprises pores exhibiting directionality, which facilitates higher flow rates through the scaffold. The scaffold can comprise any suitable material, such as ceramic, glass, metal, and so forth, as discussed elsewhere herein. In some aspects, the functional polymer particle (e.g., functional gel) is part of a polymer matrix as that term is defined elsewhere herein.

The term “internal structure” refers to the internal geometry or internal configuration in a material (e.g., within the external boundaries (e.g., external surfaces) of the material), such as the macroporous scaffold. The term internal structure does not refer to structure on an atomic length scale of a material, such as the characterization of crystallographic structure. An internal structure comprising pores or voids can be characterized as a “porous internal structure” or “macroporous structure.” The term “porous”, as used herein, refers to a material or structure within which pores are contained. Thus, for instance, in a porous material or structure, such as a macroporous scaffold, the pores are volumes within the body of the material or structure where there is no material. Pores can be characterized by a “pore characteristic” including, but not limited to, a (average) size characteristic, a geometrical parameter, a pore-type, directionality, a primary growth direction, a primary growth axis, a secondary growth axis, being a continuous through-pore, or any combination thereof. Geometrical parameters of a pore are exemplary size characteristics of a pore. An exemplary cross-sectional dimension of a pore is its hydraulic diameter, which is defined as the ratio of the cross sectional area of the pore divided by the wetted perimeter of the pore. A pore of an internal structure can be characterized by its pore-type. Exemplary pore-types include, but are not limited to, dendritic pores, cellular pores, lamellar pores, prismatic pores, isotropic pores, transitional pores, closed cell pores, or any combination thereof. In some aspects, the pores of a macroporous scaffold or composite membrane comprise a minority of closed cell pores or no closed cell pores In some aspects, a majority of the pores are not a closed cell structure (e.g., a closed cell foam), or in other words, in some aspects, a minority of pores comprise a closed cell structure or no closed cell pores are present. Porosity can be determined by Archimedes density measurements if the density of the solid is known. Amount of closed cell pores can be estimated using the density and measured porosity.

The terms “polymer matrix,” “polymer network,” and “mixed-matrix” generally are used interchangeably herein unless contradicted by context and refer to a combination of a functional polymer particle (e.g., functional gel) and a structural polymer. In this regard, a “mixed-matrix” in some aspects is not a polymer matrix, since the most generic definition of mixed-matrix is a matrix comprised of two different materials, neither of which needs to be a polymer. In some aspects, the polymer matrix is formed by polymerizing and/or crosslinking the functional polymer particle (e.g., functional gel) or a precursor thereof in the presence of dissolved structural polymer, such that, in some aspects, the functional polymer particle is interspersed in the structural polymer.

The term “functional polymer particle” refers to a polymer particle that comprises one or more functional groups capable of carrying out a desired function. For example, in some aspects, the one or more functional groups are capable of binding to an analyte of interest for, e.g., chromatography. In some aspects, the one or more functional groups are acid groups and/or chelated metals for, e.g., catalysis. In some aspects, multiple functions are possible with the one or more functional groups within a functional gel, such as both chromatography and catalysis. The term “functional polymer particle” includes, for example, a functional gel, a metal-organic framework (MOF), a covalent organic framework (COF), a nanoporous polymer, or any combination thereof.

The term “gel” refers to a polymer or polymer system that swells to at least twice (e.g., at least 2 times, at least 3 times, at least 4 times, at least 5 times, at least 6 times, at least 7 times, at least 8 times, at least 9 times, or at least 10 times), and generally less than 20 times (e.g., less than 18 times, less than 15 times, less than 10 times, or less than 5 times) its dry volume when immersed in a given fluid, liquid, solvent, solution, or the like. In some aspects, the polymer or polymer system is crosslinked. Generally, certain fluids, liquids, solvents, solutions, and the like will swell a polymer or polymer system, and certain other fluids, liquids, solvents, solutions, and the like will not swell a polymer or polymer system (or will only partially swell a polymer or polymer system to less than twice its dry volume). During operation of a composite membrane, the gel will generally be in its swollen state under the conditions under which the composite membrane is operated (e.g., for chromatography, catalysis, and/or other applications). Thus, a polymer or polymer system does not need to swell to twice its dry volume in all fluids, liquids, solvents, solutions, and the like, it need only swell under conditions that the composite membrane will be used. In this context, the fluids, liquids, solvents, solutions, and the like are encompassed by the term “working fluid.” For clarity, the term “fluid” or “working fluid” refers to a liquid, a solvent, a solution, a gas, a sub-critical fluid, a supercritical fluid, or any combination thereof. Generally, a “working fluid” is the fluid that a composite membrane herein is operated with for a given application, such as catalysis or separations. In some aspects, a “gel” is referred to as a “microgel” herein.

The term “functional gel” refers to a gel which additionally comprises one or more functional groups capable of carrying out a desired function. For example, in some aspects, the one or more functional groups are capable of binding to an analyte of interest for, e.g., chromatography. In some aspects, the one or more functional groups are acid groups and/or chelated metals for, e.g., catalysis. In some aspects, multiple functions are possible with the one or more functional groups within a functional gel, such as both chromatography and catalysis. In some aspects, a “functional gel” is referred to as a “microgel” herein or a “functional microgel” herein.

The term “hydrogel” is a gel that swells in a liquid that is or comprises water (e.g., a liquid comprising at least 50% water).

The term “functional hydrogel” is a hydrogel that also meets the definition of functional gel.

The term “structural polymer” refers to a polymer that is chemically stable under the conditions used to operate the composite membrane (e.g., for chromatography and/or catalysis) and which is also insoluble under such conditions.

The term “swelling ratio” refers to the ratio of mass of a functional polymer particle (e.g., functional gel) when swollen with a given liquid (e.g., water) to dry mass. “Dry mass” means the mass of the gel without the liquid (e.g., without water or any other liquid that is used for operating the composite membrane). Suitable methods for calculating the swelling ratio are described elsewhere herein.

The term “swollen,” “swells,” or similar terms refer to a species, such as a functional polymer particle, gel, or functional gel, that expand in size (e.g., volume) and/or mass when immersed in a given fluid (e.g., when measured in a wet state). In some aspects, the expansion in size and/or volume is at least 2 times, at least 3 times, at least 4 times, at least 5 times, at least 6 times, at least 7 times, at least 8 times, at least 9 times, or at least 10 times its dry volume.

The term “normalized crosslinking density (NCD)” is calculated using the equations described in detail elsewhere herein. Conceptually, particularly in aspects relating to ECH as crosslinker and PEI as polymer to be crosslinked (though this concept also applies to other systems), NCD refers to the ratio NB in which A is defined as the moles of crosslinker functional groups divided by moles of PEI functional groups; and B is defined as the moles of crosslinker functional groups divided by moles of PEI functional groups taken at the reference state of 1.65 g ECH to 2.58 g PEI. Soley to illustrate the calculation, for a solution comprising 5 grams of PEI and 0.85 grams ECH the NCD would be calculated as follows: A=0.018 moles ECH FG/0.13 moles PEI amines; B=0.036 moles ECH FG/0.06 moles PEI amines; NCD=A/B=0.23.

The term “crosslink density” or “crosslinking density” refers to the moles of crosslinker functional groups divided by moles of functional groups of polymer to be crosslinked. In aspects relating to PEI, for example, the functional groups of PEI would count primary amines as two different functional groups as the amine would be able to react twice, and the secondary amines would only count as one. This concept also applies to polymers other than PEI where multiple functional groups are present. Another illustration of this concept is as follows. If the reactive groups on a crosslinker are capable of forming are n bonds to available functional groups on the precursor of the functional polymer particle (e.g., microgel) and the average number reactive groups on the precursor of the functional polymer particle (e.g., microgel) could form up to m bonds covalent bonds to the crosslinker; and if n moles of crosslinker are reacted with m moles of precursor of the functional polymer particle (e.g., microgel), then the crosslinking ratio is (n bonds*n moles)/(m bonds*m moles).

The term “pore volume fraction” of the macroporous scaffold refers to the ratio of total pore volume to the total volume of the macroporous scaffold (including pores) and can be determined using the gravimetrically measured density of the macroporous material and the known density of the solid phase of the macroporous scaffold. Suitable methods for determining the volume fraction are described elsewhere herein.

The phrase “oriented along a primary axis” and the term “directionality” refer to a characteristic of pores that can be described to extend in a direction. Generally, the term “directionality” refers to an overall or average pore configuration, such as of the main channel of a pore rather than of its secondary arms (e.g., when dendritic pores are present). Orientation along a primary axis generally facilitates flowing a liquid through a composite membrane herein. Generally, there is a directionality to the pores of a macroporous scaffold over a length scale of, solely by way of example, about 300-500 μm or more, as opposed to on a nanoscale (e.g., 10 nm). For example, pores having directionality may be characterized by as having a primary growth direction. The term “primary growth direction” refers to the direction in which a directional pore, or longitudinal pore, extends. The primary growth direction of a pore is a direction of its primary growth axis (its longitudinal axis). In cellular and dendritic pores, one can determine primary growth direction by observing or measuring the axial direction of the main pore. In prismatic pores, one can determine primary growth direction by observing or measuring the long axis of the prism. The only case in which we cannot observe the orientation of an axis is the lamellar case in which orientation of the normal to an internal surface is used to characterize directional homogeneity. For example, a plurality of parallel longitudinal pores, such as cellular pores or dendritic pores, can have identical primary growth directions but unique primary growth axes (e.g., the primary growth axes have same direction but each is transposed in physical space with respect to another). In other words, two pores having identical primary growth directions is an indication that they have parallel primary growth axes. Isotropic pores and pores of a stochastic foam do not have a primary growth direction or a primary growth axis. In some aspects, dendritic pores are present and include secondary arms, where each secondary arm is characterized by its own secondary growth axis. In some aspects, dendritic pores may also include higher order arms, such as tertiary arms. In some aspects, the primary growth axis of a pore can be characterized as a straight line of best fit representing the pore geometry/configuration in its entirety. A pore having directionality is an anisotropic pore. For example, the primary growth direction and the primary growth axis can be determined from conventional micrographs that probe the relevant length scales of the pore structure, from imaging techniques such as scanning electron microscopy (SEM), or from three-dimensional imaging techniques such as X-ray (micro)tomography.

The term “infiltrating” (or other similar terms, such as infiltration) refers to a liquid passing into the pores of the macroporous scaffold whether through passive (e.g., permeation and/or diffusion) or active (e.g., via an applied force such as pumping) means, or a combination thereof. In some aspects, the liquid comprises a functional polymer particle (e.g., functional gel), a structural polymer, or a combination thereof.

The term “polymer dope solution” refer to a liquid that is infiltrated into the pores of a macroporous support. In some aspects, the polymer dope solution comprises the functional polymer particle (e.g., functional gel), a precursor of the functional polymer particle (e.g., precursor of the functional gel), the structural polymer, a precursor of the structural polymer, or any combination thereof. In some aspects, the polymer dope solution comprises a structural polymer and a precursor of the functional polymer particle (e.g., functional gel). In some aspects, the polymer dope solution comprises a structural polymer and a precursor of the functional polymer particle (e.g., precursor of the functional gel) in which the synthesis of the functional polymer particle (e.g., functional gel) has been at least initiated, and optionally completed. Components in the polymer dope solution can be fully dissolved, at least partially dissolved, insoluble, at least partially insoluble, suspended, emulsified, or any combination thereof.

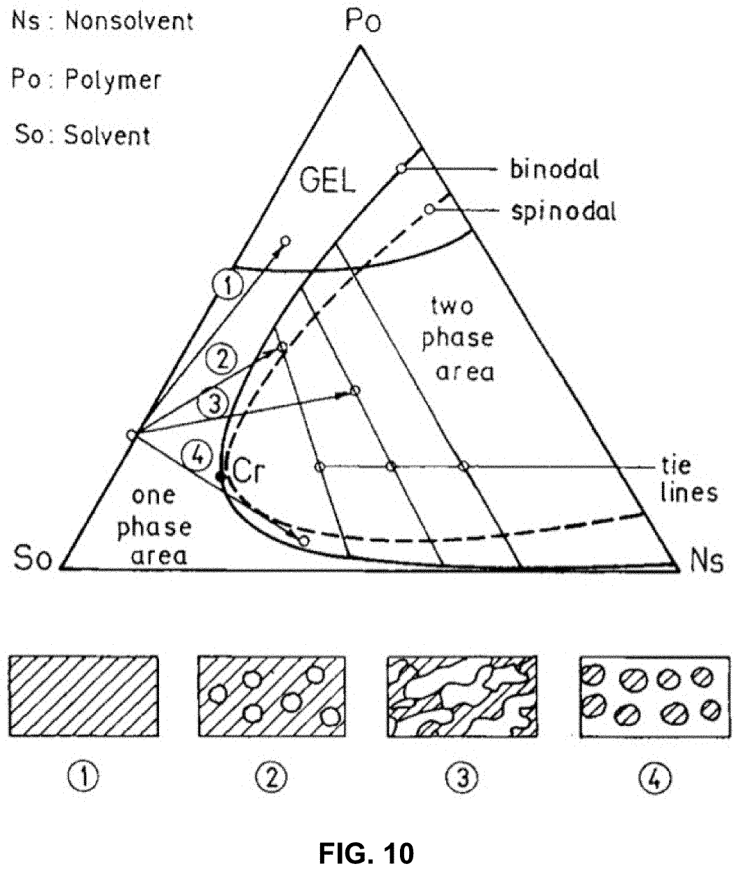

The phrase “nonsolvent induced phase separation” (NIPS) is a known term of art with the same meaning as generally used in the art. For example, the NIPS process generally begins when the homogeneous liquid polymer solution is immersed in a liquid that is incompatible with the polymer, known as a nonsolvent. “Incompatible” in this context generally means the polymer is not soluble in, or only very slightly soluble in or slightly soluble in, the liquid. As the solvent and nonsolvent interdiffuse, the composition of the casting solution changes and can follow one of four routes depending upon the rates of mass transfer follows one of the 4 routes shown in FIG. 10. Along the four routes there are two types of demixing processes to consider: liquid-liquid demixing—in which the ternary solution starts as a homogeneous solution in the one phase area and then crosses the binodal into an unstable regime that induces phase separation into two liquid phases, and solid-liquid demixing—wherein a ternary solution in either the one phase or two phase area cross into the gel region producing a solid polymer crystal phase in equilibrium with a liquid polymer-lean phase. In other words, in liquid-liquid demixing the solution phase separates as a liquid and then the polymer-rich region solidifies and crystallizes. In solid-liquid demixing the polymer crystallization and solidification drives phase separation and as a result is a slower process that is seen mostly in semi-crystalline polymers such as PVDF. The NIPS process is described in more detail elsewhere herein.

The term “fluid communication” refers to the configuration of two or more pores such that a fluid (e.g., a gas or a liquid) is capable of transport, flowing and/or diffusing from one pore to another pore, within a macroporus scaffold or composite membrane, without adversely impacting the functionality of each of the pores or of the material having said pores. In some aspects, pores, such as pores of the macroporous scaffold or composite membrane, can be in fluid communication with each other via one or more intervening pores. Pores can be direct fluid communication wherein fluid is capable of moving directly from one pore to another. Pores in fluid communication with each other can be in indirect fluid communication wherein fluid is capable of transport indirectly from one pore to another pore via one or more intervening pores that physically separate the components. The term “fluid communication” can be used to describe two or more zones of an internal structure, such as two zones are in fluid communication when one or more pores from one zone are in fluid communication with one or more pores of the other zone.

The term “freeze-casting” refers to a process suitable for forming a macroporous scaffold, wherein the process includes freezing a solvent (or, dispersion medium) of a liquid formulation and subsequently removing the solvent by sublimation or solvent extraction. Freeze-casting is described in more detail elsewhere herein.

The terms “directionally freezing” and “directional freeze casting” refer to the process of freezing, such as a freezing solvent, that is not isotropic (i.e., is anisotropic). For example, directionally freezing corresponds to a freezing front moving along a single direction (uni-directional freezing), or up to several directions. For example, freezing may initiate at a surface (e.g., a cold surface) and proceed in direction(s) substantially normal to the surface. For example, the surface can be planar or curved. In some aspects, a primary growth direction of a pore is substantially equal to the normal to the surface at which the directional freezing initiated.

The term “wet state” typically is used herein in reference to a method in which an average particle size of a plurality of particles in a swollen state is determined. In particular, a polymer matrix is incubated in a good solvent for the structural polymer (e.g., a solvent that solubilizes the structural polymer) until a suspension is formed. Once a suspension is formed, the functional polymer particles are separated out using either filtration or centrifugation. The functional polymer particles are then suspended in a fluid of interest, such as a fluid that the composite membrane is intended to operate at (e.g., a fluid that swells the functional polymer particle, such as when the functional polymer particle is or comprises a functional gel). In some aspects, the fluid is or comprises water. The average particle size of the formed suspension is then determined using dynamic light scattering (DLS).

The term “dry state” typically is used herein in reference to a method in which an average particle size of a plurality of particles is determined under conditions typically used in the art to conduct scanning electron microscopy (SEM) measurements, as would be understood in the art.

The term “average diameter of through-pores,” sometimes designed “D” herein, refers to through-pores in the scaffold alone (i.e., not containing a polymer matrix or functional polymer particle within the pores), and D is determined using porosimetry.

The term “low generation” in reference to a dendrimer, such as a polyamidoamine dendrimer (PAMAM), means a 0^{th}, 1^{st}, 2^{nd}, or 3^{rd} generation. In some aspects, the low generation dendrimer is 0^{th}, 1^{st}, or 2^{nd} generation.

The term “hyperbranched” refers to a polymer having a dendrimer-type structure, but where there are errors in the bonding such that the repeating internal structure is not uniform. Generally, such errors lead to leftover functional groups that are available in the interior of the repeating structure and a different 3D structure than would be available in a dendrimer with no errors.

The term “halide” refers to chloride, bromide, or iodide.

The term “low molecular weight PEG” refers to PEG (polyethylene glycol) having a molecular weight (e.g., weight-average molecular weight) of less than 1,000 g/mol.

The term “number average molecular weight” or “Mₙ” has its art-recognized definition. Solely by way of illustration, Mₙ is the ordinary arithmetic mean or average of the molecular masses of the individual macromolecules determined by measuring the molecular mass of n polymer molecules, summing the masses, and dividing by n. Generally, the following equation can be used:

$${\overline{M}}_{n} = \frac{{Σ}_{i} {N}_{i} {M}_{i}}{{Σ}_{i} {N}_{i}}$$

where Nᵢ is the number of molecules of molecular mass Mᵢ.

The term “weight average molecular weight” or “M_{w}” has its art-recognized definition. Solely by way of illustration, M_{w} takes into account that larger molecules have a larger contribution to the average molecular weight than smaller molecules and generally can be determined according to the following equation:

$${\overline{M}}_{w} = \frac{{Σ}_{i} {N}_{i} {M}_{i}^{2}}{{Σ}_{i} {N}_{i} {M}_{i}}$$

where Nᵢ is the number of molecules of molecular mass Mᵢ.

In some aspects, a composition or compound of the invention, such as an alloy or precursor to an alloy, is isolated or substantially purified. In an aspect, an isolated or purified compound is at least partially isolated or substantially purified as would be understood in the art. In an aspect, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In some aspects, disclosed herein are compositions of matter for membrane adsorbers (and/or catalyzers), methods of making membrane adsorbers (or and/catalyzers) that comprise such compositions, and methods of using such membrane adsorbers (and/or catalyzers). No prior study has, among other things, examined mixed-matrix membrane compositions as a pore-filling material. In some aspects, a novel pore-filling method is used with a macroporous scaffold (e.g. a ceramic) with one or more of the following advantages. 1. The scaffold (e.g., ceramic, such as silicon oxycarbide ceramic (SiOC)) is inert under the operating conditions often used in bioseparations. 2. The scaffold is mechanically robust; it has compressive strength that outperforms inert porous polymer membranes. 3. The scaffold is readily functionalized (e.g., via silanization, which is a versatile, widely-available method that does not require extreme operating conditions or exacting control). In some aspects, any such advantage, or combination of such advantages, may be associated with the disclosed compositions, scaffold, functional composite membranes, methods of making, and/or methods of using disclosed herein. In some aspects, a novel pore-filling method comprises the following steps: preparation of a polymer dope solution, infiltrating the dope solution into a scaffold (e.g., ceramic; in some aspects the scaffold may be functionalized), and using an appropriate nonsolvent to initiate a phase inversion solidification of the dope solution. Such a method is described in more detail elsewhere herein. To our knowledge phase-inversion solidification (e.g., NIPS) has not been used in a porous material. Here, in some aspects, the process produces a composite scaffold-functional polymer particle membrane (e.g., a composite ceramic-functional hydrogel membrane). Other macroporous materials with oriented through-pores may also be utilized. In some aspects, such macroporous scaffolds can be prepared via directional freeze casting to produce scaffolds made of ceramic, inorganic glass, carbon, fused metal particles, fused polymer particles, or other dispersed species present in the freeze-casting composition, or any combination thereof (see, e.g., “Freeze-Cast Ceramic Membrane for Size Based Filtration”—U.S. patent application Ser. No. 16/549,954 and International Patent Application PCT/US2019/048005, both of which are hereby incorporated by reference in their entireties for all purposes).

In some aspects, ceramic or other materials suitable for use as the scaffolds herein can be produced in a variety of ways. For example, electrochemical synthesis of ceramics with highly oriented pores of uniform size could be used to synthesize cellular pores oriented directly through the membrane. For example, Anodisc membranes originally developed by GE Healthcare, then produced by Cytiva, and currently produced by Whatman are composed of alumina and are available with cutoff sizes of 20 μm, 100 μm or 200 μm.

In some aspects, freeze casting (e.g., directional freeze casting) may be used to synthesize highly oriented pores of uniform size with greater control of the pore morphology (cellular or dendritic pores). In some aspects, suitable freeze casting for preparing a macroporous scaffold is described in “Freeze-Cast Ceramic Membrane for Size Based Filtration”—U.S. patent application Ser. No. 16/549,954 and International Patent Application PCT/US2019/048005. In freeze casting, a macroporous scaffold typically is produced by freezing a solvent (or, dispersion medium) of a liquid formulation and subsequently removing the solvent by sublimation or solvent extraction. In some aspects, the liquid formulation comprises the solvent and chemical species dispersed therein. Exemplary dispersed species include, but are not limited to, powders, such as ceramic powders, preceramic polymers, colloidal particles, micelles, salts, and any combinations of these. In some aspects, the crystallizing (freezing) and/or crystallized solvent leads to exclusion of the dispersed species therefrom, resulting in redistribution of non-solvent solids that subsequently form or template the internal structure of the porous material. The frozen/crystallized solvent is then removed from the pores of the internal structure by sublimation or solvent extraction. In some aspects, the solvent is a solvent mixture. In some aspects, the freezing is a directional freezing. In some aspects, freeze casting is characterized by forming a material with an internal structure characterized by directional pores having a cross-sectional dimension, such as diameter, selected from the range of 500 nm to 500 μm.

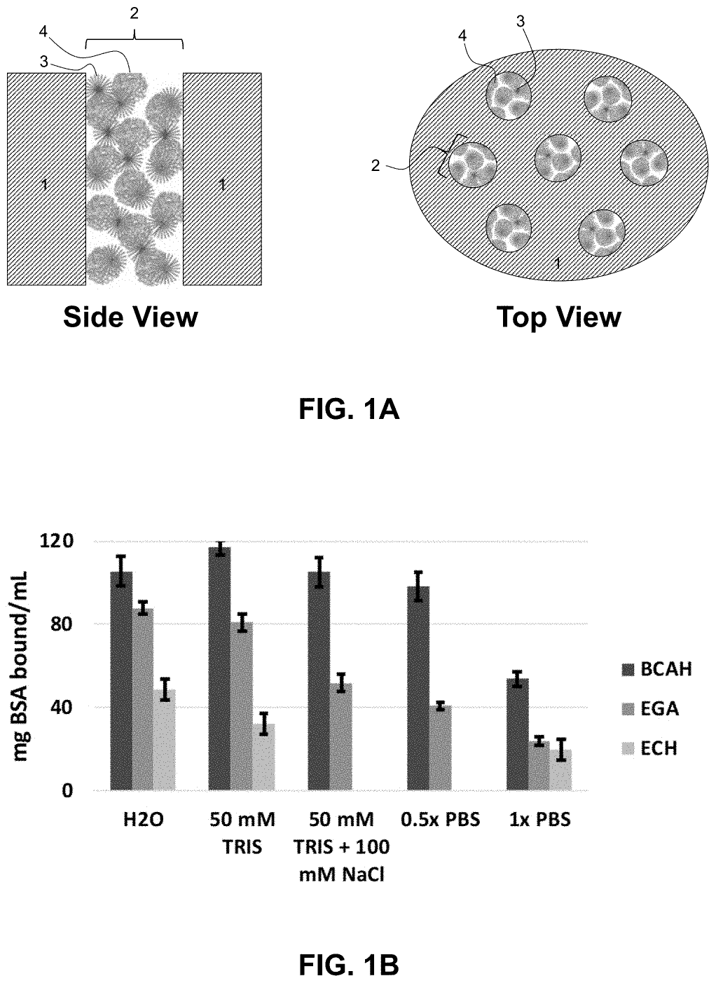

Composition of matter. In some aspects, disclosed herein is a functional composite membrane comprising a macroporous support/scaffold in which the pores are filled with a functional polymer particle (e.g., functional gel) or plurality of particles thereof interspersed with a structural polymer. FIG. 1A is a schematic diagram depicting a macroporous scaffold 1 with pores 2 containing a polymer matrix comprising a functional polymer particle (e.g., functional gel) 3 and a structural polymer 4. The macroporous scaffold, functional polymer particle (e.g., functional gel), and structural polymer are described in further detail below.

In some aspects, the macroporous scaffold comprises ceramic, metal, polymer, glass, or any combination thereof. In some aspects, the macroporous scaffold comprises a plurality of pores that exhibit directionality. In some aspects, the macroporous scaffold is or comprises a ceramic with a plurality of pores exhibiting directionality, a metal or metallic composite with a plurality of pores exhibiting directionality, a polymer glass or semi-crystalline polymer with a plurality of pores exhibiting directionality, a combination of the three, or any combination thereof. The pore surfaces of the scaffold may be bare, functionalized sparsely or incompletely with reactive functional groups, functionalized with a monolayer terminating in a reactive functional group, or a conformal coating. In some aspects, the functionalization or the conformal coating may be adsorbed or covalently bonded to the pore surface of the porous support/scaffold. In some aspects, a majority of the pores are not a closed cell structure (e.g., a closed cell foam), or in other words, in some aspects, a minority of pores comprise a closed cell structure or no closed cell pores are present.

In some aspects, the pores of the macroporous scaffold are in fluid communication with one another. For example, in some aspects, such fluid communication generally allows a fluid to flow through the composite membrane, in some aspects facilitating chromatography and/or catalysis.

In some aspects, the functional polymer particle (e.g., functional gel) comprises a polymer that swells (e.g., readily swells) in a solvent and is crosslinked to form a polymer network that swells to at least twice its dry volume when immersed in a solvent, liquid, solution, or the like. In some aspects, the functional polymer particle (e.g., functional gel) is or comprises a functional hydrogel and swells to at least twice its dry volume when used in an aqueous system. Suitable polymers of the functional polymer particle include, but are not limited to, polyethylenimine (PEI), hyperbranched PEI, poly-N-isopropylacrylamide, polyamidoamine dendrimers (PAMAM), low generation PAMAM, poly(ethylene oxide) (PEO), chitosan, gelatin, another functional biopolymer, a combination thereof, or any combination thereof. Suitable crosslinkers include but are not limited to bis(2-chloroethyl)amine hydrochloride (BCAH), (2-Chloroethyl)(3-chloropropyl)amine, 2-Chloro-N-(2-chloroethyl)-1-propanamine hydrochloride, N,N′-Bis(2-chloroethyl)ethane-1,2-diamine, epichlorohydrin (ECH), diethylene glycol diacrylate (EGA), other crosslinkers disclosed elsewhere herein, a combination thereof, or any combination thereof. In some aspects, any suitable crosslinker similar or related to those listed herein may be used, though certain crosslinkers listed herein perform surprisingly better than others, as described elsewhere herein. In addition, the crosslinker may comprise any combination of the molecules listed herein or their alternatives.

In some aspects, the low generation PAMAM can include, for example, G0, G1, G2, or G3 dendrimers. By way of example, G0 has four primary amines and a molar mass of 517 g/mol, and G1 has eight primary amines and a molar mass of 1430 g/mol. Such properties for G2 and G3 also can be readily determined.

In some aspects, the structural polymer comprises a mechanically robust polymer exhibiting chemical stability under typical conditions and is insoluble in a solvent that swells the functional polymer particle (e.g., functional gel) (i.e. insoluble in water for aqueous systems). In some aspects, the structural polymer may be composed of polyvinylidene fluoride (PVDF), cellulose acetate, polysulfone, polyvinyl chloride, poly(acrylonitrile), polyethersulfone (PES), polypropylene, polytetrafluoroethylene, polyamide imide, natural rubber, other structural polymers disclosed elsewhere herein, a combination thereof, or any combination thereof. In some aspects, any suitable polymer related to those listed herein may be used. In addition, the structural polymer may consist of a combination or any combination of the polymers listed here or their alternatives.

In some aspects, the composition may comprise a volume fraction of the macroporous support/scaffold of 15 to 59%. The volume fraction of the macroporous scaffold can be measured using the known density of the solid phase of the macroporous scaffold and the gravimetrically measured density of the macroporous material. In some aspects, the pores of the macroporous support/scaffold have a size typically in the range of 20 to 200 μm or 500 nm to 500 μm and may be polydispersed in size. Exemplary methods to measure the pore size are mercury intrusion porosimetry and electron microscopy (described in Example 5). In some aspects, the pores may be cellular or dendritic in form. In some aspects, the macroporous support/scaffold can have a lamellar or prismatic morphology.

In some aspects, the functional polymer particle (e.g., functional gel and/or functional hydrogel) interspersed with a structural polymer may comprise 20-65% w/w of structural polymer. The ratio of structural polymer and functional polymer particle can be measured by a variety of methods, some of which require the macroporous scaffold to be dissolved and other are equally applicable to insoluble macroporous supports. If the scaffold can be dissolved without degrading the functional polymer particle (e.g., functional hydrogel) or structural polymer, the ratio of structural polymer to functional polymer particle can be measured following dissolution of the macroporous scaffold and an appropriate chemical analysis method (Raman or IR spectroscopy, elemental analysis, or other method). If the scaffold is insoluble, elemental analysis may be used: for example, fluorine might serve as a useful surrogate for the structural polymer in the case of PVDF, nitrogen might serve as a surrogate for the functional polymer particle, and silicon might serve as a surrogate for the scaffold.

In some aspects, the functional polymer particle (e.g., functional gel and/or functional hydrogel) has a degree of crosslinking that is characterized by a swelling ratio of between 2 and 20 (ratio of mass when swollen with water to dry mass). In some aspects, a swelling ratio less than 2 fails to make the functional groups accessible to the species of interest, and a swelling ratio greater than 20 makes it difficult to maintain the combination of flow through gaps between functional gel particles. The swelling ratio of the functional polymer particle (e.g., functional gel) can be measured by a variety of methods, some of which require the macroporous scaffold to be dissolved and other are equally applicable to insoluble macroporous supports. If the scaffold can be dissolved without degrading the functional polymer particle (e.g., functional gel) or structural polymer, the swelling ratio of the functional polymer particle (e.g., functional gel) can be measured following dissolution of the macroporous scaffold by drying the resulting interspersed functional polymer particle (e.g., functional gel) and structural polymer. The mass uptake of water upon swelling the interspersed functional polymer particle (e.g., functional gel) and structural polymer in a relevant aqueous solution can be measured by mass. The ratio of water absorbed per mass of functional polymer particle (e.g., functional gel) can be computed using the mass of dry interspersed functional polymer particle (e.g., functional gel) and structural polymer multiplied by the mass fraction of functional polymer particle (e.g., functional gel) in the dry interspersed functional polymer particle (e.g., functional gel) and structural polymer. If the scaffold is insoluble, the swelling ratio of the functional polymer particle (e.g., functional gel) may be estimated by pulverizing the scaffold, rigorously drying the resulting particles, analyzing the ratio of functional polymer particle (e.g., functional gel) to total solids (e.g., using elemental analysis or other suitable method) and measuring mass uptake in an atmosphere with a high relative humidity. When the scaffold is insoluble, the ratio of water absorbed per mass of functional polymer particle (e.g., functional gel) can be computed using the mass of dry interspersed functional polymer particle (e.g., functional gel) and the mass fraction of functional polymer particle (e.g., functional gel) in the dry powder obtained by pulverizing the scaffold. In some aspects, the compositions have the functional polymer particle (e.g., functional gel) crosslinks interspersed with the structural polymer.

In some aspects, the swelling ratio can be measured as follows. A polymer matrix comprising PVDF and functional polymer particles is dissolved the PVDF and the PVDF and the functional polymer particles recovered using hot solvent. The PVDF can be allowed to phase separate by gradually cooling the solution to allow the PVDF to precipitate. The supernatant can be collected; dried to determine the dry mass of the recovered microgels (this need not be all of the microgels) and water (or the appropriate solvent in which the membrane is used) can be added gradually until the gel particles no longer absorb all of it. Then measure the wet mass. Use the densities of the solvent and the dry polymer to evaluate the ratio of the volume of the swollen state over the volume of the dry state.

In some aspects, the composite membrane is used under operating conditions in which the working fluid does not chemically degrade or solubilize the structural polymer, the macroporous scaffold, the functional polymer particle (e.g., functional gel), or all three, but which swells the functional polymer particle (e.g., functional gel) (such as water for aqueous systems). “Does not chemically degrade or solubilize” as used herein generally allows for a degree of chemical degradation and/or solubilization that does not detrimentally affect the structure and/or function of the composite membrane during its normal working life.

In some aspects, disclosed is a composite, comprising:
a macroporous scaffold comprising pores; and
a polymer matrix positioned within the pores;
wherein the polymer matrix comprises:
   a functional polymer particle; and
   a structural polymer.

In some aspects, the functional polymer particle comprises a functional gel.

In some aspects, the functional polymer particle comprises at least one primary amine, at least one primary ammonium, at least one secondary amine, at least one secondary ammonium, at least one tertiary amine, at least one tertiary ammonium, or any combination thereof.

In some aspects, the functional polymer particle is in a form of a plurality of particles, wherein the plurality of particles has an average particle size of 100 nm to 10 μm when measured by scanning electron microscopy (SEM) in a dry state. For example, in some aspects, the average particle size measured in a dry state by SEM is 100 nm to 10 μm, 200 nm to 9 μm, 300 nm to 8 μm, 500 nm to 6 μm, 800 nm to 5 μm, 500 nm to 3 μm, 1 μm to 3 μm, or 1 μm to 6 μm.

In some aspects, the functional polymer particle is in a form of a plurality of particles, wherein the plurality of particles has an average particle size of 0.2 to 20 μm when measured in a wet state, optionally wherein the plurality of particles is in a swollen state in the wet state. For example, in some aspects, the average particle size measured in a wet state is 0.2 μm to 20 μm, 0.5 μm to 18 μm, 0.5 μm to 15 μm, 1 μm to 10 μm, 3 μm to 8 μm, 0.5 μm to 0.8 μm, or 5 μm to 15 μm.

In some aspects, the pores comprise through-pores; the functional polymer particle is in a form of a plurality of particles; and the plurality of particles has an average particle size that is from 0.01 D to 0.2 D when measured in a wet state, wherein D is an average diameter of the through-pores, and optionally the plurality of particles is in a swollen state in the wet state. For example, in some aspects, the average particle size in a set state is 0.01 D to 0.2 D, 0.05 D to 1.5 D, 0.1 D to 0.2 D, 0.04 D to 0.08 D, 0.09 D to 0.15 D, or 0.1 D to 0.15 D.

In some aspects, the functional polymer particle comprises a functional gel comprising a hydrogel.

In some aspects, the functional polymer particle comprises polyethylenimine (PEI), branched PEI, hyperbranched PEI, poly(ethylene oxide) (PEO), poly-N-isopropylacrylamide, polyamidoamine dendrimers (PAMAM), low generation PAMAM, chitosan, gelatin, a biopolymer, a functional biopolymer, carrageenan, or any combination thereof. Any other functional polymer particle described elsewhere herein may also be employed.

In some aspects, the functional polymer particle comprises structure (1):

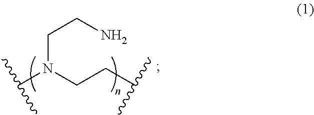

or a salt thereof;
structure (11):

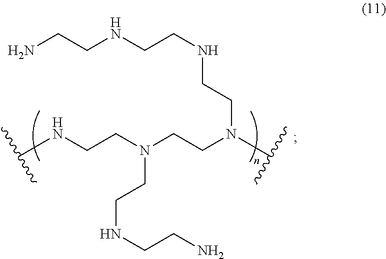

or a salt thereof,
or a combination of structure (1) or a salt thereof, and structure (11) or a salt thereof; wherein each n independently is an integer from 10 to 10,000.

In some aspects, the functional polymer particle comprises a functional gel, such as PEI, with a weight-average molecular weight (M_{w}) of 300 to 1500 g/mol. For example, in some aspects, the M_{w} of the functional gel, such as PEI, is 300 to 1500 g/mol, 300 g/mol, 600 g/mol, 1200 g/mol, 300 g/mol to 1200, 300 g/mol to 600 g/mol, 600 g/mol to 1200 g/mol, 300 g/mol to 900 g/mol, or 900 g/mol to 1500 g/mol.

In some aspects, the functional polymer particle comprises at least one functional group comprising a carboxylic acid, an acrylate, an alkyl acrylate, a methacrylate, an alkylhalide, a silane, an azide, an alkene, an alkyne, a thiol, a primary amine, a secondary amine, a tertiary amine, pyridine, bipyridine, terpyridine, an amide, an epoxide, a sulfonate, an isocyanate, an anhydride, a methyl ester, an ethyl ester, a propyl ester, a butyl ester, a hydroxyl, or any combination thereof.

In some aspects, the at least one functional group is capable of binding to a species of interest selected from a macromolecule, a peptide, a protein, a glycoprotein, barium, zinc, boron, chromium, iron, selenium, arsenic, nickel, lead, platinum, or any combination thereof. In some aspects, the species of interest is a metal, such as barium, zinc, boron, chromium, iron, selenium, arsenic, nickel, or lead, and such metal is toxic. As a result, in some aspects, the composite membrane produced with such functional groups capable of binding toxic metals can be used to purify and/or detoxify water from toxic metals. In some aspects, the at least one functional group comprises a secondary amine and the species of interest comprises platinum.

In some aspects, the functional polymer particle comprises a functional gel having a swelling ratio of 2 to 20 when immersed in a working fluid. For example, in some aspects the swelling ratio is 2 to 20, 2 to 18, 4 to 18, 4 to 16, 5 to 20, 5 to 15, 5 to 10, 8 to 20, 8 to 15, 8 to 12, 10 to 20, 10 to 15, 12 to 20, 12 to 16, 15 to 20, or 15 to 18. In some aspects, the working fluid is or comprises water. In some aspects, the working fluid is any fluid employed during the operation of the composite membrane, e.g., during chromatography and/or catalysis. In some aspects, the working fluid comprises an organic solvent, such as an alcohol (e.g., methanol, ethanol, or a combination thereof), or a halogenated solvent, such a dicholoromethane.

In some aspects, the functional polymer particle comprises a plurality of particles having an average diameter in a dry state of 0.3 μm to 3 μm, and optionally such functional polymer particles are employed in a scaffold having pores (e.g., directional pores, such as directional through-pores) with an average diameter of 30 μm to 60 μm. For example, in some aspects, the average diameter of the plurality of particles in a dry state is 0.3 μm to 3 μm, 0.3 μm to 2.5 μm, 0.5 μm to 2.5 μm, 0.5 μm to 2 μm, 0.8 μm to 3 μm, 0.8 μm to 2.5 μm, 0.8 μm to 2 μm, 0.8 μm to 1.5 μm, 1 μm to 3 μm, 1 μm to 2.5 μm, 1 μm to 2 μm, 1.5 μm to 3 μm, or 2 μm to 3 μm. Alternatively, or additionally, in some aspects the scaffold has pores (e.g., directional pores, such as directional through-pores) with an average diameter of 20 μm to 100 μm, 30 μm to 60 μm, 20 μm to 80 μm, 20 μm to 60 μm, 30 μm to 100 μm, 30 μm to 80 μm, 50 μm to 100 μm, 30 μm to 55 μm, 30 μm to 50 μm, 30 μm to 45 μm, 30 μm to 40 μm, 35 μm to 60 μm, 35 μm to 50 μm, 35 μm to 45 μm, 40 μm to 60 μm, 40 μm to 55 μm, 40 μm to 50 μm, 45 μm to 60 μm, or 45 μm to 55 μm. Any combination of the dry state average particle sizes and the scaffold pore sizes is specifically contemplated.

In some aspects, the functional polymer particle comprises a number average molecular weight (Mₙ) of 1×10³ g/mol to 1×10¹⁰ g/mol, and/or a ratio M_{w}/Mₙ of weight average molecular weight (M_{w}) to number average molecular weight (Mₙ) of 2 to 20. For example, in some aspects the Mₙ can be 1×10³ g/mol to 1×10¹⁰ g/mol, 1×10⁴ g/mol to 1×10¹⁰ g/mol, 1×10⁵ g/mol to 1×10¹⁰ g/mol, 1×10⁸ g/mol to 1×10¹⁰ g/mol, 1×10³ g/mol to 1×10⁸ g/mol, 1×10³ g/mol to 1×10⁶ g/mol, or 1×10⁵ g/mol to 1×10⁸ g/mol. Alternatively, or additionally, the M_{w}/Mₙ can be 2 to 20, 2 to 15, 2 to 10, 2 to 8, 2 to 5, 2 to 3, 3 to 5, 5 to 8, 5 to 20, 5 to 15, 5 to 10, 10 to 20, or 10 to 15. Any combination of the Mₙ and the M_{w}/Mₙ is specifically contemplated.

In some aspects, the functional polymer particle comprises G0 PAMAM, G1 PAMAM, or a combination thereof. In some aspects, wherein the G0 PAMAM has 4 primary amines and/or a molar mass of 517 g/mol. In some aspects, the G1 PAMAM has 8 primary amines and/or a molar mass of 1430 g/mol. In some aspects, the functional polymer particle comprises a G0 PAMAM, G1 PAMAM, G2 PAMAM, G3 PAMAM, or any combination thereof.

In some aspects, the functional polymer particle is crosslinked. In some aspects, the functional polymer particle is crosslinked with a crosslinker comprising at least one primary amine, at least one primary ammonium, at least one secondary amine, at least one secondary ammonium, at least one tertiary amine, at least one tertiary ammonium, or any combination thereof.

In some aspects, the functional polymer particle is crosslinked and has at least one crosslinked structure comprising formula (2), (3), (4), (5), (6), or any combination thereof:

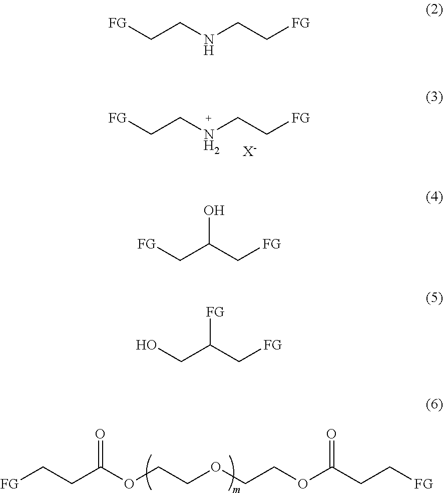

wherein FG is the functional polymer particle, X is a counterion, and m is an integer from 0 to 20. X is not particularly limited and can be any suitable counterion, such as a halide (e.g., chloride, bromide, or iodide), though any negatively charged species can serve as a counterion, including tosylate (OTs), mesylate (OMs), triflate (OTf), 2,2,2-trifluoroethanesulfonate, alkylsulfonate, benzenesulfonate, substituted benzenesulfonate, sulfate, nitrate, or phosphate. In some aspects, m is an integer from 0 to 20, 0, 1, 2, 3, 4, 5, 6, 1 to 10, 1 to 8, 1 to 5, 3 to 10, 10 to 20, 15 to 20, or 1 to 3.

In some aspects, the functional polymer particle is crosslinked from a crosslinker comprising:

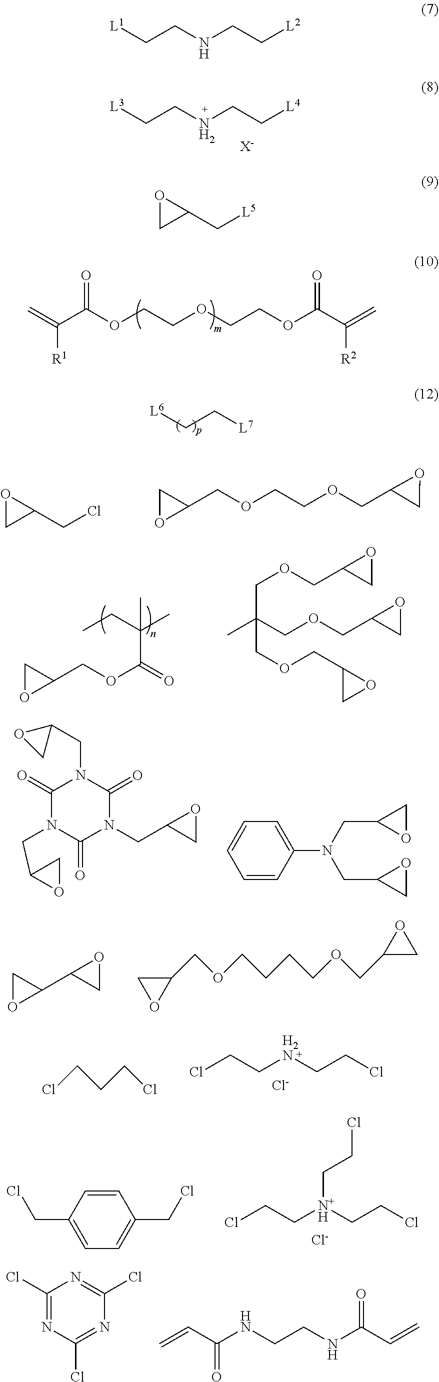

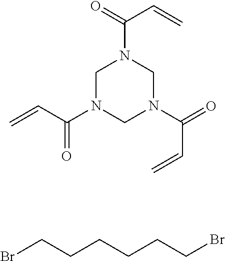 [SMILES: C=CC(=O)N1CN(C(=O)C=C)CN(C(=O)C=C)C1 ; BrCCCCCCBr]

or any combination thereof, wherein each of L¹, L², L³, L⁴, L⁵, L⁶, and L⁷, is a leaving group optionally selected from a halide, tosylate (OTs), mesylate (OMs), triflate (OTf), 2,2,2-trifluoroethanesulfonate, alkylsulfonate, benzenesulfonate, substituted benzenesulfonate, or phosphate; X is a counterion optionally selected from chloride, bromide, or iodide; each of R¹ and R² independently is hydrogen or C₁-C₆ alkyl; n is an integer from 2 to 50; m is an integer from 0 to 20; and p is an integer from 1 to 9, 4 to 6, or 5. The counterion can be any suitable counterion disclosed herein. The C₁-C₆ can be any suitable C₁-C₆ alkyl, such as methyl, ethyl, propyl, butyl, pentyl, or hexyl, including any straight or branched versions thereof. The n is any suitable integer, including 2 to 50, 2 to 10, 2 to 8, 2 to 6, 3 to 5, 5 to 10, 10 to 20, 20 to 30, 30 to 40, or 40 to 50. The m can be any suitable integer, including 0 to 20, 0, 0 to 15, 0 to 10, 0 to 5, 1 to 3, 1 to 5, 3 to 5, 5 to 10, 10 to 15, or 15 to 20. The p is any suitable integer, including 1 to 9, 4 to 6, 5, 2 to 7, 2 to 5, 3 to 6, 3 to 9, or 5 to 9.

In some aspects, the functional polymer particle is crosslinked using a crosslinker selected from bis(2-chloroethyl)amine hydrochloride (BCAH), (2-cloroethyl)(3-chloropropyl)amine, 2-chloro-N-(2-chloroethyl)-1-propanamine hydrochloride, N,N′-Bis(2-chloroethyl)ethane-1,2-diamine, epichlorohydrin (ECH), diethylene glycol diacrylate (EGA), low molecular weight polyethylene glycol diacrylate, bis(2-chloroethyl)ether, 1,4-butanediol diglycidyl ether, or any combination thereof.

In some aspects, the functional polymer particle comprises a normalized crosslinking density (NCD) of 0.01 to 0.8, such as 0.01 to 0.7, 0.01 to 0.6, 0.01 to 0.5, 0.01 to 0.4, 0.01, to 0.2, 0.05 to 0.8, 0.05 to 0.6, 0.05 to 0.3, 0.1 to 0.8, 0.1 to 0.6, 0.1 to 0.3, 0.2 to 0.8, 0.2 to 0.5, or 0.4 to 0.8.

In some aspects, the functional polymer particle comprises a crosslink density of 0.005 to 0.6, such as 0.006 to 0.6, 0.01 to 0.6, 0.01 to 0.6, 0.01 to 0.55, 0.01 to 0.5, 0.01 to 0.4, 0.01, to 0.2, 0.05 to 0.6, 0.05 to 0.4, 0.05 to 0.3, 0.1 to 0.6, 0.1 to 0.5, 0.1 to 0.3, 0.2 to 0.6, 0.2 to 0.5, or 0.4 to 0.6.

In some aspects, the functional polymer particle and/or structural polymer is covalently attached directly or indirectly to a surface of the pores. By way of example, a direct attachment is where the functional polymer particle and/or structural polymer is covalently attached to a functional group on the surface of the pores, and an indirect attachment is where another species, molecule, or polymer mediates the attachment (e.g., the functional polymer particle and/or structural polymer is directly attached to this other species, molecule, or polymer, and then this other species, molecule or polymer is directly attached to the functional group on the surface of the pores). Of course, it is contemplated that multiple other species, molecule, or polymers can mediate the bonding between the functional polymer particle and/or structural polymer and the functional group on the surface of the pores.

In some aspects, the functional polymer particle and/or structural polymer is covalently attached indirectly to the surface of the pores via an oligomer or polymer, wherein the oligomer or polymer comprises at least one primary amine, at least one primary ammonium, at least one secondary amine, at least one secondary ammonium, at least one tertiary amine, at least one tertiary ammonium, or any combination thereof.

In some aspects, the functional polymer particle and/or structural polymer is covalently attached indirectly to the surface of the pores via the polymer, and the polymer comprises PEI, amine-functionalized or -terminated polymer, amine-functionalized or -terminated polyethylene glycol (PEG), acrylate-functionalized or -terminated polymer, acrylate-functionalized or -terminated PEG, epoxide-functionalized or -terminated polymer, epoxide-functionalized or -terminated PEG, or any combination thereof.

In some aspects, the functional polymer particle and/or structural polymer is attached indirectly to the surface of the pores via at least one crosslinker, and the crosslinker can be any crosslinker (or combination of crosslinkers) disclosed elsewhere herein.

In some aspects, the functional polymer particle and/or structural polymer is indirectly attached to the surface of the pores; the functional polymer particle and/or structural polymer is crosslinked to PEI (or another polymer that comprises the functional polymer particle); and the PEI (or another polymer that comprises the functional polymer particle) is crosslinked to a functional group on the surface of the pores.

In some aspects, the functional polymer particle comprises a metal-organic framework (MOF), a covalent organic framework (COF), a nanoporous polymer, a functional gel, or any combination thereof. In some aspects, the functional polymer particle comprises a metal-organic framework (MOF), a covalent organic framework (COF), a nanoporous polymer, or any combination thereof, and the polymer matrix further comprises a functional gel.

In some aspects, the macroporous scaffold comprises ceramic, organic glass, inorganic glass, carbon, charcoal, graphene, graphite, metal, fused metal particles, polymer, crystalline polymer, semicrystalline polymer, fused polymer particles, other dispersed species, or any combination thereof. In some aspects, the macroporous scaffold comprises the ceramic or inorganic glass, and the ceramic or inorganic glass comprises silicon oxycarbide. In some aspects, the macroporous scaffold comprises a freeze-cast material, such as a ceramic (e.g., silicon oxycarbide).

In some aspects, a surface of the pores are functionalized with a functional group capable of reacting directly with a functional group on the functional polymer particle and/or structural polymer, indirectly via a crosslinker, or a combination thereof. Suitable functional groups include, for example, a carboxylic acid, an acrylate, an alkyl acrylate, a methacrylate, an alkylhalide, a silane, an azide, an alkene, an alkyne, a thiol, a primary amine, a secondary amine, a tertiary amine, pyridine, bipyridine, terpyridine, an amide, an epoxide, a sulfonate, an isocyanate, an anhydride, a methyl ester, an ethyl ester, a propyl ester, a butyl ester, a hydroxyl, or any combination thereof.

In some aspects, the macroporous scaffold comprises a pore volume fraction of 10% to 70% of the composite, such as 10 to 60%, 10 to 40%, 10 to 20%, 20 to 70%, 20 to 50%, 20 to 35%, 30 to 70%, 30 to 60%, 30 to 40%, 40 to 70%, 40 to 55%, or 50 to 70%.

In some aspects, the pores have size that spans 500 nm to 500 μm. In some aspects, the average pore size is 20 μm to 200 μm, 20 μm to 150 μm, 20 μm to 100 μm, 20 μm to 75 μm, 20 μm to 40 μm, 50 μm to 200 μm, 50 μm to 150 μm, 50 μm to 100 μm, 50 μm to 75 μm, 80 μm to 200 μm, 80 μm to 150 μm, 80 μm to 120 μm, 100 μm to 200 μm, 100 μm to 150 μm, or 150 μm to 200 μm.

In some aspects, the pores comprise a morphology comprising a cellular, dendritic, lamellar, or prismatic structure, or any combination thereof.

In some aspects, the pores of the scaffold are oriented along a primary axis. In some aspects, the pores of the scaffold have directionality.

In some aspects, the functional polymer particle comprises a functional gel, and/or the structural polymer is insoluble or slightly soluble (e.g., very slightly soluble) in a solvent capable of swelling the functional gel, optionally wherein the solvent comprises water.

In some aspects, the structural polymer comprises polyvinylidene fluoride (PVDF), cellulose acetate, polysulfone, polyvinyl chloride, poly(acrylonitrile), polyethersulfone (PES), polypropylene, polytetrafluoroethylene, polyamide imide, natural rubber, or any combination thereof.

In some aspects, the structural polymer is not covalently attached to the functional polymer particle. In some aspects, the structural polymer is covalently attached to the functional polymer particle.

In some aspects, the structural polymer is present in an amount of 20 wt. % to 80 wt. %, based on total mass of structural polymer and functional polymer particle, excluding solvent, if present; or the functional polymer particle is present in an amount of 20 wt. % to 80 wt. %, based on total mass of structural polymer and functional polymer particle, excluding solvent, if present. For example, the amount of structural polymer, based on total mass of structural polymer and functional polymer particle, excluding solvent if present, can be 20-80 wt. %, 20-70 wt. %, 20-60 wt. %, 20-50 wt. %, 20-40 wt. %, 20-30 wt. %, 30-80 wt. %, 30-70 wt. %, 30-60 wt. %, 30-50 wt. %, 30-40 wt. %, 40-80 wt. %, 40-70 wt. %, 40-60 wt. %, 40-50 wt. %, 50-80 wt. %, 50-70 wt. %, 50-60 wt. %, 60-80 wt. %, 60-70 wt. %, or 70-80 wt. %. Such amounts can also be used to calculate the amount of functional polymer particle based on total amount of structural polymer and functional polymer particle, excluding solvent if present, based on a straightforward calculation (wt. % of functional polymer particle=100%-wt. % structural polymer).

In some aspects, the functional polymer particle is present in an amount of 20 wt. % to 50 wt. % (or any other amount disclosed herein), based on total mass of structural polymer and functional polymer particle, excluding solvent, if present; and the functional polymer particle has an NCD of 0.3 to 0.8 (or any other NCD amount disclosed herein).

In some aspects, at least one metal chelated to the polymer matrix. IN some aspects, the metal comprises a transition metal optionally selected from copper, palladium, platinum, iron, rhodium, ruthenium, or any combination thereof. In some aspects, a composite membrane comprising such a metal-chelated polymer matrix can be used in various applications, such as catalysis, chromatography, sensing, and the like.

In some aspects, the composite further comprises a second composite, comprising:
a second macroporous scaffold comprising pores; and
a second polymer matrix positioned within the pores;
wherein the second polymer matrix comprises:
a second functional polymer particle; and
a second structural polymer;
wherein the pores of the second macroporous scaffold are fluidically connected to the pores of the macroporous scaffold; and
wherein each of the second composite, the second macroporous scaffold, the second polymer matrix, the second functional polymer particle, and the second structural polymer independently are the same or different from each of the composite, the macroporous scaffold, the polymer matrix, the functional polymer particle, and the structural polymer, respectively. In other words, the disclosures elsewhere herein relating to the composite, the macroporous scaffold, the polymer matrix, the functional polymer particle, and the structural polymer are applicable to the corresponding feature in the second composite. In addition, a third composite, a fourth composite, a fifth composite, and so forth may also be used in combination with the composite and the second composite so as to form a stack. Such a stack can be used, for example, to have multiple applications, such as a composite having a chromatographic functionality, and a second composite having a catalytic functionality, so as to have multiple applications performed in serial (or even in parallel).

In some aspects, disclosed is a composite comprising:
a macroporous scaffold comprising pores; and
a polymer matrix positioned within the pores;
wherein the polymer matrix comprises:
   a functional gel; and
   a structural polymer
wherein each of the macroporous scaffold, polymer matrix, pores, functional gel, and structural polymer are as defined elsewhere herein.

Method of making: In some aspects, the method of making comprises at least the following steps: prepare a liquid that includes functional polymer particle (e.g., functional gel) and dissolved structural polymer; infiltrate a macroporous scaffold with the liquid; and perform nonsolvent induced phase separation (NIPS) on the macroporous scaffold filled with liquid. In some aspects, additional steps may be incorporated to the procedure including, but not limited to: bonding the functional polymer particle (e.g., functional gel) to the surface of the macroporous scaffold, altering the functionality of the functional polymer particle (e.g., functional gel), treating the final composite with salt solutions and reducing agents, a combination thereof, or any combination thereof. In some aspects, temperature-induced phase separation (TIPS) can be employed instead or, or in addition to, NIPS, in which the polymer dope solution infused scaffold is placed in a low temperature chamber to perform TIPS. The example procedures provided in the Examples section below (see, e.g., all Examples, including Examples 1-4) are purely illustrative and is not meant to restrict the scope of the invention.

In some aspects, a procedure that does not include the three steps noted above (i.e., prepare a liquid, infiltrate the scaffold, perform NIPS) may be unsuccessful in producing a suitable composition of matter within the pores of the macroporous scaffold, and thus may be unsuccessful in producing a functional composite membrane suitable for use in certain applications, such as chromatography and/or catalysis. Several examples which have been tested and shown to fail include: Infusing the macroporous scaffold with uncrosslinked PEI followed by PVDF; infusing the macroporous scaffold with separately polymerized PEI mixed with PVDF; infusing the macroporous scaffold with a composition of matter disclosed herein, but allowing it to dry instead of performing NIPS.

In some aspects, a liquid mixture is employed that comprises a solvent selected so that it dissolves the selected structural polymer and a precursor of the functional polymer particle (e.g., functional gel); after the solution of the structural polymer is prepared, precursors of the functional polymer particle (e.g., functional gel) are added; then the synthesis of the functional polymer particle (e.g., functional gel) (prior to infiltration) is initiated thereby forming a “polymer dope solution”; then this liquid is infiltrated into the pores (the synthesis reaction that produces the functional polymer particle (e.g., functional gel) may continue during and after infiltration); in some aspects the infiltrated composition includes chemical species that covalently anchor some of the functional polymer particle (e.g., functional gel) and/or structural polymer to functional groups on the pore walls; in some aspects, it is desirable that at least some of the polymer matrix form covalent bonds to the wall to avoid the polymer matrix from sloughing or detaching from the pore walls. As used herein, “a precursor” can mean a single precursor or multiple precursors, each of which can be the same or different.

In some aspects, the method of making comprises preparing the macroporous scaffold. The macroporous scaffold can be prepared by any suitable method, as described elsewhere herein. Generally in the method of making herein, disclosures of “pores” generally are referring to pores in the scaffold and not pores in a polymer membrane, unless otherwise clearly contradicted by context.

In some aspects, disclosed is a method for making a composite, the method comprising: infiltrating the pores with a liquid comprising the polymer matrix or a precursor thereof; and performing nonsolvent induced phase separation (NIPS) on the macroporous scaffold infiltrated with the liquid.

In some aspects, the NIPS comprises a nonsolvent comprising: an alcohol having 1 to 8 carbon atoms optionally selected from methanol, ethanol, isopropyl alcohol, n-propanol, n-butanol, n-pentanol, n-hexanol, or mixtures thereof with water, or any combination thereof; or 20-80 vol. % in water of a solvent of the structural polymer optionally selected from triethyl phosphate (TEP), trimethyl phosphate (TMP), DMSO, DMF, acetone, n-methylpyrrolidone (NMP), N,N-dimethylacetamide (DMA), hexamethylphosphoramide (HMPA), or any combination thereof; or a combination thereof. The mixture of a structural polymer solvent in water can be (vol. % solvent) 20-80, 20-60, 20-40, 30-80, 30-60, 40-80, or 40-60.

In some aspects, the method further comprises, after the infiltrating step but before the performing step: incubating the macroporous scaffold infiltrated with the liquid under conditions sufficient (a) to promote crosslinking of the precursor to produce the polymer matrix, (b) to promote crosslinking within the functional polymer particle and/or with the structural polymer, (c) to promote reaction and/or crosslinking between a surface of the pores and (i) the structural polymer, (ii) the functional polymer particle, (iii) the polymer matrix, and/or (iv) any precursor thereof, or (d) any combination thereof.

In some aspects, the method further comprises preparing the polymer matrix by crosslinking a functional polymer particle precursor in the presence of the structural polymer. Suitable crosslinkers include any crosslinker disclosed elsewhere herein.

In some aspects, the method further comprises, prior to the infiltrating step: if a surface of the pores does not already contain a functional group capable of reacting with a crosslinker, functional polymer particle, precursor of functional polymer particle, and/or structural polymer; then functionalizing the surface of the pores with the functional group capable of reacting directly or indirectly with the functional polymer particle, a precursor of the functional polymer particle, the structural polymer, or any combination thereof.

In some aspects, the method further comprises, after the functionalizing step but prior to the infiltrating step: immersing the macroporous scaffold in a solution comprising an oligomer or polymer comprising at least one primary amine, at least one primary ammonium, at least one secondary amine, at least one secondary ammonium, at least one tertiary amine, at least one tertiary ammonium, or any combination thereof; and incubating the macroporous scaffold containing the solution under conditions sufficient to react the oligomer or polymer to the functional group on the surface of the pores; optionally wherein the functional group is covalently bonded to the oligomer or polymer via a crosslinker.

In some aspects, the macroporous scaffold comprises a ceramic, and the method further comprises: preparing the macroporous scaffold by a process comprising freeze casting from a solution of preceramic polymer and a crosslinking agent; or electrochemical synthesis.

In some aspects, disclosed is a method for making a composite, the method comprising: (A) infiltrating the pores with a liquid comprising (1) the structural polymer or a precursor thereof, (2) the functional polymer particle or a precursor thereof, or (3) a combination of the structural polymer or a precursor thereof and the functional polymer particle or a precursor thereof; and (B) performing nonsolvent induced phase separation (NIPS) on the macroporous scaffold infiltrated with the liquid.

In some aspects, disclosed is a method for making a composite, the method comprising: (A) infiltrating the pores with a liquid comprising (1) the structural polymer or a precursor thereof; (B) performing nonsolvent induced phase separation (NIPS) on the macroporous scaffold infiltrated with the liquid; (C) infiltrating the pores with the functional polymer particle or a precursor thereof; and (D) incubating the structure so as to crosslink or achieve another reaction so as to prepare the composite.

In some aspects, disclosed is a method for making a composite, the method comprising: infiltrating the pores of a scaffold with a liquid comprising the structural polymer or a precursor thereof (e.g., polymerizing and/or grafting if desired); performing nonsolvent induced phase separation (NIPS) on the macroporous scaffold infiltrated with the liquid; and submerging in a solution of MOF precursors and with a high pH (e.g., 8-14, 10 to 14, 12 to 14, or 10 to 12) to facilitate nucleation and growth of MOFs. In some aspects, the in situ formation of the MOFs within the pores can produce some particles that encircle the structural polymer fibers.

Method of use: In some aspects, the functional composite membranes disclosed herein may be used, for example, in ion exchange chromatography, affinity chromatography, catalysis, a combination thereof, or any combination thereof. Previous work has demonstrated that using a polymer/hydrogel matrix similar to that described herein, but not provided within a composite membrane structure, provides the capability to chelate metal ions such as Cu and Pt, with the chelated Pt being used for catalysis [15-16]. Copper is a common metal used in immobilized metal affinity chromatography (IMAC) [14]. It is expected that in some aspects, the composite will contain chelated metal ions or atoms and will be used for IMAC or catalysis. In the aspect described below in Description & Figures, the composite membrane is used as a weak anion exchange membrane for the capture of bovine serum albumin (a model protein for antibody purification processes). In another aspect, the functionality of the functional polymer particle (e.g., functional gel) would be altered to enable its use as a weak cation exchange membrane.

In some aspects, the functional group(s) of the functional polymer particles disclosed herein can bind to any suitable species of interest. Such species of interest include, for example, salt (e.g., for water reuse and desalination), a gas (e.g., for gas separations, such as natural gas purification), metal oxide nanoparticles, metallic and bimetallic nanoparticles, MOFs, COFs, carbon nanotubes, graphene, or any combination thereof. In some aspects, secondary amines as a functional group on the functional polymer particle offer different binding characteristics from primary or tertiary amines in relation to chelating metal ions. For example, secondary amine groups (˜1.9 mequiv per gram of dry membrane), are more basic and thus have higher Pt binding affinity than the tertiary amine groups. See, for example, “A Facile and Scalable Route to the Preparation of Catalytic Membranes with in Situ Synthesized Supramolecular Dendrimer Particle Hosts for Pt(0) Nanoparticles Using a Low-Generation PAMAM Dendrimer (G1-NH2) as Precursor” ACS Appl. Mater. Interfaces 2018, 10, 33238-33251 (DOI: 10.1021/acsami.8b11351), hereby incorporated by reference in its entirety for all purposes.

In some aspects, a composite disclosed herein is useful for a variety of applications, including where components contained in a liquid, gas, or supercritical fluid are passed through or into the composite, an such applications include chromatography, catalysis, sensing, gas storage, medicine, and so forth.

In some aspects, disclosed is a method for separating a component from a first mixture, the method comprising passing the first mixture containing the component through a composite (e.g., the composite disclosed elsewhere herein); and isolating the component from the first mixture. In some aspects, this method for separating is functionally equivalent to ion exchange chromatography, affinity chromatography, or a combination thereof, such that the method is a method for ion exchange chromatography, affinity chromatography, or a combination thereof. In some aspects, the functional polymer particle contains a functional group capable of binding to a species of interest, and such capability enables the separation to occur.

In some aspects, disclosed is a method for catalyzing a chemical reaction in a second mixture, the method comprising passing the second mixture through a composite (e.g., the composite disclosed elsewhere herein); wherein the chemical reaction is catalyzed by the composite. In some aspects, the functional polymer particle is chelated to a metal or other group capable of catalyzing a chemical reaction, and such metal or other group enables such catalytic activity.

In some aspects, the first mixture and/or the second mixture comprises a salt concentration of 0 to 500 mM, 0 to 400 mM, 0 to 250 mM, 0 to 150 mM, 50 to 500 mM, 50 to 250 mM, 50 to 150 mM, 100 to 500 mM, 100 to 250 mM, or 150 to 250 mM. The salt can be any salt, such as sodium chloride, potassium chloride, sodium sulfate, and so forth, or any combination thereof. Alternatively, or in addition, the first mixture and/or second mixture comprises a conductivity of 0 to 50 mS/cm, 0 to 40 mS/cm, 0 to 20 mS/cm, 0 to 25 mS/cm, 5 to 25 mS/cm, 5 to 10 mS/cm, 10 to 50 mS/cm, 10 to 30 mS/cm, 20 to 50 mS, 10 to 20 mS/cm, 20 to 30 mS/cm, 30 to 40 mS/cm, or 40 to 50 mS/cm. Any combination of salt concentration and conductivity for the first and/or second mixture is specifically contemplated herein.

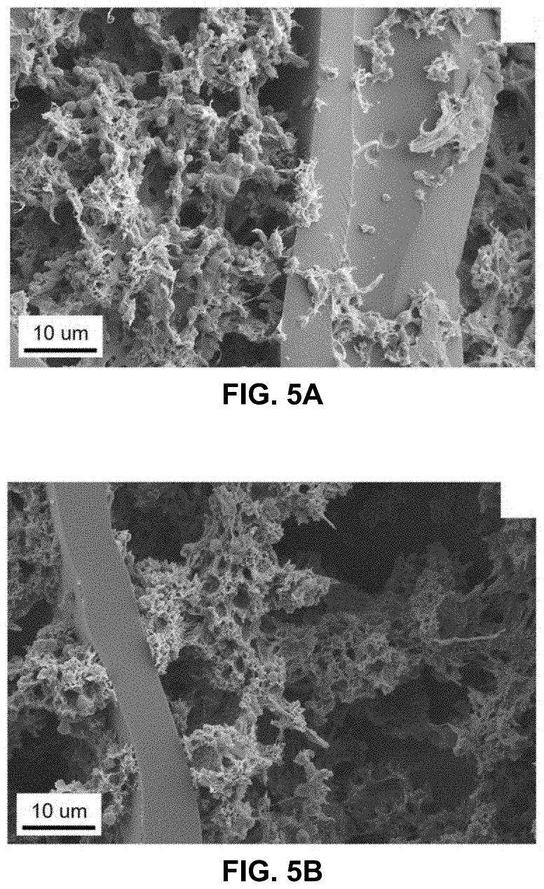

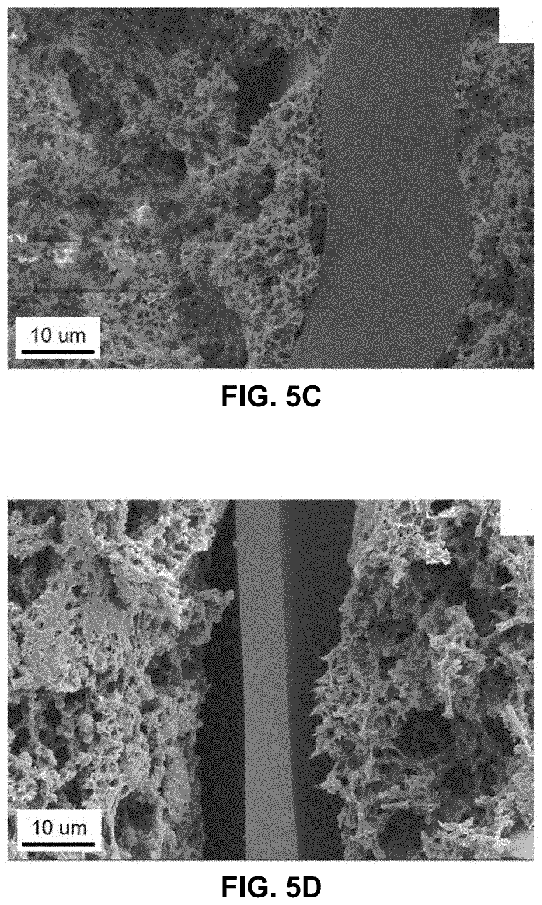

In some aspects, the compositions, composite membranes, methods of making, and methods of use disclosed herein (1) are distinguishable from what is known in the art, and (2) are associated with unexpected and surprising features. For example, a person of ordinary skill in the art would advise against reducing crosslink density from that reported for NSM-2 formulation in reference 17 of Examples 1-6 (Kotte, J. Mem. Sci. 2014): formation of the PEI particles would be expected to suffer and, consequently, the binding capacity would be expected to decrease. The ordinarily skilled person may be correct in the case of an all-polymer membrane: all of the concentrations examined that are less than 0.5×[crosslink density of NSM-2] show low capacity in the all-polymer membrane and SEM shows that the PEI particle formation did suffer (FIGS. 5A-5D). However, the ordinarily skilled person would not predict what is observed in the hybrid membrane: SEM confirms that PEI particle formation is disrupted in the composites formulated by the procedure in Sections 2.1 to 2.5 of Examples 1-4, but the binding capacity remains in the range that is useful and is much greater than the binding capacity of the all-polymer counterpart.

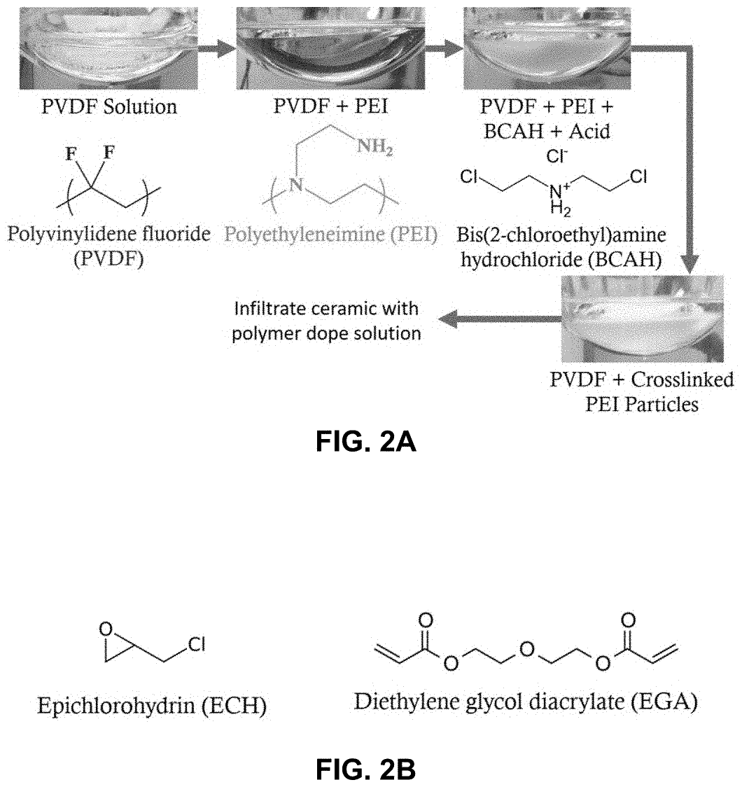

Additionally, the ordinarily skilled person might expect that replacing ECH by EGA (FIGS. 2A-2B) might increase binding by providing a spacer that is longer and is hydrophilic. The magnitude of the effect observed in water is encouraging (compare EGA to ECH). However, the effect of BCAH would not be anticipated: the length of the spacer is only 5 atoms (four C and one N), which represents a small increase relative to ECH, and the increase in the number of N in the structure is only increased by 2/15 (the ratio of N incorporated via BCAH to the N in the PEI). The chemical structures of ECH, EGA and BCAH are shown in FIGS. 2A-2B.

One of ordinary skill in the art would be surprised by the magnitude of the effect (the BCAH-crosslinked material has more than double the binding capacity relative to ECH crosslinker at the same crosslink density). Furthermore, this small increase in N content in the membrane dramatically improves the ability to retain binding in the presence of salt. However, while BCAH has some advantages in some circumstances, in some aspects the other crosslinkers disclosed herein, such as ECH and EGA, may be employed with satisfactory results.

Table 1 provides a comparison of features and differences between the references and some aspects of the compositions, composites, and methods disclosed herein. The reference numbers refer to the references listed for Example 1-6.

TABLE 1

| | | Pore-filling, | |
|---|---|---|---|
| | | hydrogel | Pore-filling |
| | Surface | formation with | with phase |
| | grafting | in-situ crosslinking | inversion |
| Porous Polymer | 10, 11 | 10, 11, 12 | |
| Porous Ceramic | 3, 4, 5, 8 | | |
| Ceramic with a | | 13 | Present |
| plurality of | | | Application |
| directional pores | | | |

As seen in Table 1, macroporous polymers and ceramics have sometimes been used as scaffolds for functional membranes using both surface grafting and pore filling hydrogels. The surface grafting approach affords little functionality, poorly utilizing the volume of the pores and, consequently having poor volumetric binding capacity. Filling pores entirely with hydrogel provides a higher binding capacity at the expense of permeability. There is no known material that provides high binding capacity and high permeability.

In some aspects, an advantage of the compositions, composites, and methods described herein is the formation of a discontinuous plurality of functional polymer particles (e.g., functional gel)—which allows fluid to flow around and between functional polymer particles. In some aspects, the discontinuous plurality of functional polymer particles is stably integrated in the macroporous scaffold by one or more structural polymers. In addition, in some aspects, the method of forming the functional polymer particles (e.g., functional gel) in-situ allows for greater control of the properties of the functional polymer particles. Without wishing to be bound by theory, the following explanation is offered in an attempt to rationalize the observed properties, but this theory should in no way be interpreted as limiting. In some aspects, by controlling the crosslinker concentration, we are in theory able to change the “density” or tightness of the polymer particles (e.g., functional hydrogels) in different regions. So, at high crosslinker concentrations, the PEI microgels for example are tight and as a result more closely resemble resins in their molecular interactions. Whereas at lower crosslinker concentrations we have in theory a more open plurality of functional polymer particles (e.g., functional gel) which may lead to a more uniform functional polymer particle (e.g., functional hydrogel) distribution across the pore. In some aspects, a more even distribution would be better for weak anion exchange membrane applications. In contrast, when crosslinking utilizing the in-situ method outlined in the references of Table 1, there is little to no control over the local hydrogel tightness. Instead the hydrogel precursor solution is well-mixed prior to polymerization and then any differences in hydrogel tightness or “density” are assumed to averaged out.

Various aspects are contemplated herein, several of which are set forth in the paragraphs below. It is explicitly contemplated that any aspect or portion thereof can be combined to form an aspect.

Aspect 1: A composite, comprising:
a macroporous scaffold comprising pores; and
a polymer matrix positioned within the pores;
wherein the polymer matrix comprises:
   a functional polymer particle; and
   a structural polymer.

Aspect 2: The composite of aspect 1, wherein the functional polymer particle comprises a functional gel.

Aspect 3: The composite of any preceding aspect, wherein the functional polymer particle comprises at least one primary amine, at least one primary ammonium, at least one secondary amine, at least one secondary ammonium, at least one tertiary amine, at least one tertiary ammonium, or any combination thereof.

Aspect 4: The composite of any preceding aspect, wherein the functional polymer particle is in a form of a plurality of particles, wherein the plurality of particles has an average particle size of 100 nm to 10 μm when measured by scanning electron microscopy in a dry state.

Aspect 5: The composite of any preceding aspect, wherein the functional polymer particle is in a form of a plurality of particles, wherein the plurality of particles has an average particle size of 0.2 to 20 μm when measured in a wet state, optionally wherein the plurality of particles is in a swollen state in the wet state.

Aspect 6: The composite of any preceding aspect, wherein the pores comprise through-pores; the functional polymer particle is in a form of a plurality of particles; and the plurality of particles has an average particle size that is from 0.01 D to 0.2 D when measured in a wet state, wherein D is an average diameter of the through-pores, and optionally the plurality of particles is in a swollen state in the wet state.

Aspect 7: The composite of any preceding aspect, wherein the functional polymer particle comprises a functional gel comprising a hydrogel.

Aspect 8: The composite of any preceding aspect, wherein the functional polymer particle comprises polyethylenimine (PEI), branched PEI, hyperbranched PEI, poly(ethylene oxide) (PEO), poly-N-isopropylacrylamide, polyamidoamine dendrimers (PAMAM), low generation PAMAM, chitosan, gelatin, a biopolymer, a functional biopolymer, carrageenan, or any combination thereof.

Aspect 9: The composite of any preceding aspect, wherein the functional polymer particle comprises structure (1):

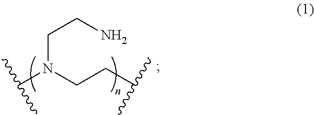

or a salt thereof;
structure (11):

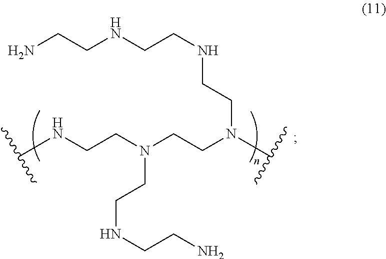

or a salt thereof,
or a combination of structure (1) or a salt thereof, and structure (11) or a salt thereof;
wherein each n independently is an integer from 10 to 10,000.

Aspect 10: The composite of any preceding aspect, wherein the functional polymer particle comprises PEI with a molecular weight of 300 to 1500 g/mol.

Aspect 11: The composite of any preceding aspect, wherein the functional polymer particle comprises at least one functional group comprising a carboxylic acid, an acrylate, an alkyl acrylate, a methacrylate, an alkylhalide, a silane, an azide, an alkene, an alkyne, a thiol, a primary amine, a secondary amine, a tertiary amine, pyridine, bipyridine, terpyridine, an amide, an epoxide, a sulfonate, an isocyanate, an anhydride, a methyl ester, an ethyl ester, a propyl ester, a butyl ester, a hydroxyl, or any combination thereof.

Aspect 12: The composite of aspect 11, or any preceding aspect, wherein the at least one functional group is capable of binding to a species of interest selected from a macromolecule, a peptide, a protein, a glycoprotein, barium, zinc, boron, chromium, iron, selenium, arsenic, nickel, lead, platinum, or any combination thereof.

Aspect 13: The composite of aspect 11 or 12, or any preceding aspect, wherein the at least one functional group comprises a secondary amine and the species of interest comprises platinum.

Aspect 14: The composite of any preceding aspect, wherein the functional polymer particle comprises a functional gel having a swelling ratio of 2 to 20 when immersed in a working fluid, optionally wherein the working fluid comprises water.

Aspect 15: The composite of any preceding aspect, wherein the functional polymer particle comprises a plurality of particles having an average diameter in a dry state of 0.3 μm to 3 μm, optionally wherein the pores have an average diameter of 30 μm to 60 μm.

Aspect 16: The composite of any preceding aspect, wherein the functional polymer particle comprises: a number average molecular weight (Mₙ) of 1×10³ g/mol to 1×10¹⁰ g/mol, and a ratio M_{w}/Mₙ of weight average molecular weight (M_{w}) to number average molecular weight (Mₙ) of 2 to 20.

Aspect 17: The composite of any preceding aspect, wherein the functional polymer particle comprises G0 PAMAM, G1 PAMAM, or a combination thereof; wherein the G0 PAMAM has 4 primary amines and a molar mass of 517 g/mol; and wherein the G1 PAMAM has 8 primary amines and a molar mass of 1430 g/mol.

Aspect 18: The composite of any preceding aspect, wherein the functional polymer particle is crosslinked.

Aspect 19: The composite of any preceding aspect, wherein the functional polymer particle is crosslinked with a crosslinker comprising at least one primary amine, at least one primary ammonium, at least one secondary amine, at least one secondary ammonium, at least one tertiary amine, at least one tertiary ammonium, or any combination thereof.

Aspect 20: The composite of any preceding aspect, wherein the functional polymer particle is crosslinked and has at least one crosslinked structure comprising formula (2), (3), (4), (5), (6), or any combination thereof:

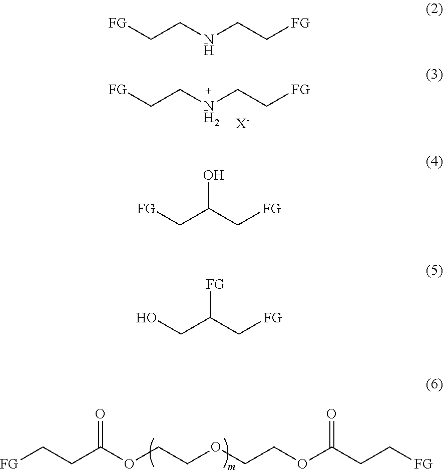

wherein:
FG is the functional polymer particle;
X is a counterion; and
m is an integer from 0 to 20.

Aspect 21: The composite of any preceding aspect, wherein the functional polymer particle is crosslinked from a crosslinker comprising:

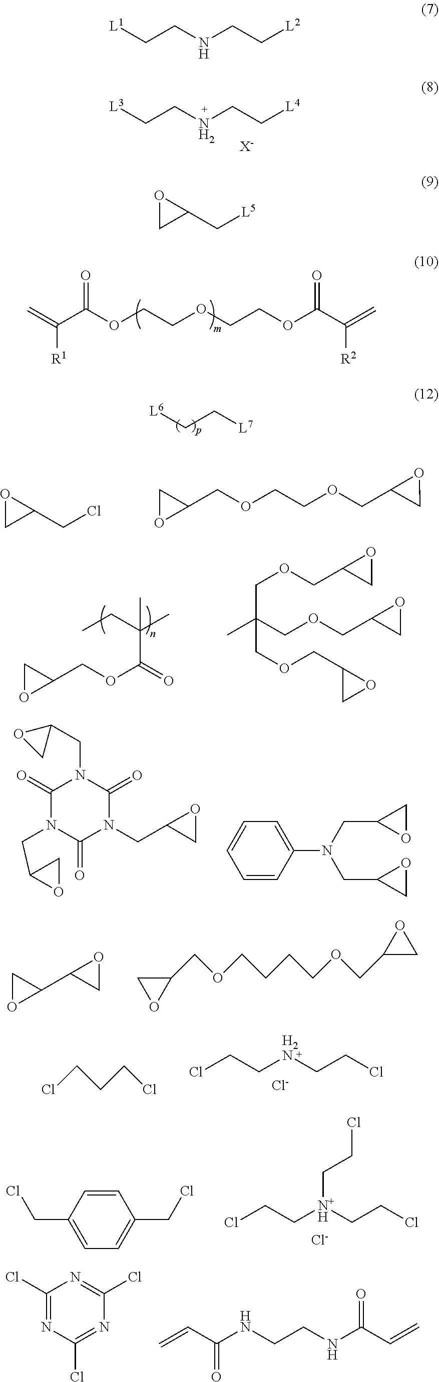

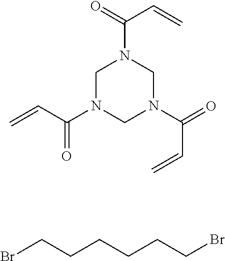 [SMILES: C=CC(=O)N1CN(C(=O)C=C)CN(C(=O)C=C)C1 ; BrCCCCCCBr]

or any combination thereof,
wherein each of L¹, L², L³, L⁴, L⁵, L⁶, and L⁷, is a leaving group optionally selected from a halide, tosylate (OTs), mesylate (OMs), triflate (OTf), 2,2,2-trifluoroethanesulfonate, alkylsulfonate, benzenesulfonate, substituted benzenesulfonate, or phosphate;
X is a counterion optionally selected from chloride, bromide, or iodide; each of R¹ and R² independently is hydrogen or C₁-C₆ alkyl;
n is an integer from 2 to 50;
m is an integer from 0 to 20; and
p is an integer from 1 to 9.

Aspect 22: The composite of any preceding aspect, wherein the functional polymer particle is crosslinked using a crosslinker selected from bis(2-chloroethyl)amine hydrochloride (BCAH), (2-cloroethyl)(3-chloropropyl)amine, 2-chloro-N-(2-chloroethyl)-1-propanamine hydrochloride, N,N′-Bis(2-chloroethyl)ethane-1,2-diamine, epichlorohydrin (ECH), diethylene glycol diacrylate (EGA), low molecular weight polyethylene glycol diacrylate, bis(2-chloroethyl)ether, 1,4-butanediol diglycidyl ether, or any combination thereof.

Aspect 23: The composite of any preceding aspect, wherein the functional polymer particle comprises a normalized crosslinking density (NCD) of 0.01 to 0.8.

Aspect 24: The composite of any preceding aspect, wherein the functional polymer particle comprises a crosslink density of 0.01 to 0.6.

Aspect 25: The composite of any preceding aspect, wherein the functional polymer particle and/or structural polymer is covalently attached directly or indirectly to a surface of the pores.

Aspect 26: The composite of aspect 25, or any preceding aspect, wherein the functional polymer particle and/or structural polymer is covalently attached indirectly to the surface of the pores via an oligomer or polymer, wherein the oligomer or polymer comprises at least one primary amine, at least one primary ammonium, at least one secondary amine, at least one secondary ammonium, at least one tertiary amine, at least one tertiary ammonium, or any combination thereof.

Aspect 27: The composite of aspect 26, or any preceding aspect, wherein the functional polymer particle and/or structural polymer is covalently attached indirectly to the surface of the pores via the polymer, and the polymer comprises PEI, amine-functionalized or -terminated polymer, amine-functionalized or -terminated polyethylene glycol (PEG), acrylate-functionalized or -terminated polymer, acrylate-functionalized or -terminated PEG, epoxide-functionalized or -terminated polymer, epoxide-functionalized or -terminated PEG, or any combination thereof.

Aspect 28: The composite of any one of aspects 25-27, or any preceding aspect, wherein the functional polymer particle and/or structural polymer is attached indirectly to the surface of the pores via at least one crosslinker.

Aspect 29: The composite of aspect 28, or any preceding aspect, the functional polymer particle and/or structural polymer is indirectly attached to the surface of the pores; the functional polymer particle and/or structural polymer is crosslinked to PEI; and the PEI is crosslinked to a functional group on the surface of the pores.

Aspect 30: The composite of any preceding aspect, wherein the functional polymer particle comprises a metal-organic framework (MOF), a covalent organic framework (COF), a nanoporous polymer, a functional gel, or any combination thereof.

Aspect 31: The composite of any preceding aspect, wherein the functional polymer particle comprises a metal-organic framework (MOF), a covalent organic framework (COF), a nanoporous polymer, or any combination thereof; and the polymer matrix further comprises a functional gel.

Aspect 32: The composite of any preceding aspect, wherein the macroporous scaffold comprises ceramic, organic glass, inorganic glass, carbon, charcoal, graphene, graphite, metal, fused metal particles, polymer, crystalline polymer, semicrystalline polymer, fused polymer particles, other dispersed species, or any combination thereof.

Aspect 33: The composite of any preceding aspect, wherein the macroporous scaffold comprises the ceramic or inorganic glass, and the ceramic or inorganic glass comprises silicon oxycarbide.

Aspect 34: The composite of any preceding aspect, wherein the macroporous scaffold comprises a freeze-cast material.

Aspect 35: The composite of any preceding aspect, wherein a surface of the pores are functionalized with a functional group capable of reacting directly with a functional group on the functional polymer particle and/or structural polymer, indirectly via a crosslinker, or a combination thereof.

Aspect 36: The composite of any preceding aspect, wherein the macroporous scaffold comprises a pore volume fraction of 10% to 70% of the composite.

Aspect 37: The composite of any preceding aspect, wherein the pores have size of 20 μm to 200 μm, or 500 nm to 500 μm.

Aspect 38: The composite of any preceding aspect, wherein the pores comprise a morphology comprising a cellular, dendritic, lamellar, or prismatic structure, or any combination thereof.

Aspect 39: The composite of any preceding aspect, wherein the pores are oriented along a primary axis.

Aspect 40: The composite of any preceding aspect, wherein the functional polymer particle comprises a functional gel; and the structural polymer is insoluble or slightly soluble in a solvent capable of swelling the functional gel, optionally wherein the solvent comprises water.

Aspect 41: The composite of any preceding aspect, wherein the structural polymer comprises polyvinylidene fluoride (PVDF), cellulose acetate, polysulfone, polyvinyl chloride, poly(acrylonitrile), polyethersulfone (PES), polypropylene, polytetrafluoroethylene, polyamide imide, natural rubber, or any combination thereof.

Aspect 42: The composite of any preceding aspect, wherein the structural polymer is not covalently attached to the functional polymer particle.

Aspect 43: The composite of any preceding aspect, wherein the structural polymer is present in an amount of 20 wt. % to 80 wt. %, based on total mass of structural polymer and functional polymer particle, excluding solvent, if present; or the functional polymer particle is present in an amount of 20 wt. % to 80 wt. %, based on total mass of structural polymer and functional polymer particle, excluding solvent, if present.

Aspect 44: The composite of any preceding aspect, wherein the functional polymer particle is present in an amount of 20 wt. % to 50 wt. %, based on total mass of structural polymer and functional polymer particle, excluding solvent, if present; and the functional polymer particle has an NCD of 0.3 to 0.8.

Aspect 45: The composite of any preceding aspect, further comprising: at least one metal chelated to the polymer matrix; optionally wherein the metal comprises a transition metal optionally selected from copper, palladium, platinum, iron, rhodium, ruthenium, or any combination thereof.

Aspect 46: The composite of any preceding aspect, further comprising:
a second composite comprising:
   a second macroporous scaffold comprising pores; and
   a second polymer matrix positioned within the pores;
   wherein the second polymer matrix comprises:
      a second functional polymer particle; and
      a second structural polymer;
wherein the pores of the second macroporous scaffold are fluidically connected to the pores of the macroporous scaffold; and
wherein each of the second composite, the second macroporous scaffold, the second polymer matrix, the second functional polymer particle, and the second structural polymer independently are the same or different from each of the composite, the macroporous scaffold, the polymer matrix, the functional polymer particle, and the structural polymer, respectively.

Aspect 47: A method for making the composite of any preceding aspect, the method comprising: infiltrating the pores with a liquid comprising the polymer matrix or a precursor thereof; and performing nonsolvent induced phase separation (NIPS) on the macroporous scaffold infiltrated with the liquid.

Aspect 48: The method of aspect 47, or any preceding aspect, wherein the NIPS comprises a nonsolvent comprising: an alcohol having 1 to 8 carbon atoms optionally selected from methanol, ethanol, isopropyl alcohol, n-propanol, n-butanol, n-pentanol, n-hexanol, or mixtures thereof with water, or any combination thereof; or 20-80 vol. % in water of a solvent of the structural polymer optionally selected from triethyl phosphate (TEP), trimethyl phosphate (TMP), DMSO, DMF, acetone, n-methylpyrrolidone (NMP), N,N-dimethylacetamide (DMA), hexamethylphosphoramide (HMPA), or any combination thereof; or a combination thereof.

Aspect 49: The method of aspect 47 or 48, or any preceding aspect, further comprising: after the infiltrating step but before the performing step: incubating the macroporous scaffold infiltrated with the liquid under conditions sufficient (a) to promote crosslinking of the precursor to produce the polymer matrix, (b) to promote crosslinking within the functional polymer particle and/or with the structural polymer, (c) to promote reaction and/or crosslinking between a surface of the pores and (i) the structural polymer, (ii) the functional polymer particle, (iii) the polymer matrix, and/or (iv) any precursor thereof, or (d) any combination thereof.

Aspect 50: The method of any one of aspects 47-49, or any preceding aspect, further comprising: preparing the polymer matrix by crosslinking a functional polymer particle precursor in the presence of the structural polymer.

Aspect 51: The method of any one of aspects 47-50, or any preceding aspect, further comprising:

prior to the infiltrating step:
if a surface of the pores does not already contain a functional group capable of reacting with a crosslinker, functional polymer particle, precursor of functional polymer particle, and/or structural polymer; then
functionalizing the surface of the pores with the functional group capable of reacting directly or indirectly with the functional polymer particle, a precursor of the functional polymer particle, the structural polymer, or any combination thereof.

Aspect 52: The method of 51, or any preceding aspect, further comprising:

after the functionalizing step but prior to the infiltrating step:
immersing the macroporous scaffold in a solution comprising an oligomer or polymer comprising at least one primary amine, at least one primary ammonium, at least one secondary amine, at least one secondary ammonium, at least one tertiary amine, at least one tertiary ammonium, or any combination thereof; and
incubating the macroporous scaffold containing the solution under conditions sufficient to react the oligomer or polymer to the functional group on the surface of the pores;
optionally wherein the functional group is covalently bonded to the oligomer or polymer via a crosslinker.

Aspect 53: The method of any one of aspects 47-52, or any preceding aspect, wherein the macroporous scaffold comprises a ceramic, and the method further comprises: preparing the macroporous scaffold by a process comprising: freeze casting from a solution of preceramic polymer and a crosslinking agent; or electrochemical synthesis.

Aspect 54: A method for separating a component from a first mixture, the method comprising: passing the first mixture containing the component through the composite of any one of aspects 1-46, or any preceding aspect, and isolating the component from the first mixture.

Aspect 55: The method of aspect 54, or any preceding aspect, wherein the method is functionally equivalent to ion exchange chromatography, affinity chromatography, or a combination thereof.

Aspect 56: A method for catalyzing a chemical reaction in a second mixture, the method comprising: passing the second mixture through the composite of any one of aspects 1-46, or any preceding aspect, wherein the chemical reaction is catalyzed by the composite.

Aspect 57: The method of any one of aspects 54-56, or any preceding aspect, wherein: the first mixture and/or the second mixture comprises a salt concentration of 0 to 500 mM; the first mixture and/or the second mixture comprises a conductivity of 0 to 50 mS/cm; or a combination thereof.

Aspect 58: A composite, comprising:
a macroporous scaffold comprising pores; and
a polymer matrix positioned within the pores;
wherein the polymer matrix comprises:
   a functional gel; and
   a structural polymer
wherein each of the macroporous scaffold, polymer matrix, pores, functional gel, and structural polymer are as defined in any preceding aspect.

The invention can be further understood by the following non-limiting examples.

### Example 1: Polymer Dope Solution Synthesis

2.1 Chemicals and Materials

Polyvinylidene fluoride (PVDF) [Kynar 761] was provided by Arkema (King of Prussia, Pa.). Hyperbranched polyethylenimine (PEI) was procured from Polysciences. Epichlorohydrin (ECH), diethylene glycol diacrylate (EGA), bovine serum albumin (BSA), Bis(2-chloroethyl)amine hydrochloride (BCAH), triethyl phosphate (TEP), isopropanol (IPA), dimethyl sulfoxide (DMSO), (3-Aminopropyl) trimethoxysilane (ATMS), and TRIS hydrochloride were purchased from Millipore Sigma. Hydrochloric acid was purchased from EMD Millipore. Phosphate buffered saline (PBS), with a 1× concentration, was purchased from Corning. All chemicals and materials were used as received. Buffers were prepared using indicated chemicals and distilled water.

2.2 Polymer Dope Synthesis

The synthesis of the polymer dope solution was initiated by dissolving the structural polymer, PVDF, in TEP at 80° C. The polymer solution was then put under a nitrogen atmosphere at ambient pressure and the indicated amount of functional particle precursor, PEI, dissolved in TEP was added. Next, a catalytic amount (˜450 uL) of concentrated HCl was added to the solution. After 15 minutes of mixing, the crosslinker was added to the casting solution followed by a 4-hour crosslinking reaction. The solution was then put under vacuum for 10 minutes prior to infiltrating the ceramic pores. The compositions of the four polymer dope solutions used for this study are provided in Table 1 with the reported normalized crosslink density (NCD). The NCD represents the ratio of crosslinker functional groups (Fₑ) divided by the total number of possible functional groups on PEI (Fₚ), the resulting ratio is normalized by ratio calculated for the reference composition. The calculation of NCD is shown in the following equation:

$$NCD = \frac{{F}_{e}}{{F}_{p}} \div 0.55$$

Several polymeric membranes with the same compositions were prepared as static protein adsorption references. The dope solution was prepared following the steps above. Once the solution was removed from the vacuum, it was cast on a glass plate at a blade height of 300 um and was left in room temperature air for 30 seconds before being immersed in IPA for 2 hours. The solidified membrane was then removed from the IPA bath and stored in a fresh water bath or dried for further characterization.

TABLE 2

| Dope | PVDF | PEI | BCAH | DMSO | |
|---|---|---|---|---|---|
| solution | (g) | (g) | (g) | (mL) | NCD |
| A | 5.91 | 5 | 3.1 | 5 | 0.5 |
| B | 5.91 | 5 | 1.55 | 2.5 | 0.25 |
| C | 5.91 | 5 | 0.78 | 1.25 | 0.125 |
| D | 5.91 | 5 | 0.39 | 0.625 | 0.0625 |

### Example 2: Scaffold Fabrication: Ceramic Scaffold

2.3 Ceramic Fabrication

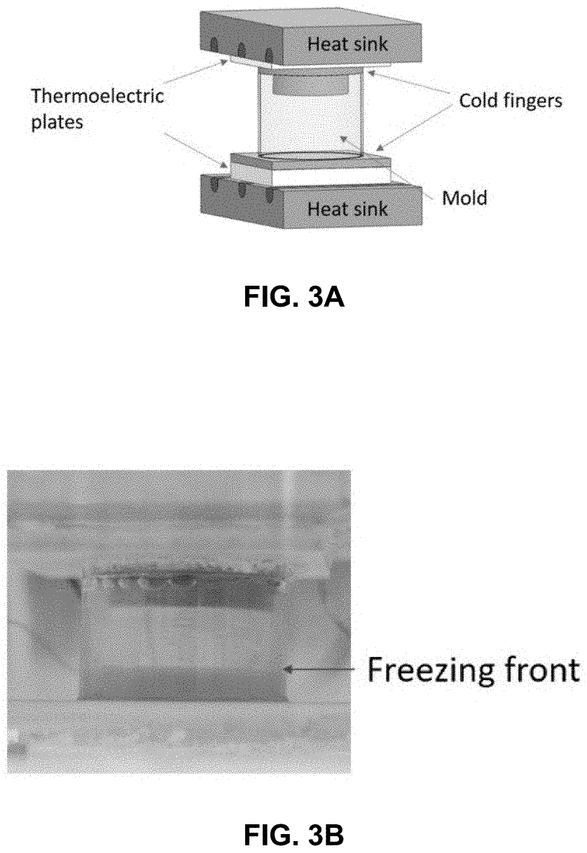

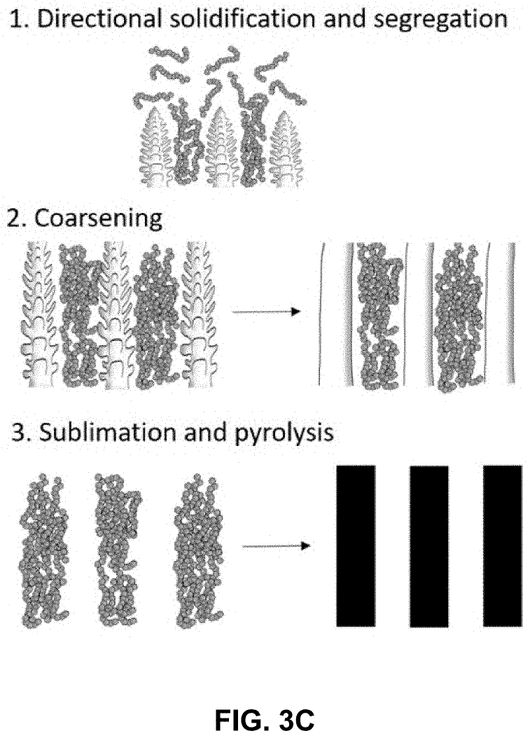

A polymer solution was prepared by dissolving a polysiloxane (CH3-SiO1.5, Silres® MK Powder, Wacker Chemie) preceramic polymer in cyclohexane (C₆H₁₂, Sigma-Aldrich), with concentration of preceramic polymer of 20 wt. %. Once a homogenized solution was obtained, a cross-linking agent (Geniosil® GF 91, Wacker Chemie) was added in concentrations of 1 wt. % and stirred for an 5 minutes and degassed for 10 min to avoid air bubbles during solidification. The freeze-casting was done by pouring the polymer solution into the glass mold (h=20 mm, 0=25 mm) which sat on a PID-controlled thermoelectric plate. Another thermoelectric was placed on top of the mold to control both freezing front velocity and temperature gradient, a similar configuration as the work by Zeng et al. [18] (FIGS. 3A-3C). A cold finger with smaller diameter than the mold was inserted into the glass mold such that the created space acted as a reservoir for the solution as the solution shrunk by solidification. The freezing front velocity and temperature gradient were measured by taking pictures every 30 seconds using a camera and intervalometer. The temperature gradient, G was defined by the following equation:

$$G = \frac{{T}_{r} - {T}_{f}}{d}$$

where Tₜ is the temperature of top cold finger, T_{f} is the temperature at the freezing front and d is the distance between the top cold finger and the freezing front. The temperature of the freezing front was assumed to be at the liquidus temperature of the solution, and the value was taken from the work by Naviroj [19]. All samples were frozen at freezing front velocities of 15 μm/s, and temperature gradients of 2.5 K/mm to maintain homogeneous pore structures.

Once the structure was completely frozen, isothermal coarsening was initiated by setting the top and bottom thermoelectrics to 4° C. After the structure was coarsened for 3 hours, the sample was re-froze. After the sample was completely frozen, it was sublimated in a freeze drier (VirTis AdVantage 2.0) where the solvent crystals were completely removed. After freeze drying, the polymer scaffold was pyrolyzed in argon at 1100° C. for four hours to convert the preceramic polymer into silicon oxycarbide (SiOC). This resulted in a porosity of ˜77%.

The pyrolyzed sample was machined into a disk with thickness of ˜1.6 mm and diameter of ˜13 mm for further processing of the composite.

### Example 3: Surface Functionalization of Ceramic

2.4 Surface Functionalization of Ceramic

In preparation for injection molding, the ceramic surface was activated using a procedure derived from (citation). The porous SiOC disc was first immersed in concentrated NaOH for 90 minutes. It was then washed in water before being incubated in a 0.1 M HCl solution for 30 minutes. The ceramic was then washed in water again, before being dried at 100° C. for 1 hour. Once the ceramic was dried, it was added to a 2 v % solution of ATMS in isopropanol and incubated for 3 hours at 60° C. The sample was then washed thoroughly in water and isopropanol before being cured at 110° C. for 30 minutes.

Upon completion of the steps above, the ceramic surface was amine terminated. In order to crosslink the amine-terminated surface with available amine groups in the dope solution, the ceramic was immersed in a solution of PEI and ECH with a stoichiometric ratio of crosslinker to amine greater than 1. The ceramic was incubated in this solution overnight at room temperature to form a crosslinking gel layer. After the overnight incubation, DMSO was added to the vessel containing the sample and the resulting solution was heated to 80° C. for 1 hour to remove excess functionalized PEI. The sample was then washed with IPA and dried at room temperature for one hour prior to the addition of the polymer dope solution.

### Example 4: Phase Inversion Molding

2.5 Phase Inversion Molding

The dried ceramic was loaded into the infiltration device, comprised of laser cut acrylic sheets and silicon gaskets, and the polymer dope solution was injected using a syringe pump. The solution was pumped at a rate of 100 μL/min until the ceramic and all dead volume within the infiltration device was filled. The device was then incubated at 80° C. for 1 hour to promote the crosslinking reaction between the ceramic gel layer and amine groups in the dope solution. Following the incubation, the samples were removed from the infiltration device and placed in IPA for an overnight incubation. The following day, the samples were moved to water baths to remove trace solvent and IPA in preparation for BSA binding characterization.

### Example 5: Membrane Properties: Physical

2.6 Membrane Properties Characterization

2.6.1 SEM

The microstructure of ceramic scaffolds and polymer/ceramic composites were observed using a scanning electron microscope (SEM; Zeiss 1550VP, Carl Zeiss AG, Oberkochen, Germany). Each sample was dried at 70° C. overnight. The surfaces and cross-sections of the samples were coated with a Pt/Pd conductive layer and then imaged. The sample cross-sections were prepared by snapping the membrane in half at ambient conditions.

Results & Discussion

3.1 Phase Inversion Molding

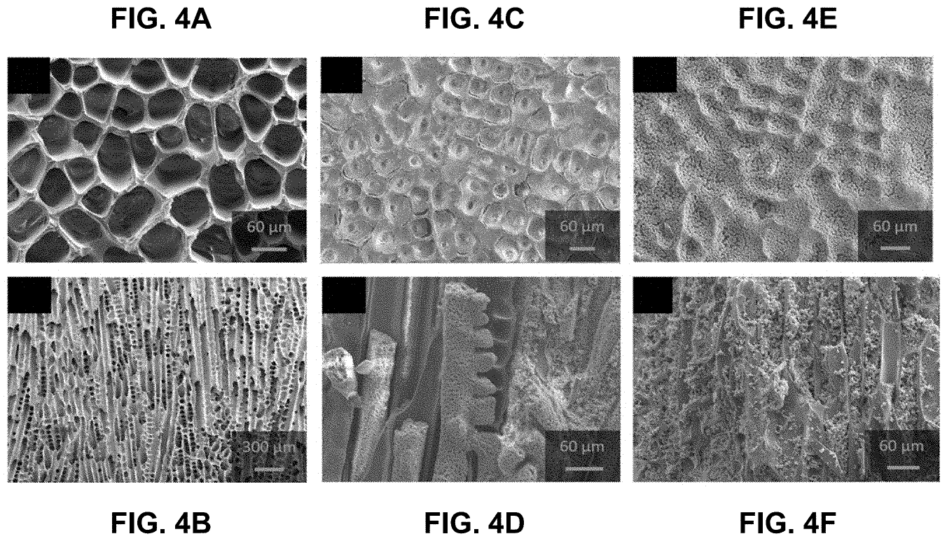

In order to utilize functional polymer gels that have high protein binding capacities, but are mechanically weak, it is essential to be able to combine them with a mechanically strong scaffold. Furthermore, the scaffold may advantageously have consistently sized pores to form a composite suitable for membrane chromatography. FIGS. 4A-4B demonstrate the pore morphology of the ceramic scaffold used throughout this study. The functional polymer matrix was combined with the ceramic scaffold using injection molding and subsequent phase inversion of the polymer matrix. FIGS. 4C-4D demonstrate that the pore-filling achieved using this method is excellent. It may be observed in FIG. 4D, that even the smaller pockets arranged along the main pore are infiltrated. While FIGS. 4C-4D demonstrate that pore morphology is matched quite well, they also show a critical drawback—namely large gaps between the polymer matrix and the ceramic wall. In membrane chromatography applications it is essential that there are no large voids or gaps within the bulk membrane. Such voids are especially detrimental if they break both membrane surfaces, thereby creating an undesired region where fluid may flow through without coming into contact with the functional groups within the membrane. These gaps are most likely due to shrinkage of the polymer matrix during the drying process prior to imaging; however, there is a remote possibility that they exist even when the sample is in the wet state. To ensure that there are no voids or gaps within the composite, the ceramic surface was functionalized with a reactive PEI based gel. FIG. 4E demonstrates the positive impact of the gel layer on the cohesiveness between the polymer matrix and the ceramic surface. Furthermore, a comparison of FIGS. 4D and 4F it may be seen that the ceramic wall in d is bare while the ceramic wall in FIG. 4F is decorated with PEI particles and portions of the polymer matrix. The decoration of the ceramic suggests that the polymer matrix is covalently bonded to the wall.

Changing the crosslinker concentration influences both morphology and protein binding capacity. The SEM cross-sections in FIGS. 5A-5D demonstrate the changes in morphology as the crosslinker concentration is decreased from composition A to D. It may be seen that FIG. 5D, corresponding to composition D, no longer has a decorated ceramic wall. It is possible that this is due to “unbound” PEI from the dope solution saturating the PEI reactive layer resulting in a passivated surface. This prevents the polymer matrix from bonding to the ceramic and leads to the same gap seen in FIGS. 5C-5D.

### Example 6: Protein Adsorption Studies

2.6.2 Protein Adsorption Studies

BSA was used as the model protein in both static and dynamic binding measurements. Initial tests were done using BSA in distilled water at a concentration of 2 mg/m L. To measure the static binding of the polymeric references, a known volume of membrane was immersed in a 2 mg/mL BSA solution and gently mixed for 48 hours. The absorbance of the solution was then measured using a UV-vis spectrometer (details) and the reported value of absorbance at 280 wavenumbers was used to determine the mass of BSA bound per volume of membrane. To account for the thickness of the formulated composites, the static binding capacity was determined by recirculating a 2 mg/mL BSA solution through the ceramic for 4 hours at a flow rate of 300 uL/min. The salt tolerance of composite B & C was determined by measuring the volumetric binding capacity using BSA solutions with the following compositions: distilled water, 50 mM TRIS buffer, 50 mM TRIS buffer with 100 mM NaCl, 0.5×PBS, and 1×PBS.

Dynamic binding measurements using BSA in H₂O were conducted using Composites B & C. Further experiments utilizing the other BSA solutions described above were performed using only sample B. To conduct the measurement, the sample was first loaded into the sample holder and was equilibrated using the solvent of the BSA solution. The BSA solution was then introduced via a syringe pump to the device at a rate of 300 uL/min (or 3 membrane volumes/min). The filtrate was analyzed with time-resolved measurements on the UV-vis spectrometer. The 10% breakthrough curve method was used to determine the dynamic binding capacity.

3.2 Protein Binding Studies

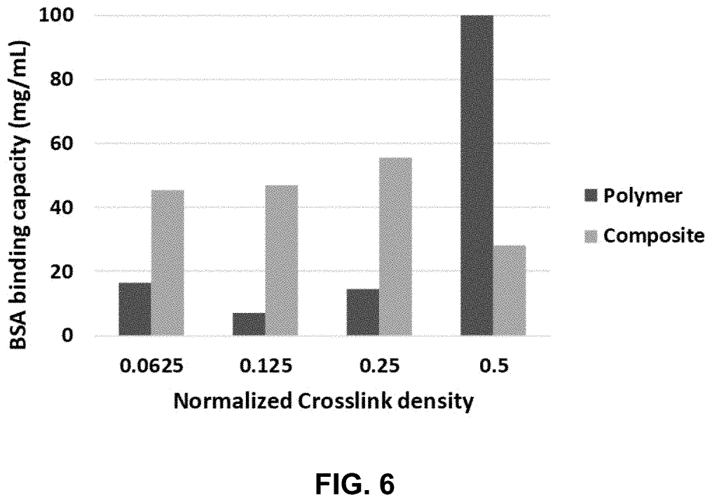

When developing these composites, we expected there to be a clear trade-off between mechanical stability/modularity and volumetric binding capacity. Initial estimates predicted that the total volumetric binding capacity of the composite would be ˜70% of the polymer matrix binding capacity (accounting for the volume occupied by the ceramic and the reduced number of amines after binding to the surface). FIG. 6 presents the reported total volumetric binding capacity for both the polymer matrix and composites at different NCD values. There are two key points demonstrated in this figure. First, at high NCD, the composite binding capacity, 30 mg/mL, is significantly lower than 70% of the polymer matrix binding capacity, 100 mg/m L. This observation suggests that there is an additional interaction between the polymer matrix and the ceramic not accounted for above. One possible explanation is the rigid nature of the ceramic containing the swelling of the functional particles, and thereby reducing the volume in which proteins may interact with available amines.

The second key point is seen at lower NCD values, where the composite outperforms the polymer matrix by more than a factor of 2. The improvement in binding capacity may be explained by the interactions of the polymer matrix/particle precursors and the surface functionalized ceramic. When the ratio of crosslinker to PEI decreases, as reflected in the NCD, the average number of bonds formed by each PEI molecule is reduced (Table 3). As a result, the percentage of PEI molecules which are not sufficiently crosslinked to be “captured” by the polymer matrix increases. In the case of the polymer matrix alone, these PEI molecules may escape into the nonsolvent bath leading to fewer amines available to interact with BSA. However, when preparing the composites, these “escapee” PEI molecules have an additional opportunity to bond to the functionalized surface of the ceramic. Once bonded to the surface, they provide additional amines for protein adsorption. In addition, with fewer bonds between PEI molecules, the functional microgels are enabled to swell to a greater degree leading to more opportunities for free amines to interact with proteins.

TABLE 3

| | Average |
|---|---|
| | number of |
| | bonds/PEI |
| NCD | molecule |
| 0.5 | 4.1 |
| 0.25 | 2.1 |
| 0.125 | 1.0 |
| 0.0625 | 0.52 |

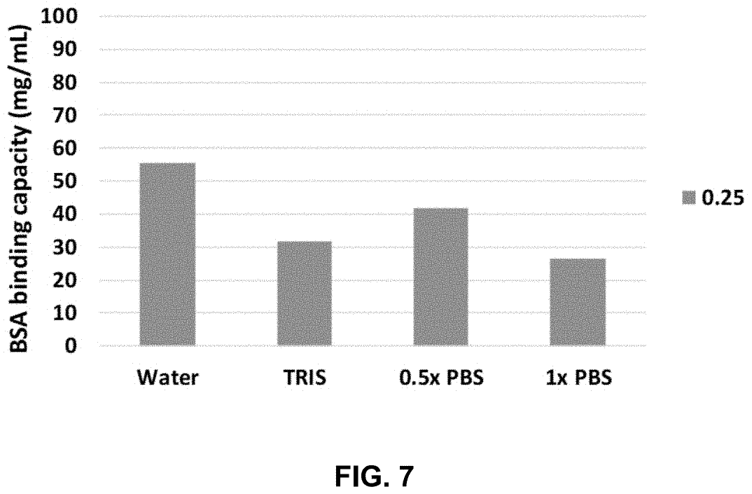

Composite B was chosen for the static salt tolerance experiments because of its high binding capacity and desirable morphology. The current results provided in FIG. 7 demonstrate an 80% retention of binding capacity up to a salt concentration of 125 mM. This is slightly lower than the 90% retention reported in (M2P2 paper) but may be attributed to the influence of the covalent bonding to the ceramic surface. The consistency between samples suggests that the reported protein adsorption is due to the presence of weak base amines. As the salt concentration is further increased to 250 mM, in 1×PBS, the binding capacity has been reduced by 50%.

References corresponding to Background, Summary, Detailed Description, and Examples 1-6.
1. R. Ghosh, Protein separation using membrane chromatography: opportunities and challenges, J. Chromatogr. A 952 (2002) 13-27
2. P. Madadkar, Q. Wu, R. Ghosh, A laterally-fed membrane chromatography model, Journal of Membrane Science 487 (2015) 173-179
3. B. V. Bhut, S. R. Wickramasinghe, S. M. Husson, Preparation of high-capacity, weak anion-exchange membranes for protein separations using surface-initiated atom transfer radical polymerization, Journal of Membrane Science 325 (2008) 176-183
4. B. V. Bhut, S. M. Husson, Dramatic performance improvement of weak anion-exchange membranes for chromatographic bioseparations
5. L. Sun, J. Dai, G. Baker, M. Bruening, High-capacity, protein-binding membranes based on polymer brushes grown in porous substrates, Chem. Mater. 2006, 18, 4033-4039
6. S. Fischer-Frühholz, D. Zhou, M. Hirai, Sartobind STIC salt-tolerant membrane chromatography, Nat Methods 7, 12-13 (2010)
7. V. Orr, L. Zhong, M. Moo-Young, C. P. Chou, Recent advances in bioprocessing application of membrane chromatography, Biotechnology Advances 31 (2013) 450-465
8. J. J. Keating, J. Imbrogno, G. Belfort, Polymer Brushes for Membrane separations: A review, ACS Appl. Mater. Interfaces 2016, 8, 28383-28399. J. Fan, J. Luo, X. Chen, Y. Wan, Facile preparation of salt-tolerant anion-exchange membrane adsorber using hydrophobic membrane as substrate, J. Chromatogr. A 1490 (2017) 54-62
9. W. Riordan, S. Heilmann, K. Brorson, K. Seshadri, Y. He, M. Etzel, Design of salt-tolerant membrane adsorbers for viral clearance, Biotechnology and Bioengineering 103 (2009) 920-929
10. Q. Yang, et al., Composites of functional polymeric hydrogels and porous membranes, J. Mater. Chem., 2011, 21, 2783-2811, DOI: 10.1039/c0jm02234a
11. N. Adrus, M. Ulbricht, Novel hydrogel pore-filled composite membranes with tunable and temperature-responsive size-selectivity, J. Mater. Chem., 2012, 22, 3088, DOI: 10.1039/c2jm 15022k
12. A. Doring, W. Birnbaum, D. Kuckling, Responsive hydrogels—structurally and dimensionally optimized smart frameworks for applications in catalysis, micro-system technology and material science, Chem. Soc. Rev., 2013, 42, 7391-7420, DOI: 10.1039/C3CS60031A
13. M. Petrini, M. Ferrante, B. Su, Fabrication and characterization of biomimetic ceramic/polymer composite materials for dental restoration, Dental Materials 29 (2013) 375-381, http://dx.doi.org/10.1016/j.dental.2012.12.004
14. R. C. F. Cheung, J. H. Wong, T. B. Ng, Immobilized metal ion affinity chromatography: a review of its applications, Appl Microbiol Biotechnol (2012) 96:1411-1420, DOI: 10.1007/s00253-012-4507-0
15. M. R. Kotte, et al., Mixed Matrix PVDF Membranes With in Situ Synthesized PAMAM Dendrimer-Like Particles: A New Class of Sorbents for Cu(II) Recovery from Aqueous Solutions by Ultrafiltration, Environ. Sci. Technol. 2015, 49, 9431-9442, DOI: 10.1021/acs.est.5b01594\
16. M. R. Kotte, et al., A Facile and Scalable Route to the Preparation of Catalytic Membranes with in Situ Synthesized Supramolecular Dendrimer Particle Hosts for Pt(0) Nanoparticles Using a Low-Generation PAMAM Dendrimer (G1-NH2) as Precursor, ACS Appl. Mater. Interfaces 2018, 10, 39, 33238-33251, DOI: 10.1021/acsami.8b11351
17. M. R. Kotte, et al., A facile route to the preparation of mixed matrix polyvinylidene fluoride membranes with in-situ generated polyethyleneimine particles, Journal of Membrane Science 450 (2014) 93-102, DOI: 10.1016/j.memsci.2013.08.025
18. X. Zeng, N. Arai, K. T. Faber, Robust Cellular Shape-Memory Ceramics via Gradient-Controlled Freeze Casting, Adv. Eng. Mater. 21 (2019) 1900398. https://doi.org/10.1002/adem.201900398.
19. Maninpat Naviroj, Silicon-based Porous Ceramics via Freeze Casting of Preceramic Polymers (Ph.D. thesis), Northwestern University, 2017. https://search.proquest.com/openview/556ab 0b3ba48bd5ad64aa2c67a18ff27/1?pq-origsite=gscholar&cb1=18750&diss=y (accessed May 23, 2018).

### Example 7: Membrane Design and Application—Background

1 Introduction

Examples 7-10 generally focus on two topics: the interplay between nonsolvent and membrane composition during phase inversion (Example 8), and the application of mixed-matrix materials to membrane chromatography (Examples 9 and 10).

1.1 Introduction to Membrane Technology

Membranes are semi-permeable barriers between two phases that allow selective transport from one phase to the other. The inherent selectivity of membranes allows them to perform separations more energy efficiently than competing methods. The improved energy efficiency and tailorable selectivity have facilitated the use of membranes in several fields including water purification¹⁻⁴, bioseparations⁵⁻⁷, catalysis^{8,9}, and resource recovery^{10,11}. The mechanism of selectivity stems from both the membrane's physical structure and its chemical composition.

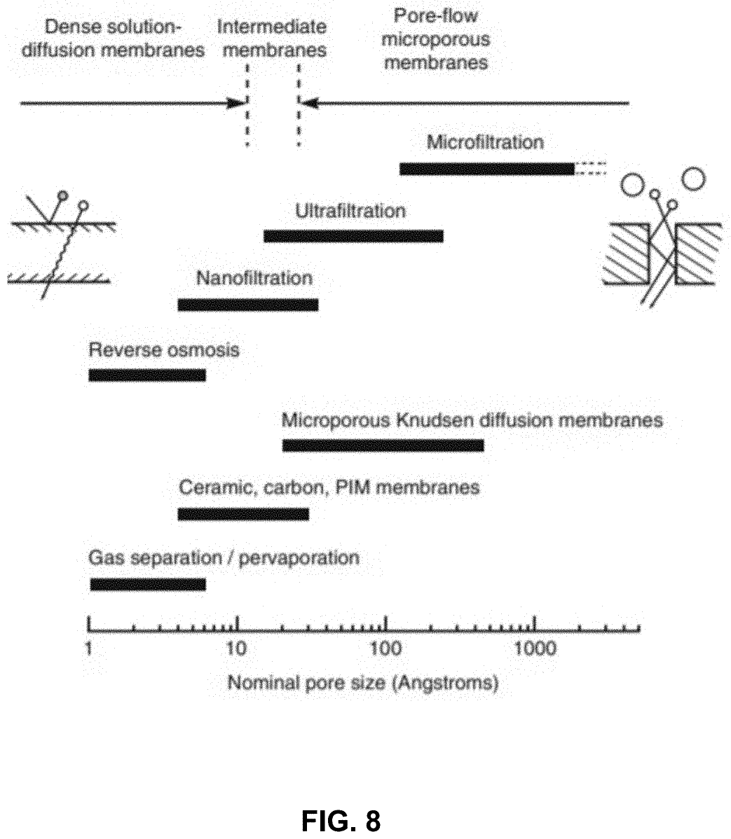

The morphology of a membrane may be classified as being either symmetric (homogeneous) or asymmetric (heterogeneous)¹². Symmetric morphologies are further differentiated as porous or dense structures; wherein the mechanisms for mass transfer are pore-flow and solution-diffusion for porous and dense membranes respectively. Asymmetric membranes are characterized by having a dense skin layer supported by a porous sublayer and therefore demonstrate a mixture of pore-flow and solution-diffusion mass transfer. If the skin layer and porous support are not fabricated from the same material, the membrane is considered a composite¹³. FIG. 8 shows the range of size-based separations that is compatible with the different mechanisms of mass transfer highlighted for each membrane morphology. As the mass transfer transitions from pore-flow to solution-diffusion, the size of particles that are able to permeate the membrane decreases. In addition to the influence of the membrane structure, selectivity is also impacted by membrane composition including any additional functionalization of the base material.

Membranes may be fabricated using either biological or synthetic materials, with the latter covering both inorganic and organic compounds^{12,13}. Of particular interest to the work presented in this thesis are polymeric and ceramic membranes; both materials have been used as membranes in several fields and have corresponding advantages and drawbacks. Considering first the benefits of polymeric membranes: First, literature has demonstrated how to easily fabricate both symmetric and asymmetric membranes with a range of size selectivity from a polymer solution using a method known as Nonsolvent Induced Phase Separation (NIPS)¹⁴⁻¹⁶. Second, there have been over 100 different types of polymers tested as membrane materials and of those several have been demonstrated to have—among polymers—great chemical and thermal resistance as well as good mechanical strength. These materials include poly(vinylidene Fluoride) (PVDF), polysulfone (PS), polytetrafluoroethylene (PTFE), polyethylene (PE), polypropylene (PP), and polyimide (PI)^{12,17}. Third, several polymeric membranes are amenable to further surface functionalization to tailor properties such as hydrophilicity, surface charge, and fouling resistance^{12,18-20}. Fourth, the fabrication of membranes from polymer dope solutions facilitates the inclusion of functional ‘additives’ that are used to influence morphology^{3,18}. Consider next the drawbacks associated with polymeric membranes: First, polymeric membranes used in separation processes frequently suffer from a build-up of unwanted material on the membrane surface through a process known as fouling^{19,21}. As the build-up thickness increases the filtration process suffers a decrease in the permeate flux, which eventually requires cleaning of the membrane surface through backwashing or chemical treatments. Second, they are easily degraded in solutions that contain a good solvent for the polymer as well as many cleaning solutions¹⁷. For example, membranes fabricated using PVDF have been demonstrated to lose mechanical integrity after treatment in caustic or amine rich solutions. Third, polymeric membranes have a lower maximum operating temperature than equivalent inorganic materials¹².

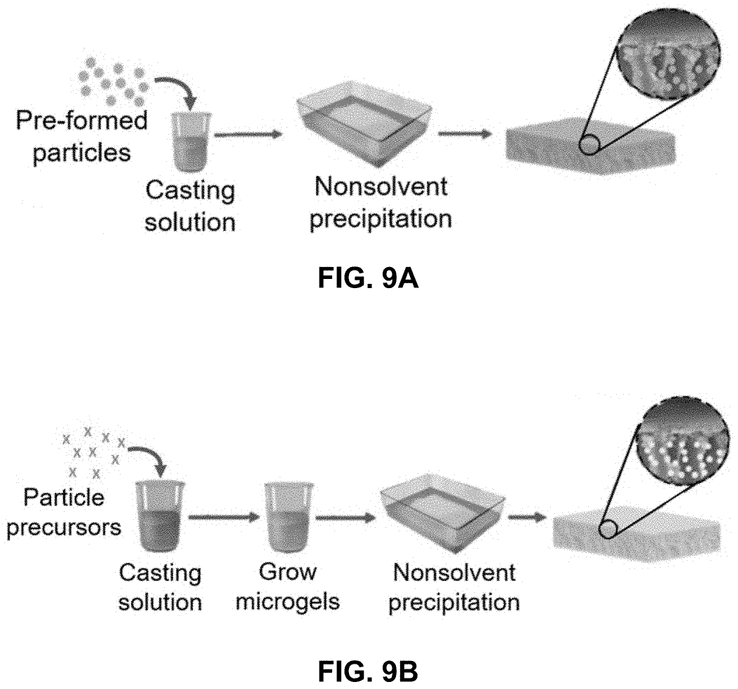

A noteworthy subclass of polymeric membranes known as mixed-matrix membranes (MMM) are identified by the incorporation of functional materials into the matrix of the structural polymer. The inclusion of functional materials has facilitated improved performance in several fields including water purification^{3,19}, gas separations²², and resource recovery^{10,11}. FIGS. 9A-9B display two common methods used to incorporate the functional particles into the polymer matrix. The first route adds preformed particles to the dope solution and uses a variety of methods to achieve a homogeneous distribution prior to casting the membrane. However, the methods used to encourage mixing are frequently detrimental to the structural polymer and are often not successful in evenly distributing the functional polymeric particles²³. In the case when the resulting membrane does have functional particles dispersed throughout, these particles are only bound to the polymer matrix through physical interactions. As a result, the pre-formed functional particles tend to leach out over time leading to a decrease in membrane performance. The route displayed in FIG. 9B is an alternative designed to avoid the drawbacks associated with the direct addition of pre-formed particles. By generating the functional particles in situ, the particles will be both more evenly distributed and more tightly incorporated into the polymer matrix. Furthermore, a method pioneered by Diallo and coworkers demonstrated the successful inclusion of in situ generated polymeric functional particles into a MMM. Although mixed-matrix membranes provide several benefits in the form of improved performance and novel applications, they still share the drawbacks of sensitivity to tough cleaning solutions and lower operating temperatures with the neat polymer membranes²³.

Ceramic membranes are in many ways a natural counter to polymeric membranes. Consider the advantages of ceramic membranes: First, ceramic membranes have demonstrated a lower propensity to fouling in water purification and bioseparations. Second, they exhibit excellent chemical resistivity and retention of mechanical integrity in a variety of extreme environments such as caustic, bleach, and concentrated acidic solutions^{24,25}. Third, they are compatible with operations at temperatures over 200° C., a temperature range in which most polymeric membranes would be in the melt state¹². Fourth, there are well documented methods to further functionalize the ceramic surfaces as required²⁶. The following are considered the key drawbacks to ceramic membranes: First, they are significantly more expensive—anywhere from 3 to 5 times—to produce than polymeric membranes¹². Second, it is more difficult to obtain an asymmetric ceramic structure with a dense selective layer than it is to form an asymmetric polymer membrane. This drawback is somewhat mitigated by the use of ceramic membranes as the support for an asymmetric composite. Third, the selection of additives that may be incorporated into the ceramic structure is limited by the harsh processing conditions used during fabrication. Ceramic and polymeric membranes have different advantages that tailor their capabilities towards different applications.

1.2 Nonsolvent Induced Phase Separation—Literature Review

1.2.1 Brief History of Synthetic Membranes

The first synthetic membrane was fabricated using nitrocellulose by Adolph Fick in 1855²⁷. The introduction of cellulose based synthetic membranes provided a level of reproducibility that was unobtainable with animal-based membranes. The field was further advanced by Bechhold in 1907, who introduced a method to control pore size and measure pore diameters as well as coining the term ‘ultrafiltration’. By the 1940s commercial cellulose membranes were used to determine the safety of drinking water as well as the removal of contaminants in research applications. Over the next couple of decades, several additional polymers were tested, but the applications of synthetic membranes were limited due to difficulty in fabrication and low fluxes. Then, in 1963 Suorirajan and Loeb published their discovery of ‘immersion precipitation’—a novel precipitation method that produced defect free asymmetric membranes¹⁷. The unique morphology of the asymmetric membrane enabled both high selectivity and high flux. The selectivity stems from the dense skin layer, while the porous sublayer facilitates higher fluxes by reducing the mass transfer resistance across the majority of the membrane. Following the Loeb-Sourirajan discovery, the field of membrane technology underwent a revolution and grew rapidly through the 1980s. In the 1970s there was a transition from cellulose based membranes to a composite membrane comprised of polysulfone and polyamide, which demonstrated better thermal and chemical resistance. Since the 1970s, there has been extensive literature research done on ‘immersion precipitation’ with a variety of different polymers²⁷. This example will focus on systems that use PVDF as the structural polymer.

1.2.2 Overview of NIPS Mechanism

Phase inversion is the process of solidifying a homogeneous liquid polymer solution under controlled conditions. There are several methods to induce the phase separation leading to polymer solidification including nonsolvent induced phase separation (NIPS), thermally induced phase separation (TIPS), polymerization induced phase separation (PIPS), and vapor induced phase separation (VIPS). NIPS and its derivatives are the most commonly used methods in the literature and commercially.

The NIPS process begins when the homogeneous liquid polymer solution is immersed in a liquid that is incompatible with the polymer, known as a nonsolvent. As the solvent and nonsolvent interdiffuse, the composition of the casting solution changes and depending upon the rates of mass transfer follows one of the four routes shown in FIG. 10²⁸. Along the four routes there are two types of demixing processes to consider: liquid-liquid demixing—wherein the ternary solution starts as a homogeneous solution in the one phase area and then crosses the binodal into an unstable regime that induces phase separation into two liquid phases, and solid-liquid demixing—wherein a ternary solution in either the one phase or two phase area cross into the gel region producing a solid polymer crystal phase in equilibrium with a liquid polymer-lean phase¹⁷. In other words, in liquid-liquid demixing the solution phase separates as a liquid and then the polymer-rich region solidifies and crystallizes. In solid-liquid demixing the polymer crystallization and solidification drives phase separation and as a result is a slower process that is seen mostly in semi-crystalline polymers such as PVDF.

In route 1, the rate of solvent leaving the dope solution is faster than the rate of nonsolvent entering. This imbalance in fluxes results in the polymer concentration increasing until it surpasses the gelation concentration. Once the ternary system has entered the gelation concentration regime the polymer undergoes solid-liquid demixing and solidifies via gelation and/or crystallization into a dense and compact structure¹².

In route 2, the interdiffusion of solvent and nonsolvent produces a ternary solution that is in a metastable region in-between the spinodal and binodal lines. In the metastable region concentration fluctuations lead to the nucleation and growth of the polymer-lean phase via liquid-liquid demixing. If the growth of the polymer-lean phase reaches the point of phase coalescence (the droplets of polymer-lean phase begin coalescing) the resulting membrane will have an open-cellular morphology. When the growth is interrupted by solidification of the polymer-rich matrix before reaching domain coalescence the resulting membrane will have a closed-cell morphology¹².

For route 3, the interdiffusion of solvent and nonsolvent results in a ternary solution that passes the metastable region and enters the two phase region. Upon reaching the two phase region, even small concentration fluctuations motivate the solution to separate via spinodal decomposition into two continuous phases with concentrations determined by the tie-lines depicted in FIG. 10. The spinodal decomposition is very fast and so the final morphology is determined by the competition between phase coalescence of polymer solidification. Therefore, rapid solidification results in a membrane with high pore interconnectivity being produced; whereas, slower solidification of the polymer-rich phase produces a membrane analogous to a nucleation and growth mechanism.

Route 4 has several similarities with route 2 in that the ternary solution enters a metastable region and growing concentration fluctuations leads to the formation of nuclei and growth. However, in the case of route 4, the nonsolvent diffuses into the membrane faster than solvent leaves resulting in a decreasing polymer concentration. As the ternary solution moves into the metastable region, liquid-liquid demixing motivates the nucleation and growth of the polymer-rich, thereby producing a nodular morphology consisting of loosely connected polymer aggregates.

The brief explanation above provides an introduction to the thermodynamics behind NIPS, but it is important to note that different sections across the thickness of the casting solution are at different points on the ternary phase diagram¹⁷. For example, while the top surface of the casting solution may already be undergoing spinodal decomposition, the middle of the polymer solution may just be reaching the binodal line, with the bottom layer still being comfortably in the one phase region. The path to phase separation of the lower sections in the polymer solution will be influenced by the changes in solvent and nonsolvent mass transfer arising from the phase separation and polymer solidification higher up in the membrane¹⁴. As a result, the driving forces behind phase separation at the nonsolvent-dope solution interface and the bottom of the membrane could be completely different; indeed, this is the very phenomenon that gives rise to asymmetric membranes.

1.2.3 Effect of Nonsolvent

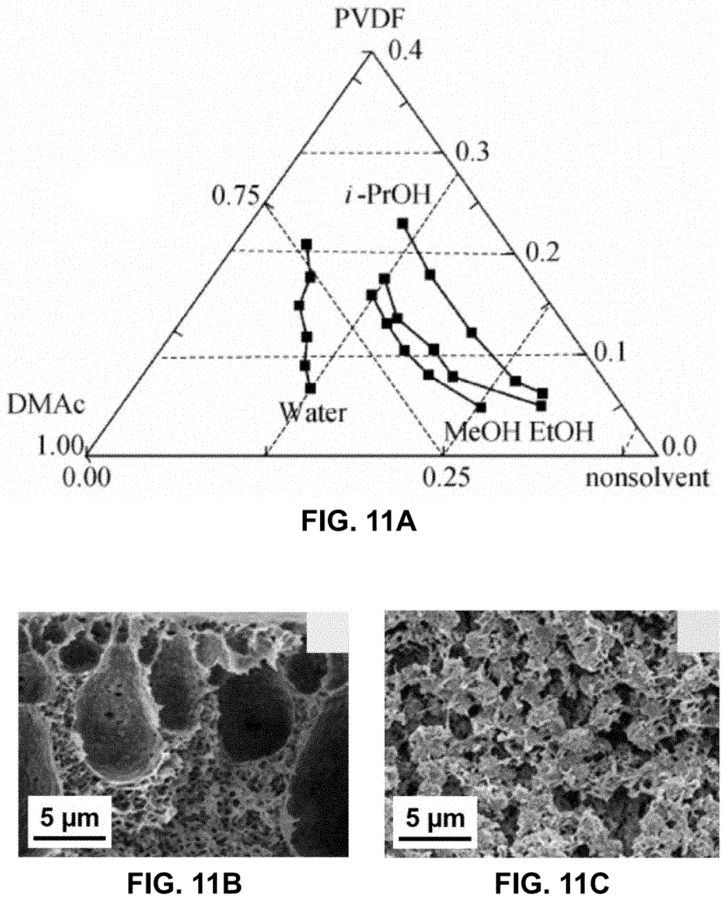

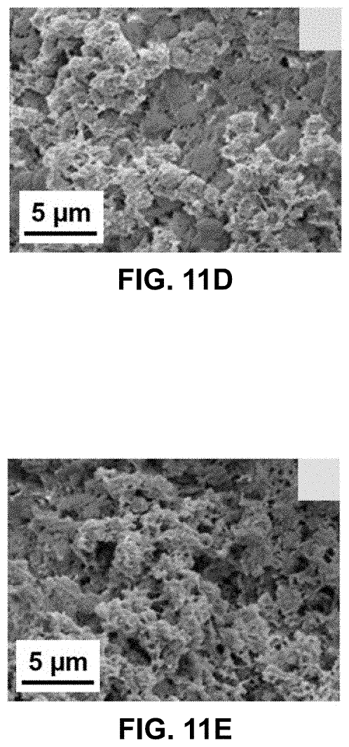

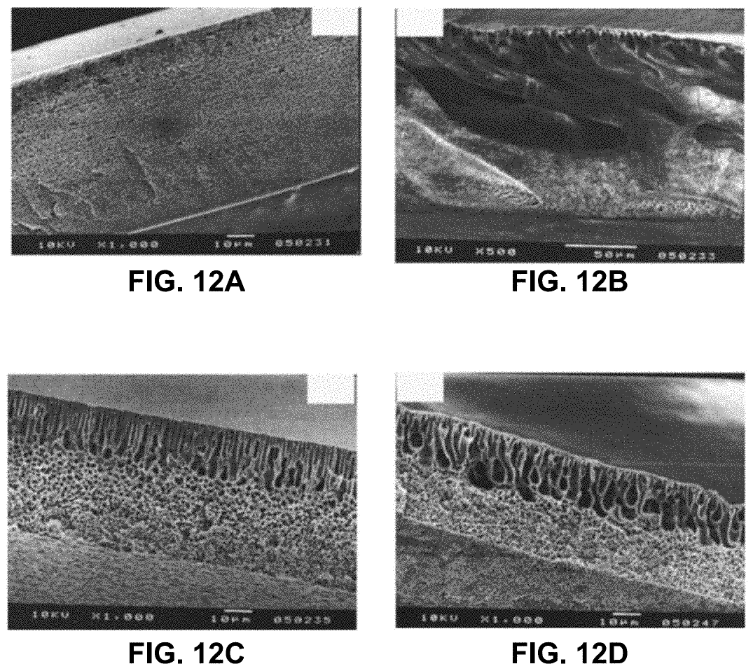

As shown in FIG. 11A, using different nonsolvents shifts the placement of the binodal line in the ternary phase diagram. The placement of the binodal line correlates to the time required for the nonsolvent to diffuse and reach a high enough concentration to induce phase separation. Nonsolvents with binodal lines that are closer to the left side of the diagram, such as water in FIG. 11A, are known as hard nonsolvents because they are not tolerated by the casting solution resulting in instantaneous demixing of the ternary solution¹⁵. The speed of the phase separation favors liquid-liquid demixing, which leads to the formation of asymmetric membranes. The morphology of the porous sublayer changes depending on the solvent used. Bottino et al. published an excellent study on the role of different solvents in determining membrane morphology when using water as the nonsolvent¹⁴. They reported a good correlation between solvent-nonsolvent mutual diffusivity and membrane morphology. FIGS. 12A-12D present the different morphologies obtained from polymer solutions of four common solvents for PVDF²⁹. Of particular interest to the work presented in this thesis, was the behavior of membranes prepared using Triethyl phosphate (TEP). The TEP-water mutual diffusivity is low, producing asymmetric membranes with sponge-like layers that do not contain macorvoids¹⁴.

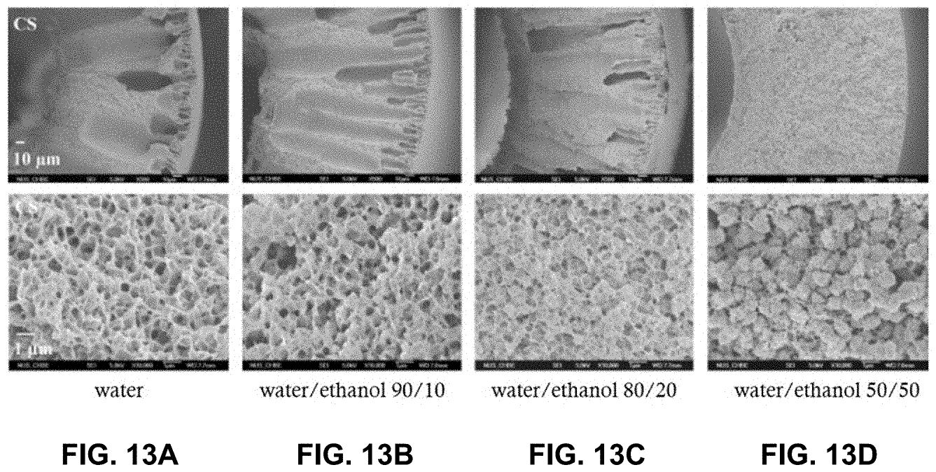

Nonsolvents with binodal lines towards the right of the phase diagram (such as ethanol and isopropanol in FIG. 11A) are known as soft nonsolvents because they require a higher concentration to induce phase separation. The system typically needs a longer diffusion time to reach the necessary concentrations to induce phase separation resulting in delayed demixing of the ternary solution. The delay in demixing has several critical impacts on membrane structure¹⁵. First, the slower demixing at the surface changes the dynamics of skin layer formation. For example, when membranes are cast in reaction grade isopropanol or ethanol the skin layer formation is completely disrupted producing a symmetric membrane with a surface morphology consistent with the bulk structure. Second, the longer time required to initiate phase separation increases the contribution of solid-liquid demixing, thereby suppressing the formation of macrovoids and producing a sponge-like structure. Both of these effects are influenced by the ‘softness’ of the nonsolvent. Nonsolvent ‘softness’ may be tailored by making either water-soft nonsolvent or water-solvent mixtures. Sukitpaneenit et al. investigated the changes in membrane structure and performance when prepared using nonsolvents comprising mixtures of water and ethanol³⁰. As the ethanol concentration increased, the formation of the skin layer was disrupted and the bulk membrane structure transitioned from fingerlike pores and macrovoids to a globular sponge-like morphology (FIGS. 13A-13D)³⁰.

1.2.4 Effect of Additives

Up to this point the NIPS process has only been considered in the context of a polymer solution comprising a single polymer dissolved in a solvent. However, one of the focuses of this work is to determine how in situ generated functional microparticles interact with the other components of the polymer solution and nonsolvent to influence membrane morphology. This section summarizes the current literature on incorporating different additives into PVDF casting solutions and their influence on membrane structure. These additives fall within 3 categories: low molecular weight (MW) compounds including inorganic salts and small molecules, high molecular weight polymers, and inorganic particles.

Membranes prepared using additives in the first category are not considered MMMs because the low MW compounds are not incorporated into the polymer matrix, but rather diffuse into the nonsolvent bath upon casting. Although they do not contribute to the long term functionality of the membrane, these compounds have been demonstrated to facilitate distinctive changes to membrane morphology and performance. For example, work by Bottino et al. demonstrated that inclusion of low concentrations of lithium chloride (LiCl) into the dope solution produced a more porous polymer structure with larger cavities³¹. As the concentrations of LiCl are increased, macrovoid formation is suppressed and the support layer becomes more sponge-like. The change in morphology was attributed to a higher rate of polymer precipitation driven by the casting solution being less thermodynamically stable and LiCl mixing with water^{18,31}. Another common low MW additive studied by Yeow et al. is lithium perchlorate (LiClO₄), which at concentrations of 1%-3% increase the mean pore size and narrow the pore size distribution. However, they demonstrated that increasing the concentration of LiClO₄ above 3% lead to the formation of macrovoids when using N,N-Dimethylacetamide as solvent³². They concluded that the changes in morphology rose from a reduction in nonsolvent tolerance upon the addition of the salt leading to faster phase separation. In addition to the ionic additives, several small molecules have also been used as pore forming additives. One such example is glycerol, which was shown by Shih et al. to increase the mean pore size of PVDF membranes with increasing concentration³³.

The influence of additives in the second category changes both with polymer chemistry and molecular weight. Typically, the higher MW polymers are trapped in the membrane during phase inversion, while the lower MW polymers are able to diffuse out. The entrapment of the higher MW polymers influences the flux of nonsolvent into the membrane, thereby changing the morphology. Wang et al. demonstrated this phenomenon by comparing membranes prepared using the same concentrations of polyvinyl pyrrolidone (PVP) at molecular weights of 10 kg/mol and 340 kg/mol³⁴. The higher molecular weight PVP produced a membrane with a thicker skin layer and larger pores. The thicker skin layer was ascribed to the entrapped hydrophilic PVP polymers facilitating faster diffusion of water into the casting solution. They also investigated the influence of different concentrations of low MW PVP in the range of 2% and 5%, but did not observe a noticeable change in morphology.

The MMMs produced using inorganic functional particles have been demonstrated to improve performance with minimal changes to the physical structure of the membrane^{24,35-37}. Work reported by Cao et al. added <2 wt. % TiO₂ nanoparticles to the casting solution. The resulting mixed-matrix membrane demonstrated an improved water flux and fouling resistance, with a minor change in pore size determined by the size of the TiO₂ nanoparticle³⁷. Another study demonstrated the incorporation of silica particles into the casting solution, which increased the viscosity of the casting solution enabling the formation of membranes with lower polymer concentrations. Membranes prepared using silica demonstrated comparable water flux and improved retention of Dextran 40k, with no significant changes in membrane morphology reported³⁵. In an investigation conducted by Yan et al., MMMs were fabricated through the inclusion of nano-sized alumina (Al₂O₃) particles. Membranes with a concentration of 2 wt % alumina particles showed improvements to both fouling resistance and tensile strength with no observable changes to membrane morphology or pore size.

Although the work discussed in the preceding paragraph focuses on several different types of particles, all of them are at low particle loadings 5 wt. %) and only use water as the nonsolvent. At these particle loadings there are negligible changes to the membrane structure due to the low concentration of functional particles having a minimal impact on the interactions of the NIPS process.

1.3 Membrane Chromatography

Chromatographic materials are distinguished by their separation chemistries, which belong to one of three classes: affinity, ion-exchange (IEX), and hydrophobic interaction & reverse phase (HI & RP). This work will generally focus more on IEX chromatography, which is further divided into cation exchange (CEX) and anion exchange (AEX) chromatography; however other types of chromatography can also be suitably employed. The binding behavior of IEX materials are characterized using a variety of well documented model proteins including: bovine serum albumin (BSA), lysozyme, myoglobin, ovalbumin, and conalbumins.

1.3.1 Resin and Membrane Protein Chromatography

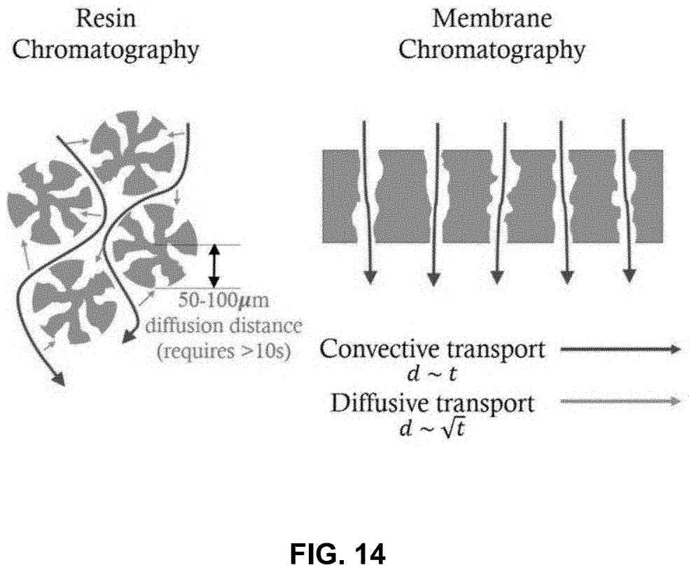

Prior to the late 1990s, the gold standard in high-resolution protein separation and analysis was resin-based packed beds⁵. Although packed beds demonstrate excellent selectivity, they also suffer from a few major limitations that make scale-up of chromatographic processes challenging. First, packed beds rely on diffusive mass transport to bring the solute in contact with the binding sites within the resin pores as seen in FIG. 14. Transport via diffusion is quite slow and necessitates longer processing times to fully use the bed's binding capabilities. Second, the pressure drop across packed beds is frequently high and often increases during operation as the functional media deforms and induces bed consolidation³⁸. Third, defects in the packing of the bed—such as cracking—produce flow passages that lead to channeling of the material flow resulting in poor bed utilization. Some research has been done to investigate the use of rigid, monodisperse, nonporous media in packed beds to address the drawbacks identified here⁵. Although the newer media does resolve several of the limitations of packed beds, it retains the high pressure drop across the column while also showing a reduction in protein binding capacity³⁹.

The leading method to circumvent the limitations outlined above is the use of microporous membranes as the base of the chromatographic material^{5,7,38,40}. Membrane-based chromatography relies primarily on convective mass transport, FIG. 14, to convey molecules of interest to available binding sites. The reliance on convection enables faster processing time and decouples operating flow rate and binding capacity. The use of microporous membranes also reduces the pressure drop across the column as the total membrane volume may be spread out over a large area with a small thickness—while still maintaining uniform fluid flow. Membrane adsorbers also have the added advantage of frequently being faster and cheaper to produce. Although the use of microporous membranes in protein separations have addressed the limitations of packed beds, they have also introduced a new set of drawbacks related to binding capacity. Due to the fluid flow being restricted to the pores of the membrane, the only surfaces available to interact with the solutes are the pore walls. The limited surface area leads to a reduction in the binding capacity, similar to what was seen when using nonporous media in packed beds^{5,39}. Membrane-based chromatography was also shown to lose its binding efficacy at lower salt concentrations in comparison to resin-based systems. Sections 1.3.2 and 1.3.3 summarize the advances in membrane chromatography related to increasing binding capacity and improving salt tolerance respectively.

1.3.2 Efforts to Increase Binding Capacity

Recent work on improving the binding capacity of membrane chromatography materials has focused on overcoming the limited pore surface area through functionalization of the porous support with polymer chains, polymer electrolytes, or polymer brushes⁴¹⁻⁴³. The added polymers extend into the protein solution and enable the formation of a 3-dimensional ‘scaffold’ that facilitates protein adsorption, thereby increasing the binding capacity of the membrane⁷. To further increase the density of available binding sites typically can be achieved by tailoring of the various functionalization methods to maximize the density of available binding sites. For example, a study by Bhut et al. functionalized the surface of regenerated cellulose membranes using surface-initiated atom transfer radical polymerization (ATRP) of 2-(dimethylamino)ethyl methacrylate (DMAEMA). The density and MW of the resulting poly(DMAEMA) chains were controlled independently using the initiator grafting density and polymerization time respectively⁷. The binding behavior of the functionalized membranes was investigated using BSA. They demonstrated that, at short polymerization times and low initiator grafting densities, the modified membranes had low binding capacities (˜20 mg/mL). As the polymerization time and/or the initiator grafting densities increased, the static binding capacities increased as well until reaching a plateau at ˜140 mg/mL. Furthermore, surface initiated ATRP has been shown to be a versatile method that is effective in functionalizing porous ceramic membranes in work by Sun et al. The resulting affinity chromatography membrane was reported to have a static BSA binding capacity of 150 mg/mL⁴⁰.

Surface initiated graft copolymerization, a derivative of UV-initiated graft copolymerization, is an alternative method used by Ulbricht and coworkers to functionalize the pore surface of polypropylene membranes for protein bining⁴¹. They used benzophenone (BP) as the initiator and acrylic acid (AA) as the functional monomer and investigated copolymerizations with acryl amide (AAm). The resulting CEX membranes were characterized using lysozyme as the model protein. The highest dynamic lysozyme binding of 20 mg/mL was observed in the membrane with the highest grafting density of acrylic acid.

In addition to the standard polymerization techniques discussed above, there have been other methods developed to improve the functionality of the pore surface. Work by Nova et al. reported the development of a chitosan/ceramic hybrid membrane for affinity chromatography⁴². The hybrid was fabricated by deposition of chitosan onto a ceramic support followed by a crosslinking reaction to immobilize the chitosan layer. The chitosan was then further functionalized with iminodiacetic acid, a carboxylic ligand that binds Cu²⁺. The hybrid demonstrated a BSA binding capacity more than double that of the ceramic support alone. Liu et al. investigated the fabrication of IEX membranes using layer-by-layer deposition of polyelectrolytes onto a porous regenerated cellulose support⁴³. They reported an increase from 11 mg/mL to 16 mg/mL in the dynamic lysozyme binding capacity as the number of polyelectrolyte layers was increased from 3 to 7. They also noted that using polyelectrolyte layers led to a higher permeability than commercially available membranes.

1.3.3 Efforts to Improve Salt Tolerance

The ligands used in IEX chromatography are often classified into strong and weak ion exchangers. A strong IEX has the same charge over the 0-14 pH range, with strong anion exchangers (such as quaternary amines) being positively charged and strong cation exchangers (such as sulfonates and sulfopropyls) being negatively charged. In contrast, weak ion exchangers are pH dependent and only demonstrate optimal performance over a small pH range. Weak anion exchangers (such as primary and secondary amines) begin to lose their ionization above a pH of 9, while weak cation exchangers (such as carboxymethyl) perform poorly below a pH of 6.

For many years, strong ion exchangers were the recommended functional groups for both resin and membrane chromatography because their electrostatic charges were not dependent on pH. However, one of the consistent shortcomings of strong IEX chemistries was their sensitivity to salt in the protein solution. This limitation was illustrated in a study by Fischer-Frühholz and coworkers, which revealed that adding 150 mM of NaCl reduced the binding capacity of a strong AEX membrane by 90%⁴⁴. The sharp drop in binding capacity in the presence of salt motivated the inclusion of costly buffer exchange steps in commercial protein separations. Removing the buffer exchange steps would require a membrane that demonstrated consistent binding capacities across a range of solution conductivities. In the same study, Fischer-Frühholz and coworkers demonstrated that using a weak anion exchange ligand comprising mostly primary amines on the same porous support enabled consistent binding at both 0 mM and 150 mM added NaCl corresponding to conductivities of 1.8 mS/cm and 16.8 mS/cm respectively⁴⁴.

Work by Riordan et al. screened several ligands as alternatives to quaternary amines in strong AEX membranes⁴⁵. They reported four ligands that demonstrated better salt tolerance than the quaternary amine ligand they used as a control. They concluded that ligand performance was determined by three factors: the ligand density of the membrane, the net charge of the ligand molecule, and the molecular structure of the ligand with an emphasis on the presence of available hydrogens. The third factor was shown to be critical in achieving high salt tolerance by testing derivatives of the four ligands that replaced the hydrogens on primary amines with methyl groups. The derivatives had a reduced salt tolerance, with the extent of the reduction depending on the number of primary amine hydrogens that were replaced. It was determined that primary and, to a lesser extent, secondary amines are able to interact with the solutes using both electrostatic interactions and hydrogen bonding; whereas, both quaternary and tertiary amines are only able to interact via electrostatic interactions⁴⁵. Therefore, as the concentration of salt goes up, the electrostatic interactions are screened leading to poor binding capacities of quaternary and tertiary amines. In contrast, the primary and secondary amines are still able to effectively bind proteins through hydrogen bonding over a range of salt concentrations.

1.4 Size-Based Separations

1.4.1 Size Based Separation Membranes

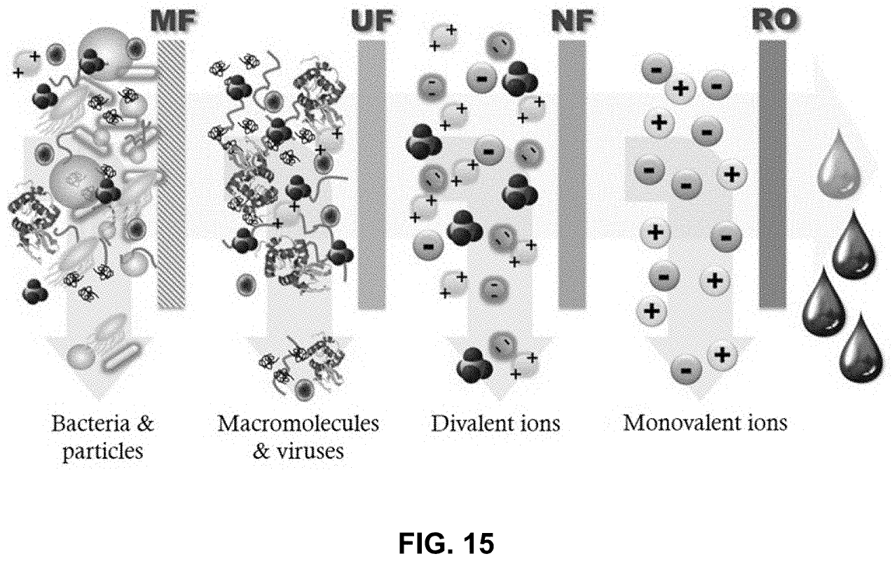

A classic example of size separation using membrane technology is water purification, as demonstrated in FIG. 15. Membrane materials in the microfiltration (MF), ultrafiltration (UF), and nanofiltration (NF) regimes operate at least partially on a basis of rejecting particles that are too large to pass through the membrane pores⁴⁶. However, one of the major drawbacks of size separations using membrane technology is fouling, the process of unwanted material building up on the membrane surface²³. Fouling reduces membrane performance and can even lead to membrane failure if not treated properly. Furthermore, fouling becomes an increasingly difficult problem to address as the solution being separated becomes more complex. While much of the prior literature in membrane science focuses on designing membranes with improved anti-fouling capabilities^{21,23,47,48,} recent work in microfluidics has revealed several techniques to avoid fouling altogether and still achieve high efficiency size based separation⁴⁹⁻⁵¹.

1.4.2 Size Based Separations Using Inertial Microfluidics

Although microfluidic devices are not membranes, they are discussed here due to the relevance of their applications in size based separations in complex fluids. In a review article by Professor Di Carlo, he summarizes two effects of inertial microfluidics that may be used in size based separations⁵¹. The first is secondary flows (also called Dean flow or dean vortices) in curved channels which arise from a velocity mismatch between fluid in the center and near-wall regions in the downstream direction. Due to the velocity difference, the fluid elements near the channel centerline have greater inertia than the fluid near the walls and tend to flow outward. The outward movement creates a pressure gradient in the radial direction of the curved channel. However, the channel is enclosed and so the fluid near the walls is re-circulated inwards by the pressure gradient resulting in two symmetric vortices⁵¹. Work by Seo et al. demonstrated the efficacy of using Dean flow to separate particles from a mixture containing 10 μm and 6 μm particles into two outlet streams, with an over 80% efficiency for both particle sizes. When the experiment was repeated using a mixture of 10 μm and 3 μm particles, the 3 μm particles were evenly distributed between the two outlet streams indicating that this separation method is only effective for larger particles⁵². Another study by Warkiani et al. used dean vortices to isolate circulating tumor cells and achieved a capture efficiency of 80%, while significantly reducing the concentrations of unwanted cells (white blood cells).

The second effect discussed by Professor Di Carlo is inertial migration of particles, wherein particles in flow within a bounded channel experience a lift force from the fluid shear gradient as well as a lift force from the wall⁵¹. These forces move the particles across undisturbed streamlines until the particles reach an ‘equilibrium’ position where the two forces are equal. A study by Che et al. demonstrated selective capture of cancer cells using inertial migration of particles in a straight channel followed by the fluid flowing past a reservoir with vortices⁵³. An imbalance between the wall lift and shear lift forces that scales with particle diameter led to the larger cancer cells being captured by the vortices while the smaller white and red blood cells are allowed to flow past. Their technique demonstrated a capture efficiency of 83% at a processing speed of 800 μL/min whole blood⁵³. The formation of vortices by fluid flowing past a reservoir or cavity will be discussed further in the next section.

1.4.3 Vortices in Confined Cavities

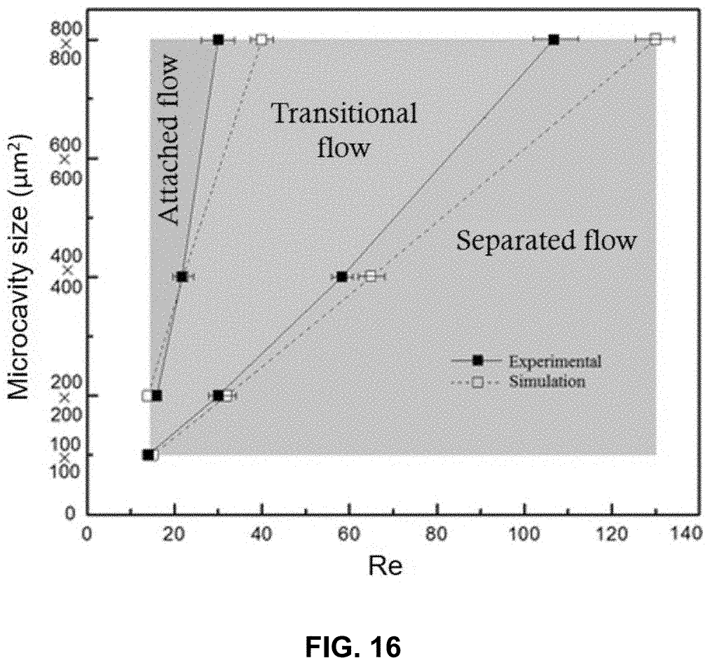

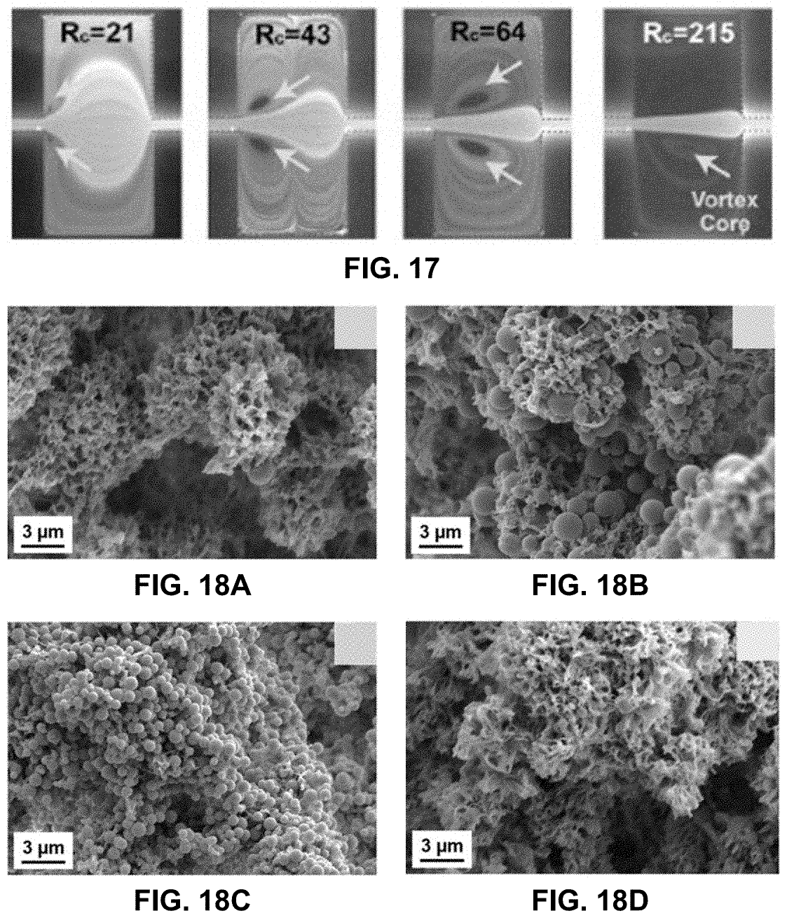

Prior literature has shown that when a fluid flowing in a channel with finite inertia encounters a microcavity (a bounded volume with cross-sectional dimension larger than the channel) a region of recirculating flow may form dependent on the fluid inertia represented by the Reynolds number (Re)^{54,55}. As seen in FIG. 16, at very low Re the fluid is in a regime called attached flow where there is no recirculation in the microcavity. As Re increases, the fluid encounters a transitory regime where the recirculating flow is not fully formed. Once Re passes a critical value, determined by the dimensions of the cavity, the fluid is in a separated flow regime and the microcavity shows signs of recirculating flow. As Re continues to increase the recirculating flow will eventually expand to fill the entire microcavity (FIG. 17)⁵⁶. Microvortices have been shown to be a versatile and powerful tool in the literature⁵⁵ and it is important to understand the conditions required to form them in novel geometries, such as a dendritic ceramic membrane.

1.5 One Aspect of the Focus of this Work

1.5.1 Influence of In Situ Generated Microparticles and Nonsolvent on Membrane Morphology

In the literature, investigation of the influence of incorporated functional materials on MMM structure has been limited to low particle concentrations and/or using water as the only nonsolvent^{24,35-37}. Due to their low concentration in these studies, the functional particles have little influence on the phase inversion process as seen by the minimal changes to membrane structure. However, the low concentrations are necessary to avoid particle aggregation during casting and leaching during operation³⁷. Similarly, if a softer nonsolvent than water was used, the functional materials would not be as tightly entrapped within the polymer matrix leading to leaching and loss of membrane functionality. In Example 8, we use a promising strategy for stably incorporating functional polymer particles in a structural polymer matrix to investigate a wider range of functional particle loadings (6 wt %-60 wt %). Furthermore, with the functional particles being stably incorporated into the polymer matrix we are able to use different nonsolvents to help unravel the interactions between the solvent, structural polymer, functional particles, and nonsolvent that govern phase separation and subsequent membrane morphology.

1.5.2 Mixed-Matrix Membrane Chromatography

Prior to this work, the majority of published studies improved binding capacity by adding functional polymers to the porous support surface^{7,40,41}. An alternative method that has received limited attention is to incorporate materials with IEX capabilities into mixed-matrix membranes⁵⁷. Using mixed-matrix membranes with in situ generated functional particles provides several advantages over pore surface functionalization including: maintaining polymer matrix integrity, even distribution of functional material, and inherent 3-dimensional binding of proteins. Furthermore, the functional material may be tailored to include a higher concentration of primary and secondary amines, thereby improving the salt tolerance of the resulting mixed-matrix membrane adsorber^{44,45}. In this thesis we investigate the design and fabrication of novel AEX membrane adsorbers through modifying MMMs (Example 9) and developing ceramic-MMM composites (Example 10).

1.5.3 Size Based Isolation of Bacteria Using Dendritic Ceramics

The rapid isolation and detection of pathogens from blood has received increased attention in the literature over the past two decades due to rising rates of sepsis and antibiotic resistance^{56,58,59}. In recent years, the speed of pathogen detection and identification has been improved by several advances in digital quantitative detection^{60,61}. In regards to isolation of pathogens from complex fluids, prior literature has demonstrated a variety of microfluidic based techniques for size based separation of pathogens^{52,56,58,59}. However, many of these techniques operate at low flow rates and have limited scalability. Thus, the field is still in need of a fast and scalable method to isolate and then concentrate pathogens prior to detection.

### References Corresponding to Example 7

(1) Geise, G. M.; Lee, H.-S.; Miller, D. J.; Freeman, B. D.; McGrath, J. E.; Paul, D. R. Water Purification by Membranes: The Role of Polymer Science. Journal of Polymer Science Part B: Polymer Physics 2010, 48 (15), 1685-1718. https://doi.org/10.1002/polb.22037.
(2) Geise, G. M.; Paul, D. R.; Freeman, B. D. Fundamental Water and Salt Transport Properties of Polymeric Materials. Progress in Polymer Science 2014, 39 (1), 1-42. https://doi.org/10.1016/j.progpolymsci.2013.07.001.
(3) Kotte, M. R.; Cho, M.; Diallo, M. S. A Facile Route to the Preparation of Mixed Matrix Polyvinylidene Fluoride Membranes with In-Situ Generated Polyethyleneimine Particles. Journal of Membrane Science 2014, 450, 93-102. https://doi.org/10.1016/j.memsci.2013.08.025.
(4) Werber, J. R.; Osuji, C. O.; Elimelech, M. Materials for Next-Generation Desalination and Water Purification Membranes. Nature Reviews Materials 2016, 1 (5), 1-15.
(5) Ghosh, R. Protein Separation Using Membrane Chromatography: Opportunities and Challenges. Journal of Chromatography A 2002, 952 (1), 13-27. https://doi.org/10.1016/S0021-9673(02)00057-2.
(6) Chen, G.; Umatheva, U.; Alforque, L.; Shirataki, H.; Ogawa, S.; Kato, C.; Ghosh, R. An Annular-Flow, Hollow-Fiber Membrane Chromatography Device for Fast, High-Resolution Protein Separation at Low Pressure. Journal of Membrane Science 2019, 590, 117305. https://doi.org/10.1016/j.memsci.2019.117305.
(7) Bhut, B. V.; Husson, S. M. Dramatic Performance Improvement of Weak Anion-Exchange Membranes for Chromatographic Bioseparations. Journal of Membrane Science 2009, 337 (1), 215-223. https://doi.org/10.1016/j.memsci.2009.03.046.
(8) Kotte, M. R.; Kuvarega, A. T.; Talapaneni, S. N.; Cho, M.; Coskun, A.; Diallo, M. S. A Facile and Scalable Route to the Preparation of Catalytic Membranes with in Situ Synthesized Supramolecular Dendrimer Particle Hosts for Pt(0) Nanoparticles Using a Low-Generation PAMAM Dendrimer (G1-NH 2) as Precursor. ACS Appl. Mater. Interfaces 2018, 10 (39), 33238-33251. https://doi.org/10.1021/acsami.8b11351.
(9) Ghosh, S. K.; Alam, Md. M.; Mandal, D. The in Situ Formation of Platinum Nanoparticles and Their Catalytic Role in Electroactive Phase Formation in Poly(Vinylidene Fluoride): A Simple Preparation of Multifunctional Poly(Vinylidene Fluoride) Films Doped with Platinum Nanoparticles. RSC Adv. 2014, 4 (79), 41886-41894. https://doi.org/10.1039/C4RA06334A.
(10) Kotte, M. R.; Kuvarega, A. T.; Cho, M.; Mamba, B. B.; Diallo, Mamadou. S. Mixed Matrix PVDF Membranes With in Situ Synthesized PAMAM Dendrimer-Like Particles: A New Class of Sorbents for Cu(II) Recovery from Aqueous Solutions by Ultrafiltration. Environ. Sci. Technol. 2015, 49 (16), 9431-9442. https://doi.org/10.1021/acs.est.5b01594.
(11) Park, M. J.; Nisola, G. M.; Vivas, E. L.; Limjuco, L. A.; Lawagon, C. P.; Seo, J. G.; Kim, H.; Shon, H. K.; Chung, W.-J. Mixed Matrix Nanofiber as a Flow-through Membrane Adsorber for Continuous Li+ Recovery from Seawater. Journal of Membrane Science 2016, 510, 141-154. https://doi.org/10.1016/j.memsci.2016.02.062.
(12) Abed, M.; Reza, M. Poly(Vinylidene Fluoride) (PVDF) Based Hydrophilic Hollow Fibre Membranes:Prospects for Wastewater Treatment. Ph.D., Imperial College London, 2012. https://doi.org/10.25560/9830.
(13) Baker, R. W. Membrane Technology and Applications, 3rd ed.; John Wiley & Sons: Chichester, West Sussex; Hoboken, 2012.
(14) Bottino, A.; Camera-Roda, G.; Capannelli, G.; Munari, S. The Formation of Microporous Polyvinylidene Difluoride Membranes by Phase Separation. Journal of membrane science 1991, 57 (1), 1-20.
(15) Zuo, D.; Zhu, B.; Cao, J.; Xu, Y. Influence of Alcohol-Based Nonsolvents on the Formation and Morphology of PVDF Membranes in Phase Inversion Process. Chinese journal of polymer science 2006, 24 (03), 281-289.
(16) Jung, J. T.; Kim, J. F.; Wang, H. H.; di Nicolo, E.; Drioli, E.; Lee, Y. M. Understanding the Non-Solvent Induced Phase Separation (NIPS) Effect during the Fabrication of Microporous PVDF Membranes via Thermally Induced Phase Separation (TIPS). Journal of Membrane Science 2016, 514, 250-263. https://doi.org/10.1016/j.memsci.2016.04.069.
(17) Sun, C. Poly(Vinylidene Fluoride) Membranes: Preparation, Modification, Characterization and Applications. 2009.
(18) Liu, F.; Hashim, N. A.; Liu, Y.; Abed, M. R. M.; Li, K. Progress in the Production and Modification of PVDF Membranes. Journal of Membrane Science 2011, 375 (1), 1-27. https://doi.org/10.1016/j.memsci.2011.03.014.
(19) Farahani, M. H. D. A.; Vatanpour, V. A Comprehensive Study on the Performance and Antifouling Enhancement of the PVDF Mixed Matrix Membranes by Embedding Different Nanoparticulates: Clay, Functionalized Carbon Nanotube, SiO2 and TiO2. Separation and Purification Technology 2018, 197, 372-381. https://doi.org/10.1016/j.seppur.2018.01.031.
(20) Fontananova, E.; Bahattab, M. A.; Aljlil, S. A.; Alowairdy, M.; Rinaldi, G.; Vuono, D.; Nagy, J. B.; Drioli, E.; Di Profio, G. From Hydrophobic to Hydrophilic Polyvinylidenefluoride (PVDF) Membranes by Gaining New Insight into Material's Properties. RSC Advances 2015, 5 (69), 56219-56231.
(21) Mi, Y.-F.; Xu, G.; Guo, Y.-S.; Wu, B.; An, Q.-F. Development of Antifouling Nanofiltration Membrane with Zwitterionic Functionalized Monomer for Efficient Dye/Salt Selective Separation. Journal of Membrane Science 2020, 601, 117795. https://doi.org/10.1016/j.memsci.2019.117795.
(22) Aroon, M. A.; Ismail, A. F.; Matsuura, T.; Montazer-Rahmati, M. M. Performance Studies of Mixed Matrix Membranes for Gas Separation: A Review. Separation and Purification Technology 2010, 75 (3), 229-242. https://doi.org/10.1016/j.seppur.2010.08.023.
(23) Li, X.; Sotto, A.; Li, J.; Van der Bruggen, B. Progress and Perspectives for Synthesis of Sustainable Antifouling Composite Membranes Containing in Situ Generated Nanoparticles. Journal of Membrane Science 2017, 524, 502-528. https://doi.org/10.1016/j.memsci.2016.11.040.
(24) Yan, L.; Li, Y. S.; Xiang, C. B. Preparation of Poly(Vinylidene Fluoride)(Pvdf) Ultrafiltration Membrane Modified by Nano-Sized Alumina (Al₂O₃) and Its Antifouling Research. Polymer 2005, 46 (18), 7701-7706. https://doi.org/10.1016/j.polymer.2005.05.155.
(25) Sorarù, G. D.; Modena, S.; Guadagnino, E.; Colombo, P.; Egan, J.; Pantano, C. Chemical Durability of Silicon Oxycarbide Glasses. Journal of the American Ceramic Society 2002, 85 (6), 1529-1536. https://doi.org/10.1111/j.1151-2916.2002.tb00308.x.
(26) Acres, R. G.; Ellis, A. V.; Alvino, J.; Lenahan, C. E.; Khodakov, D. A.; Metha, G. F.; Andersson, G. G. Molecular Structure of 3-Am inopropyltriethoxysilane Layers Formed on Silanol-Terminated Silicon Surfaces. J. Phys. Chem. C 2012, 116 (10), 6289-6297. https://doi.org/10.1021/jp212056s.
(27) Smith, K. Development of Membrane Processes. In Membrane Processing; Tamime, A. Y., Ed.; Blackwell Publishing Ltd.: Oxford, U K, 2012; pp 1-16. https://doi.org/10.1002/9781118457009.ch1.
(28) Stropnik, C.; Germič, L.; Žerjal, B. Morphology Variety and Formation Mechanisms of Polymeric Membranes Prepared by Wet Phase Inversion. Journal of Applied Polymer Science 1996, 61 (10), 1821-1830. https://doi.org/10.1002/(SICI)1097-4628(19960906)61:10<1821::AID-APP24>3.0.CO; 2-3.
(29) Yeow, M. L.; Liu, Y. T.; Li, K. Morphological Study of Poly(Vinylidene Fluoride) Asymmetric Membranes: Effects of the Solvent, Additive, and Dope Temperature. Journal of Applied Polymer Science 2004, 92 (3), 1782-1789. https://doi.org/10.1002/app.20141.
(30) Sukitpaneenit, P.; Chung, T.-S. Molecular Elucidation of Morphology and Mechanical Properties of PVDF Hollow Fiber Membranes from Aspects of Phase Inversion, Crystallization and Rheology. Journal of Membrane Science 2009, 340 (1), 192-205. https://doi.org/10.1016/j.memsci.2009.05.029.
(31) Bottino, A.; Capannelli, G.; Munari, S.; Turturro, A. High Performance Ultrafiltration Membranes Cast from LiCl Doped Solutions. Desalination 1988, 68 (2), 167-177. https://doi.org/10.1016/0011-9164(88)80052-3.
(32) Yeow, M. L.; Liu, Y.; Li, K. Preparation of Porous PVDF Hollow Fibre Membrane via a Phase Inversion Method Using Lithium Perchlorate (LiClO4) as an Additive. Journal of Membrane Science 2005, 258 (1), 16-22. https://doi.org/10.1016/j.memsci.2005.01.015.
(33) Shih, H. C.; Yeh, Y. S.; Yasuda, H. Morphology of Microporous Poly(Vinylidene Fluoride) Membranes Studied by Gas Permeation and Scanning Electron Microscopy. Journal of Membrane Science 1990, 50 (3), 299-317. https://doi.org/10.1016/S0376-7388(00)80627-4.
(34) Wang, D.; Li, K.; Teo, W. K. Preparation and Characterization of Polyvinylidene Fluoride (PVDF) Hollow Fiber Membranes. Journal of Membrane Science 1999, 163 (2), 211-220. https://doi.org/10.1016/S0376-7388(99)00181-7.
(35) Bottino, A.; Capannelli, G.; D′Asti, V.; Piaggio, P. Preparation and Properties of Novel Organic-Inorganic Porous Membranes. Separation and Purification Technology 2001, 22-23, 269-275. https://doi.org/10.1016/S1383-5866(00)00127-1.
(36) Bottino, A.; Capannelli, G.; Comite, A. Preparation and Characterization of Novel Porous PVDF-ZrO2 Composite Membranes. Desalination 2002, 146 (1), 35-40. https://doi.org/10.1016/S0011-9164(02)00469-1.
(37) Cao, X.; Ma, J.; Shi, X.; Ren, Z. Effect of TiO2 Nanoparticle Size on the Performance of PVDF Membrane. Applied Surface Science 2006, 253 (4), 2003-2010. https://doi.org/10.1016/j.apsusc.2006.03.090.
(38) Bhut, B. V.; Wickramasinghe, S. R.; Husson, S. M. Preparation of High-Capacity, Weak Anion-Exchange Membranes for Protein Separations Using Surface-Initiated Atom Transfer Radical Polymerization. Journal of Membrane Science 2008, 325 (1), 176-183. https://doi.org/10.1016/j.memsci.2008.07.028.
(39) Hashimoto, T. Non-Porous Hydrophilic Resin-Based Packings for the Separation of Biopolymers. Journal of Chromatography A 1991, 544, 257-265. https://doi.org/10.1016/S0021-9673(01)83990-X.
(40) Sun, L.; Dai, J.; Baker, G. L.; Bruening, M. L. High-Capacity, Protein-Binding Membranes Based on Polymer Brushes Grown in Porous Substrates. Chem. Mater. 2006, 18 (17), 4033-4039. https://doi.org/10.1021/cm060554m.
(41) Ulbricht, M.; Yang, H. Porous Polypropylene Membranes with Different Carboxyl Polymer Brush Layers for Reversible Protein Binding via Surface-Initiated Graft Copolymerization. Chem. Mater. 2005, 17 (10), 2622-2631. https://doi.org/10.1021/cm0485714.
(42) Nova, C. J. M.; Paolucci-Jeanjean, D.; Belleville, M.-P.; Barboiu, M.; Rivallin, M.; Rios, G. Elaboration, Characterization and Study of a New Hybrid Chitosan/Ceramic Membrane for Affinity Membrane Chromatography. Journal of Membrane Science 2008, 321 (1), 81-89. https://doi.org/10.1016/j.memsci.2007.12.002.
(43) Liu, G.; Dotzauer, D. M.; Bruening, M. L. Ion-Exchange Membranes Prepared Using Layer-by-Layer Polyelectrolyte Deposition. Journal of Membrane Science 2010, 354 (1), 198-205. https://doi.org/10.1016/j.memsci.2010.02.047.
(44) Fischer-Frühholz, S.; Zhou, D.; Hirai, M. Sartobind STIC® Salt-Tolerant Membrane Chromatography. Nature Methods 2010, 7 (12), 12-13.
(45) Riordan, W.; Heilmann, S.; Brorson, K.; Seshadri, K.; He, Y.; Etzel, M. Design of Salt-Tolerant Membrane Adsorbers for Viral Clearance. Biotechnology and Bioengineering 2009, 103 (5), 920-929. https://doi.org/10.1002/bit.22314.
(46) Read “Water Reuse: Potential for Expanding the Nation's Water Supply Through Reuse of Municipal Wastewater” at NAP.Edu. https://doi.org/10.17226/13303.
(47) Akthakul, A.; Salinaro, R. F.; Mayes, A. M. Antifouling Polymer Membranes with Subnanometer Size Selectivity. Macromolecules 2004, 37 (20), 7663-7668. https://doi.org/10.1021/ma048837s.
(48) Su, Y. P.; Sim, L. N.; Li, X.; Coster, H. G. L.; Chong, T. H. Anti-Fouling Piezoelectric PVDF Membrane: Effect of Morphology on Dielectric and Piezoelectric Properties. Journal of Membrane Science 2021, 620, 118818. https://doi.org/10.1016/j.memsci.2020.118818.
(49) Ebrahimi Warkiani, M.; Guan, G.; Bee Luan, K.; Cheng Lee, W.; S. Bhagat, A. A.; Chaudhuri, P. K.; Shao-Weng Tan, D.; Teck Lim, W.; Chin Lee, S.; Y. Chen, P. C.; Teck Lim, C.; Han, J. Slanted Spiral Microfluidics for the Ultra-Fast, Label-Free Isolation of Circulating Tumor Cells. Lab on a Chip 2014, 14 (1), 128-137. https://doi.org/10.1039/C3LC50617G.
(50) Wu, Z.; Willing, B.; Bjerketorp, J.; K. Jansson, J.; Hjort, K. Soft Inertial Microfluidics for High Throughput Separation of Bacteria from Human Blood Cells. Lab on a Chip 2009, 9 (9), 1193-1199. https://doi.org/10.1039/B817611F.
(51) Carlo, D. D. Inertial Microfluidics. Lab on a Chip 2009, 9 (21), 3038-3046. https://doi.org/10.1039/B912547G.
(52) Seo, J.; Lean, M. H.; Kole, A. Membraneless Microseparation by Asymmetry in Curvilinear Laminar Flows. Journal of Chromatography A 2007, 1162 (2), 126-131. https://doi.org/10.1016/j.chroma.2007.05.110.
(53) Che, J.; Yu, V.; Dhar, M.; Renier, C.; Matsumoto, M.; Heirich, K.; Garon, E. B.; Goldman, J.; Rao, J.; Sledge, G. W.; Pegram, M. D.; Sheth, S.; Jeffrey, S. S.; Kulkarni, R. P.; Sollier, E.; Di Carlo, D. Classification of Large Circulating Tumor Cells Isolated with Ultra-High Throughput Microfluidic Vortex Technology. Oncotarget 2016, 7 (11), 12748-12760. https://doi.org/10.18632/oncotarget.7220.
(54) Khojah, R.; Lo, D.; Tang, F.; Di Carlo, D. The Evolution of Flow and Mass Transport in 3D Confined Cavities. arXiv:2101.07242 [physics] 2021.
(55) Shen, F.; Xu, M.; Zhou, B.; Wang, Z.; Liu, Z. Effects of Geometry Factors on Microvortices Evolution in Confined Square Microcavities. Microfluid Nanofluid 2018, 22 (4), 36. https://doi.org/10.1007/s10404-018-2056-2.
(56) Hur, S. C.; Mach, A. J.; Di Carlo, D. High-Throughput Size-Based Rare Cell Enrichment Using Microscale Vortices. Biomicrofluidics 2011, 5 (2), 022206. https://doi.org/10.1063/1.3576780.
(57) Avramescu, M.-E.; Borneman, Z.; Wessling, M. Mixed-Matrix Membrane Adsorbers for Protein Separation. Journal of Chromatography A 2003, 1006 (1), 171-183. https://doi.org/10.1016/S0021-9673(03)00562-4.
(58) Ohlsson, P.; Evander, M.; Petersson, K.; Mellhammar, L.; Lehmusvuori, A.; Karhunen, U.; Soikkeli, M.; Seppä, T.; Tuunainen, E.; Spangar, A.; von Lode, P.; Rantakokko-Jalava, K.; Otto, G.; Scheding, S.; Soukka, T.; Wittfooth, S.; Laurell, T. Integrated Acoustic Separation, Enrichment, and Microchip Polymerase Chain Reaction Detection of Bacteria from Blood for Rapid Sepsis Diagnostics. Anal. Chem. 2016, 88 (19), 9403-9411. https://doi.org/10.1021/acs.analchem.6b00323.
(59) Faridi, M. A.; Ramachandraiah, H.; Banerjee, I.; Ardabili, S.; Zelenin, S.; Russom, A. Elasto-Inertial Microfluidics for Bacteria Separation from Whole Blood for Sepsis Diagnostics. J Nanobiotechnol 2017, 15 (1), 3. https://doi.org/10.1186/s12951-016-0235-4.
(60) Schoepp, N. G.; Khorosheva, E. M.; Schlappi, T. S.; Curtis, M. S.; Humphries, R. M.; Hindler, J. A.; Ismagilov, R. F. Digital Quantification of DNA Replication and Chromosome Segregation Enables Determination of Antimicrobial Susceptibility after Only 15 Minutes of Antibiotic Exposure. Angewandte Chemie International Edition 2016, 55 (33), 9557-9561. https://doi.org/10.1002/anie.201602763.
(61) Schlappi, T. S.; McCalla, S. E.; Schoepp, N. G.; Ismagilov, R. F. Flow-through Capture and in Situ Amplification Can Enable Rapid Detection of a Few Single Molecules of Nucleic Acids from Several Milliliters of Solution. Anal. Chem. 2016, 88 (15), 7647-7653. https://doi.org/10.1021/acs.analchem.6b01485.

### Example 8: Influence of Nonsolvent and Mixed-Matrix Composition on Membrane Morphology

2.1 Introduction

Mixed-matrix membranes (MMM) are a versatile class of membranes that combine the structural and flow properties of polymeric membranes with the functionality of a separate material dispersed in the polymer matrix. The improved functionality enables MMMs in academic studies to surpass performance of neat polymeric membranes in several fields including gas separations¹⁻⁴, water purification⁵⁻¹¹, catalysis¹²⁻¹⁴, and resource recovery¹⁵⁻¹⁷. One complication associated with this type of multicomponent membrane is the behavior of the functional particles when they are not properly incorporated into the polymer matrix. Insufficiently integrating the functional material into the polymer matrix frequently leads to an inhomogeneous distribution and in extreme cases complete expulsion of the particles from the membrane during processing. Various methods have been developed to resolve this issue for both ex situ and in situ generated functional particles providing several routes to a homogeneous distribution throughout the membrane^{11,15,18}.

To capitalize upon the benefits of MMMs, it is advantageous to control and tailor membrane morphology to meet requirements of the end application^{1,10}. For example, polyvinylidene fluoride (PVDF) is a commonly used polymer in many membrane applications due to its excellent chemical robustness, mechanical strength, and electrical properties¹⁹. The chemical robustness is an inherent property of PVDF and is therefore consistent for any PVDF membrane that has not been chemically modified. In contrast, both the mechanical strength and piezoelectric character depend on the morphology of the membrane. In the case of mechanical strength, features such as macrovoids and fingerlike pores are detrimental to the membrane's mechanical properties. As a result, suppressing the formation of these features is an integral part of membrane fabrication for applications that operate at higher pressures such as water purification¹⁸⁻²⁰. In other applications, the porosity of these features are beneficial and outweigh the detriment to the mechanical properties leading to the development of methods that promote their formation²¹. PVDF's piezoelectric character depends on both the microscopic morphology and the crystal phase, with the β-phase being electroactive and the α being electrically inert. To optimize the piezoelectric behavior, it is advantageous to increase the concentration of β-phase PVDF and align the electrical dipoles across the membrane often done through a process known as poling. Whether it be to optimize mechanical strength or electrical performance, having the capability to tailor and control membrane morphology is essential.

Morphology control in polymeric membranes, including the degree of crystallinity, crystalline phase, and pore structure, is achieved by manipulating the kinetic trapping of a partially-phase separated state. For example, using different nonsolvents to drive liquid-liquid and solid-liquid demixing during Nonsolvent Induced Phase Separation (NIPS) enables the formation of distinct morphologies^{5,22-25}. In MMMs the presence of the functional particles increases the complexity of the phase separation process by adding new interactions with the solvent, nonsolvent, and structural polymer^{11,16}.

Here, we use a method pioneered by Professor Diallo to stably incorporate functional polymer particles in a structural polymer matrix and investigate the role of the particles and nonsolvent chemistry during NIPS. The interplay of functional polymer particle loading and nonsolvent induced phase separation are examined using x-ray diffraction (to deduce the crystal phase adopted by polyvinylidene difluoride, PVDF) and scanning electron microscopy (to observe membrane morphology and the size and distribution of functional particles). We discover that the interaction between nonsolvent and functional particles enables a shift in crystal phase and the formation of a unique morphology usually not attainable with our solvent.

2.2 Experimental Methods

2.2.1 Materials

Polyvinylidene Fluoride (PVDF; Kynar 761, 400 kg/mol) was donated by Arkema. Hyperbranched polyethylenimine (PEI; 600 g/mol) was purchased from Polysciences. The following chemicals were purchased from Sigma Aldrich: Epichlorohydrin (ECH), Isopropanol (IPA), Triethyl phosphate (TEP), and N-methylpyrrolidone (NMP). Nonwoven PET support was purchased from Hollytex. All chemicals were used as received.

2.2.2 Membrane Synthesis

To begin a typical membrane synthesis, 5.91 g of PVDF was added to an empty 3-neck flask. The flask was then outfitted with an overhead mechanical stirrer and the necessary greased connectors. Next, 30 mL of TEP was added to the flask and then the remaining openings were sealed using rubber septa. The PVDF/TEP mixture was heated to 80° C. for an hour with no mixing before the mixing speed was set to 60 rpm. The resulting solution was mixed overnight at 60 rpm and 80° C. During the heating of the PVDF/TEP mixture, the PEI/TEP solution was prepared by adding the desired mass of PEI (Table 4) to a scintillation vial followed by 5 mL of TEP. The mixture was vortexed until the solution was homogeneous and clear, and then it was left to equilibrate overnight at room temperature.

TABLE 4
Membrane formulation for different PEI loadings

| Formulation | PVDF (g) | PEI (g) | TEP (mL) | ECH (mL) |
|---|---|---|---|---|
| Neat | 5.66 | — | 30 | — |
| 06 | 5.91 | 0.26 | 35 | 0.14 |
| 21 | 5.91 | 1.1 | 35 | 0.60 |
| 38 | 5.91 | 2.6 | 35 | 1.4 |
| 48 | 5.91 | 3.9 | 35 | 2.1 |
| 54 | 5.91 | 5.0 | 35 | 2.7 |
| 60 | 5.91 | 6.5 | 35 | 3.5 |

Once the solutions were equilibrated, the flask was purged with N₂ for 7 minutes, and the mixing speed was increased to 250 rpm. With the N₂ flow still on, the PEI solution was added dropwise to the flask using a glass Pasteur pipette over the course of 4 minutes. The resulting solution was left to mix for 5 minutes before adding 17 drops of concentrated HCl (37% solution), after which the solution turned cloudy. Following the addition of the HCl, the flask was incubated for 15 minutes at 80° C. with the mixing speed maintained at 250 rpm. The required amount of ECH (Table 4) was then added to the flask, the N₂ flow was turned off, and the polymerization reaction was allowed to proceed for 4 hours.

After the 4-hour reaction time, the flask was put under in-house vacuum for 10 minutes to remove entrapped gas. The dope solution was then cast either on glass to prepare samples for structural characterization (SEM and x-ray scattering) or on a nonwoven PET support for transport measurements. The mixture was spread uniformly using a doctor blade with a blade height of 300 μm. The cast mixture was left at room temperature for 30 seconds before immersion into a coagulation bath at room temperature. The coagulation bath comprised one of the following: distilled water, Isopropanol, or 50 v % N-methylpyrrolidone solution in water (abbreviated as NMP:H₂O here after). After two hours, the solidified membranes were moved to distilled water baths prior to storage in distilled water.

2.2.3 SEM Characterization

The membrane top surface and cross-section were imaged using a Field Emission Scanning Electron Microscope (FE SEM-Zeiss 1550 VP). In preparation for imaging, the membrane samples were first dried at room temperature for 24 hours. Next, the samples were dried under house vacuum for 24 hours. To prepare the cross-section view, the chosen samples were immersed in liquid nitrogen for 2 minutes and then fractured. All samples were then coated with a Pt/Pd conductive layer on the surface of interest prior to imaging. The resulting micrographs were used to characterize sample morphology and, for cross-sections, estimate sample thickness. Mean particle size and particle size distribution of each condition was then determined by measuring the diameter of 100 particles in the cross-section images.

2.2.4 X-Ray Scattering

X-ray scattering measurements were performed at beamline 5-ID-D of the Advanced Photon Source at Argonne National Laboratory. The beamline collects both wide-angle x-ray scattering (WAXS) and small-angle x-ray scattering (SAXS) patterns simultaneously. The optimum exposure time for the samples scanned being 0.5 s and 0.005s, respectively. The membrane samples were cut into coupons approximately 10 mm×10 mm and mounted onto a backing board in preparation for the measurements, five point on each sample. The first measurement near the center of the sample, and the next four at points on a circle of radius 2.5 mm about the center in 90° increments, moving clockwise. Background scans as empty sample openings were taken at regular intervals.

2.2.5 Water Flux Measurements

Samples for flux measurements were prepared by cutting a 45 mm×90 mm rectangular coupon from a membrane cast on the nonwoven PET support. The samples were then loaded into a cross-flow filtration chamber with an active area of 18.75 cm². The membranes were conditioned for 90 minutes at a pressure of 3 bar and a cross-flow rate of 1.7 L/min using distilled water to permit any compaction to occur and stabilize prior to measurement. Following membrane compaction, the operating pressure was changed to 2 bar while the cross-flow rate was maintained constant. The permeate mass was measured every 5 minutes for 90 minutes, and recorded values were used to calculate membrane flux. All samples were tested using distilled water as feed.

2.3 Results and Discussion

2.3.1 Morphological Characteristics Observed in SEM

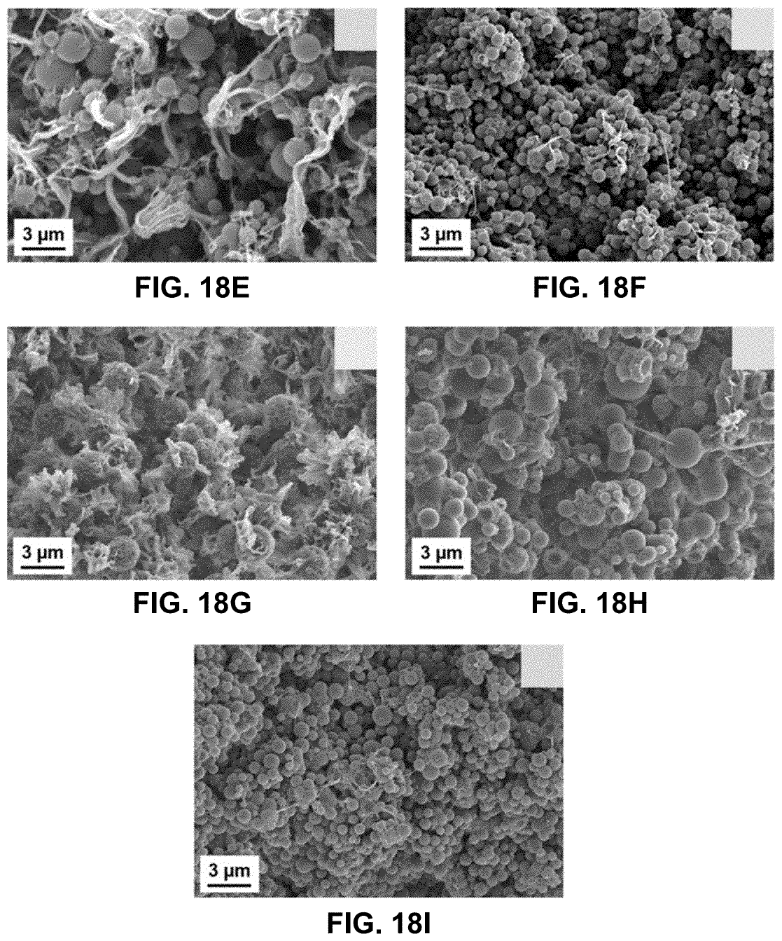

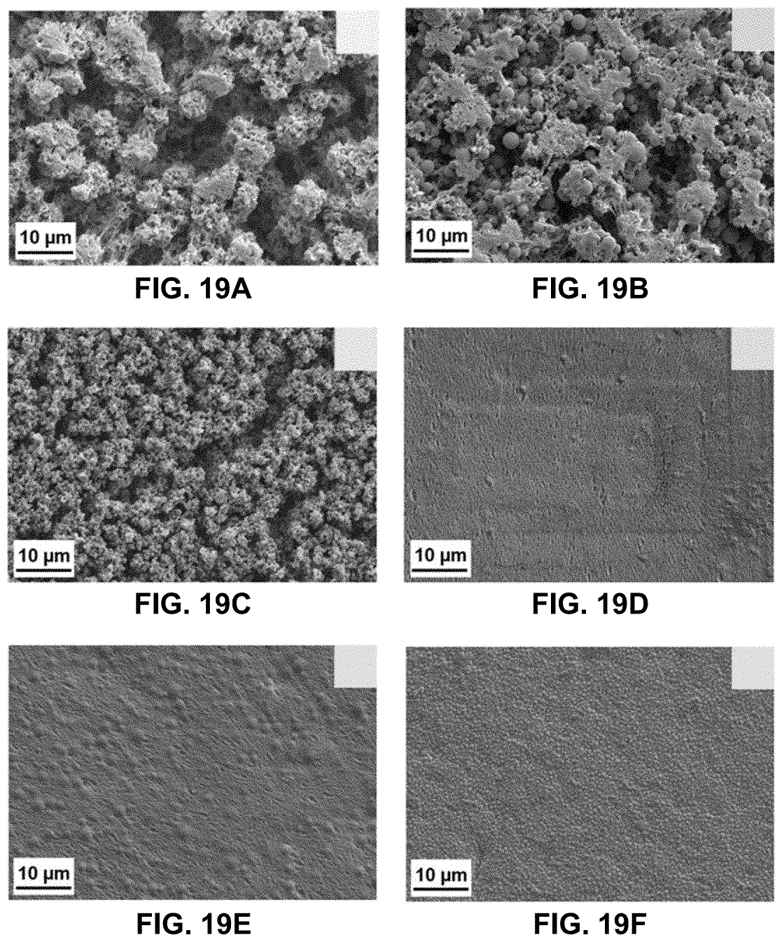

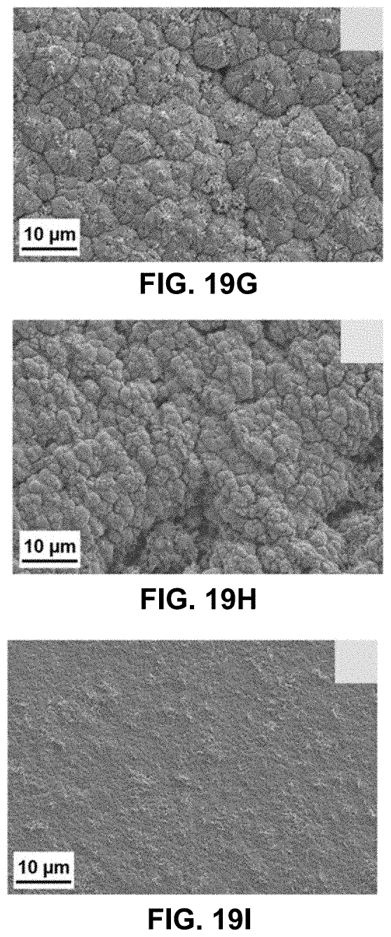

The SEM micrographs presented in FIGS. 18A-18I and 19A-19I provide insight into the influence of particle loading and nonsolvent composition on final membrane morphology. Consider first the average particle size and particle size distribution depicted in FIGS. 18A-18I and summarized in Table 5 (particle size data for 6 wt. % PEI and 21 wt. % PEI membranes prepared in NMP:H₂O are not included due to difficulties in clearly distinguishing between PVDF and PEI particles). In Table 5, it is observed that the average particle size and corresponding distribution are independent of nonsolvent indicating that the particle dimensions are determined prior to casting the dope solution. In a second trend it is observed that at low PEI concentrations the particle size increases with increasing PEI loading. This positive correlation continues until reaching a threshold between PEI loadings of 38 wt. % and 48 wt. %, after which the average particle size decreases to 0.9 microns and the particle size distribution (PSD) narrows.

TABLE 5
Mean particle diameter (μm) and standard deviation for membranes
with different particle loadings prepared using indicated nonsolvent

| | Wt. % PEI | | | | | |
|---|---|---|---|---|---|---|
| Nonsolvent | 06 | 21 | 38 | 48 | 54 | 60 |
| IPA | 1.1 ± 0.4 | 1.6 ± 0.5 | 1.8 ± 0.7 | 0.9 ± 0.2 | 0.9 ± 0.2 | 0.9 ± 0.1 |
| H₂O | 1.2 ± 0.4 | 1.5 ± 0.6 | 1.8 ± 0.8 | 0.9 ± 0.2 | 0.9 ± 0.1 | 0.9 ± 0.2 |
| NMP:H₂O | — | — | 1.9 ± 0.7 | 0.9 ± 0.1 | 0.9 ± 0.1 | 0.9 ± 0.2 |

The trend in particle size is attributed to the coalescence and breakup of phase separated PEI prior to completion of the crosslinking reaction. The phase separation of PEI from the rest of the dope, begins when the catalytic hydrochloric acid is added to the casting solution. The added HCl protonates some of the PEI molecules giving them a positive net charge. The charged PEI is no longer compatible with TEP leading the casting solution to phase separate and form PEI rich droplets. Early during the crosslinking reaction the PEI rich droplets are free to breakup or coalesce as the solution is stirred. At low PEI concentrations this process is transitory leading to the formation of a broad distribution of particle sizes. At high PEI concentrations, droplet coalescence and breakup is at a dynamic steady-state resulting in a narrower size distribution. As the crosslinking continues, the polymerization of PEI eventually leads to the formation of stable particles that are covalently bound and no longer undergo coalescence or breakup.

Next, consider aspects of membrane morphology that are affected by both the nonsolvent and PEI particle loading. FIGS. 18A-18C show the cross-sections of membranes cast in IPA and prepared using 6 wt. %, 38 wt. %, and 54 wt. % PEI loading respectively. In FIGS. 18A and 18B it may be seen that the PVDF forms spherulitic features with the PEI particles found along the edges of the spherulites. In contrast, the cross-section in FIG. 18C is dominated by PEI particles with little of the PVDF structure visible. The observations from FIGS. 18A-18C are complimented by the corresponding surface SEM micrographs shown in FIGS. 19A-19C. FIGS. 19A and 19B present similar PVDF structures with an open surface with several pores on the order of 10 microns. Although the two figures deviate in the number of PEI particles visible on the surface, the particles retain their positioning on the edges of the PVDF structures in agreement with the cross-section images. While FIG. 19C shares the high density of PEI particles found in FIG. 18C, the surface image also highlights both a reduction in the size of the PVDF spherulites and a higher density of smaller pores that was not evident in the cross-section image.

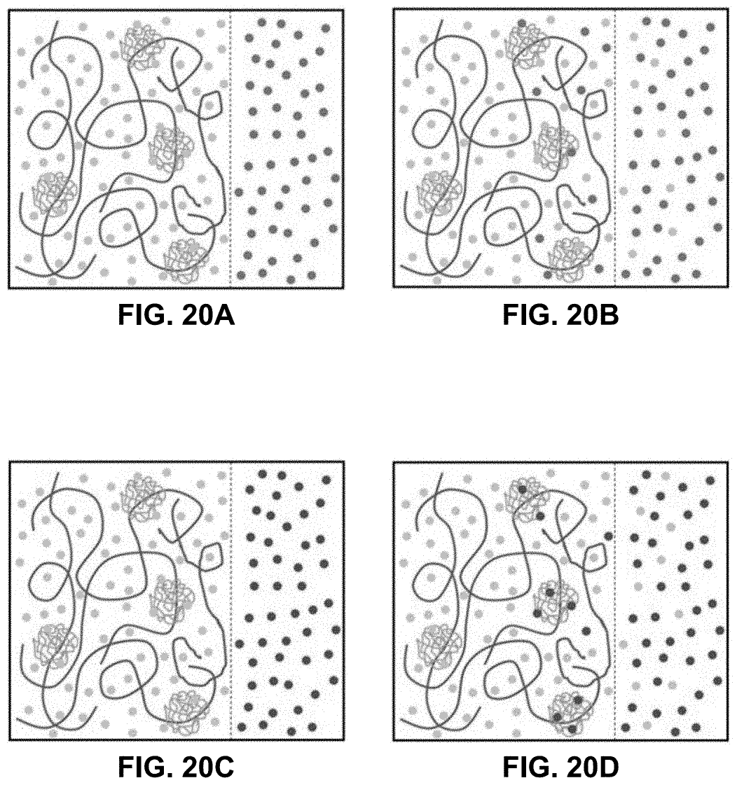

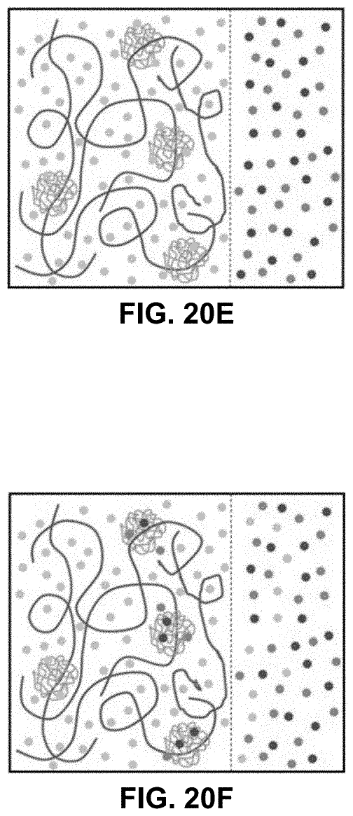

Several molecular interactions govern the final morphology of mixed-matrix membranes prepared using the NIPS process. The four interactions addressed within this study are: nonsolvent-solvent, nonsolvent-PVDF, nonsolvent-PEI particles, and PVDF-PEI particles. The interaction between IPA and TEP is relatively minor because the two molecules are readily miscible and have similar solubility parameters and polarities. Furthermore, IPA exhibits the same behavior as TEP when interacting with polymerized PEI (FIGS. 20A-20B), indicating that the IPA-PEI particles and IPA-TEP interactions do not significantly contribute to the final morphology of the membrane. Continuing to the IPA-PVDF interaction, IPA is well known in the literature as a soft nonsolvent for PVDF²³. Being a soft nonsolvent indicates that a higher concentration of IPA is typically used to force PVDF out of solution and therefore the ternary solution formed during NIPS is more likely to undergo solid-liquid demixing. Indeed, the observations in FIGS. 18A-18C and 19A-19C support a solid-liquid demixing mechanism. The presence of ordered PVDF spherulites throughout the membrane indicates that the crystallization of PVDF drove the phase separation into polymer-rich and polymer lean phases. To further support the solid-liquid demixing mechanism, consider the PVDF-PEI particles interaction. If the phase separation was initiated by liquid-liquid demixing with the polymer crystallizing after the phase separation, then there would be no driving force for the PEI particles to be located solely on the edge of the PVDF spherulites. In contrast, the polymer crystallization driving the phase separation would push particles in the polymer-rich phase to the phase boundary.

FIGS. 180-18F show the cross-sections of membranes cast in water prepared using 6 wt. %, 38 wt. %, and 54 wt. % PEI loading respectively. The cross-section presented in FIG. 18D has a similar structure to FIG. 18A in the presence of spherulitic PVDF with the PEI particles being located on the edges of the PVDF regions. As the PEI loading is increased to 38 wt. % (FIG. 18E) there are several changes in membrane morphology. First, the PVDF loses the spherulitic shape observed at lower PEI loadings and is exhibits a lace-like structure. Second, the PEI particles are now interspersed with the PVDF and, in some cases, the PVDF appears to coat sections of the particles. At the final PEI loading of 54 wt. %, shown in FIG. 18F, the PVDF maintains the lace-like structure even with the high PEI particle density. Furthermore, when compared to FIG. 18C, the PVDF regions of FIG. 18F are readily more visible, suggesting that the PVDF and PEI are still interspersed. The complimentary surface micrographs in FIGS. 19D-19F show the presence of a tight skin layer for all three PEI loadings. The only notable difference between the three samples is the number and size of the particles visible beneath the surface.

As noted above, the behavior of the H₂O/TEP/PVDF system during the NIPS process has been studied extensively in the literature^{18,19,23-27}. The observations and conclusions drawn from these studies provide a useful framework for addressing the morphology observed at the lowest PEI loading of 6 wt % due to the similarities in their structures. In the literature, membranes prepared using the H₂O/TEP/PVDF system exhibit a PVDF skin layer that is supported on a tight sponge layer that evolves into interconnected spherulities as the distance from the water/dope interface increases. In addition, following the phase inversion process the thicknesses of the membranes were found to be less than the casting height. Each of these observations may be explained using the four abovementioned interactions and the competition between kinetic and thermodynamic forces present in the H₂O-PVDF interaction. The kinetic forces are relevant under these conditions because of water's classification as a hard nonsolvent for PVDF²³. To begin, consider the formation of the PVDF skin layer. Upon initial contact with the water bath, TEP and water rapidly interdiffuse resulting in PVDF precipitating out of solution at the water/dope interface. The rapid kinetics of this process leads to the formation of a tight PVDF skin layer, which then regulates the mass transfer between the dope and the nonsolvent bath²⁴. The hydrophobic nature of the PVDF skin layer reduces the diffusion rate of water into the dope solution. The reduced rate gives rise to a transitionary region, the thickness of which scales as t_{sld}^{1/2} or the time required for the skin layer to solidify, where the kinetic forces eventually give way to thermodynamic forces resulting in a shift in morphology.

The morphology of the 6 wt. % membrane cast in water deviates from these observations only in the placement of the PEI particles. It was expected that the hydrophilic nature of the PEI particles would lead to a significant contribution from the H₂O-PEI interaction; however, these observations indicate that a PEI loading of 6 wt. % is not enough to have a noticeable impact. In the spherulitic portion of the membrane the PVDF-PEI particle interaction behaved similarly to that observed when using IPA, which supports the shift to thermodynamic driven phase separation with increasing distance from the dope/water interface.

The literature framework outlining the contribution of the interactions to the final morphology proves to be of limited use at higher PEI loadings. Although the framework still explains how the relevant mechanisms lead to the formation of the PVDF skin layer, it fails to capture the lace-like structure of the PVDF throughout the rest of the membrane. The shift in the PVDF structure indicates that the contributions from the H2O-PEI and PVDF-PEI interactions play a central role in determining the final morphology. In the literature framework, the PVDF skin layer reduced the diffusion of water into the dope solution²⁴. The reduced diffusion rate leads to a transition from kinetic (liquid-liquid demixing) to thermodynamic (solid-liquid demixing) forces dictating the polymer structure and subsequent overall membrane morphology.

Conversely, at a higher PEI loading the H₂O-PEI interaction increases the overall hydrophilicity of the skin layer allowing water to diffuse more quickly into the dope solution. As water diffuses into the membrane, it is attracted to the hydrophilic PEI leading to water-rich regions around the PEI particles (FIGS. 20C-20D). The higher water concentrations near the PEI particles promotes kinetically driven liquid-liquid demixing of the nearby PVDF, producing the lace-like structure. If the concentration of PEI particles is high enough, as seen in the 38 wt. % and 54 wt. % cases, the improved hydrophilicity and subsequent promotion of liquid-liquid demixing may extend throughout the entire thickness of the casting solution. In this situation, the direct PVDF-PEI interaction is replaced by the indirect PEI/H₂O/PVDF interaction that represents a blending of the PVDF-PEI, H₂O-PEI, and H₂O-PVDF interactions. If the PEI loading is not high enough, as seen in the 6 wt. % case, the diffusion of water into the bulk of the casting solution is too slow to promote liquid-liquid demixing resulting in thermodynamic forces determining the final morphology.

FIGS. 18G-18I show cross-sections of membranes cast in NMP:H₂O prepared using 6 wt. %, 38 wt. %, and 54 wt. % PEI loading respectively. In FIG. 18G, there appears to be dense globular PVDF structures out of which the beginnings of spherulitic structures are observed. This unique structure is a divergence from the anticipated spherulitic structure with PEI particles located along the edge observed in FIGS. 18A and 18D. In the corresponding surface micrograph (FIG. 19G), it may be seen that there are visible polymer grains that exhibit an unusual transition in structure. Towards the grain's center, a region is consistent with the dense globular PVDF structures identified in the cross-section (FIG. 18G). Towards the grain boundaries the dense structure gives way to a loose spherulitic structure, similar to those observed with IPA as nonsolvent.

Proceeding to the cross-section image corresponding to 38 wt. % PEI loading (FIG. 18H), it may be seen that only the globular PVDF structures remain with some PVDF taking the string-like structure observed in FIG. 18E. It is noteworthy that the globular structures appear to adhere to the PEI particles and, in some cases, appear to hold multiple PEI particles together. It should also be noted that several globular structures that have smooth bowl-shaped features that could feasibly be an interface with an absent PEI particle. The corresponding surface image (FIG. 19H) shows a rough surface with a higher density of grains than FIG. 19G. There is also an absence of spherulitic PVDF in the surface grains, which agrees with the observations from the cross-section. The cross-section presented in FIG. 18I demonstrates the continued presence of both globular and string-like PVDF amidst the high density of PEI particles. The corresponding surface micrograph in FIG. 19I shows the suppression of the grains observed in FIGS. 19G and 19H and more closely resembles the PVDF skin layer obtained with water as nonsolvent.

The morphology obtained using the mixed NMP:H₂O nonsolvent is unique and provides an interesting contrast to the morphologies obtained through thermodynamically driven (IPA) and kinetically driven (H₂O) PVDF solidification. Although NMP and H₂O are both polar and fully miscible, their interactions with TEP, PEI, and PVDF range from being similar (miscibility in TEP) to vastly different (solvent and nonsolvent for PVDF respectively). The differences in interactions between the two components of the nonsolvent provide additional interactions to consider, including the separation of the mixed nonsolvent into H₂O and NMP.

Consider the scenario of PVDF dissolved in TEP with no PEI, as found in both the neat membrane and regions far from the PEI microgels at lower PEI loadings. In the absence of PEI, the two contributing interactions are NMP:H₂O-TEP and NMP:H₂O-PVDF. With both NMP and water being miscible with TEP, the main contribution to polymer morphology stems from the latter of the two interactions. Upon immersion of the cast solution into the nonsolvent, water and NMP interdiffuse with TEP initiating the phase separation process. In contrast to the rapid solidification of the PVDF skin layer when using water alone, the mixed nonsolvent slows the NIPS process in two ways. The first is that NMP is a better solvent for PVDF than TEP²⁴, resulting in a higher local concentration of water typically being used to induce the phase separation. The second stems from the nonsolvent being a 50 v % mixture of water and NMP resulting in the concentration of water in the dope solution increasing more slowly than when using pure water. These combined effects facilitate solid-liquid demixing and produce a membrane morphology similar to that observed when using IPA in the absence of PEI particles.

In the absence of PEI particles, the fundamental interactions were addressed in the context of a mixed nonsolvent that didn't separate. Upon the addition of PEI to the dope solution, there is a higher likelihood that the mixed nonsolvent will separate when exposed to the different chemical environments. Therefore, this analysis first considers the interactions between the small molecules, PVDF, and polymerized PEI. For PVDF the most favorable interactions are with NMP, followed by TEP, with water being incompatible. For the PEI microgels the most favorable interactions (and highest swelling ratio) are with water, followed by NMP, with TEP not swelling the gel at all.

Upon immersing the dope solution into the mixed nonsolvent, the NMP:H₂O solution is attracted to the PEI particles and readily replaces the remaining TEP. As the concentration of the mixed nonsolvent increases in the PEI microgel, NMP moves to the interface between the PEI-rich and TEP/PVDF-rich regions due to its compatibility with both (FIGS. 20E-20F). The movement of NMP produces an interfacial region around the PEI microgel with a higher concentration of NMP. The increasing water concentration in the PEI-rich phase drives the phase separation of the PVDF in the interfacial region, but the phase separation process is once again slower than when water alone is used and as a result the morphology is dictated by thermodynamic forces. However, due to the higher concentration of NMP in the interfacial region and the polar influence of the PEI microgels, the phase separation close to the PEI particles produce a form of polar PVDF that appears as a condensed globular structure instead of the loose spherulites. As the phase separation proceeds beyond this interfacial area, the polar influence is lost and the remaining PVDF forms loose spherulites off of the globular structures. As the PEI particle loading increases, the NMP rich regions start to overlap leading to the formation of only globular PVDF as seen in FIGS. 18G-18H. Similar to using water alone, there is a unique interplay between the four interactions highlighted at the start of this section. The combination of the nonsolvent-PVDF, nonsolvent-PEI particles, and PVDF-PEI particles interactions alongside a mixed nonsolvent miscible with TEP produced a distinct and unique mixed-matrix membrane morphology.

2.3.2 Crystalline Behavior of PVDF

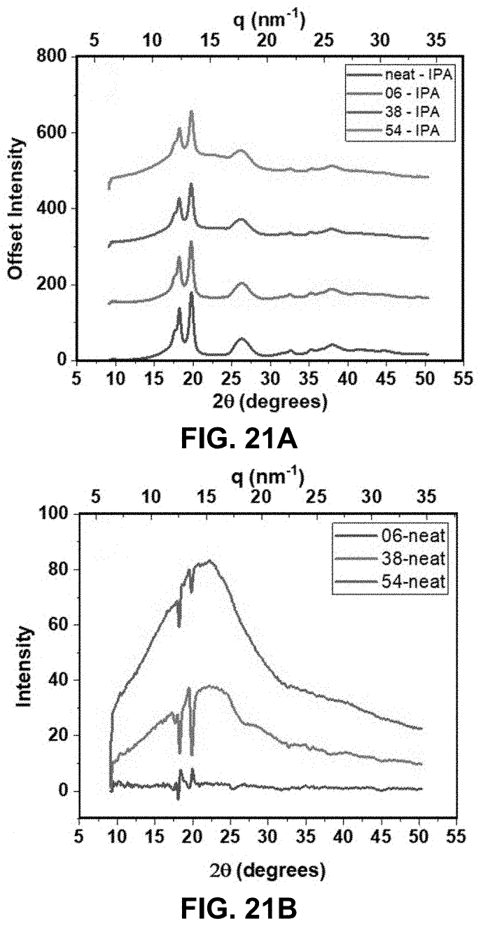

An essential component of membrane morphology (and subsequent performance) when using semi-crystalline PVDF is the crystalline phase and the percent crystallinity. All references to x-ray scattering scans or sample intensity signals from this point forward will be referring to WAXS scans that have had the background signal subtracted off unless otherwise indicated. Each scan in FIG. 21A exhibits crystalline peaks associated with the α-phase of PVDF (see Table 6 for 28 values corresponding to the different crystal phases) regardless of the PEI loading, indicating that the crystal phase of membranes prepared with Isopropanol is independent of particle loading.

TABLE 6
Peaks associated with different
crystal phases of PVDF. Peaks
provided are obtained using Cu-kα
radiation with wavelength 0.154 nm.^{22,25,28}

| Crystal | 2θ peaks |
|---|---|
| phase | (degrees) |
| α | 17.6, 18.3, 19.9, & 26.5 |
| β | 20.6, 36, & 40 |

The independence of crystal phase from particle loading when using IPA as the nonsolvent, is in agreement with the observations from the SEM micrographs. As noted above, IPA is a soft nonsolvent for PVDF and does not interact strongly with PEI resulting in solidification through solid-liquid demixing. The solid-liquid demixing encourages the formation of the most thermodynamically favored crystal phase—the α-phase. Although the PVDF-PEI interaction does influence the size and spacing of the PVDF spherulites, it does not affect the balance of kinetic and thermodynamic forces. Therefore, the crystalline phase is independent of PEI loading.

In order to tease out the amorphous PEI contribution to the overall signal, it was decided to subtract the scan of the neat sample from the scans of samples with different PEI loadings. To account for different concentrations of PVDF being present in each sample, the intensities of the neat sample were multiplied by the ratio of PVDF concentrations (Eq 2.1) before being subtracted from the sample scans. The resulting curves are presented in FIG. 21B, where it may be seen that there is a broad peak centered around 22° with a tail extending out to higher angles. The curves from the samples with higher PEI loading (38-neat and 54-neat) both show valleys at 18.3° and 19.9°. The curve produced using 6 wt. % PEI composition shows two small peaks at the same angles.

$$r = \frac{wt {\%}_{PVDFsample}}{wt {\%}_{PVDF , neat}} \qquad ( 2.1 )$$

The method of subtracting off the PVDF concentration corrected neat membrane scan was used to analyze the amorphous PEI contribution because the subtraction removes the bulk of the PVDF contributions (amorphous and crystalline) and any additional environmental background contributions not captured in the background scan. Furthermore, by using the scans of samples cast in IPA any confounding effects from different crystal phases were avoided thereby allowing additional information on changes in percent crystallinity of PVDF (fraction of PVDF that is crystalline) to be obtained. The changes in PVDF crystallinity between the samples were illustrated by the presence of peaks or valleys in the calculated curves at the position of the α-phase peaks.

The peaks in the 6 wt. % PEI curve depicted in FIG. 21B represent an increase in crystallinity from the neat PVDF membrane as the sample with PEI has a higher intensity than can be accounted for by the broad amorphous PEI halo. Similarly, the presence of valleys in the 38 wt. % and 54 wt. % PEI curves, FIG. 21B, indicate that the crystalline PVDF fraction has decreased as the signal intensity at 18.3° and 19.9° is lower than could be attributed to dilution by PEI. The deduced reduction in percent crystallinity of the 38 wt. % and 54 wt. % PEI membranes are attributed to a combination of PVDF being entrapped in the functional particles and the functional particles perturbing the polymer crystallization. As the PEI concentration rises a larger number of particles are formed, which increases the fraction of PVDF that is entrapped in the PEI particles. The entrapped PVDF is unable to crystallize due to the physical constraints of the gel and instead contributes to the amorphous phase. Similarly, as the number and size of particles increases there are fewer opportunities for crystals to grow without running into obstacles. The increasing number of obstacles frustrates polymer crystallization leading to an increase in the amorphous polymer halo. The observation that the decrease in crystallinity was more prevalent at a PEI loading of 38 wt. % needs further investigation, but may stem from the differences in particle size and PSD between the middle and high PEI concentrations.

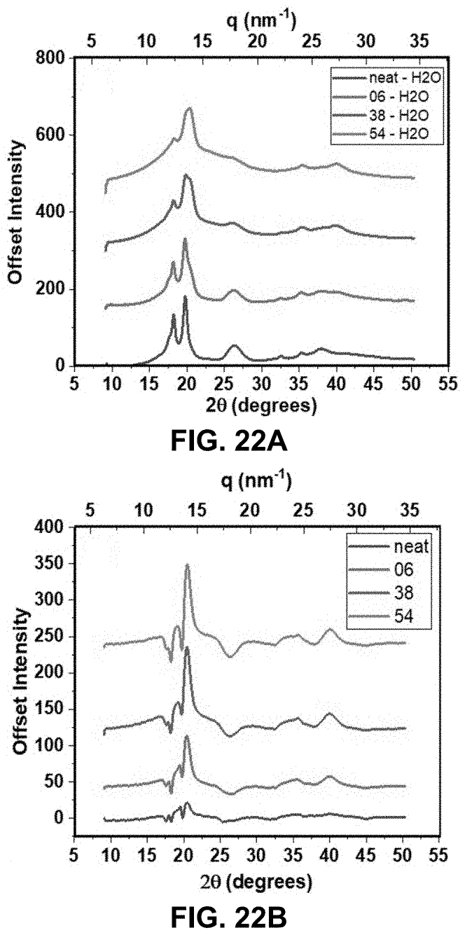

FIG. 22A shows the x-ray scattering scans for membranes prepared using water as the nonsolvent. The changes in the crystalline phase exhibits a similar dependence on particle loading as that observed in the SEM analysis. In the absence of PEI and at low particle loadings the membrane is predominantly in the α-phase with a small shoulder visible on the 19.9° peak at 6 wt. % PEI. The shoulder representing the β-phase peak continues to grow as the PEI concentration increases and, at a PEI loading of 54%, surpasses the 19.9° α-phase peak. FIG. 22B shows several curves calculated by subtracting the IPA cast scan from the water cast scan at a given dope composition. The neat membrane curve depicts small valleys at the angles associated with the α crystal phase. The curves for membranes prepared with PEI have the same valleys, albeit more distinct, as the neat curve as well as local peaks at angles corresponding to the β crystal phase. The differences between the local maximum at 20.6° and the local minimum at 19.9° (DPV) are 20, 47, 71, and 98 for the neat, 6 wt. %, 38 wt. %, and 54 wt. % compositions respectively.

When TEP is used as a solvent, rapid liquid-liquid demixing in the presence of water leads to PVDF being kinetically trapped during solidification producing a mixture of β-phase and α-phase²². Without PEI particles present in the membrane, this phenomenon is restricted to the membrane skin layer because of the mass transfer limitations imposed by the formation of said layer. The reduced mass transfer of water facilitates a thermodynamically driven phase separation that favors the formation of the α crystal phase when using TEP as the nonsolvent. A similar crystal phase behavior is observed at low PEI loadings. The only indication of β-phase PVDF being the shoulder on the α-phase 19.9° peak, indicating that there was not enough driving force to fully push kinetically driven phase separation of the bulk. As the PEI loading continues to increase the mass transfer of water into the casting solution is improved and a greater percentage of the dope solidifies via kinetically induced phase separation resulting in increasing concentrations of β-phase.

The subtraction of the IPA cast scan from the water cast scan accomplished three things: First, subtracting off the IPA signal at the same membrane composition removes the contributions of PEI and any other environmental background sources not accounted for in the recorded background scan. Second, identification of changes in the crystal phase as a function of PEI loading through the location and intensity of local peaks and valleys. As noted above, the difference between the valley minimum at 19.9° and the peak maximum at 20.6° has a positive correlation with PEI loading, thereby supporting the qualitative conclusions drawn from FIG. 22A. Third, develop a method to analyze x-ray scattering measurements of complex materials while limiting operator bias. There are still several opportunities to improve the quantitative capabilities of this method to more fully characterize the x-ray scattering of complex materials.

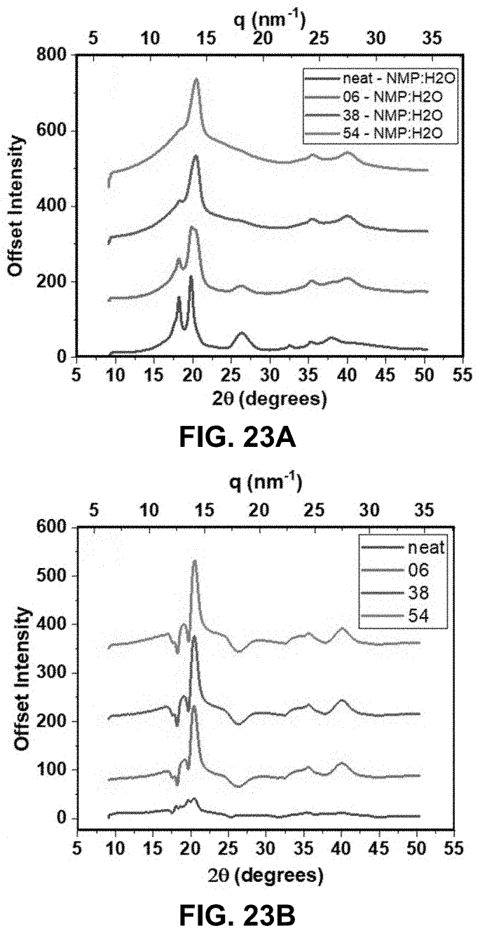

FIG. 23A shows the x-ray scattering scans for membranes prepared using the mixed nonsolvent NMP:H₂O. The mixed nonsolvent scans share several similarities with membranes cast in water, such as the α-phase dominated neat membrane and the shift from the α to β crystal phase. However, the shift in crystal phase occurs more rapidly when using the mixed nonsolvent, having already produced a plateau between the 19.9° and 20.6° peaks at a particle loading of 6%. This trend continues until reaching a particle loading of 54 wt. %, at which point the membrane is predominantly in the β crystal phase as seen by the almost complete suppression of the 18.3° and 26.5° α-phase peaks. Similar to FIG. 22B, FIG. 23B shows several curves calculated by subtracting the IPA cast scan from the NMP:H₂O cast scan at a given dope composition. The neat membrane curve does not have any clearly discernable valleys or peaks, just a general increase in signal intensity between 17° and 20°. The curves for membranes prepared with PEI have valleys at angles corresponding to diffraction peaks of the α crystal phase as well as local peaks at angles corresponding to the β crystal phase. The DPV for membranes cast using the mixed nonsolvent are 6, 93, 117, and 138 for the neat, 6 wt. %, 38 wt. %, and 54 wt. % compositions respectively.

The rapid shift in crystal phase when using the NMP:H₂O mixed nonsolvent, seen in FIG. 23A, agrees with the observations and conclusions drawn from the SEM analysis. In the absence of PEI the mixed nonsolvent acts similar to a soft nonsolvent and produces the α crystal phase, which is thermodynamically favorable under these conditions. Once PEI is added to the dope solution, PVDF near the PEI microgels form polar globular structures that are predominantly β-phase crystallites. As the PEI loading and number of PEI particles increase, the NMP rich interfacial regions start to overlap leading to increased formation of the globular PVDF and higher β-phase crystal content.

A brief comparison of the DPV for the water cast and NMP:H₂O cast membranes provides additional insight into the crystalline behavior of PVDF under the two conditions. Starting with the neat membranes, the water cast membrane has a value of 20 with the mixed nonsolvent cast has a value of 6, indicating that in the absence of PEI the membrane produced by the mixed nonsolvent most closely resembles the IPA cast sample. This validates the claim that the mixed nonsolvent acts like a soft nonsolvent of PVDF in the absence of PEI. Upon the addition of PEI, the DPV of the water cast samples and the NMP:H₂O cast samples increases with increasing PEI loading. While membranes prepared in both conditions have increasing concentrations of β-phase, at each composition, the membranes prepared with the mixed nonsolvent have a higher concentration of the β crystal phase. This phenomenon stems from the way the β-phase is formed in the different conditions during casting. As summarized above, the β-phase PVDF in water cast membranes stems from kinetically driven phase separation. Using this method inherently limits the maximum amount of β crystal phase because there will always be a portion of the crystalline material that will form the thermodynamically favored α-phase. In contrast, using the mixed nonsolvent changes the casting conditions in such a way that the formation of the β crystal phase is thermodynamically favored near the PEI particles. Therefore, increasing the PEI particle concentration increases the thermodynamically favored β-phase content and suppresses the formation of the α crystal phase such that it is theoretically feasible to have essentially pure β-phase PVDF.

2.3.3 Flux Measurements

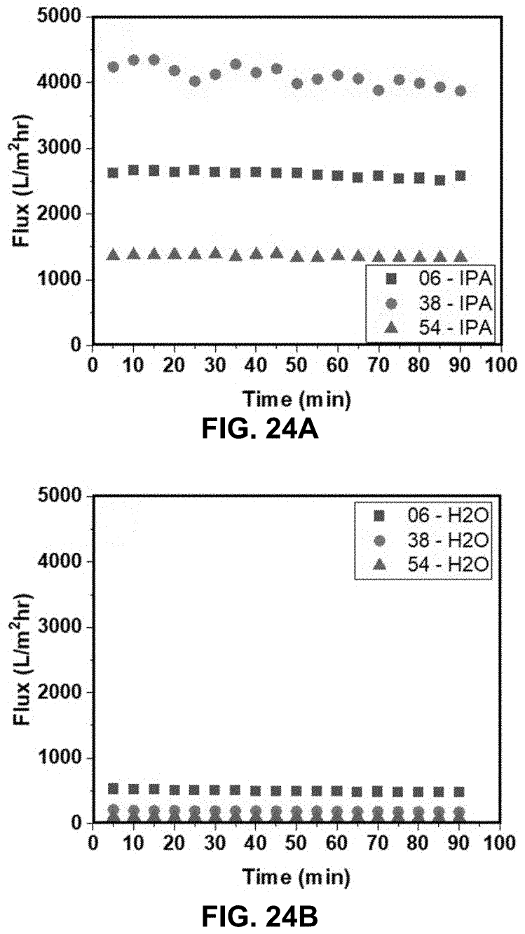

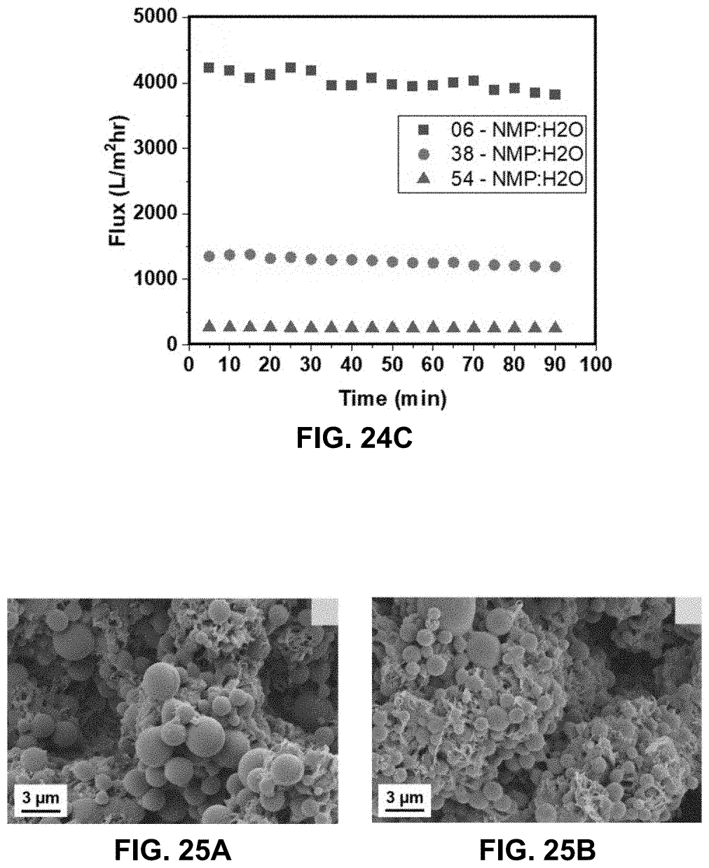

The flux measurements presented in FIGS. 24A-24C provide both validation of the SEM analysis of membrane morphology and additional information on the microgels influence on membrane performance when wet. For example, the membranes cast in IPA are expected to have the highest water fluxes of the three casting conditions due to the open morphology with large pores. The membranes prepared in IPA, FIG. 24A, are in agreement with the predicted behavior having fluxes of 1400, 2600, and 4000 Lm⁻²h⁻¹ corresponding to particle loadings of 54%, 6%, and 38% respectively. Similarly, membranes cast in water have a tight skin layer that should impede fluid flow leading to significantly lower fluxes under the same operating conditions. FIG. 24B provides supporting evidence with the fluxes for all three particle loadings being below 600 Lm⁻²h⁻¹. Using the mixed nonsolvent produced membranes with an unusual morphology that lacks both the skin layer and the open pores observed in the surface micrographs of the other two casting conditions (FIGS. 19A-19I). Since the surface prepared with the mixed nonsolvent has a pseudo skin layer that is initially porous and then loses its porosity with increasing particle loading, the NMP:H₂O membrane fluxes for a given particle loading were expected to be in-between the corresponding fluxes reported in FIGS. 24A-24B. The fluxes obtained for 38 wt. % and 54 wt. % follow the expected trend, but the 6 wt. % PEI loading flux deviated and at 4000 Lm⁻²h⁻¹ was the highest flux observed for this composition.

For membranes with different compositions prepared under the same casting conditions, it was initially proposed that higher PEI loadings would lead to higher fluxes due to the increasing hydrophilicity of the membrane. However, the reported fluxes in FIGS. 24A-24C demonstrate that flux and PEI loading negatively correlate with the notable exception of membranes cast in IPA, which have a maximum flux at the middle PEI loading of 38 wt. %. This maximum is attributed to optimizing the balance between membrane hydrophilicity and available pore volume. At low PEI loadings the MMMs have a higher mass fraction of PVDF that makes the overall membrane less hydrophilic resulting in reduced water flux through the membrane at a set operating pressure. As the mass fraction of PEI is increased to 38 wt. % and 54 wt. % the overall membrane becomes more hydrophilic facilitating improved water transport. Acting in opposition to the higher membrane flux encouraged by improved hydrophilicity, the increasing concentration of PEI microgels impedes mass transfer through the membrane by decreasing the pore volume available for transport. Therefore, at a PEI composition of 38 wt. % the membrane is more hydrophilic than a 6 wt. % membrane while having fewer microgels reducing transport pore volume than a 54 wt. % membrane producing the optimum conditions for a maximum membrane flux. Although the flux of the 54 wt. % PEI membrane is the lowest of the three compositions presented here, it has the highest concentration of functional particles while still being capable of operating in the microfiltration regime. As a result, the 54 wt. % PEI composition membranes cast in IPA were used as the baseline material for the membrane chromatography experiments discussed in Example 9.

The negative correlation between PEI loading and membrane flux observed in FIGS. 24B-24C indicates that another interaction contributes to the flux that is absent in membranes prepared with IPA. In the surface SEM micrographs presented in FIGS. 19A-19I, it is shown that membranes prepared in both water and the mixed nonsolvent have a visible surface layer with varying continuity/porosity across the membrane surface depending on casting conditions. At low PEI concentrations—such as the 6 wt. % PEI composition—the mass percent of solids in the dope is low enough (Table 4) to allow defects in the surface layer during casting. These defects enable faster mass transfer through the membrane resulting in higher fluxes even with the lower membrane hydrophilicity. As the PEI loading and mass percent of solids increases to the 38 wt. % PEI composition the surface layer changes such that membranes cast in water demonstrate a continuous tight skin layer and those prepared using the mixed nonsolvent have a more homogeneous surface layer with reduced porosity. These changes in the surface layer result in decreased water fluxes for both sets of membranes. The reduction in membrane flux of samples with 38 wt. % and 54 wt. % PEI that were cast in water is attributed to the interaction between hydrophilicity and pore volume discussed above with the skin layer making minimal changes between the two conditions. In contrast, membranes prepared with the mixed nonsolvent once again demonstrate large changes in surface structure by increasing PEI loading from 38 wt. % to 54 wt. %. The limitations to mass transfer from a less porous surface combined with the contributions of membrane hydrophilicity and pore volume lead to a membrane flux comparable to the corresponding membrane cast in water.

2.4 Conclusions

This study demonstrates the manipulation of mixed-membrane morphology by controlling functional particle loading and nonsolvent chemistry. Several important interactions and their resulting influence on final morphology were reported. First, the correlation between particle size, PSD, and particle loading was attributed to reaching a steady state in droplet coalescence and break-up. Second, the interplay between the nonsolvent-solvent, nonsolvent-PVDF, nonsolvent-PEI particles, and PVDF-PEI particles interactions was found to be the deciding factor in determining the final membrane morphology when using a pure nonsolvent. Third, using a mixed nonsolvent solution compromising a harsh nonsolvent mixed with a secondary solvent provided additional interactions to consider and resulted in novel membrane structures. The membrane characterization via x-ray scattering scans and flux measurements supported the conclusions drawn from the SEM micrographs.

### References Corresponding to Example 8

(1) Mahajan, R.; Koros, W. J. Factors Controlling Successful Formation of Mixed-Matrix Gas Separation Materials. Ind. Eng. Chem. Res. 2000, 39 (8), 2692-2696. https://doi.org/10.1021/ie990799r.
(2) Aroon, M. A.; Ismail, A. F.; Matsuura, T.; Montazer-Rahmati, M. M. Performance Studies of Mixed Matrix Membranes for Gas Separation: A Review. Sep. Purif. Technol. 2010, 75 (3), 229-242. https://doi.org/10.1016/j.seppur.2010.08.023.
(3) Shen, Y.; Lua, A. C. Preparation and Characterization of Mixed Matrix Membranes Based on PVDF and Three Inorganic Fillers (Fumed Nonporous Silica, Zeolite 4A and Mesoporous MCM-41) for Gas Separation. Chem. Eng. J. 2012, 192, 201-210.
(4) Dong, G.; Li, H.; Chen, V. Challenges and Opportunities for Mixed-Matrix Membranes for Gas Separation. J Mater Chem A 2013, 1, 4610-4630. https://doi.org/10.1039/C3TA00927K.
(5) Geise, G. M.; Lee, H.-S.; Miller, D. J.; Freeman, B. D.; McGrath, J. E.; Paul, D. R. Water Purification by Membranes: The Role of Polymer Science. J. Polym. Sci. Part B Polym. Phys. 2010, 48 (15), 1685-1718. https://doi.org/10.1002/polb.22037.
(6) Geise, G. M.; Paul, D. R.; Freeman, B. D. Fundamental Water and Salt Transport Properties of Polymeric Materials. Prog. Polym. Sci. 2014, 39 (1), 1-42. https://doi.org/10.1016/j.progpolymsci.2013.07.001.
(7) Sagle, A.; Freeman, B. Fundamentals of Membranes for Water Treatment. 17.
(8) Werber, J. R.; Osuji, C. O.; Elimelech, M. Materials for Next-Generation Desalination and Water Purification Membranes. Nat. Rev. Mater. 2016, 1 (5), 1-15.
(9) Werber, J. R.; Deshmukh, A.; Elimelech, M. The Critical Need for Increased Selectivity, Not Increased Water Permeability, for Desalination Membranes. Environ. Sci. Technol. Lett. 2016, 3 (4), 112-120. https://doi.org/10.1021/acs.estlett.6b00050.
(10) Wang, P.; Chung, T.-S. A New-Generation Asymmetric Multi-Bore Hollow Fiber Membrane for Sustainable Water Production via Vacuum Membrane Distillation. Environ. Sci. Technol. 2013, 47 (12), 6272-6278. https://doi.org/10.1021/es400356z.
(11) Li, X.; Sotto, A.; Li, J.; Van der Bruggen, B. Progress and Perspectives for Synthesis of Sustainable Antifouling Composite Membranes Containing in Situ Generated Nanoparticles. J. Membr. Sci. 2017, 524, 502-528. https://doi.org/10.1016/j.memsci.2016.11.040.
(12) Ghosh, S. K.; Alam, Md. M.; Mandal, D. The in Situ Formation of Platinum Nanoparticles and Their Catalytic Role in Electroactive Phase Formation in Poly(Vinylidene Fluoride): A Simple Preparation of Multifunctional Poly(Vinylidene Fluoride) Films Doped with Platinum Nanoparticles. RSC Adv 2014, 4 (79), 41886-41894. https://doi.org/10.1039/C4RA06334A.
(13) Dai, B.; Yu, Y.; Chen, Y.; Huang, H.; Lu, C.; Kou, J.; Zhao, Y.; Xu, Z. Construction of Self-Healing Internal Electric Field for Sustainably Enhanced Photocatalysis. Adv. Funct. Mater. 2019, 29 (16), 1807934. https://doi.org/10.1002/adfm.201807934.
(14) Liu, Z.; Yu, X.; Li, L. Piezopotential Augmented Photo- and Photoelectro-Catalysis with a Built-in Electric Field. Chin. J. Catal. 2020, 41 (4), 534-549. https://doi.org/10.1016/S1872-2067(19)63431-5.
(15) Kotte, M. R.; Kuvarega, A. T.; Cho, M.; Mamba, B. B.; Diallo, Mamadou. S. Mixed Matrix PVDF Membranes With in Situ Synthesized PAMAM Dendrimer-Like Particles: A New Class of Sorbents for Cu(II) Recovery from Aqueous Solutions by Ultrafiltration. Environ. Sci. Technol. 2015, 49 (16), 9431-9442. https://doi.org/10.1021/acs.est.5b01594.
(16) Liu, G.; Hung, W.-S.; Shen, J.; Li, Q.; Huang, Y.-H.; Jin, W.; Lee, K.-R.; Lai, J.-Y. Mixed Matrix Membranes with Molecular-Interaction-Driven Tunable Free Volumes for Efficient Bio-Fuel Recovery. J. Mater. Chem. A 2015, 3 (8), 4510-4521. https://doi.org/10.1039/C4TA05881J.
(17) Park, M. J.; Nisola, G. M.; Vivas, E. L.; Limjuco, L. A.; Lawagon, C. P.; Seo, J. G.; Kim, H.; Shon, H. K.; Chung, W.-J. Mixed Matrix Nanofiber as a Flow-through Membrane Adsorber for Continuous Li+ Recovery from Seawater. J. Membr. Sci. 2016, 510, 141-154. https://doi.org/10.1016/j.memsci.2016.02.062.
(18) Rao Kotte, M.; Hwang, T.; Han, J.-I.; Diallo, M. S. A One-Pot Method for the Preparation of Mixed Matrix Polyvinylidene Fluoride Membranes with in Situ Synthesized and PEGylated Polyethyleneimine Particles. J. Membr. Sci. 2015, 474, 277-287. https://doi.org/10.1016/j.memsci.2014.09.044.
(19) Sun, C. Poly (Vinylidene Fluoride) Membranes: Preparation, Modification, Characterization and Applications. 2009.
(20) Zuo, D. Y.; Li, H. J. Membrane Formation Mechanism for Water-Solvent-PVDF Systems and Membrane Structure for Ultrafiltration Application. Adv. Mater. Res. 2012, 578, 129-132. https://doi.org/10.4028/www.scientific.net/AMR.578.129.
(21) Zuo, J.; Chung, T.-S. PVDF Hollow Fibers with Novel Sandwich Structure and Superior Wetting Resistance for Vacuum Membrane Distillation. Desalination 2017, 417, 94-101. https://doi.org/10.1016/j.desa1.2017.05.022.
(22) Buonomenna, M. G.; Macchi, P.; Davoli, M.; Drioli, E. Poly(Vinylidene Fluoride) Membranes by Phase Inversion: The Role the Casting and Coagulation Conditions Play in Their Morphology, Crystalline Structure and Properties. Eur. Polym. J. 2007, 43 (4), 1557-1572. https://doi.org/10.1016/j.eurpolymj.2006.12.033.
(23) Zuo, D.; Zhu, B.; Cao, J.; Xu, Y. Influence of Alcohol-Based Nonsolvents on the Formation and Morphology of PVDF Membranes in Phase Inversion Process. Chin. J. Polym. Sci. 2006, 24 (03), 281-289.
(24) Bottino, A.; Camera-Roda, G.; Capannelli, G.; Munari, S. The Formation of Microporous Polyvinylidene Difluoride Membranes by Phase Separation. J. Membr. Sci. 1991, 57 (1), 1-20.
(25) Tao, M.; Liu, F.; Ma, B.; Xue, L. Effect of Solvent Power on PVDF Membrane Polymorphism during Phase Inversion. Desalination 2013, 316, 137-145.
(26) Su, Y. P.; Sim, L. N.; Li, X.; Coster, H. G. L.; Chong, T. H. Anti-Fouling Piezoelectric PVDF Membrane: Effect of Morphology on Dielectric and Piezoelectric Properties. J. Membr. Sci. 2021, 620, 118818. https://doi.org/10.1016/j.memsci.2020.118818.
(27) Lin, D.-J.; Chang, H.-H.; Chen, T.-C.; Lee, Y.-C.; Cheng, L.-P. Formation of Porous Poly(Vinylidene Fluoride) Membranes with Symmetric or Asymmetric Morphology by Immersion Precipitation in the Water/TEP/PVDF System. Eur. Polym. J. 2006, 42 (7), 1581-1594. https://doi.org/10.1016/j.eurpolymj.2006.01.027.
(28) Gregorio Jr, R. Determination of the α, β, and γ Crystalline Phases of Poly (Vinylidene Fluoride) Films Prepared at Different Conditions. J. Appl. Polym. Sci. 2006, 100 (4), 3272-3279.

### Example 9: Mixed-Matrix Membrane Chromatography

3.1 Introduction

The field of membrane chromatography has expanded rapidly as an alternative to the conventional packed bed chromatography in pharmaceutical separations¹⁻⁴. The shift in technology has been motivated by a need to reduce downstream bioprocessing costs associated with long processing times and high operating pressures. Membrane chromatography reduces the processing time and operating pressure by utilizing convective, as opposed to diffusive, mechanisms to transport molecules of interest to the associated binding sites. The change in transport mechanism enables the system to operate at faster flow rates while maintaining a low operating pressure. In addition, the use of convective transportation allows for processing to be operated at a wide range of flow rates with minimal impact on the binding capacity of the membrane. These flow properties are amenable to the scale-up of the separation processes and complement the easy mass production of membrane-based materials thereby further reducing downstream costs¹². The adoption of membrane chromatography has also benefited from drawing on the experience of related fields in membrane science, i.e., identification of porous polymeric membranes with good chemical and physical stability to act as supports. As a result, many membrane adsorbers are derivatives of membranes used in other separation processes¹⁻⁴.

In order to capitalize on the advantages of fast flow rates and low operating pressures outlined above, recent work has focused on addressing the key drawbacks of membrane chromatography. Two such drawbacks are the low volumetric binding capacity of membrane adsorbers in comparison to resins and limited salt tolerance^{1,3-7}. While resins have a high binding surface area per volume ratio due to the tortuosity of the resin beads, early membrane adsorbers rely solely on the pore surface area as the active binding area resulting in low volumetric binding capacity. A promising method to overcome this barrier is to use various polymerization techniques to graft polyelectrolyte chains or polymer brushes with appropriate functionalities onto the porous membrane supports^{3-5,8}. The resulting membranes benefit from the porosity of the support while increasing the available binding surface area to improve volumetric binding capacity. However, the improvement in volumetric binding capacity has only been shown for solutions with low (<100 mM) salt concentrations³⁻⁵.

Operating pharmaceutical separations in solutions with low ionic strength typically requires a buffer exchange step which increases processing costs⁷. In order to reduce the extent of the buffer exchange step, it has typically been desirable to develop membranes which demonstrate consistent binding over a range of salt concentrations. The improvement of the salt tolerance of membrane adsorbers generally requires a reduction in the ionic sensitivity of the binding ligand through manipulation of the ligand chemistry. Recent work replacing ligands with quaternary amine-based chemistry with those containing predominantly primary amine chemistry demonstrated volumetric binding capacities which were essentially constant over the conductivity range 1.8 mS/cm to 16.8 mS/cm^{6,9-12}. Although these membranes achieved high salt tolerance, the reported volumetric binding capacities were low. While recent work in the field reliably addresses one of the drawbacks mentioned above, there is still a need for a membrane adsorber which provides a consistently high volumetric binding capacity over a wide range of salt concentrations.

In this study we build upon results from Example 8 and investigate the relationship between crosslinker chemistry, crosslinker density, and volumetric binding capacity. We chose three unique crosslinkers with different reactive groups, connecting chain chemistry, and lengths to characterize the influence of crosslinker chemistry in diverse conditions. Epichlorohydrin (ECH) is the shortest crosslinker and provides only an alcohol functionality post-reaction, Bis(2-chloroethyl)amine hydrochloride (BCAH) has a medium length and provides an additional secondary amine post-reaction, and Di(ethylene glycol) diacrylate (EGA) is the longest crosslinker and provides glycol functionality post-reaction. All three crosslinkers were used in varying concentrations to obtain information on the interplay between crosslinker chemistry and crosslink density and the resulting impact on volumetric binding capacity. The binding capacity was measured in both static and dynamic configurations to demonstrate the capabilities of mixed-matrix membranes adsorbers.

3.2 Experimental Methods

3.2.1 Materials

Polyvinylidene Fluoride (PVDF; Kynar 761, 400 kg/mol) was donated by Arkema. Hyperbranched polyethylenimine (PEI; 600 g/mol) was purchased from Polysciences. The following chemicals were purchased from Sigma Aldrich: Epichlorohydrin (ECH), Di(ethylene glycol) diacrylate (EGA), Bis(2-chloroethyl)amine hydrochloride (BCAH), Isopropanol (IPA), Triethyl phosphate (TEP), Dimethyl sulfoxide (DMSO), TRIShydrochloride (TRIS), Glycerol, and Bovine Serum Albumin (BSA). The l×PBS solution (Corning 21-040-CV) was purchased from VWR. All chemicals and materials were used as received. All buffers were prepared using indicated chemicals and distilled water.

3.2.2 Membrane Synthesis

To begin a typical membrane synthesis, 5.91 g of PVDF was added to an empty 3-neck round bottom flask. The flask was fitted with an overhead mechanical stirrer and the necessary greased connectors. Thirty mL of TEP was then added to the flask and the remaining openings were sealed using rubber septa. The PVDF/TEP mixture was heated to 80° C. for an hour before the mixing speed was set to 60 rpm. The resulting solution was left to equilibrate overnight. A PEI/TEP solution was prepared by adding 5 g of PEI to a scintillation vial followed by 5 mL of TEP. The mixture was vortexed until a homogeneous clear solution was obtained and then it was left to equilibrate overnight at room temperature. For membranes with BCAH as the crosslinker, the crosslinker solution was prepared by weighing the required amount of BCAH into a scintillation vial and then adding the corresponding volume of DMSO (Table 7). DMSO was chosen as the solvent due to its compatibility with the other components of the dope solution and TEP's inability to dissolve BCAH. The resulting mixture was incubated overnight at room temperature to fully dissolve the BCAH.

$$NCD = \frac{\frac{[ crosslinker ]}{[ PEI ]}}{\frac{{[ crosslinker ]}_{ref}}{{[ PEI ]}_{ref}}} \qquad ( 3.1 )$$

TABLE 7
Crosslinker solution composition with the corresponding Normalized
Crosslink Density and membrane formulation.

| | | Crosslinker | | Volume of |
|---|---|---|---|---|
| Formulation | Crosslinker | mass(g) | NCD | DMSO (mL) |
| 54A | ECH | 3.2 | 1 | — |
| 54B | ECH | 1.6 | 0.5 | — |
| 54C | ECH | 0.8 | 0.25 | — |
| 54D | EGA | 7.4 | 1 | — |
| 54E | EGA | 3.7 | 0.5 | — |
| 54F | EGA | 1.8 | 0.25 | — |
| 54G | BCAH | 6.2 | 1 | 8 |
| 54H | BCAH | 3.1 | 0.5 | 5 |
| 54I | BCAH | 1.6 | 0.25 | 2.5 |

Once the solutions were equilibrated, the flask was purged with N₂ for 7 minutes and the mixing speed was increased to 250 rpm. With the N₂ flow still on, the PEI solution was then added dropwise to the flask using a glass Pasteur pipette over the course of 4 minutes. The resulting solution was left to mix for 5 minutes before adding 0.43 mL of concentrated HCl (37% solution). Following the addition of the HCl, the flask was incubated for 15 minutes at 80° C. with the mixing speed maintained at 250 rpm. The crosslinker solution corresponding to the desired normalized crosslink density (NCD) in Table 3.1, calculated using equation 3.1, was then added to the flask and the polymerization reaction was allowed to proceed for 4 hours. After the 4-hour reaction time, the flask was put under in-house vacuum for 10 minutes to remove entrapped air. The membranes were then cast on glass plates using a doctor blade with a blade height of 300 μm. The cast membranes were left at room temperature for 30 seconds before being immersed in an Isopropanol coagulation bath. After two hours, the solidified membranes were moved to distilled water baths prior to storage.

3.2.3 SEM Characterization

Please see the corresponding section in Example 8 for detailed description on SEM characterization method.

3.2.4 Protein Binding Experiments

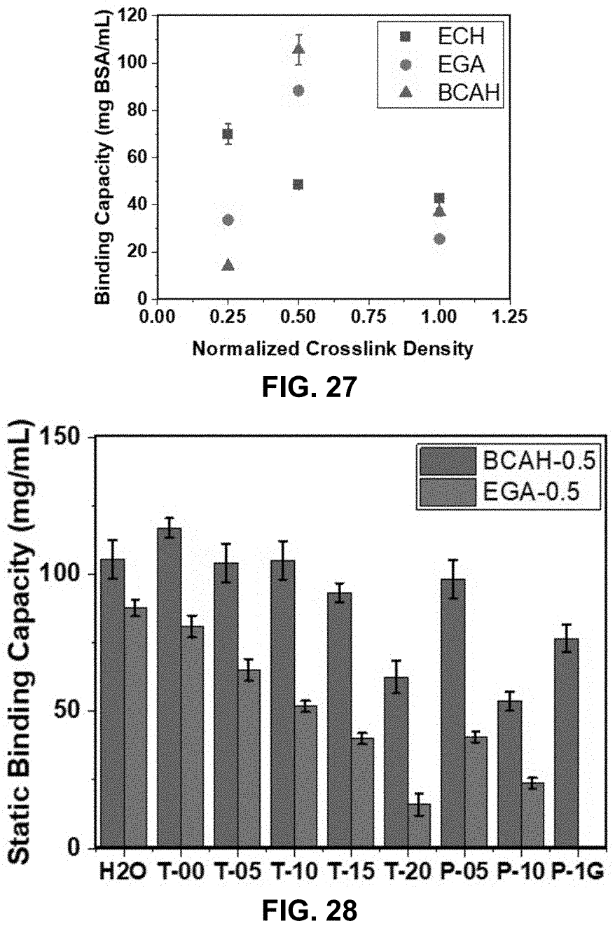

Static protein binding experiments were performed for all formulations in Table 7. The two formulations with the highest binding (54E & 54H) were then used to test salt tolerance in water and the buffers listed in Table 8. The static binding capacity (SBC) experiment operated as follows. A 2 mg BSA/mL solution was prepared by dissolving BSA in the appropriate solvent as outlined in Table 8. A 12 mm×12 mm sample token was then cut out of the membrane of interest and immersed in 5 mL of the BSA solution. The solution was rocked gently for 48 hours before the absorbance at 280 nm was measured using an Agilent 8453 UV/vis spectrometer. The concentration of BSA in the solution was then estimated using an absorbance/concentration calibration curve. The mass of BSA bound was then determined using a mass balance, while the membrane volume was calculated using the sample thickness determined via SEM imaging. Replicates of each formulation were tested with the average binding capacity and standard deviations reported in FIG. 27.

TABLE 8
Composition of buffers used during salt tolerance measurements. Each buffer had a pH
of 7.4 and the following concentrations of the buffer chemistry: 1-50 mM TRIS,
2-0.5× PBS, and 3-1× PBS.

| | T-00 | T-05 | T-10 | T-15 | T-20 | P-05 | P-10 | P-1G |
|---|---|---|---|---|---|---|---|---|
| Buffer | TRIS¹ | TRIS¹ | TRIS¹ | TRIS¹ | TRIS¹ | PBS² | PBS³ | PBS³ |
| chemistry | | | | | | | | |
| Added NaCl | 0 | 50 | 100 | 150 | 200 | — | — | — |
| (mM) | | | | | | | | |
| Glycerol | — | — | — | — | — | — | — | 50 |
| (mM) | | | | | | | | |
| Conductivity | 4.6 | 9.8 | 15 | 19.5 | 24.7 | 9.1 | 17.6 | 18 |
| (mS/cm) | | | | | | | | |

Dynamic protein binding experiments used membranes with formulation 54H because they demonstrated the best binding capacity in the presence of salt. The dynamic binding measurements were performed using a precision adsorption flow-through cell with operating volume of 80 μL from Hellmanex and the UV-vis' time resolved module. The measurements were performed as follows.

A 2 mg/mL BSA solution was prepared by dissolving BSA in 50 mM TRIS buffer with varying salt concentrations (0, 50, 100, 150, and 200 mM respectively). Flat sheet membranes were cut into circle tokens with a diameter of 25.4 mm, hereafter referred to as samples, while they were still wet. The prepared samples were stored in 50 mM TRIS buffer. A nonwoven PET support was also cut into circles with a diameter of 25.4 mm. A control measurement was taken by loading one layer of the PET support into the sample holder and then introducing the BSA feed solution at a constant flowrate. The time-resolved absorbance at 280 nm was captured using a UV-vis spectrometer. The sample was then loaded into the sample holder on top of a fresh PET support to account for any nonspecific binding to the nonwoven support. The sample was equilibrated to the feed solution using the appropriate buffer. Once the sample was equilibrated, the BSA feed solution was introduced at a constant flowrate using a syringe pump. The flowrates investigated in these experiments were 0.3, 0.6, 1.2, & 1.5 mL/min, corresponding to 2, 4, 8, & 10 membrane volumes/minute, respectively. The lowest flowrate (0.3 mL/min, 2 MV/min) was only measured in TRIS buffer with 0 mM NaCl. The mass of BSA bound by the membrane was then calculated by taking the difference in the mass of BSA loaded between the sample and the control at 10% breakthrough.

3.3 Results and Discussion

3.3.1 Changes in Morphology

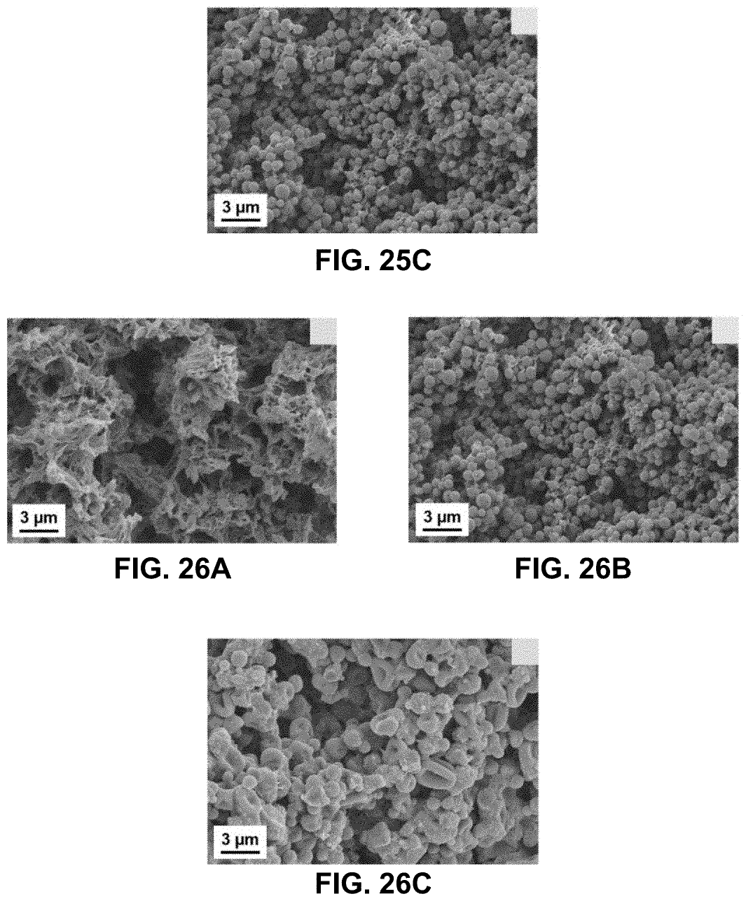

FIGS. 25A-25C show SEM micrographs of the membrane cross-sections prepared with different crosslinkers at NCD 0.5. Using ECH as the crosslinker, FIG. 25A, produces microgels that form distinct spheres with a large size distribution (0.5-3 microns) when dried. This is a notable deviation from the tight size distribution of the spherical microgels when ECH has an NCD of 1.0 (FIG. 19C). In contrast to the regular spheres obtained with ECH, using EGA as the crosslinker produces microgels that are interconnected thereby losing the distinct spherical shape. The tightest distribution of microgel sizes in the dry state is seen in FIG. 25C, when BCAH is used as the crosslinker. The tight size distribution and smaller average particle size is equivalent to those produced when using ECH at an NCD of 1. This finding is particularly interesting for two reasons: First, EGA does not produce a similar morphology at any of the NCDs investigated in these experiments. Second, the concentration of halide bonds in the crosslinker is the same for ECH at NCD of 1 and BCAH at NCD of 0.5. These two observations seem to indicate that the halide concentration plays an essential role in determining particle morphology.

FIGS. 26A-26C display SEM micrographs of membrane cross-sections prepared using BCAH at NCDs of 0.25, 0.5, and 1 (FIGS. 26A-26C respectively). At a low BCAH concentration few functional particles are visible indicating that formation of the microgels is suppressed at low NCD. The microgel suppression is attributed to PEI which is not entangled with PVDF escaping the gel during casting. At a high BCAH concentration, NCD of 1, the functional microgels exhibit a structure consistent with a collapsed hollow sphere in the dry state. The transition to forming hollow spheres at high crosslink densities has not been fully investigated at this time, but one documented contribution is the interaction between dissolved PEI and droplets of BCAH/DMSO. The change in particle morphology is also accompanied by a shift in PVDF structure. The membranes prepared at NCDs of 0.25 and 0.5 both show PVDF structures consistent with using IPA as the nonsolvent Example 8, Section 2.3.1), while the membrane with NCD of 1 shows PVDF structures more consistent with using H₂O:NMP as nonsolvent. The change in PVDF morphology stems from the addition of DMSO, which at high enough concentrations influences the PVDF structure in a similar way as NMP does when using the mixed nonsolvent.

3.3.2 Static Binding

The static binding capacities depicted in FIG. 27 provide key insights to the relationship between crosslinker chemistry, crosslink density, and SBC. For instance, the SBC of membranes prepared with EGA and BCAH both have a local maximum at NCD of 0.5, which is significantly higher than the binding capacities at NCDs of 1 or 0.25. Noting that both BCAH and EGA are both homofunctional molecules, the local maximum is attributed to the influence of crosslink density on gel tightness and cohesion. At high crosslinker concentrations the gel is tightly crosslinked, which limits its ability to swell in water. The limited swelling leads to a low SBC because only a portion of the BSA binding sites are available to interact (due to the binding sites being dispersed throughout the entire gel and not just located on the surface). In contrast, low crosslinker concentrations negatively influences both gel cohesiveness and microgel concentration resulting in a lower binding capacity. At an NCD of 0.25 there are few enough crosslinks such that an uneven distribution of crosslinks per PEI leads to several PEI molecules which are not covalently bound to the gel prior to casting the membrane. The non-crosslinked PEI molecules get removed during the coagulation and washing steps, leading to a lower number of binding sites in the membrane and a lower overall binding capacity. At NCD 0.5 the gel is open enough to maximize availability of functional sites while having sufficient crosslinks to maintain gel cohesion and PEI concentration. The balance of these two contributions gives rise to the observed maximums corresponding to membranes 54E and 54H. These observations are also validated by the reduction in particle size and change in particle shape observed in the SEM micrographs of FIGS. 26A-26C.

Changes in crosslink density provides a feasible explanation for the observed trends in membranes prepared using both EGA and BCAH, but consideration of crosslinker chemistry is needed to account for the differences between these membranes at a given crosslink density. Membranes synthesized with BCAH have higher binding capacities at NCDs0.5, while at an NCD of 0.25 using EGA results in higher BSA binding. The higher binding when using BCAH is attributed to the additional secondary amine incorporated into the gel during the PEI polymerization reaction. The higher binding of membranes prepared with EGA at NCD 0.25 has not yet been fully explored but may be explained by using BCAH leading to a lower gel cohesion because of its slower reaction kinetics and short length. Crosslinker chemistry also plays a critical role in explaining why membranes prepared with ECH exhibit a decreasing SBC with increasing NCD. In contrast to BCAH and EGA, ECH is a short heterofunctional molecule with one functional group (epoxide) that reacts significantly more quickly than the other (halide). The difference in reaction rates could lead to a more even distribution of crosslinks at lower NCD thereby reducing the percentage of the PEI which escapes the membrane. As the crosslink density increases the even distribution of crosslinks and shorter length of ECH results in a tighter gel leading to a low BSA binding.

3.3.3 Salt Tolerance

As discussed in the previous section, membranes 54H and 54E were used for the salt tolerance measurements due to their high BSA binding capacity in water. The static binding capacities for 54H and 54E, depicted in FIG. 28, demonstrate that both crosslinker chemistry and buffer composition (excluding added salts) influence membrane salt tolerance. The influence of crosslinker chemistry is demonstrated by comparing the percent decrease in binding capacity with increasing buffer conductivity. Using the BSA binding capacity in water as the reference, membranes prepared with EGA, 54E, have a reduction in binding capacity of 25%, 40%, & 80% when using buffers T-05, T-10, and T-20. In comparison, membranes prepared using BCAH, 54H, have binding capacities within error of the reference when using T-05 and T-10 buffers. In the T-20 buffer, membrane 54H demonstrates a BC reduction of 40%. Similar behavior is observed when the PBS buffers are used. However, a comparison between binding capacities in different buffers with similar conductivities reveals that using a PBS based buffer has a detrimental effect on SBC. While this trend holds true for both 54H and 54E membranes, the effect is more pronounced when using EGA as the crosslinker. For example, the percent reduction from T-05 to P-05 is 5% when 54H is used and 37% when using 54E. FIG. 28 also demonstrates that incorporating glycerol (P-1G) at the same concentration as the TRIS buffer improves the SBC in the PBS buffer.

The divergence in SBC of membranes 54E and 54H at higher conductivities (using the same base buffer) indicates that there is a contribution to the binding mechanism that is more pronounced when using BCAH as the crosslinker. Prior literature has explained the source of this contribution by demonstrating that incorporating primary and secondary amines enables binding in high salt environments through alternative binding mechanisms, such as hydrogen bonding^{6,9}. It is also noted in both studies that tertiary and quaternary amines do not demonstrate binding in solutions with higher conductivities because they only use electrostatic interactions, which are increasingly screened by salt ions as the conductivity rises. During the PEI polymerization reaction primary and secondary amines are consumed by the crosslinkers producing secondary and tertiary amines respectively. Therefore, when EGA and ECH are used as crosslinkers some of the secondary amines of PEI will be consumed and turned into tertiary amines. Due to each tertiary amine being a site that can only bond through electrostatic interactions, the tertiary amines produced may be considered a loss of binding capability at higher salt concentrations. Similarly, using BCAH as the crosslinker produces new tertiary amines during the PEI crosslinking; however, each BCAH molecule also adds a new secondary amine to the polymerized PEI gel. As a result, BCAH has significantly more sites capable of hydrogen bonding for a given crosslinker molar concentration.

Prior literature has provided several examples of salt tolerance in TRIS^{6,9,11-14} and PBS^{3,4,15} based buffers, but to our knowledge no study has compared binding performance between the two buffers to determine buffer sensitivity of a given membrane. The buffer sensitivity revealed in FIG. 28 highlights the importance of identifying a suitable buffer for a given pairing of membrane material and protein of interest. Under these specific conditions, the observed behavior is a result of differences in buffer chemistry that leads to changes in the interactions between the buffer, PEI microgels, and BSA. TRIS, being an organic molecule consisting of an amine group and three hydroxyl groups, can interact with the binding ligand and BSA through both electrostatic interactions and hydrogen bonding. The ability of TRIS buffer to form hydrogen bonds may magnify the PEI microgel's hydrogen bonding contribution, thereby reducing the effects of electrostatic screening at higher conductivities. In contrast, PBS is composed of monohydrogen and dihydrogen phosphate salts as well as NaCl and KCl. As a result, the PBS buffer has little impact on the hydrogen bonding contribution and predominantly screens the electrostatic binding interactions leading to a lower volumetric binding capacity. In order to confirm that hydrogen bonding of the buffer makes a significant contribution to SBC, static measurements were conducted using 1×PBS with 50 mM glycerol. Glycerol was chosen both for its propensity to form hydrogen bonds and its property of remaining neutral in water. The combination of these two properties verifies that any improvement in BSA binding between P-10 and P-1 G is solely due to hydrogen bonding form glycerol. The improvement of over 40% supports the claim that hydrogen bonding of the buffer makes a significant contribution to SBC. The difference in capacity between P-1G and T-15 has still not been fully determined, but there is feasibly a synergistic effect between charge and hydrogen bonding on TRIS that facilitates higher BSA binding.

3.3.4 Dynamic Binding

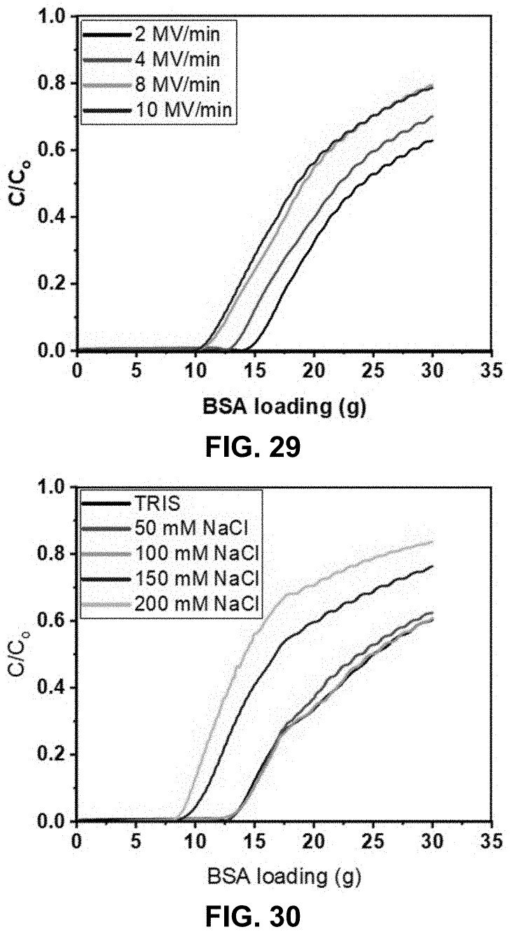

One of the key advantages of traditional membrane chromatography is independence from flowrate that allows membranes to be used at high flowrates without sacrificing binding capacity^{1-5,9}. As seen in FIG. 29, the combination of resin and membrane properties exhibited by mixed-matrix membranes introduces a regime of flowrate dependence. At low flowrates (FIGS. 29, 2 and 4 Membrane Volumes/min) the additional time BSA has to penetrate and bind with the functional microgel is inversely proportional to the volumetric flow rate leading to decreasing binding capacity as the flowrate is increased. As the flowrate continues to increase (FIGS. 29, 8 & 10 Membrane Volumes/min) the binding capacity plateaus and is no longer flowrate dependent indicating that the mass transfer to binding is dominated by convective forces similar to other membrane-based adsorbers.

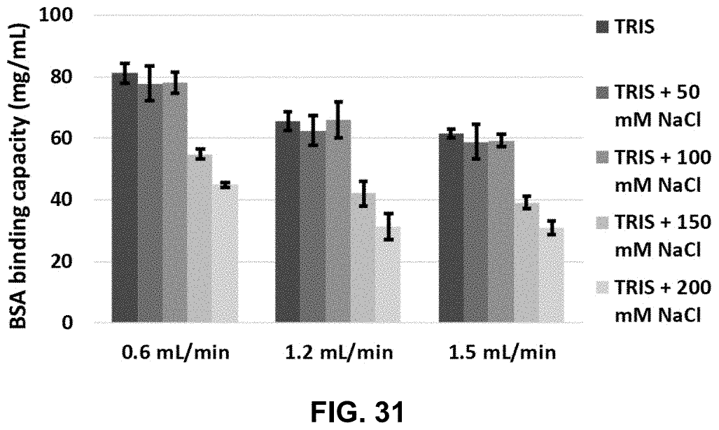

The modified mixed-matrix membrane's salt tolerance under flow agrees with the trend observed in the static binding measurements. As seen in FIG. 30, the BSA breakthrough curves are essentially constant in the presence of TRIS buffers with up to 100 mM NaCl added. As the salt concentration increases past 100 mM NaCl, the curves shift to the left indicating that the DBC decreases. Noting how the trend in DBC with respect to salt concentrations changes across the different flowrates tested (FIG. 31) is important to characterizing the binding behavior of the PEI microgels. As noted above, at low flowrates BSA is able to more fully penetrate the functional microgels leading to a higher binding capacity. In contrast, at high flowrates BSA diffuses a shorter distance and therefore interacts with a smaller portion of the various microgels. By comparing how the DBC reacts to salt concentration at different flowrates, it is possible to draw qualitative comparisons to the binding interactions of different portions of the microgels. For example, if the salt tolerance at lower flowrates showed a smaller percentage reduction between binding in TRIS buffer alone and TRIS buffer with 200 mM NaCl than the difference at a higher flowrate, it would suggest that the edges of the microgel have a lower salt tolerance than the interior of the microgels. Comparing the percent reduction in DBC of the flowrates presented in FIG. 31 provides the following: 0.6 mL/min—45±3%, 1.2 mL/min—52±9%, and 1.5 mL/min—50±5%. There is not a statistically significant difference between the three flowrates suggesting that the binding interactions across the microgel are equivalent (p-value 0.05).

3.4 Conclusions for Example 9

The influence of crosslinker chemistry and crosslink density on volumetric binding capacity and salt tolerance was investigated during this study. It was determined that the volumetric binding capacity has a nonlinear relationship to crosslink density that is a function of the crosslinker chemistry. It was demonstrated that by changing the crosslinker chemistry from heterofunctional to homofunctional a maximum binding capacity of >100 mg/mL could be achieved at an NCD of 0.5. In contrast, the hetereofunctional crosslinker demonstrated a decrease in binding capacity as the NCD increases. The optimum membrane formulation was then used for the salt tolerance and dynamic binding measurements. In these measurements, membrane 54H demonstrated consistent binding (>90% of maximum binding) up to 100 mM added salt in 50 mM TRIS buffer. The dynamic binding measurements revealed a flowrate dependent regime while operating at low flowrates (2-4 MV/min). Once the flowrate surpassed 8 MV/min the DBC plateaued and the dependence on flowrate was lost. The first regime at low flowrates demonstrated a flowrate dependence similar to that seen in resin chromatography. In addition, the DBC measurements validated membrane salt tolerance under flow with >90% of the binding capacity maintained up to 100 mM NaCl added at all flowrates tested.

### References Corresponding to Example 9

(1) Ghosh, R. Protein Separation Using Membrane Chromatography: Opportunities and Challenges. J. Chromatogr. A 2002, 952 (1), 13-27. https://doi.org/10.1016/S0021-9673(02)00057-2.
(2) Madadkar, P.; Wu, Q.; Ghosh, R. A Laterally-Fed Membrane Chromatography Module. J. Membr. Sci. 2015, 487, 173-179. https://doi.org/10.1016/j.memsci.2015.03.056.
(3) Bhut, B. V.; Wickramasinghe, S. R.; Husson, S. M. Preparation of High-Capacity, Weak Anion-Exchange Membranes for Protein Separations Using Surface-Initiated Atom Transfer Radical Polymerization. J. Membr. Sci. 2008, 325 (1), 176-183. https://doi.org/10.1016/j.memsci.2008.07.028.
(4) Bhut, B. V.; Husson, S. M. Dramatic Performance Improvement of Weak Anion-Exchange Membranes for Chromatographic Bioseparations. J. Membr. Sci. 2009, 337 (1), 215-223. https://doi.org/10.1016/j.memsci.2009.03.046.
(5) Sun, L.; Dai, J.; Baker, G. L.; Bruening, M. L. High-Capacity, Protein-Binding Membranes Based on Polymer Brushes Grown in Porous Substrates. Chem. Mater. 2006, 18 (17), 4033-4039. https://doi.org/10.1021/cm060554m.
(6) Fischer-Frühholz, S.; Zhou, D.; Hirai, M. Sartobind STIC® Salt-Tolerant Membrane Chromatography. Nat. Methods 2010, 7 (12), 12-13.
(7) Orr, V.; Zhong, L.; Moo-Young, M.; Chou, C. P. Recent Advances in Bioprocessing Application of Membrane Chromatography. Biotechnol. Adv. 2013, 31 (4), 450-465. https://doi.org/10.1016/j.biotechadv.2013.01.007.
(8) Keating, J. J.; Imbrogno, J.; Belfort, G. Polymer Brushes for Membrane Separations: A Review. ACS Appl. Mater. Interfaces 2016, 8 (42), 28383-28399. https://doi.org/10.1021/acsami.6b09068.
(9) Riordan, W.; Heilmann, S.; Brorson, K.; Seshadri, K.; He, Y.; Etzel, M. Design of Salt-Tolerant Membrane Adsorbers for Viral Clearance. Biotechnol. Bioeng. 2009, 103 (5), 920-929. https://doi.org/10.1002/bit.22314.
(10) Fan, J.; Luo, J.; Chen, X.; Wan, Y. Facile Preparation of Salt-Tolerant Anion-Exchange Membrane Adsorber Using Hydrophobic Membrane as Substrate. J. Chromatogr. A 2017, 1490, 54-62. https://doi.org/10.1016/j.chroma.2017.02.016.
(11) Han, X.; Hong, T.; Lutz, H.; Becerra-Arteaga, A.; Blanchard, M.; Zhao, X.; Hewig, A.; Natarajan, V. Performance of a Salt-Tolerant Membrane Adsorber in Flow-through Mode. BioProcess Int 2013, 11 (2), 28-39.
(12) Champagne, J.; Balluet, G.; Gantier, R.; Toueille, M. “Salt Tolerant” Anion Exchange Chromatography for Direct Capture of an Acidic Protein from CHO Cell Culture. Protein Expr. Purif. 2013, 89 (2), 117-123. https://doi.org/10.1016/j.pep.2013.03.005.
(13) Woo, M.; Khan, N. Z.; Royce, J.; Mehta, U.; Gagnon, B.; Ramaswamy, S.; Soice, N.; Morelli, M.; Cheng, K.-S. A Novel Primary Amine-Based Anion Exchange Membrane Adsorber. J. Chromatogr. A 2011, 1218 (32), 5386-5392. https://doi.org/10.1016/j.chroma.2011.03.068.
(14) Chen, G.; Umatheva, U.; Alforque, L.; Shirataki, H.; Ogawa, S.; Kato, C.; Ghosh, R. An Annular-Flow, Hollow-Fiber Membrane Chromatography Device for Fast, High-Resolution Protein Separation at Low Pressure. J. Membr. Sci. 2019, 590, 117305. https://doi.org/10.1016/j.memsci.2019.117305.
(15) Qian, X.; Fan, H.; Wang, C.; Wei, Y. Preparation of High-Capacity, Weak Anion-Exchange Membranes by Surface-Initiated Atom Transfer Radical Polymerization of Poly(Glycidyl Methacrylate) and Subsequent Derivatization with Diethylamine. Appl. Surf. Sci. 2013, 271, 240-247. https://doi.org/10.1016/j.apsusc.2013.01.167.

### Example 10: Design of Polymer-Ceramic Composites for Membrane Chromatography

4.1 Introduction

Up to this point we have considered the mixed-matrix membranes with in situ generated functional particles as free standing or on top of a nonwoven support. However, as we have investigated higher particle loadings and lower crosslinker concentrations, the material has shown signs of losing its mechanical stability and uniformity on length scales >1 mm. The decreasing mechanical integrity stems from the intrinsic properties of the functional microgels. The PEI microgels are a subset of hydrogels, a class of soft matter materials that are comprised of hydrophilic polymer networks that swell, but do not dissolve, in the presence of water. The intrinsic properties of hydrogels stemming from their unique composition provide both improved functionality and reduced mechanical robustness. As a result of this dichotomy, several methods have been developed to incorporate hydrogels into composites with ceramics and stiff porous polymers.

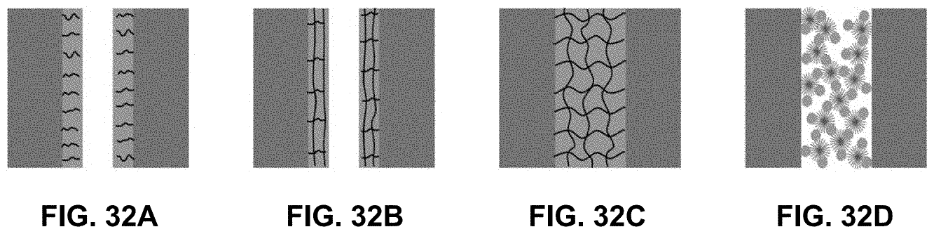

FIGS. 32A-32C depict three methods of incorporating functional hydrogels into a porous scaffold that have been well documented in the literature¹⁻⁷. For example, Anuraj and coworkers disclosed a route to integrate a functional hydrogel layer into a porous ceramic using polymer brushes, thereby producing a structure similar to FIG. 32A. The resulting composite demonstrated efficient capture and purification of proteins from complex mixtures². In another example, Yang et al. investigated the role of crosslink density on the performance of crosslinked polymer chain hydrogels (FIG. 32B). They discovered that a lower concentration of crosslinks between polymer chains improved the reaction time of environmentally responsive hydrogels³. An example of the pore-filling method shown in FIG. 32C was demonstrated by Adrus et al. using an in situ photopolymerization to grow a hydrogel inside a porous support. The resulting hydrogel mesh size was temperature sensitive, which provided tunable control over the size selectivity of the composite membrane.

Both functionalizing the pore wall and pore-filling are effective methods in combining the mechanical strength of porous supports with the functionality of hydrogels. However, there has been a growing trend in the field emphasizing the use of hydrogel particles, instead of layers, on both the micro and nano scale⁸. The shift in focus stems from the hydrogel particles having faster swelling and stimuli responsive kinetics as well as a 3D porous structure useful in biosensing and bioseparations⁹. Here we discuss a novel method to form composite membranes with stably incorporated microgels as depicted in FIG. 32D. The composite membrane is fabricated by infiltrating a silicon oxycarbide (SiOC) ceramic scaffold with the polymer dope solution used to fabricate the mixed-matrix membranes Examples 8 and 9. Following infiltration, the structural polymer in the dope solution is solidified using phase inversion micromolding. The morphology of the solidified polymer matrix was tailored using the conclusions of Example 8. The ceramic scaffold, developed by Dr. Arai, is fabricated via freeze casting techniques that provide both mechanical robustness and a plurality of oriented pores¹⁹.

The composite membrane was characterized using SEM to demonstrate process feasibility. Static and dynamic BSA binding experiments were conducted to probe performance of the composite membranes in bioseparations.

4.2 Experimental Methods

4.2.1 Chemicals and Materials

Polyvinylidene Fluoride (PVDF) [Kynar 761] was provided by Arkema (King of Prussia, Pa.). Hyperbranched polyethylenimine (PEI) was procured from Polysciences. Cyclohexane (C6H12), Epichlorohydrin (ECH), 3-Aminopropyltrimethoxysilane (ATMS), Bovine Serum Albumin (BSA), Bis(2-chloroethyl)amine hydrochloride (BCAH), Triethyl Phosphate (TEP), Isopropanol (IPA), Dimethyl sulfoxide (DMSO), and TRIS hydrochloride (TRIS) were purchased from Millipore Sigma. Hydrochloric acid was purchased from EMD Millipore. Polysiloxane (CH3-SiO1.5, Silres® MK Powder) and Geniosil® GF 91 were purchased from Wacker Chemie. All chemicals and materials were used as received. Buffers were prepared using indicated chemicals and distilled water.

4.2.2 Ceramic Fabrication

A polymer solution was prepared by dissolving the polysiloxane preceramic polymer in cyclohexane, with concentration of preceramic polymer of 20 wt. %. Once a homogenized solution was obtained, a cross-linking agent (Geniosil® GF 91) was added in concentrations of 1 wt. % and stirred for 5 minutes and then degassed for 10 min to avoid air bubbles during solidification. The freeze-casting was done by pouring the polymer solution into a glass mold (h=20 mm, Ø=25 mm) that was located on a PID-controlled thermoelectric plate. Another thermoelectric was placed on top of the mold to control both freezing front velocity and temperature gradient. A cold finger with smaller diameter than the mold was inserted into the glass mold such that the created spaces act as reservoir for the solution as the solution shrunk by solidification. The freezing front velocity and temperature gradient were measured by taking pictures every 30 seconds using a camera and intervalometer. The temperature gradient, G was defined by the following equation:

$$G = \frac{{T}_{r} - {T}_{f}}{d} \qquad ( 4.1 )$$

where Tₜ is the temperature of top cold finger, T_{f} is the temperature of freezing front and d is the distance between the top cold finger and the freezing front. The temperature of the freezing front was assumed to be at the liquidus temperature of the solution, and the value was taken from the work by Naviroj¹². All samples were frozen at freezing front velocities of 15 μm/s, and temperature gradients of 2.5 K/mm to maintain homogeneous pore structures.

Once the structure was completely frozen, the isothermal coarsening was initiated by setting the top and bottom thermoelectrics to 4° C. After the structure was coarsened for 3 hours, the sample was re-froze¹³. After the sample was completely frozen, the sample was sublimated in a freeze drier (VirTis AdVantage 2.0) where the solvent crystals were completely removed. After freeze drying, the polymer scaffold was pyrolyzed in argon at 1100° C. for four hours to convert the preceramic polymer into silicon oxycarbide (SiOC). This resulted in a porosity of ˜77%. The pyrolyzed sample was machined into a disk with thickness of ˜1.6 mm and diameter of ˜13 mm prior to infiltration.

4.2.3 Polymer Dope Synthesis

The polymer dope synthesis was initiated by adding 5.91 g of PVDF to an empty 3-neck round bottom flask. The flask was then outfitted with an overhead mechanical stirrer and the necessary greased connectors. Next, 30 mL of TEP was added to the flask and then the remaining openings were sealed using rubber septa. The PVDF/TEP mixture was heated to 80° C. and incubated for 1 hour before the mixing speed was set to 60 rpm. The resulting solution was left to equilibrate overnight. A PEI solution was prepared by adding the mass of PEI indicated in Table 9 to a scintillation vial followed by 5 mL of TEP. The mixture was vortexed and shaken until no concentration gradients were visible and then it was left to equilibrate overnight at room temperature. The crosslinker solution was prepared by weighing the required amount of BCAH into a scintillation vial and then adding the corresponding volume of DMSO (Table 9). The resulting mixture was incubated overnight at room temperature to fully dissolve the BCAH.

TABLE 9
Composition of polymer dope solution and associated Normalized
Crosslink Density (NCD). Naming scheme goes as wt. %
PEI in the dry polymeric membrane - NCD.

| Dope | | | | DMSO | |
|---|---|---|---|---|---|
| solution | PVDF (g) | PEI (g) | BCAH (g) | (mL) | NCD |
| 54-0.5 | 5.91 | 5 | 3.1 | 5 | 0.5 |
| 54-0.25 | 5.91 | 5 | 1.55 | 2.5 | 0.25 |
| 54-0.125 | 5.91 | 5 | 0.78 | 1.25 | 0.125 |
| 54-0.0625 | 5.91 | 5 | 0.39 | 0.625 | 0.0625 |
| 54-0.4 | 5.91 | 5 | 2.5 | 4 | 0.4 |
| 38-0.4 | 5.91 | 2.58 | 1.28 | 2 | 0.4 |

The next day, the reaction flask was purged with N₂ for 7 minutes and the mixing speed was increased to 250 rpm. With the N₂ flow still on, the PEI solution was then added dropwise to the flask using a glass Pasteur pipette over the course of 4 minutes. The resulting solution was left to mix for 5 minutes before adding 0.43 mL of concentrated HCl (37% solution). Following the addition of the HCl, the flask was incubated for 15 minutes at 80° C. with the mixing speed maintained at 250 rpm. The crosslinker solution was then added to the flask and the polymerization reaction was allowed to proceed for 4 hours. After the 4-hour reaction time, the flask was put under in-house vacuum for 10 minutes to remove entrapped air. The resulting dope solution was then added to the ceramic using the steps outlined in Section 4.2.5 of this example.

Several polymeric membranes were prepared at wt % PEI in the dry polymeric membrane—NCD compositions of 54-0.5, 54-0.25, 54-0.125, 54-0.0625 as controls for the static binding measurements. The same steps outlined above were followed until completing the incubation under vacuum. The resulting dope solution was then cast on glass plates at a blade height of 300 μm. The cast membranes were left at room temperature for 30 seconds before being immersed in an isopropanol coagulation bath. After two hours, the solidified membranes were moved to distilled water baths prior to storage.

4.2.4 Surface Functionalization of Ceramic

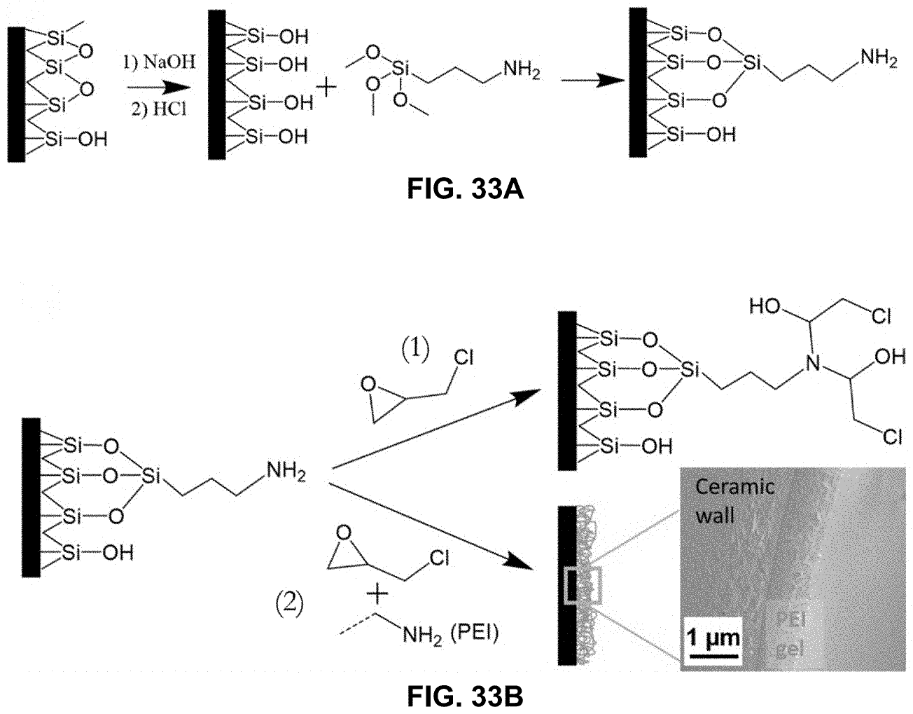

Prior to adding the polymer dope solution, the ceramic surface was activated and functionalized (FIG. 33A) using a procedure derived from prior literature¹⁴⁻¹⁷. The SiOC scaffold was first immersed and incubated in 1 M NaOH for 90 minutes. It was then washed in water before being incubated in a 0.1 M HCl solution for 30 minutes. The ceramic was then washed in water again, before being dried at 110° C. for 1 hour. Once the ceramic was dried, it was added to a 2 v % solution of ATMS in isopropanol and incubated for 3 hours at 60° C. The sample was then washed thoroughly in water and isopropanol before being cured at 110° C. for 30 minutes.

Following the curing of the aminosilane layer, the ceramic surface was further functionalized following the two reaction schemes presented in FIG. 33B. Ceramics prepared using the top route were incubated in an IPA/ECH solution overnight. Following the overnight incubation, the samples were thoroughly washed with IPA and then left to dry at room temperature before the addition of the polymer solution. The resulting surface was expected to be terminated in chloride groups, which would react readily with the primary and secondary amines in the polymer solution.

The second further functionalization route was designed to increase the number of functional groups on the wall available to interact with amines in the PEI microgels. The functionalization proceeded as follows: the functionalization solution was prepared by dissolving PEI with IPA at a molar ratio of 1:37.4, respectively. Ten minutes before adding the solution to the ceramic, ECH was added to the solution at a molar ratio of 1 mole PEI for 16.5 moles ECH—corresponding to 1.1 ECH molecules for every available amine. This ratio was chosen to minimize the crosslinking between PEI molecules and thereby maximize the number of reactive sites. The ceramic was incubated in the IPA/PEI/EHC solution overnight at room temperature. After the overnight incubation, DMSO was added to the vial and the resulting solution was heated to 80° C. for 1 hour to remove the leftover reactants and unbound products. The sample was then washed with IPA and dried at room temperature for one hour prior to the addition of the polymer dope solution.

4.2.5 Phase Inversion Micromolding

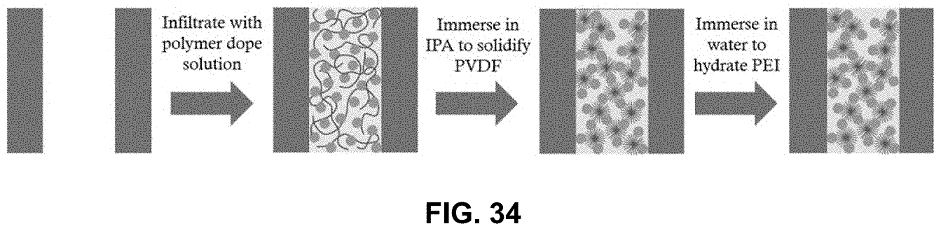

The phase inversion micromolding process shown in FIG. 34 was used for both neat ceramic samples and ceramics functionalized using the pathways described above. The ceramic scaffold was loaded into the infiltration device and the polymer dope solution was injected using a syringe pump. The solution was pumped at a rate of 100 μL/min until the ceramic and infiltration device were filled. The device was then incubated at 80° C. for 1 hour for both the functionalized and the neat ceramic samples. Following the incubation, the samples were removed from the infiltration device and placed in IPA for an overnight incubation. The following day, the samples were moved to water baths to remove trace solvent and IPA in preparation for BSA binding characterization.

4.2.6 Membrane Properties Characterization

4.2.6.1 SEM

The microstructure of ceramic scaffolds and polymer/ceramic composites were observed using a field emission scanning electron microscope (FE SEM-Zeiss 1550 VP). In preparation for imaging, the samples were dried at 70° C. overnight. To prepare the cross-sectional view, the membranes were broken by hand at ambient conditions. The surfaces and cross-sections of interest were then coated with a Pt/Pd conductive layer (10 nm) using a sputter coater and then imaged.

4.2.6.2 Protein Adsorption Studies

BSA was used as the model protein in both static and dynamic binding measurements. Initial tests were done using BSA in distilled water at a concentration of 2 mg/mL. To measure the static binding of the polymeric membrane references, a membrane with a known volume was immersed in a 2 mg/mL BSA solution and gently mixed for 48 hours. The absorbance of the solution was then measured using an Agilent 8453 UV/vis and the reported absorbance at 280 nm was used to determine the concentration of BSA in the solution. The mass of BSA bound was then determined using a mass balance.

A similar process was used to measure the static binding capacity (SBC) of the composite membranes. The samples were immersed in a 2 mg/mL BSA solution and gently rocked for 48 hours. The absorbance was then measured and the binding capacity calculated before the samples were rocked for another 72 hours. The absorbance was then measured again and the binding capacity calculated. The addition of the second absorbance measurement was to account for the increased thickness and reduced mass transfer in the composites. The initial experiments for comparison to the polymeric membranes used BSA dissolved in H₂O, all subsequent measurements used BSA dissolved in 50 mM TRIS.

Dynamic binding measurements using 2 mg/mL BSA in 50 mM TRIS buffer were conducted using composites with formulations of 54-0.25 and 38-0.4. To run the measurement, the sample was first loaded into a Swinney filter holder (Pall Corp.) and was equilibrated using 50 mM TRIS buffer. The BSA solution was then introduced via a syringe pump to the device at a rate of 150 μL/min (or 2 membrane volumes/min). The filtrate was analyzed with time-resolved measurements on the Agilent 8453 UV/vis. The 10% breakthrough curve method, as described in Example 9, was used to determine the dynamic binding capacity.

4.3 Results and Discussion

4.3.1 Phase Inversion Micromolding Feasibility

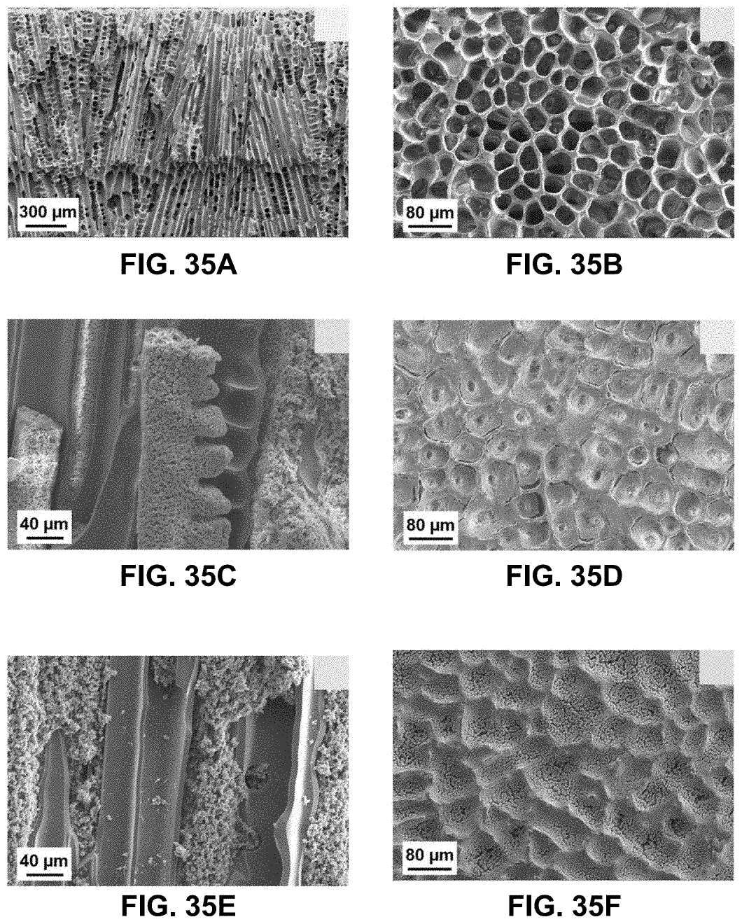

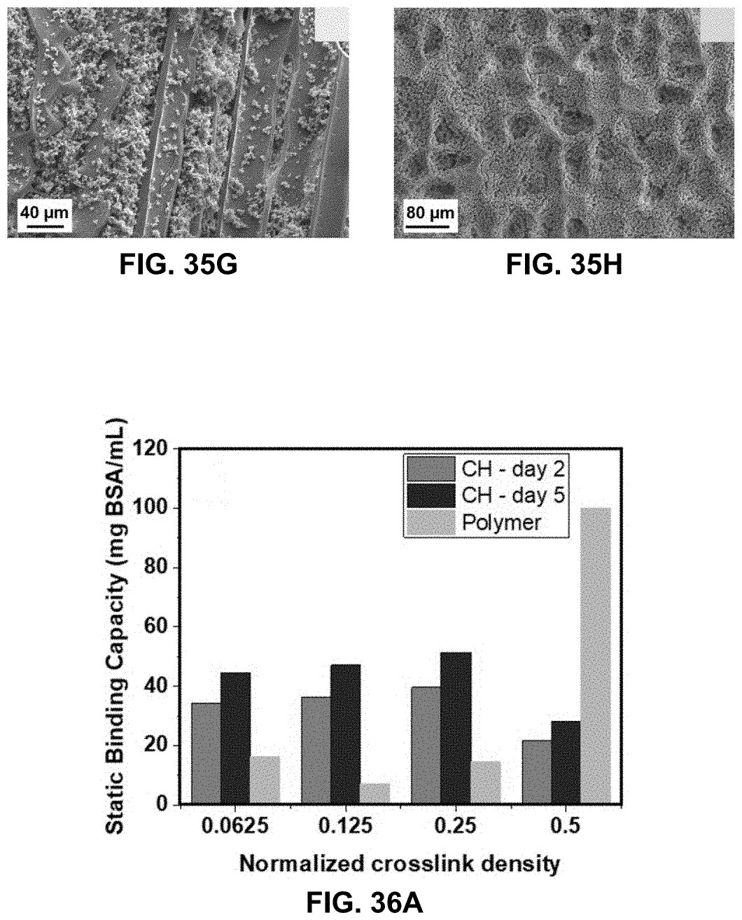

FIGS. 35A-35H show SEM micrographs of the cross-section and surfaces of the ceramic scaffold and composite membranes with different surface functionality. The longitudinal cross-sectional image of the neat ceramic scaffold, FIG. 35A, shows the highly oriented pores that transverse the entire membrane. The corresponding surface (perpendicular to the freeze-casting direction), FIG. 35B, demonstrates the morphology of the oriented pores as well as the average pore diameter of 20 μm. The composite presented in FIGS. 35C-35D was infiltrated without modifying the surface of the ceramic. In panel c, there is a segment of the polymer matrix in the middle of the micrograph that has a morphology that closely matches the contours of the nearby ceramic pore wall. It is also noteworthy that the ceramic surfaces that are visible are all bare. In the surface view from panel d the pores are mostly filled with the polymer matrix, but there are many cases where there is a debonded interface between the polymer matrix and one side of the pore.

The composite presented in FIGS. 35E-35F had the surface modified using reaction (1) from FIG. 33B prior to the infiltration and phase inversion micromolding. The polymer matrix once again fills the pores in panel e and the ceramic walls that are visible are lightly decorated in microparticles from the polymer matrix. FIG. 35F shows that the ceramic pores are completely filled and there are no visible gaps between the polymer matrix and the pore wall. The composite presented in FIGS. 35G-35H had the surface modified using reaction (2) from FIG. 4.3b, producing a functional PEI gel layer prior to infiltration and phase inversion micromolding. The polymer matrix fills the pores in FIG. 35G and the ceramic walls that are visible are decorated with a higher density of microparticles/polymer matrix than FIG. 35E. FIG. 35H shows that the ceramic pores are once again completely filled and there are no visible gaps between the polymer matrix and the pore wall.

The observations of the behavior of the polymer matrix in FIGS. 35C-35D, provide several key insights on phase inversion micromolding and how to stably integrate the mixed-matrix membrane with the ceramic scaffold. The match between the morphology of the polymer matrix and the contours of the scaffold wall in FIG. 35C indicates that phase inversion micromolding is capable of replicating features on the order of 10 μm. However, the phase inversion process does not seem to prevent debonding of the polymer matrix from the pore wall as seen in both the bare pore walls in FIG. 35C and the gaps in between the polymer matrix and ceramic scaffold in FIG. 35D. While there is a significant probability that the gaps between the polymer matrix and ceramic scaffold in panel d are due to the drying process, the presence of any gaps or debonding in the wet state could lead to channeling and result in poor membrane performance. Therefore, it was decided to covalently bind the polymer matrix to the ceramic scaffold to suppress debonding.

The surfaces in FIGS. 35F and 35H show pores that are filled with no indications of gaps or debonding from the ceramic scaffold. Similarly, the cross-sectional images in FIGS. 35E and 35G both exhibit ceramic walls that are decorated with PEI particles and small sections of the polymer matrix. However, the density of decorating material on FIG. 35E is less than half of what is observed in FIG. 35G. It was concluded that the decorating material in FIG. 35E stems from functionalizing the surface because of the complete absence of decorating particles when the ceramic surface has not been modified (FIG. 35C). The discrepancy in the density of adhered PEI particles and polymer matrix is attributed to the difference in the number of reactive sites available from the surface functionalization.

Consider first a single pore that is assumed to be a perfect cylinder with diameter of 20 μm and height of 1.6 mm. The corresponding surface area and volume are 1*10⁵ μm² and 5*10⁵ μm³, respectively. Assuming a monolayer density of 4 ATMS molecules/nm² on silicon dioxide¹⁸ and that only 50% of the SiOC ceramic scaffold is silicon dioxide¹⁹, there are approximately 3*10⁻¹³ moles of ATMS per pore. Using reaction (1) from FIG. 33B to further functionalize the surface and assuming any side reactions of ECH may be ignored at room temperature, there are 6*10⁻¹³ moles of halide per pore in the ceramic scaffold available to react with the amines in the polymer solution. This concentration should be considered an upper bound due to secondary reactions, such as the halide on an already bound ECH molecule reacting with a nearby amine, reducing the actual number of halides.

Using reaction (2) from FIG. 33B as the second functionalization step produces a conformal PEI gel layer with an average thickness of 500 nm. Following a similar analysis as above, the interface of the gel layer is assumed to form a perfect cylinder with a diameter of 19 microns and height of 1.6 mm. The corresponding surface area and volume are 0.96*10⁵ μm² and 4.5*10⁵ μm³, respectively. Assuming that the concentration of halides may be approximated as a monolayer of ECH that covers the entire gel layer, the monolayer density was estimated to be 8 ECH molecules/nm² from the topological polar surface area of 0.125 nm²/ECH molecule²⁰. The resulting concentration of halides is approximately 13*10⁻¹³ moles of halide per pore. Although the calculated halide concentrations for the two reaction sequences are of the same order of magnitude, the value from reaction (1) is an upper bound that ignores a multitude of side and secondary reactions. In contrast, the value calculated for reaction (2) should be considered a lower bound due to TEP swelling the PEI molecules at the gel interface. The swelling of the interfacial region leads to more reactive sites being accessible further improving the bonding between the gel layer and the PEI microgels in the polymer solution. Due to the superior adhesion between the polymer matrix and ceramic scaffold when using the PEI gel layer, all composites used for BSA binding experiments were fabricated with a PEI gel layer unless otherwise indicated.

4.3.2 Protein Binding and the Role of the PEI Gel Layer

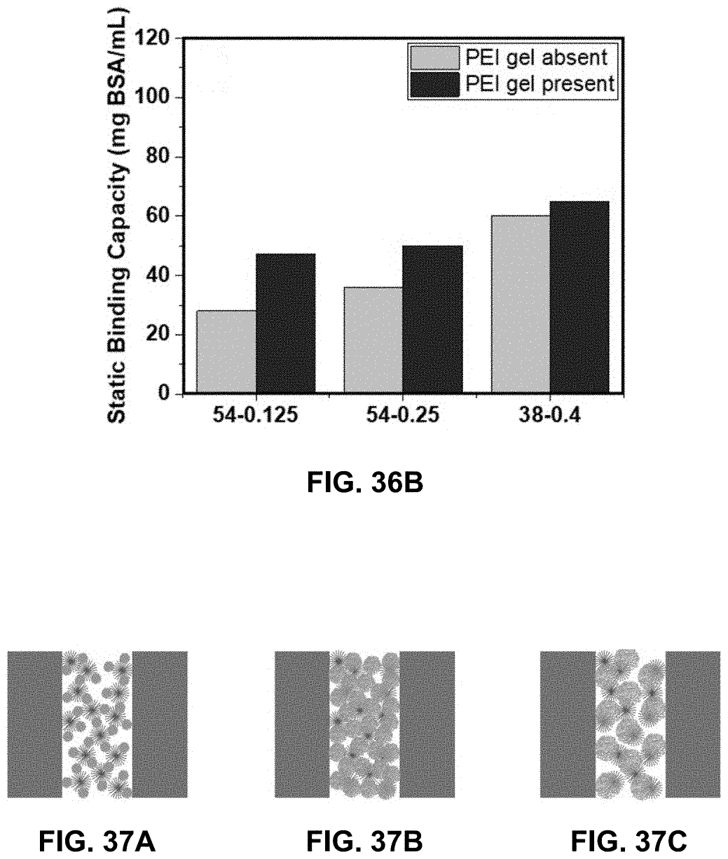

FIG. 36A shows the static binding capacities, in H₂O, of both the composite and polymeric membranes as a function of crosslink density. The composite membranes have little fluctuation in binding capacities for NCDs ≤0.25, with a drop in the reported binding capacity when the NCD is increased to 0.5. The polymeric membranes show the opposite behavior with excellent binding at NCD of 0.5 and very low binding at NCDs ≤0.25. The binding capacities of the composite membranes are also presented at two different time points. The first reported SBC was measured after 48 hours and for all compositions was lower than the second reported SBC measured after 120 hours. FIG. 36B shows the static binding capacity, in TRIS buffer, of composite membranes with the same polymer composition and different ceramic surface functionality (with PEI gel layer or not functionalized ceramic). The difference in binding capacity between the two conditions decreases as the crosslink density is increased.

The superior performance of the composite membranes at low crosslink densities validated our hypothesis that integrating the dope solution with a ceramic scaffold would broaden our operating space. Surprisingly the benefit of the ceramic did not come from the mechanical failure of PVDF, but rather from the failure to form the PEI microgels. Table 10 outlines the average number of bonds (not accounting for the different amine reactivity or steric hindrance) a single PEI molecule would have at each crosslink density. Note that at a crosslink density of 0.0625 there is less than 1 bond per PEI molecule on average, indicating that not all of the PEI that was added to the dope solution initially will be polymerized. As a result, there is a fraction of the PEI molecules that either do not react or form small oligomers. When the solution is cast as a polymeric membrane, the casting solution is immersed in IPA and any unbound PEI—in the form of single molecules or low MW oligomers—is able to diffuse out of the dope solution into the nonsolvent bath, or into the following water bath. The resulting membrane has a lower concentration of amines to interact with BSA and therefore has a lower binding capacity.

TABLE 10
Average number of bonds per PEI molecule
not accounting for differences in
reactivity of amines or steric hindrance.

| NCD | Bonds/PEI molecule |
|---|---|
| 0.5 | 4.1 |
| 0.4 | 3.3 |
| 0.25 | 2.1 |
| 0.125 | 1.0 |
| 0.0625 | 0.5 |

Next, consider a membrane composite prepared using the same dope solution as described for the polymeric membrane case. Upon infiltrating the ceramic scaffold with the polymer solution there are a number of low molecular weight PEI molecules in the solution. However, prior to phase inverting the dope solution in the ceramic there is sufficient time given to react the functional microgels in the dope with the PEI gel layer on the wall. During this time period, the unbound PEI in the dope solution may react with the exposed functional groups on the wall. The newly bound PEI molecules will not be removed during the phase inversion and subsequent washing steps thereby increasing the number of BSA binding sites in comparison to the polymeric membrane. The role of the gel layer in capturing unbound PEI was validated, shown in FIG. 36B, where a comparison between composite membranes prepared using the same dope solution to infiltrate ceramics both with and without the PEI gel layer. At lower crosslink densities when the concentration of unbound PEI is higher, the composite prepared without the PEI gel layer is 40% lower. At higher crosslink densities where the concentration of unbound PEI should be lower, the composite prepared without the PEI gel layer is only 10% lower.

4.3.3 PEI Swelling at High NCD

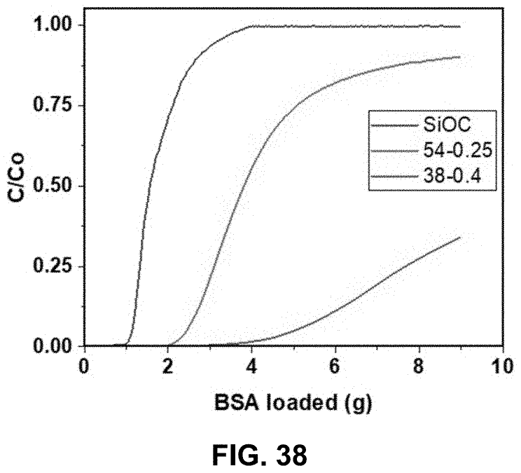

The BSA binding behavior at NCD of 0.5 in FIG. 36A was also surprising because the binding capacity of the composite was less than 30% of the capacity of the corresponding polymer membrane. The composite was expected to have approximately 70% of the polymeric membrane SBC at NCD 0.5, with the other 30% accounting for the volume occupied by the ceramic scaffold as well as the amines consumed by covalently bonding the polymer matrix to the ceramic scaffold. The large difference between the predicted and actual SBCs is caused by the swelling of PEI microgels in a constrained volume. FIGS. 37A-37C present a visual representation of PEI swelling in a constrained volume under three different conditions. Pictures demonstrating the volume change of the polymeric membrane due to microgel swelling are shown in FIG. 38.

In FIG. 37A, the pore is filled with a nonswelling liquid thereby leaving the PEI microgels in an unswollen state. This condition is reminiscent of the behavior of the PEI particles in IPA following the phase inversion micromolding. FIG. 37B depicts microgels that are in water, but are only able to reach a semi-swollen state due to physical interference by the pore wall and other nearby microgels. The semi-swollen microgels are detrimental to BSA binding through limiting both the number of available binding sites and the mass transfer through the ceramic pore. FIG. 37C also depicts microgels that are in water; however, these microgels are at a lower concentration and as a result they do not interact with other microgels allowing them to reach the thermodynamic swelling equilibrium. The fully swollen PEI particles have the largest number of available binding sites due to the reduction in interference. As a result, the highest PEI concentration that still enables the microgels to be fully swollen is the optimum condition for BSA binding. The composites with dope compositions of 54-0.4 and 38-0.4 were 54% and 38% PEI with the same crosslink density. The reported SBC of the 54-0.4 and 38-0.4 composites were 35 and 65 mg BSA/mL respectively, with the latter being the highest static binding capacity of the composite membranes investigated in this study.

4.3.4 Dynamic Binding Measurements

FIG. 38 presents representative breakthrough curves for an empty SiOC ceramic scaffold, composite membranes prepared using 54 wt. % PEI and NCD of 0.25, and composite membranes prepared using 38 wt. % PEI and NCD of 0.4. Using the 10% breakthrough method described in Example 9, BCs of 19 mg/mL and 61 mg/mL were calculated for 54-0.25 and 38-0.4 respectively. The reduction in binding capacity of 54-0.25 between the static (51 mg/mL) and dynamic (19 mg/mL) experiments is 63%, which is higher than the reduction of 25% in binding observed when using just the polymer membranes with 54 wt. % PEI and NCD of 0.5. This discrepancy is ascribed to the contributions of “unbound” PEI in the dope solution that is captured by the PEI gel layer before it can diffuse out of the ceramic. The PEI molecules captured by the wall readily contribute to the static binding capacity due to the additional time provided for BSA to reach the pore wall. In a dynamic binding setting however, only a small portion of the fluid has time to interact with the wall. The rest of the protein solution flows through the polymer matrix, which has a lower than expected PEI concentration. The lower PEI concentration in the polymer matrix leads to rapid saturation of the available binding sites resulting in a low binding capacity. In contrast, the 38-0.4 composite exhibits a DBC that is only 7% less than its SBC. This small reduction indicates that the method used to test the static binding capacity of the 38-0.4 membrane was most likely insufficient to fully saturate all the available binding sites.

The larger DBC of 38-0.4 stems from the optimization of PEI loading and crosslink density. At higher PEI concentrations the gel swells in water to such an extent that it restricts mass transfer through the composite. At lower PEI concentrations mass transfer through the composite is uninhibited, but there are fewer available binding sites leading to lower binding capacities. Similarly, as outlined previously, at low crosslink densities PEI has a higher chance to diffuse out of the composite during the casting process. Whereas at high crosslink densities, the PEI microgels are tightly crosslinked leading to various forms of steric hindrance and reduced interactions between the binding sites and the molecules of interest. The 38-0.4 composite sits in a “Goldilocks Zone” where the different interactions balance each other allowing for high binding capacity with uninhibited mass transfer.

4.4 Conclusions

Here we have documented a novel method to incorporate functional microgels into a ceramic scaffold via surface functionalization and phase inversion micromolding. The resulting composite was characterized using SEM analysis and BSA binding measurements. The SEM characterization demonstrated that functionalizing the ceramic surface with a reactive conformal PEI gel layer improved the adherence of the polymer matrix to the pore wall in the dry state. The PEI gel layer was also shown to have a beneficial impact on BSA binding at low crosslink densities, with the SBCs of the composite membranes being more than double the binding of the corresponding polymeric membrane. The relationship between PEI concentration, swelling, and BSA binding was also investigated. At high PEI concentrations, the swelling of the microgels led to lower SBC and membrane permeability. Reducing the PEI concentration in the dope solution to account for swelling resulted in the highest reported static binding capacity of 65 mg BSA/mL.

### References Corresponding to Example 10

(1) Yang, Q.; Adrus, N.; Tomicki, F.; Ulbricht, M. Composites of Functional Polymeric Hydrogels and Porous Membranes. J. Mater. Chem. 2011, 21 (9), 2783-2811. https://doi.org/10.1039/C0JM02234A.
(2) Anuraj, N.; Bhattacharjee, S.; Geiger, J. H.; Baker, G. L.; Bruening, M. L. An All-Aqueous Route to Polymer Brush-Modified Membranes with Remarkable Permeabilites and Protein Capture Rates. J. Membr. Sci. 2012, 389, 117-125. https://doi.org/10.1016/j.memsci.2011.10.022.
(3) Ulbricht, M.; Yang, H. Porous Polypropylene Membranes with Different Carboxyl Polymer Brush Layers for Reversible Protein Binding via Surface-Initiated Graft Copolymerization. Chem. Mater. 2005, 17 (10), 2622-2631. https://doi.org/10.1021/cm 0485714.
(4) Adrus, N.; Ulbricht, M. Novel Hydrogel Pore-Filled Composite Membranes with Tunable and Temperature-Responsive Size-Selectivity. J. Mater. Chem. 2012, 22 (7), 3088-3098. https://doi.org/10.1039/C2JM15022 K.
(5) Ulbricht, M. Advanced Functional Polymer Membranes. Polymer 2006, 47 (7), 2217-2262. https://doi.org/10.1016/j.polymer.2006.01.084.
(6) Liu, J.; Qu, S.; Suo, Z.; Yang, W. Functional Hydrogel Coatings. Natl. Sci. Rev. 2021, 8 (2). https://doi.org/10.1093/nsr/nwaa254.
(7) Huang, R.; Kostanski, L. K.; Filipe, C. D. M.; Ghosh, R. Environment-Responsive Hydrogel-Based Ultrafiltration Membranes for Protein Bioseparation. J. Membr. Sci. 2009, 336 (1), 42-49. https://doi.org/10.1016/j.memsci.2009.03.002.
(8) Vermonden, T.; Klumperman, B. The Past, Present and Future of Hydrogels. Eur. Polym. J. 2015, 72, 341-343. https://doi.org/10.1016/j.eurpolymj.2015.08.032.
(9) Kim, J. J.; Park, K. Smart Hydrogels for Bioseparation. 8.
(10) Arai, N. Freeze Casting of Ceramics: Pore Design from Solidification Principles. PhD Thesis, California Institute of Technology, 2021.
(11) Zeng, X.; Arai, N.; Faber, K. T. Robust Cellular Shape-Memory Ceramics via Gradient-Controlled Freeze Casting. Adv. Eng. Mater. 2019, 21 (12), 1900398. https://doi.org/10.1002/adem.201900398.
(12) Maninpat Naviroj. Silicon-Based Porous Ceramics via Freeze Casting of Preceramic Polymers (Ph.D. Thesis), Northwestern University, 2017.
(13) Arai, N.; Stan, T.; Macfarland, S.; Voorhees, P. W.; Muyanja, N. S.; Shahani, A. J.; Faber, K. T. Coarsening of Dendrites in Solution-Based Freeze-Cast Ceramic Systems. Acta Mater. 2021, 215, 117039. https://doi.org/10.1016/j.actamat.2021.117039.
(14) Rostami, S.; Talebpour, Z.; Yeganeh, H. Preparation, Optimization and Application of Poly(Ethylene Glycol)Methyl Ether Methacrylate/Urethane Methacrylate as a New Polar Phase for Stir Bar Sorptive Extraction. Anal. Methods 2014, 6 (19), 7722-7732. https://doi.org/10.1039/C4AY01113A.
(15) Acres, R. G.; Ellis, A. V.; Alvino, J.; Lenahan, C. E.; Khodakov, D. A.; Metha, G. F.; Andersson, G. G. Molecular Structure of 3-Am inopropyltriethoxysilane Layers Formed on Silanol-Terminated Silicon Surfaces. J. Phys. Chem. C 2012, 116 (10), 6289-6297. https://doi.org/10.1021/jp212056s.
(16) Chauhan, A. K.; Aswal, D. K.; Koiry, S. P.; Gupta, S. K.; Yakhmi, J. V.; Sürgers, C.; Guerin, D.; Lenfant, S.; Vuillaume, D. Self-Assembly of the 3-Aminopropyltrimethoxysilane Multilayers on Si and Hysteretic Current-Voltage Characteristics. Appl. Phys. A 2008, 90 (3), 581-589. https://doi.org/10.1007/s00339-007-4336-7.
(17) Zhu, M.; Lerum, M. Z.; Chen, W. How To Prepare Reproducible,
Homogeneous, and Hydrolytically Stable Aminosilane-Derived Layers on Silica. Langmuir 2012, 28 (1), 416-423. https://doi.org/10.1021/1a203638g.
(18) Karade, V. C.; Sharma, A.; Dhavale, R. P.; Dhavale, R. P.; Shingte, S. R.; Patil, P. S.; Kim, J. H.; Zahn, D. R. T.; Chougale, A. D.; Salvan, G.; Patil, P. B. APTES Monolayer Coverage on Self-Assembled Magnetic Nanospheres for Controlled Release of Anticancer Drug Nintedanib. Sci. Rep. 2021, 11 (1), 5674. https://doi.org/10.1038/s41598-021-84770-0.
(19) Saha, A.; Raj, R.; Williamson, D. L. A Model for the Nanodomains in Polymer-Derived SiCO. J. Am. Ceram. Soc. 2006, 89 (7), 2188-2195. https://doi.org/10.1111/j.1551-2916.2006.00920.x.
(20) Khlebtsov, B. N.; Khanadeev, V. A.; Burov, A. M.; Le Ru, E. C.; Khlebtsov, N. G. Reexamination of Surface-Enhanced Raman Scattering from Gold Nanorods as a Function of Aspect Ratio and Shape. J. Phys. Chem. C 2020, 124 (19), 10647-10658.

### Example 11

### I. Introduction

The field of membrane chromatography has expanded rapidly as an alternative to the conventional packed bed chromatography in pharmaceutical separations [1-5]. The shift in technology has been motivated by a need to reduce downstream bioprocessing costs associated with long processing times and high operating pressures. Membrane chromatography reduces the processing time and operating pressure by utilizing convective, as opposed to diffusive, mechanisms to transport molecules of interest to the associated binding sites. The change in transport mechanism enables the system to operate at faster flow rates while maintaining a low operating pressure. In addition, the use of convective transportation allows for processing to be operated at a wide range of flow rates with minimal impact on the binding capacity of the membrane. These flow properties are amenable to the scale up of the separation processes and complement the easy mass production of membrane based materials thereby further reducing downstream costs [1-2]. The adoption of membrane chromatography has also benefited from drawing on the experience of related fields in membrane science, i.e. identification of porous polymeric membranes with good chemical and physical stability to act as supports. As a result, many membrane adsorbers are derivatives of membranes used in other separation processes [1-4].

In order to capitalize on the advantages outlined above, recent work has focused on addressing the key drawbacks of membrane chromatography. Two such drawbacks are the low volumetric binding capacity of membrane adsorbers in comparison to resins and limited salt tolerance [1,3-7]. While resins have a high binding surface area per volume ratio due to the tortuosity of the resin beads, membrane adsorbers initially relied solely on the pore surface area as the active binding area resulting in low volumetric binding capacity. One promising method to overcome this barrier is to use various polymerization techniques to graft polyelectrolyte chains or polymer brushes with appropriate functionalities onto the porous membrane supports [3-5,8]. The resulting membranes benefit from the porosity of the support while increasing the available binding surface area to improve volumetric binding capacity. However, the improvement in volumetric binding capacity has only been demonstrated for solutions with low salt concentrations [3-5]. Operating pharmaceutical separations in solutions with low ionic strength typically requires a buffer exchange step which increases processing costs [7]. In order to reduce the extent of the buffer exchange step, it has been desired to develop membranes which demonstrate consistent binding over a range of salt concentrations. The improvement of the salt tolerance of membrane adsorbers typically requires a reduction in the ionic sensitivity of the binding ligand through manipulation of the ligand chemistry. Recent work replacing ligands with quaternary amine based chemistry with those containing predominantly primary amine chemistry demonstrated volumetric binding capacities which were essentially constant over a range of conductivities. [6,9]. Although these membranes achieved high salt tolerance, the reported volumetric binding capacities were low.

An alternative method is the pore-filling of the porous membrane supports with a functional hydrogel [10,11,12]. The functional hydrogel may bring a host of beneficial properties to the composite including responsiveness to environmental stimuli, hydrophilicity, unique binding chemistry [10,11]. However, many of these functional hydrogels do not have the mechanical properties required to be useful in separations or similar processes. Placing the functional hydrogels within an appropriate porous membrane support provides the necessary robustness, reduces swelling, and preserves the useful properties of the hydrogel. Current work which demonstrates the pore-filling method with both polymeric and ceramic porous supports has focused predominately on using in-situ polymerization to develop these functional composites [10-13].

In our current study we selected to use a novel pore-filling method and use a macroporous ceramic scaffold with three key advantages. First, the Silicon Oxycarbide ceramic (SiOC) is inert under the operating conditions often used in bioseparations. Second, the ceramic is mechanically robust and in terms of compressive strength outperforms the inert porous polymer membranes. Third, functionalizing the ceramic via silanization utilizes a widely available pathway which is versatile and does not require intense operating conditions or exacting control. The novel pore-filling method constitutes the following steps: preparation of a polymer dope solution, injection molding of the dope solution into the ceramic scaffold (the scaffold may be functionalized), and using an appropriate nonsolvent to initiate the phase inversion solidification of the dope solution. The method is described in more detail below. To our knowledge utilizing a phase inversion solidification process to form a composite ceramic-hydrogel membrane has not been presented before.

### II. Experimental Methods

II.1 Chemicals and Materials

Polyvinylidene Fluoride (PVDF) [Kynar 761] was provided by Arkema (King of Prussia, Pa.). Hyperbranched polyethylenimine (PEI) was procured from Polysciences. Cyclohexane (C6H12), Epichlorohydrin (ECH), 3-Aminopropyltrimethoxysilane (ATMS), Bovine Serum Albumin (BSA), Bis(2-chloroethyl)amine hydrochloride (BCAH), Triethyl Phosphate (TEP), Isopropanol (IPA), Dimethyl sulfoxide (DMSO), and TRIS hydrochloride (TRIS) were purchased from Millipore Sigma. Hydrochloric acid was purchased from EMD Millipore. Polysiloxane (CH3-SiO1.5, Silres® MK Powder) and Geniosil® GF 91 were purchased from Wacker Chemie. All chemicals and materials were used as received. Buffers were prepared using indicated chemicals and distilled water.

II.2.2 Ceramic Fabrication

A polymer solution was prepared by dissolving the polysiloxane preceramic polymer in cyclohexane, with concentration of preceramic polymer of 20 wt. %. Once a homogenized solution was obtained, a cross-linking agent (Geniosil® GF 91) was added in concentrations of 1 wt. % and stirred for 5 minutes and then degassed for 10 min to avoid air bubbles during solidification. The freeze-casting was done by pouring the polymer solution into a glass mold (h=20 mm, 0=25 mm) that was located on a PID-controlled thermoelectric plate. Another thermoelectric was placed on top of the mold to control both freezing front velocity and temperature gradient. A cold finger with smaller diameter than the mold was inserted into the glass mold such that the created spaces act as reservoir for the solution as the solution shrunk by solidification. The freezing front velocity and temperature gradient were measured by taking pictures every 30 seconds using a camera and intervalometer. The temperature gradient, G was defined by the following equation:

$$G = \frac{{T}_{r} - {T}_{f}}{d}$$

where Tₜ is the temperature of top cold finger, T_{f} is the temperature of freezing front and d is the distance between the top cold finger and the freezing front. The temperature of the freezing front was assumed to be at the liquidus temperature of the solution, and the value was taken from the work by Naviroj¹². All samples were frozen at freezing front velocities of 15 μm/s, and temperature gradients of 2.5 K/mm to maintain homogeneous pore structures.

Once the structure was completely frozen, the isothermal coarsening was initiated by setting the top and bottom thermoelectrics to 4° C. After the structure was coarsened for 3 hours, the sample was re-froze¹³. After the sample was completely frozen, the sample was sublimated in a freeze drier (VirTis AdVantage 2.0) where the solvent crystals were completely removed. After freeze drying, the polymer scaffold was pyrolyzed in argon at 1100° C. for four hours to convert the preceramic polymer into silicon oxycarbide (SiOC). This resulted in a porosity of ˜77%. The pyrolyzed sample was machined into a disk with thickness of ˜1.6 mm and diameter of ˜13 mm prior to infiltration.

II.2.3 Polymer Dope Synthesis

The polymer dope synthesis was initiated by adding 5.91 g of PVDF to an empty 3-neck round bottom flask. The flask was then outfitted with an overhead mechanical stirrer and the necessary greased connectors. Next, 30 mL of TEP was added to the flask and then the remaining openings were sealed using rubber septa. The PVDF/TEP mixture was heated to 80° C. and incubated for 1 hour before the mixing speed was set to 60 rpm. The resulting solution was left to equilibrate overnight. A PEI solution was prepared by adding the mass of PEI indicated in Table 11 to a scintillation vial followed by 5 mL of TEP. The mixture was vortexed and shaken until no concentration gradients were visible and then it was left to equilibrate overnight at room temperature. The crosslinker solution was prepared by weighing the required amount of BCAH into a scintillation vial and then adding the corresponding volume of DMSO (Table 11). The resulting mixture was incubated overnight at room temperature to fully dissolve the BCAH.

TABLE 11
Composition of polymer dope solution and associated
Normalized Crosslink Density (NCD). Naming scheme
goes as wt % PEI in the dry polymeric membrane-NCD.

| Dope | PVDF | PEI | BCAH | DMSO | |
|---|---|---|---|---|---|
| solution | (g) | (g) | (g) | (mL) | NCD |
| 54-0.5 | 5.91 | 5 | 3.1 | 5 | 0.5 |
| 54-0.25 | 5.91 | 5 | 1.55 | 2.5 | 0.25 |
| 54-0.125 | 5.91 | 5 | 0.78 | 1.25 | 0.125 |
| 54-0.0625 | 5.91 | 5 | 0.39 | 0.625 | 0.0625 |
| 54-0.4 | 5.91 | 5 | 2.5 | 4 | 0.4 |
| 38-0.4 | 5.91 | 2.58 | 1.28 | 2 | 0.4 |

The next day, the reaction flask was purged with N₂ for 7 minutes and the mixing speed was increased to 250 rpm. With the N₂ flow still on, the PEI solution was then added dropwise to the flask using a glass Pasteur pipette over the course of 4 minutes. The resulting solution was left to mix for 5 minutes before adding 0.43 mL of concentrated HCl (37% solution). Following the addition of the HCl, the flask was incubated for 15 minutes at 80° C. with the mixing speed maintained at 250 rpm. The crosslinker solution was then added to the flask and the polymerization reaction was allowed to proceed for 4 hours. After the 4-hour reaction time, the flask was put under in-house vacuum for 10 minutes to remove entrapped air. The resulting dope solution was then added to the ceramic using the steps outlined in 11.5 in this example.

Several polymeric membranes were prepared at wt % PEI in the dry polymeric membrane—NCD compositions of 54-0.5, 54-0.25, 54-0.125, 54-0.0625 as controls for the static binding measurements. The same steps outlined above were followed until completing the incubation under vacuum. The resulting dope solution was then cast on glass plates at a blade height of 300 μm. The cast membranes were left at room temperature for 30 seconds before being immersed in an isopropanol coagulation bath. After two hours, the solidified membranes were moved to distilled water baths prior to storage.

II.4 Surface Functionalization of Ceramic

Prior to adding the polymer dope solution, the ceramic surface was activated and functionalized (FIG. 33A) using a procedure derived from prior literature¹⁴⁻¹⁷. The SiOC scaffold was first immersed and incubated in 1 M NaOH for 90 minutes. It was then washed in water before being incubated in a 0.1 M HCl solution for 30 minutes. The ceramic was then washed in water again, before being dried at 110° C. for 1 hour. Once the ceramic was dried, it was added to a 2 v % solution of ATMS in isopropanol and incubated for 3 hours at 60° C. The sample was then washed thoroughly in water and isopropanol before being cured at 110° C. for 30 minutes.

Following the curing of the aminosilane layer, the ceramic surface was further functionalized following the two reaction schemes presented in FIG. 33B. Ceramics prepared using the top route were incubated in an IPA/ECH solution overnight. Following the overnight incubation, the samples were thoroughly washed with IPA and then left to dry at room temperature before the addition of the polymer solution. The resulting surface was expected to be terminated in chloride groups, which would react readily with the primary and secondary amines in the polymer solution.

The second further functionalization route was designed to increase the number of functional groups on the wall available to interact with amines in the PEI microgels. The functionalization proceeded as follows: the functionalization solution was prepared by dissolving PEI with IPA at a molar ratio of 1:37.4, respectively. Ten minutes before adding the solution to the ceramic, ECH was added to the solution at a molar ratio of 1 mole PEI for 16.5 moles ECH—corresponding to 1.1 ECH molecules for every available amine. This ratio was chosen to minimize the crosslinking between PEI molecules and thereby maximize the number of reactive sites. The ceramic was incubated in the IPA/PEI/EHC solution overnight at room temperature. After the overnight incubation, DMSO was added to the vial and the resulting solution was heated to 80° C. for 1 hour to remove the leftover reactants and unbound products. The sample was then washed with IPA and dried at room temperature for one hour prior to the addition of the polymer dope solution.

II.5 Phase Inversion Micromolding

The phase inversion micromolding process shown in FIG. 34 was used for both neat ceramic samples and ceramics functionalized using the pathways described above. The ceramic scaffold was loaded into the infiltration device and the polymer dope solution was injected using a syringe pump. The solution was pumped at a rate of 100 μL/min until the ceramic and infiltration device were filled. The device was then incubated at 80° C. for 1 hour for both the functionalized and the neat ceramic samples. Following the incubation, the samples were removed from the infiltration device and placed in IPA for an overnight incubation. The following day, the samples were moved to water baths to remove trace solvent and IPA in preparation for BSA binding characterization.

II.6 Membrane Properties Characterization

II.6.1 SEM

The microstructure of ceramic scaffolds and polymer/ceramic composites were observed using a field emission scanning electron microscope (FE SEM-Zeiss 1550 VP). In preparation for imaging, the samples were dried at 70° C. overnight. To prepare the cross-sectional view, the membranes were broken by hand at ambient conditions. The surfaces and cross-sections of interest were then coated with a Pt/Pd conductive layer (10 nm) using a sputter coater and then imaged.

II.6.2 Protein Adsorption Studies

BSA was used as the model protein in both static and dynamic binding measurements. Initial tests were done using BSA in distilled water at a concentration of 2 mg/mL. To measure the static binding of the polymeric membrane references, a membrane with a known volume was immersed in a 2 mg/mL BSA solution and gently mixed for 48 hours. The absorbance of the solution was then measured using an Agilent 8453 UV/vis and the reported absorbance at 280 nm was used to determine the concentration of BSA in the solution. The mass of BSA bound was then determined using a mass balance.

A similar process was used to measure the static binding capacity (SBC) of the composite membranes. The samples were immersed in a 2 mg/mL BSA solution and gently rocked for 48 hours. The absorbance was then measured and the binding capacity calculated before the samples were rocked for another 72 hours. The absorbance was then measured again and the binding capacity calculated. The addition of the second absorbance measurement was to account for the increased thickness and reduced mass transfer in the composites. The initial experiments for comparison to the polymeric membranes used BSA dissolved in H₂O, all subsequent measurements used BSA dissolved in 50 mM TRIS.

Dynamic binding measurements using 2 mg/mL BSA in 50 mM TRIS buffer were conducted using composites with formulations of 54-0.25 and 38-0.4. To run the measurement, the sample was first loaded into a Swinney filter holder (Pall Corp.) and was equilibrated using 50 mM TRIS buffer. The BSA solution was then introduced via a syringe pump to the device at a rate of 150 μL/min (or 2 membrane volumes/min). The filtrate was analyzed with time-resolved measurements on the Agilent 8453 UV/vis. The 10% breakthrough curve method, as described in Example 9, was used to determine the dynamic binding capacity.

### III Results and Discussion

III.1 Phase Inversion Micromolding Feasibility

FIGS. 35A-35H show SEM micrographs of the cross-section and surfaces of the ceramic scaffold and composite membranes with different surface functionality. The longitudinal cross-sectional image of the neat ceramic scaffold, FIG. 35A, shows the highly oriented pores that transverse the entire membrane. The corresponding surface (perpendicular to the freeze-casting direction), FIG. 35B, demonstrates the morphology of the oriented pores as well as the average pore diameter of 20 μm. The composite presented in FIGS. 35C-35D was infiltrated without modifying the surface of the ceramic. In FIG. 35C, there is a segment of the polymer matrix in the middle of the micrograph that has a morphology that closely matches the contours of the nearby ceramic pore wall. It is also noteworthy that the ceramic surfaces that are visible are all bare. In the surface view from FIG. 35D the pores are mostly filled with the polymer matrix, but there are many cases where there is a debonded interface between the polymer matrix and one side of the pore.

The composite presented in FIGS. 35E-35F had the surface modified using reaction (1) from FIG. 33B prior to the infiltration and phase inversion micromolding. The polymer matrix once again fills the pores in FIG. 35E and the ceramic walls that are visible are lightly decorated in microparticles from the polymer matrix. FIG. 35F shows that the ceramic pores are completely filled and there are no visible gaps between the polymer matrix and the pore wall. The composite presented in FIGS. 35G-35H had the surface modified using reaction (2) from FIG. 33B, producing a functional PEI gel layer prior to infiltration and phase inversion micromolding. The polymer matrix fills the pores in FIG. 35G and the ceramic walls that are visible are decorated with a higher density of microparticles/polymer matrix than FIG. 35E. FIG. 35H shows that the ceramic pores are once again completely filled and there are no visible gaps between the polymer matrix and the pore wall.

The observations of the behavior of the polymer matrix in FIGS. 35C-35D, provide several key insights on phase inversion micromolding and how to stably integrate the mixed matrix membrane with the ceramic scaffold. The match between the morphology of the polymer matrix and the contours of the scaffold wall in FIG. 35C indicates that phase inversion micromolding is capable of replicating features on the order of 10 μm. However, the phase inversion process does not seem to prevent debonding of the polymer matrix from the pore wall as seen in both the bare pore walls in FIG. 35C and the gaps in between the polymer matrix and ceramic scaffold in FIG. 35D. While there is a significant probability that the gaps between the polymer matrix and ceramic scaffold in FIG. 35D are due to the drying process, the presence of any gaps or debonding in the wet state could lead to channeling and result in poor membrane performance. Therefore, it was decided to covalently bind the polymer matrix to the ceramic scaffold to suppress debonding.

The surfaces in FIGS. 35F and 35H show pores that are filled with no indications of gaps or debonding from the ceramic scaffold. Similarly, the cross-sectional images in FIGS. 35E and 35G both exhibit ceramic walls that are decorated with PEI particles and small sections of the polymer matrix. However, the density of decorating material on FIG. 35E is less than half of what is observed in FIG. 35G. It was concluded that the decorating material in FIG. 35E stems from functionalizing the surface because of the complete absence of decorating particles when the ceramic surface has not been modified (FIG. 35C). The discrepancy in the density of adhered PEI particles and polymer matrix is attributed to the difference in the number of reactive sites available from the surface functionalization.

Consider first a single pore that is assumed to be a perfect cylinder with diameter of 20 μm and height of 1.6 mm. The corresponding surface area and volume are 1*10⁵ μm² and 5*10⁵ μm³, respectively. Assuming a monolayer density of 4 ATMS molecules/nm² on silicon dioxide¹⁸ and that only 50% of the SiOC ceramic scaffold is silicon dioxide¹⁹, there are approximately 3*10⁻¹³ moles of ATMS per pore. Using reaction (1) from FIG. 33B to further functionalize the surface and assuming any side reactions of ECH may be ignored at room temperature, there are 6*10⁻¹³ moles of halide per pore in the ceramic scaffold available to react with the amines in the polymer solution. This concentration should be considered an upper bound due to secondary reactions, such as the halide on an already bound ECH molecule reacting with a nearby amine, reducing the actual number of halides.

Using reaction (2) from FIG. 33B as the second functionalization step produces a conformal PEI gel layer with an average thickness of 500 nm. Following a similar analysis as above, the interface of the gel layer is assumed to form a perfect cylinder with a diameter of 19 microns and height of 1.6 mm. The corresponding surface area and volume are 0.96*10⁵ μm² and 4.5*10⁵ μm³, respectively. Assuming that the concentration of halides may be approximated as a monolayer of ECH that covers the entire gel layer, the monolayer density was estimated to be 8 ECH molecules/nm² from the topological polar surface area of 0.125 nm²/ECH molecule²⁰. The resulting concentration of halides is approximately 13*10⁻¹³ moles of halide per pore. Although the calculated halide concentrations for the two reaction sequences are of the same order of magnitude, the value from reaction (1) is an upper bound that ignores a multitude of side and secondary reactions. In contrast, the value calculated for reaction (2) should be considered a lower bound due to TEP swelling the PEI molecules at the gel interface. The swelling of the interfacial region leads to more reactive sites being accessible further improving the bonding between the gel layer and the PEI microgels in the polymer solution. Due to the superior adhesion between the polymer matrix and ceramic scaffold when using the PEI gel layer, all composites used for BSA binding experiments were fabricated with a PEI gel layer unless otherwise indicated.

III.2 Protein Binding and the Role of the PEI Gel Layer

FIG. 36A shows the static binding capacities, in H₂O, of both the composite and polymeric membranes as a function of crosslink density. The composite membranes have little fluctuation in binding capacities for NCDs ≤0.25, with a drop in the reported binding capacity when the NCD is increased to 0.5. The polymeric membranes show the opposite behavior with excellent binding at NCD of 0.5 and very low binding at NCDs ≤0.25. The binding capacities of the composite membranes are also presented at two different time points. The first reported SBC was measured after 48 hours and for all compositions was lower than the second reported SBC measured after 120 hours. FIG. 36B shows the static binding capacity, in TRIS buffer, of composite membranes with the same polymer composition and different ceramic surface functionality (with PEI gel layer or not functionalized ceramic). The difference in binding capacity between the two conditions decreases as the crosslink density is increased.

The superior performance of the composite membranes at low crosslink densities validated our hypothesis that integrating the dope solution with a ceramic scaffold would broaden our operating space. Surprisingly the benefit of the ceramic did not come from the mechanical failure of PVDF, but rather from the failure to form the PEI microgels. Table 12 outlines the average number of bonds (not accounting for the different amine reactivity or steric hindrance) a single PEI molecule would have at each crosslink density. Note that at a crosslink density of 0.0625 there is less than 1 bond per PEI molecule on average, indicating that not all of the PEI that was added to the dope solution initially will be polymerized. As a result, there is a fraction of the PEI molecules that either do not react or form small oligomers. When the solution is cast as a polymeric membrane, the casting solution is immersed in IPA and any unbound PEI—in the form of single molecules or low MW oligomers—is able to diffuse out of the dope solution into the nonsolvent bath, or into the following water bath. The resulting membrane has a lower concentration of amines to interact with BSA and therefore has a lower binding capacity.

TABLE 12
Average number of bonds per PEI molecule
not accounting for differences in
reactivity of amines or steric hindrance.

| NCD | Bonds/PEI molecule |
|---|---|
| 0.5 | 4.1 |
| 0.4 | 3.3 |
| 0.25 | 2.1 |
| 0.125 | 1.0 |
| 0.0625 | 0.5 |

Next, consider a membrane composite prepared using the same dope solution as described for the polymeric membrane case. Upon infiltrating the ceramic scaffold with the polymer solution there are a number of low molecular weight PEI molecules in the solution. However, prior to phase inverting the dope solution in the ceramic there is sufficient time given to react the functional microgels in the dope with the PEI gel layer on the wall. During this time period, the unbound PEI in the dope solution may react with the exposed functional groups on the wall. The newly bound PEI molecules will not be removed during the phase inversion and subsequent washing steps thereby increasing the number of BSA binding sites in comparison to the polymeric membrane. The role of the gel layer in capturing unbound PEI was validated, shown in FIG. 36B, where a comparison between composite membranes prepared using the same dope solution to infiltrate ceramics both with and without the PEI gel layer. At lower crosslink densities when the concentration of unbound PEI is higher, the composite prepared without the PEI gel layer is 40% lower. At higher crosslink densities where the concentration of unbound PEI should be lower, the composite prepared without the PEI gel layer is only 10% lower.

III.3 PEI Swelling at High NCD

The BSA binding behavior at NCD of 0.5 in FIG. 36A was also surprising because the binding capacity of the composite was less than 30% of the capacity of the corresponding polymer membrane. The composite was expected to have approximately 70% of the polymeric membrane SBC at NCD 0.5, with the other 30% accounting for the volume occupied by the ceramic scaffold as well as the amines consumed by covalently bonding the polymer matrix to the ceramic scaffold. The large difference between the predicted and actual SBCs is caused by the swelling of PEI microgels in a constrained volume. FIGS. 37A-37C present a visual representation of PEI swelling in a constrained volume under three different conditions.

In FIG. 37A, the pore is filled with a nonswelling liquid thereby leaving the PEI microgels in an unswollen state. This condition is reminiscent of the behavior of the PEI particles in IPA following the phase inversion micromolding. FIG. 37B depicts microgels that are in water, but are only able to reach a semi-swollen state due to physical interference by the pore wall and other nearby microgels. The semi-swollen microgels are detrimental to BSA binding through limiting both the number of available binding sites and the mass transfer through the ceramic pore. FIG. 37C also depicts microgels that are in water; however, these microgels are at a lower concentration and as a result they do not interact with other microgels allowing them to reach the thermodynamic swelling equilibrium. The fully swollen PEI particles have the largest number of available binding sites due to the reduction in interference. As a result, the highest PEI concentration that still enables the microgels to be fully swollen is the optimum condition for BSA binding. The composites with dope compositions of 54-0.4 and 38-0.4 were 54% and 38% PEI with the same crosslink density. The reported SBC of the 54-0.4 and 38-0.4 composites were 35 and 65 mg BSA/mL respectively, with the latter being the highest static binding capacity of the composite membranes investigated in this study.

III.4 Dynamic Binding Measurements

FIG. 38 presents representative breakthrough curves for an empty SiOC ceramic scaffold, composite membranes prepared using 54 wt. % PEI and NCD of 0.25, and composite membranes prepared using 38 wt. % PEI and NCD of 0.4. Using the 10% breakthrough method described in Example 9, BCs of 19 mg/mL and 61 mg/mL were calculated for 54-0.25 and 38-0.4 respectively. The reduction in binding capacity of 54-0.25 between the static (51 mg/mL) and dynamic (19 mg/mL) experiments is 63%, which is higher than the reduction of 25% in binding observed when using just the polymer membranes with 54 wt. % PEI and NCD of 0.5. This discrepancy is ascribed to the contributions of “unbound” PEI in the dope solution that is captured by the PEI gel layer before it can diffuse out of the ceramic. The PEI molecules captured by the wall readily contribute to the static binding capacity due to the additional time provided for BSA to reach the pore wall. In a dynamic binding setting however, only a small portion of the fluid has time to interact with the wall. The rest of the protein solution flows through the polymer matrix, which has a lower than expected PEI concentration. The lower PEI concentration in the polymer matrix leads to rapid saturation of the available binding sites resulting in a low binding capacity. In contrast, the 38-0.4 composite exhibits a DBC that is only 7% less than its SBC. This small reduction indicates that the method used to test the static binding capacity of the 38-0.4 membrane was most likely insufficient to fully saturate all the available binding sites.

The larger DBC of 38-0.4 stems from the optimization of PEI loading and crosslink density. At higher PEI concentrations the gel swells in water to such an extent that it restricts mass transfer through the composite. At lower PEI concentrations mass transfer through the composite is uninhibited, but there are fewer available binding sites leading to lower binding capacities. Similarly, as outlined previously, at low crosslink densities PEI has a higher chance to diffuse out of the composite during the casting process. Whereas at high crosslink densities, the PEI microgels are tightly crosslinked leading to various forms of steric hindrance and reduced interactions between the binding sites and the molecules of interest. The 38-0.4 composite sits in a “Goldilocks Zone” where the different interactions balance each other allowing for high binding capacity with uninhibited mass transfer.

### IV Conclusions for Example 11

Here we have documented a novel method to incorporate functional microgels into a ceramic scaffold via surface functionalization and phase inversion micromolding. The resulting composite was characterized using SEM analysis and BSA binding measurements. The SEM characterization demonstrated that functionalizing the ceramic surface with a reactive conformal PEI gel layer improved the adherence of the polymer matrix to the pore wall in the dry state. The PEI gel layer was also shown to have a beneficial impact on BSA binding at low crosslink densities, with the SBCs of the composite membranes being more than double the binding of the corresponding polymeric membrane. The relationship between PEI concentration, swelling, and BSA binding was also investigated. At high PEI concentrations, the swelling of the microgels led to lower SBC and membrane permeability. Reducing the PEI concentration in the dope solution to account for swelling resulted in the highest reported static binding capacity of 65 mg BSA/mL.

### References Corresponding to Example 11

1. R. Ghosh, Protein separation using membrane chromatography: opportunities and challenges, J. Chromatogr. A 952 (2002) 13-27
2. P. Madadkar, Q. Wu, R. Ghosh, A laterally-fed membrane chromatography model, Journal of Membrane Science 487 (2015) 173-179
3. B. V. Bhut, S. R. Wickramasinghe, S. M. Husson, Preparation of high-capacity, weak anion-exchange membranes for protein separations using surface-initiated atom transfer radical polymerization, Journal of Membrane Science 325 (2008) 176-183
4. B. V. Bhut, S. M. Husson, Dramatic performance improvement of weak anion-exchange membranes for chromatographic bioseparations
5. L. Sun, J. Dai, G. Baker, M. Bruening, High-capacity, protein-binding membranes based on polymer brushes grown in porous substrates, Chem. Mater. 2006, 18, 4033-4039
6. S. Fischer-Frühholz, D. Zhou, M. Hirai, Sartobind STIC salt-tolerant membrane chromatography, Nat Methods 7, 12-13 (2010)
7. V. Orr, L. Zhong, M. Moo-Young, C. P. Chou, Recent advances in bioprocessing application of membrane chromatography, Biotechnology Advances 31 (2013) 450-465
8. J. J. Keating, J. Imbrogno, G. Belfort, Polymer Brushes for Membrane separations: A review, ACS Appl. Mater. Interfaces 2016, 8, 28383-28399 J. Fan, J. Luo, X. Chen, Y. Wan, Facile preparation of salt-tolerant anion-exchange membrane adsorber using hydrophobic membrane as substrate, J. Chromatogr. A 1490 (2017) 54-62
9. W. Riordan, S. Heilmann, K. Brorson, K. Seshadri, Y. He, M. Etzel, Design of salt-tolerant membrane adsorbers for viral clearance, Biotechnology and Bioengineering 103 (2009) 920-929
10. Q. Yang, et al., Composites of functional polymeric hydrogels and porous membranes, J. Mater. Chem., 2011, 21, 2783-2811, DOI: 10.1039/c0jm02234a
11. N. Adrus, M. Ulbricht, Novel hydrogel pore-filled composite membranes with tunable and temperature-responsive size-selectivity, J. Mater. Chem., 2012, 22, 3088, DOI: 10.1039/c2jm 15022k
12. A. Doring, W. Birnbaum, D. Kuckling, Responsive hydrogels—structurally and dimensionally optimized smart frameworks for applications in catalysis, micro-system technology and material science, Chem. Soc. Rev., 2013, 42, 7391-7420, DOI: 10.1039/C3CS60031A
13. M. Petrini, M. Ferrante, B. Su, Fabrication and characterization of biomimetic ceramic/polymer composite materials for dental restoration, Dental Materials 29 (2013) 375-381, http://dx.doi.org/10.1016/j.dental.2012.12.004
14. R. C. F. Cheung, J. H. Wong, T. B. Ng, Immobilized metal ion affinity chromatography: a review of its applications, Appl Microbiol Biotechnol (2012) 96:1411-1420, DOI: 10.1007/s00253-012-4507-0
15. M. R. Kotte, et al., Mixed Matrix PVDF Membranes With in Situ Synthesized PAMAM Dendrimer-Like Particles: A New Class of Sorbents for Cu(II) Recovery from Aqueous Solutions by Ultrafiltration, Environ. Sci. Technol. 2015, 49, 9431-9442, DOI: 10.1021/acs.est.5b01594\
16. M. R. Kotte, et al., A Facile and Scalable Route to the Preparation of Catalytic Membranes with in Situ Synthesized Supramolecular Dendrimer Particle Hosts for Pt(0) Nanoparticles Using a Low-Generation PAMAM Dendrimer (G1-NH2) as Precursor, ACS Appl. Mater. Interfaces 2018, 10, 39, 33238-33251, DOI: 10.1021/acsami.8b11351
17. X. Zeng, N. Arai, K. T. Faber, Robust Cellular Shape-Memory Ceramics via Gradient-Controlled Freeze Casting, Adv. Eng. Mater. 21 (2019) 1900398. https://doi.org/10.1002/adem.201900398.
18. Maninpat Naviroj, Silicon-based Porous Ceramics via Freeze Casting of Preceramic Polymers (Ph.D. thesis), Northwestern University, 2017. https://search.proquest.com/openview/556ab 0b3ba48bd5ad64aa2c67a18ff27/1?pq-origsite=gscholar&cb1=18750&diss=y (accessed May 23, 2018).

### Example 12

A. Abstract

The rapid growth of the therapeutic antibody market has led to a need for improved downstream bioprocessing. Membrane chromatography has become the favored method for improving downstream bioprocessing due to the low pressure requirements, fast processing time, and favorable flow dynamics. Despite these advantages, membrane chromatography suffers from both low salt tolerance and comparatively low binding capacity. Here we synthesize mixed matrix membranes that provide improved salt tolerance while simultaneously providing high protein binding capacity. Optimization of the membrane formulation was performed to balance mechanical integrity with binding capacity and salt tolerance. It was found that the composition and structure of the embedded particles strongly influenced the final membrane performance. The binding capacity of the mixed matrix membranes was tested in static and dynamic configurations using bovine serum albumin (or BSA). The optimized mixed matrix membranes are capable of binding over 100 mg/mL of BSA in solutions with salt concentrations up to 120 mM under static conditions. With a higher salt concentration of 250 mM, the BSA binding capacity is reduced by approximately 50%. Similar results were obtained during the dynamic binding studies. The improved salt tolerance and high binding capacity reduces the total number of purification steps, thereby reducing processing time and resource expenditures.

### I. Introduction

The field of membrane chromatography has expanded rapidly as an alternative to the conventional packed bed chromatography in pharmaceutical separations¹⁻⁵. The shift in technology has been motivated by a need to reduce downstream bioprocessing costs associated with long processing times and high operating pressures. Membrane chromatography reduces the processing time and operating pressure by utilizing convective, as opposed to diffusive, mechanisms to transport molecules of interest to the associated binding sites. The change in transport mechanism enables the system to operate at faster flow rates while maintaining a low operating pressure. In addition, the use of convective transportation allows for processing to be operated at a wide range of flow rates with minimal impact on the binding capacity of the membrane. These flow properties are amenable to the scale-up of the separation processes and complement the easy mass production of membrane-based materials thereby further reducing downstream costs¹². The adoption of membrane chromatography has also benefited from drawing on the experience of related fields in membrane science, i.e. identification of porous polymeric membranes with good chemical and physical stability to act as supports. As a result, many membrane adsorbers are derivatives of membranes used in other separation processes¹⁻⁴.

In order to capitalize on the advantages of fast flow rates and low operating pressures outlined above, recent work has focused on addressing the key drawbacks of membrane chromatography. Two such drawbacks are the low volumetric binding capacity of membrane adsorbers in comparison to resins and limited salt tolerance^{1,3-7}. While resins have a high binding surface area per volume ratio due to the tortuosity of the resin beads, early membrane adsorbers rely solely on the pore surface area as the active binding area resulting in low volumetric binding capacity. A promising method to overcome this barrier is to use various polymerization techniques to graft polyelectrolyte chains or polymer brushes with appropriate functionalities onto the porous membrane supports^{3-5,8}. The resulting membranes benefit from the porosity of the support while increasing the available binding surface area to improve volumetric binding capacity. However, the improvement in volumetric binding capacity has only been demonstrated for solutions with low salt concentrations³⁻⁵. Operating pharmaceutical separations in solutions with low ionic strength typically requires a buffer exchange step which increases processing costs⁷. In order to reduce the extent of the buffer exchange step, it has been necessary to develop membranes which demonstrate consistent binding over a range of salt concentrations. The improvement of the salt tolerance of membrane adsorbers typically requires a reduction in the ionic sensitivity of the binding ligand through manipulation of the ligand chemistry. Recent work replacing ligands with quaternary amine-based chemistry with those containing predominantly primary amine chemistry demonstrated volumetric binding capacities which were essentially constant over a range of conductivities^{6,9,10}. Although these membranes achieved high salt tolerance, the reported volumetric binding capacities were low. While recent work in the field reliably addresses one of the drawbacks mentioned above, there is still a need for a membrane adsorber which provides a consistently high volumetric binding capacity over a wide range of salt concentrations.

In this study we describe the modification of a mixed matrix membrane to produce a salt-tolerant weak anion-exchange membrane with high volumetric binding capacity. Mixed matrix membranes provide a unique opportunity as ion-exchange membranes due to the presence of embedded microparticles in the polymer matrix enabling three-dimensional capture of proteins, similar to that found in resins, while still retaining the beneficial flow properties of membrane chromatography. In this paper we narrow our focus to mixed matrix membranes with in situ generated organic microparticle gels. Using functional organic microparticle gels which swell in the presence of water, the active surface of the membrane may be increased with minimal changes to the volume, resulting in an improved volumetric binding capacity. The in situ nature of the particles allow adaptive functionalization through appropriate choice of crosslinker to improve salt tolerance and tailor microgel behavior. The casting conditions of the mixed matrix membrane were modified in order to promote a porous structure with reliable placement of the functional microparticles. The porous membrane morphology and location of the microparticles was validated using SEM imaging. The microparticle composition and concentration was varied to determine optimum “particle packing” and formulation. Static and dynamic protein binding studies were performed to characterize binding properties of the synthesized membranes.

### II. Methods

2.1 Chemicals and Materials

Polyvinylidene Fluoride (PVDF) [Kynar 761] was provided by Arkema (King of Prussia, Pa.). Hyperbranched polyethylenimine (PEI) was procured from Polysciences. Epichlorohydrin (ECH), Diethylene glycol diacrylate (EGA), Bovine Serum Albumin (BSA), Bis(2-chloroethyl)amine hydrochloride (BCAH), Triethyl Phosphate (TEP), Isopropanol (IPA), Dimethyl sulfoxide (DMSO), and TRIS hydrochloride were purchased from Millipore Sigma. Hydrochloric acid was purchased from EMD Millipore. Phosphate Buffered Saline (PBS), with a 1× concentration, was purchased from Corning. All chemicals and materials were used as received. The following buffers were prepared using indicated chemicals and distilled water: buffer A—50 mM TRIS, buffer B—50 mM TRIS+50 mM NaCl, buffer C—50 mM TRIS+100 mM NaCl, buffer D—50 mM TRIS+150 mM NaCl, buffer E—50 mM TRIS+200 mM NaCl, buffer F—0.5×PBS, buffer G—1×PBS.

2.2 Membrane Fabrication

Synthesis of the mixed matrix membrane was derived from work done by Kotte et al^{11,12}. For a typical membrane the structural polymer, PVDF, was first dissolved in TEP at 80 C. The polymer solution was then put under a nitrogen atmosphere and the indicated amount of functional particle precursor, PEI, dissolved in TEP was added. Next, a catalytic amount (× uL) of concentrated HCl was added to the solution. After 15 minutes of mixing, the crosslinker was added to the casting solution followed by a 4-hour crosslinking reaction. The solution was then put under vacuum for 10 minutes in preparation for membrane casting. The solution was then cast on a glass plate at a blade height of 300 um and was left in room temperature air for 30 seconds before being immersed in the appropriate nonsolvent for 2 hours. The solidified membrane was removed from the nonsolvent bath and stored in a fresh water bath or dried for further characterization.

2.3 Varying Particle Loading

Membranes with different PEI particle loadings, as seen in Table 13, were prepared in this study. The PEI particle loading was adjusted by increasing the mass of PEI and ECH in the casting solution, while the mass of PVDF, the mass of solvent, and the ratio between PEI and the crosslinker were held constant. Membranes at each composition reported in Table 13 were cast using both water and isopropanol as nonsolvents.

TABLE 13

| | | | | | Weight % PEI in |
|---|---|---|---|---|---|
| Membrane | PVDF (g) | TEP (g) | PEI (g) | ECH (g) | final membrane |
| 1 | 5.91 | 37.45 | 0.26 | 0.17 | 6 |
| 2 | 5.91 | 37.45 | 1.10 | 0.71 | 21 |
| 3 | 5.91 | 37.45 | 2.58 | 1.65 | 38 |
| 4 | 5.91 | 37.45 | 3.88 | 2.48 | 48 |
| 5 | 5.91 | 37.45 | 5.00 | 3.20 | 54 |
| 6 | 5.91 | 37.45 | 6.50 | 4.16 | 60 |

2.4 Varying Crosslinker and Crosslink Density

Several membranes with modified functional particles were prepared by changing the crosslinker (ECH, EGA, or BCAH) or crosslink density. In membranes made using ECH or EGA, only the mass of crosslinker was adjusted to modify the functional particles while the mass of PVDF, TEP, and PEI were all held at the values reported in formulation 5 of Table 13. The ratio of PEI to ECH in formulation 5 was treated as the “base case” and the reported crosslink densities in Table 14 were normalized to this ratio. The same steps were used for membranes made with BCAH with the notable exception of dissolving the crosslinker in DMSO prior to its addition to the casting solution. The amount of DMSO required is reported in Table 14. All membranes reported in membrane 2 were cast with IPA as the nonsolvent.

TABLE 14

| Membrane | Crosslinker | Crosslink density | DMSO (mL) |
|---|---|---|---|
| 5A | ECH | 0.25 | NA |
| 5B | | 0.5 | NA |
| 5C | | 1.0 | NA |
| 5D | BCAH | 0.25 | 3 |
| 5E | | 0.5 | 5 |
| 5F | | 1.0 | 8 |
| 5G | EGA | 0.25 | NA |
| 5H | | 0.5 | NA |
| 5I | | 1.0 | NA |

2.5 Membrane Properties Characterization

2.5.1 SEM

The top surface and cross section of the samples was imaged using an SEM. All samples were coated with a Pt/Pd conductive layer on the surface of interest prior to imaging. The cross-section samples were prepared by immersing them in liquid nitrogen and then fracturing them. The resulting SEM micrographs were used to characterize sample morphology and estimate sample thickness.

2.5.2 Protein Adsorption Studies

The static binding capacities of membranes 5A-I were measured. To measure the binding capacities, a known volume of membrane was immersed in a 2 mg/mL BSA solution and gently mixed for 48 hours. The absorbance of the solution was then measured using a UV-vis spectrometer and the reported value of absorbance at 280 wavenumbers was used to determine the mass of BSA bound per volume of membrane. Each membrane was tested in BSA solutions with the following composition: distilled water (0.01 mS/cm), 50 mM TRIS buffer (4.7 mS/cm), and 1×PBS (17 ms/cm) with a salt concentration of 250 mM. Membranes 5E and 5H were also tested in 0.5×PBS (9.1 mS/cm) and 50 mM TRIS with 100 mM NaCl (15 mS/cm) to measure the salt tolerance of these two formulations. Dynamic binding experiments were then conducted on 5E membranes in a dead-end filtration configuration at a variety of flow rates (4, 8, & 10 membrane volumes/minute). The membranes were tested using solutions of BSA in 50 mM TRIS buffer with varying concentrations of NaCl (50, 100, 150, and 200 mM). The 10% breakthrough method was used to determine the binding capacity.

### III. Results & Discussion

3.1 Variation of Particle Loading

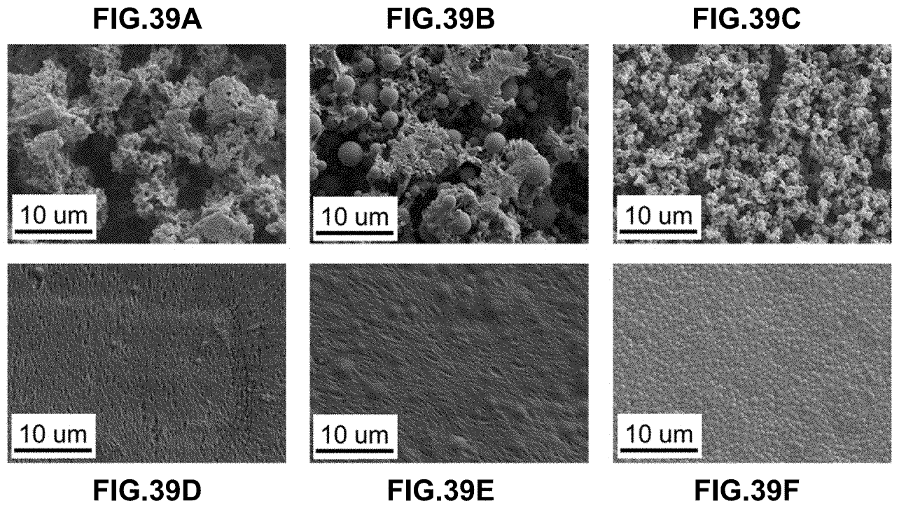

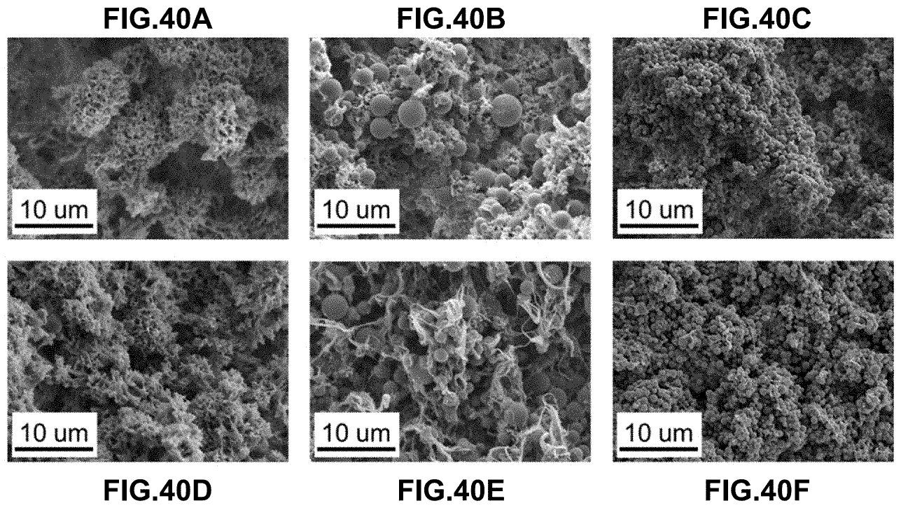

The aim of varying the PEI loading was to identify the formulation which optimized number of binding amines while maintaining desired membrane stability. FIGS. 39A-39F and 40A-40F present surface and cross-section SEM micrographs, respectively, of samples prepared in IPA and water. Combining information from these two figures, we determined that the size and number of functional PEI particles are mostly a function of PEI loading and may be loosely classified into two regimes. The first regime is observed at low PEI loadings with the number and size of the particles both increasing as the initial concentration of PEI increases. The second regime begins when the PEI concentration reaches approximately 48%. Within this regime the particle number continues to increase as the particle size remains essentially constant with increasing PEI concentration. The change in particle density and size is due to differences between the rates of nucleation and growth with different PEI concentrations. At low PEI concentrations the nucleation and growth rate are of the same order of magnitude resulting in an increase of both particle size and number with increasing PEI concentration. At higher PEI concentrations a large number of particle nuclei form in close proximity resulting in rapid depletion of free PEI in the surrounding solution thereby halting particle growth. This phenomenon leads to the particle size within the second regime being approximately constant while the number of particles increased. Having a larger number of smaller particles increases the ratio of active surface area to volume, which is advantageous for bioseparations.

Membrane morphology and location of the functional particles was further controlled by manipulating the coagulation bath. Through comparing the surface of samples prepared with the same PEI loading in different nonsolvents, we observed that a PVDF skin layer is only present in membranes prepared with water regardless of PEI loading (FIGS. 39A-39F). This is in agreement with the trends observed in literature using both neat PVDF membranes¹³⁻¹⁹ and mixed matrix membranes prepared with preformed particles^{11,12}. The presence of the skin layer is due to the rapid demixing and subsequent solidification of PVDF at the nonsolvent/polymer solution interface. This tight skin layer provides a barrier to effective mass transport through the membrane and would remove the benefits of membrane chromatography. In contrast, the open structure obtained in membranes prepared with IPA should facilitate rapid mass transfer through the membrane.

In addition to the differences in surface morphology, the cross-section micrographs in FIGS. 40A-40F provide key insight into the location of the functional particle in relation to the structural polymer matrix. With samples prepared in water, the particles tend to be intertwined with the polymer matrix. Meanwhile, samples prepared with IPA have the particles located on the outside of the spherulitic crystals formed during the phase inversion process. The location of the particles has important implications when operating in a dynamic flow through setting, wherein removing any mass transfer limitations is critical to achieve high binding capacity. Having the particles located on the edge of the particle matrix ensures minimal interference from the structural polymer. Based upon this analysis, formulation 5 from Table 13 prepared in IPA was chosen as the base case for the protein studies.

3.2 Variation of Crosslinker and Crosslink Density

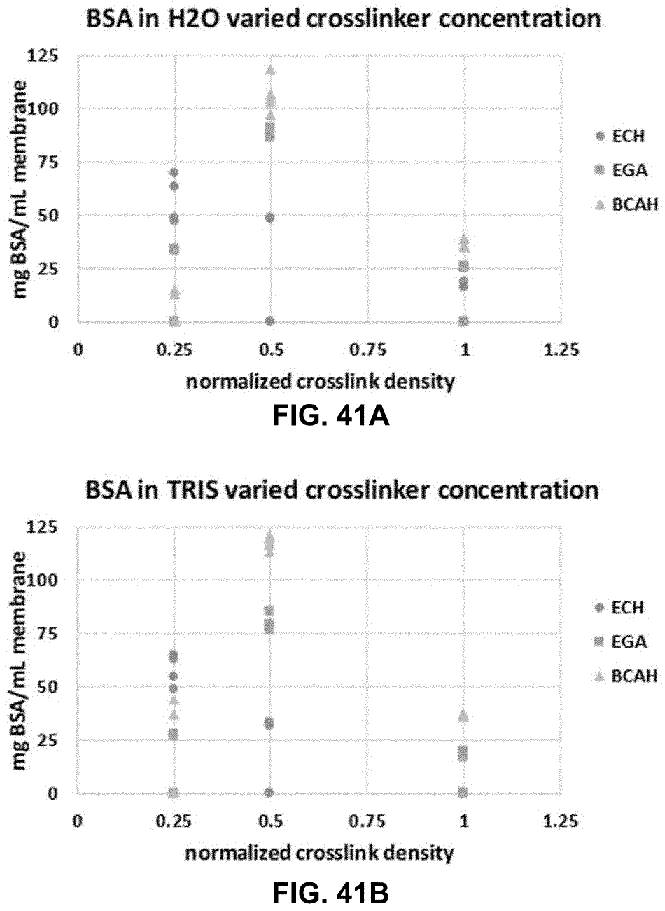

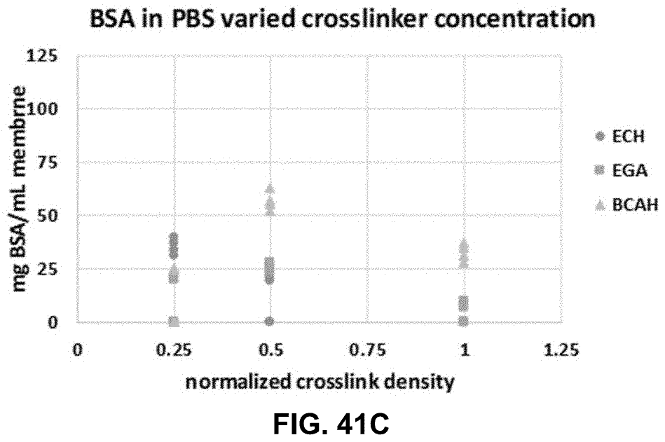

To achieve a high binding capacity and improved salt tolerance, a set of membranes with different crosslinkers and crosslink density were formulated. The static binding capacities, depicted in FIGS. 41A-41C, demonstrate a few interesting trends. Considering first the case of ECH as the crosslinker with varying crosslink density. There appears to be a negative correlation between crosslink density and binding capacity in each of the three different BSA solvents. This negative correlation is similar to the relationship between the number of binding amines and the crosslink density. With an increase in crosslink density many amines are covalently bonded into the particle and as a result suffer from increased steric hindrance and changes in electron distribution. These changes reduce the ability of the amines to electrostatically interact with BSA and ultimately lead to a lower volumetric binding capacity. Another possible contribution is the “looseness” of the microgel at low crosslink densities which would potentially allow for a greater active surface area. However, with a low crosslink density it would seem likely that not all of the PEI is incorporated into the functional microparticles and some portion of the particle precursor would have been removed from the membrane during the casting process. As a result, we would expect to see a reduction in the number of amines present within the membrane leading to a reduction in the volumetric binding capacity. It is possible that the percentage of PEI molecules not incorporated into the functional particles would be small and would therefore not have a strong influence on the overall trend.

The analysis above is further complicated by considering membranes prepared with EGA or BCAH, which both exhibit a maximum binding capacity at a normalized crosslink density of 0.5. The presence of this maximum does support the assertion that there is a balance between number of free amines—to bind BSA—and crosslink density—to secure the functional PEI particles/gels in place. However, the presence of the maximum does suggest that there is another contribution which is sensitive to using ECH as the crosslinker. Whether that contribution is simply a function of crosslinker length or chemistry is still to be determined.

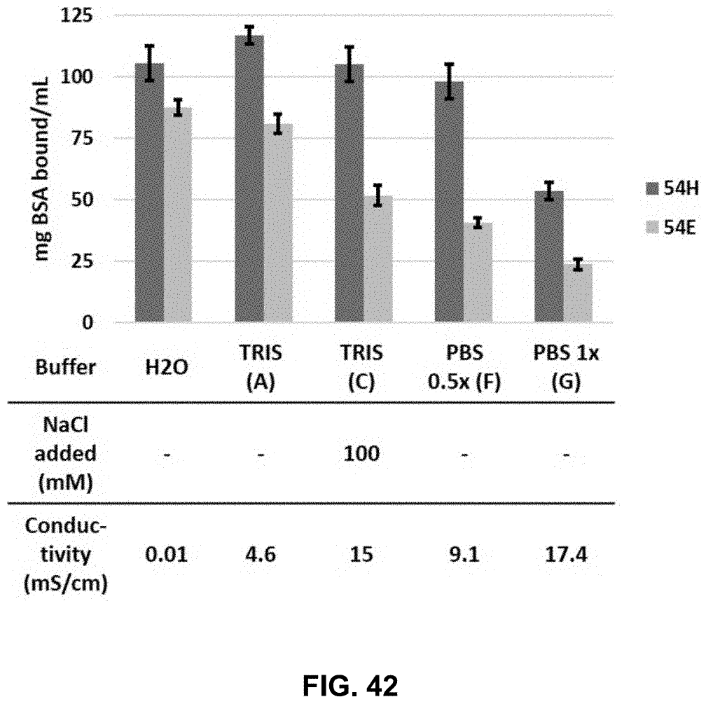

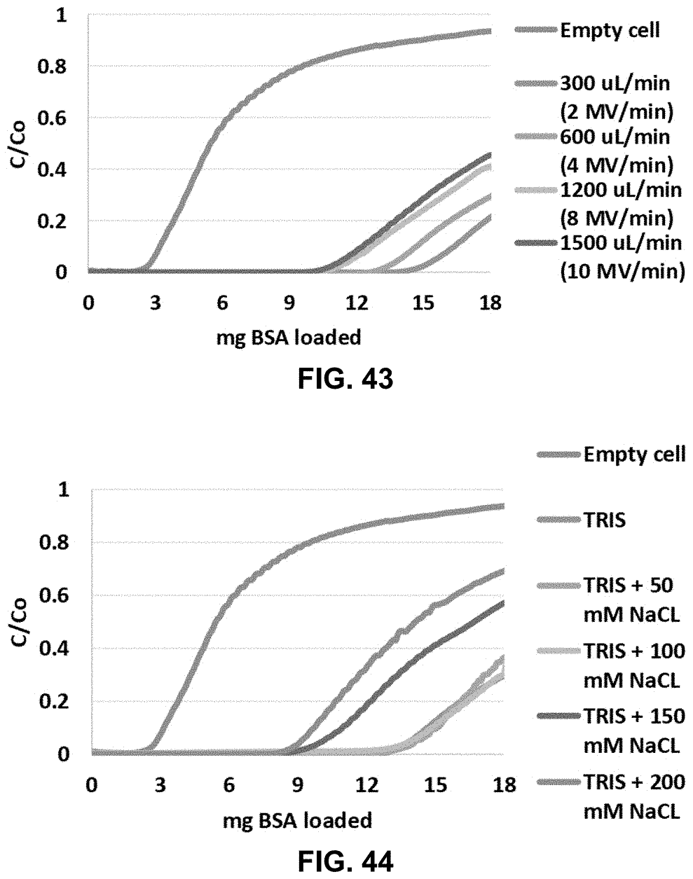

Samples 5E and 5H demonstrate the highest binding capacities in both water and the TRIS buffer and are therefore used in the salt tolerance experiments. During the experiment the two modified mixed matrix membranes were incubated in 5 different BSA solutions. FIG. 42 presents a compelling case that the membrane prepared with BCAH is salt tolerant producing good binding (>90% of maximum binding) up to 120 mM of NaCl. After which the ability to bind starts decreasing with a 50% drop in overall binding capacity at 250 mM of salts. In addition to achieving the desired salt tolerance, membrane 5E also achieves a high volumetric binding capacity of ˜100 mg/mL at a salt concentration of 120 mg/mL which is approximately 2× higher than reported in the literature. In contrast the membrane prepared using EGA as the crosslinker has been reduced by 33% and 67% at 100 and 250 mM of salt respectively. The large disparity in performance between 5E and 5H is due to the chemistry of the crosslinker. While EGA is hydrophilic and provides limited interference to the binding amines, it doesn't contribute to the total number of binding sites in the membrane either. In contrast, BCAH is not only hydrophilic, but provides an additional binding site once it has been incorporated into the functional particle thereby providing additional opportunities to reduce the ionic screening of the BSA buffers.

3.3 Dynamic Binding Influenced by Flowrate and Salt Concentration

The results from the static binding measurements indicated that membrane 5E had the best salt tolerance and volumetric binding capacity, so this formulation was used for the dynamic binding experiments. The first relationship investigated was the influence of flowrate on the dynamic binding capacity. In contrast to the typical behavior of membrane chromatography, where the dynamic binding capacity is essentially independent of flow rate, the modified mixed matrix membranes demonstrate a resin-like flowrate dependence at slow fluid velocities (<8 MV/min) as seen in FIG. 5. In contrast, at higher fluid velocities (>8 MV/min) the dynamic binding capacity reaches a plateau region where it is no longer dependent on the flowrate. These observations may be explained by considering the mechanisms of binding used by the functional groups within the microgels. Similar to the behavior seen in resins, at lower flowrates (2-8 MV/min) the BSA molecules have additional time to penetrate the gel allowing for access to a larger number of binding sites. As the flowrate increases, fewer BSA molecules are able to interact with the interior of the gel leading to a reduction in the “perceived” binding sites and a lower binding capacity. Eventually, a regime is reached (8+ MV/min) where the only microgel binding sites that have sufficient time to bind are those on the peripheries. Within this regime the modified mixed matrix membrane exhibits behavior similar to traditional membrane chromatography, with dynamic binding capacity being independent of flowrate.

In addition to the influence of flowrate, the dynamic binding salt tolerance of the 5E membrane was also investigated. The measurements presented in FIG. 44 demonstrate the relatively constant BSA binding achieved in buffer alone and low salt concentrations (<150 mM) solutions, which agrees nicely with the trends observed in the static binding measurements. Maintaining a good binding capacity in the presence of NaCl may be attributed to the significant presence of primary and secondary amines. The primary and secondary amines act as weak bases, which allow them to interact both electrostatically and through intermolecular forces with the BSA molecules. In contrast, strong bases (such as quaternary amines) only interact electrostatically. As a result, the electrostatic interactions screened at low salt concentrations result in a significant decrease in the binding capacity of ion exchange membranes that use strong base functionalities, while having a negligible impact on those using weak base functionalities. As the salt concentration increases, the presence of so many dissolved ions interfere with both electrostatic interactions and intermolecular forces leading to a reduction in dynamic binding capacity for weak base AEX membranes. Such a reduction is observed in the dynamic binding measurements of the 5E membrane with the binding capacity initially decreasing once the salt concentration passes 100 mM. Upon reaching a NaCl concentration of 200 mM, the dynamic binding capacity drops from 81 mg BSA/mL to 45 mg BSA/m L, a reduction of ˜44%. It was also found that the salt tolerance trend observed at 4 MV/min extended to measurements at 8&10 MV/min (FIG. 31). The consistency in salt tolerance behavior suggests that the binding interactions both on the gel peripheries and within the bulk are the same.

### IV. Conclusion

Maintaining high protein binding capacity over a range of solution conductivities is a critical step in reducing downstream bioprocessing costs. We show that these properties are obtainable by modifying the morphology and chemistry of mixed matrix membranes with in situ grown functional microparticles. Membrane morphology was tested at different particle loadings to maximize the number of functional microparticles while maintaining membrane strength and durability. The optimized membrane formulation was further modified by changing the crosslink density and crosslinker chemistry. The resulting membranes were used in static protein binding experiments. Membranes prepared with BCAH and EGA at 0.5 normalized crosslink density demonstrated the best static binding and were tested in 5 solutions with conductivities ranging from 0 to 20 mS/cm. Using BCAH resulted in membranes which retained over 90% of the initial 100 mg/mL binding capacity at 10 mS/cm and ˜50% at 20 ms/cm. Dynamic binding measurements revealed that the modified mixed matrix membranes exhibit resin-like behavior at flowrates below 8 MV/min and approach membrane-like behavior at flowrates above 8 MV/min. At the lowest flowrate of 2 MV/min a binding capacity of 89 mg BSA/mL was achieved in 50 mM TRIS. In the plateau region, the dynamic binding capacity was found to be 62 mg BSA/mL. The dynamic binding salt tolerance exhibited trends similar to those identified in the static binding measurements, with high retention of binding capacity up to 15 mS/cm followed by a 50% decrease in binding capacity at 30 mS/cm.

### References Corresponding to Example 12

(1) Ghosh, R. Protein Separation Using Membrane Chromatography: Opportunities and Challenges. J. Chromatogr. A 2002, 952 (1), 13-27. https://doi.org/10.1016/S0021-9673(02)00057-2.
(2) Madadkar, P.; Wu, Q.; Ghosh, R. A Laterally-Fed Membrane Chromatography Module. J. Membr. Sci. 2015, 487, 173-179. https://doi.org/10.1016/j.memsci.2015.03.056.
(3) Bhut, B. V.; Wickramasinghe, S. R.; Husson, S. M. Preparation of High-Capacity, Weak Anion-Exchange Membranes for Protein Separations Using Surface-Initiated Atom Transfer Radical Polymerization. J. Membr. Sci. 2008, 325 (1), 176-183. https://doi.org/10.1016/j.memsci.2008.07.028.
(4) Bhut, B. V.; Husson, S. M. Dramatic Performance Improvement of Weak Anion-Exchange Membranes for Chromatographic Bioseparations. J. Membr. Sci. 2009, 337 (1), 215-223. https://doi.org/10.1016/j.memsci.2009.03.046.
(5) Sun, L.; Dai, J.; Baker, G. L.; Bruening, M. L. High-Capacity, Protein-Binding Membranes Based on Polymer Brushes Grown in Porous Substrates. Chem. Mater. 2006, 18 (17), 4033-4039. https://doi.org/10.1021/cm060554m.
(6) Fischer-Frühholz, S.; Zhou, D.; Hirai, M. Sartobind STIC® Salt-Tolerant Membrane Chromatography. Nat. Methods 2010, 7 (12), 12-13.
(7) Orr, V.; Zhong, L.; Moo-Young, M.; Chou, C. P. Recent Advances in Bioprocessing Application of Membrane Chromatography. Biotechnol. Adv. 2013, 31 (4), 450-465. https://doi.org/10.1016/j.biotechadv.2013.01.007.
(8) Keating, J. J.; Imbrogno, J.; Belfort, G. Polymer Brushes for Membrane Separations: A Review. ACS Appl. Mater. Interfaces 2016, 8 (42), 28383-28399. https://doi.org/10.1021/acsami.6b09068.
(9) Riordan, W.; Heilmann, S.; Brorson, K.; Seshadri, K.; He, Y.; Etzel, M. Design of Salt-Tolerant Membrane Adsorbers for Viral Clearance. Biotechnol. Bioeng. 2009, 103 (5), 920-929. https://doi.org/10.1002/bit.22314.
(10) Fan, J.; Luo, J.; Chen, X.; Wan, Y. Facile Preparation of Salt-Tolerant Anion-Exchange Membrane Adsorber Using Hydrophobic Membrane as Substrate. J. Chromatogr. A 2017, 1490, 54-62. https://doi.org/10.1016/j.chroma.2017.02.016.
(11) Kotte, M. R.; Cho, M.; Diallo, M. S. A Facile Route to the Preparation of Mixed Matrix Polyvinylidene Fluoride Membranes with In-Situ Generated Polyethyleneimine Particles. J. Membr. Sci. 2014, 450, 93-102. https://doi.org/10.1016/j.memsci.2013.08.025.
(12) Rao Kotte, M.; Hwang, T.; Han, J.-I.; Diallo, M. S. A One-Pot Method for the Preparation of Mixed Matrix Polyvinylidene Fluoride Membranes with in Situ Synthesized and PEGylated Polyethyleneimine Particles. J. Membr. Sci. 2015, 474, 277-287. https://doi.org/10.1016/j.memsci.2014.09.044.
(13) Cheng, L.-P.; Lin, D.-J.; Shih, C.-H.; Dwan, A.-H.; Gryte, C. C. PVDF Membrane Formation by Diffusion-Induced Phase Separation-Morphology Prediction Based on Phase Behavior and Mass Transfer Modeling. J. Polym. Sci. Part B Polym. Phys. 1999, 37 (16), 2079-2092. https://doi.org/10.1002/(SICI)1099-0488(19990815)37:16<2079::AID-POLB11>3.0.CO; 2-Q.
(14) Jung, J. T.; Kim, J. F.; Wang, H. H.; di Nicolo, E.; Drioli, E.; Lee, Y. M. Understanding the Non-Solvent Induced Phase Separation (NIPS) Effect during the Fabrication of Microporous PVDF Membranes via Thermally Induced Phase Separation (TIPS). J. Membr. Sci. 2016, 514, 250-263. https://doi.org/10.1016/j.memsci.2016.04.069.
(15) Young, T.-H.; Cheng, L.-P.; Lin, D.-J.; Fane, L.; Chuang, W.-Y. Mechanisms of PVDF Membrane Formation by Immersion-Precipitation in Soft (1-Octanol) and Harsh (Water) Nonsolvents. Polymer 1999, 40 (19), 5315-5323. https://doi.org/10.1016/S0032-3861(98)00747-2.
(16) Thomas, R.; Bilad, M.; Arafat, H. PVDF Membranes for Membrane Distillation: Controlling Pore Structure, Porosity, Hydrophobicity, and Mechanical Strength. Membr Fabr 2015, 249-284.
(17) Lin, D.-J.; Chang, H.-H.; Chen, T.-C.; Lee, Y.-C.; Cheng, L.-P. Formation of Porous Poly(Vinylidene Fluoride) Membranes with Symmetric or Asymmetric Morphology by Immersion Precipitation in the Water/TEP/PVDF System. Eur. Polym. J. 2006, 42 (7), 1581-1594. https://doi.org/10.1016/j.eurpolymj.2006.01.027.
(18) Liu, F.; Hashim, N. A.; Liu, Y.; Abed, M. R. M.; Li, K. Progress in the Production and Modification of PVDF Membranes. J. Membr. Sci. 2011, 375 (1), 1-27. https://doi.org/10.1016/j.memsci.2011.03.014.
(19) Gu, M.; Zhang, J.; Wang, X.; Ma, W. Crystallization Behavior of PVDF in PVDF-DMP System via Thermally Induced Phase Separation. J. Appl. Polym. Sci. 2006, 102 (4), 3714-3719.

### Example 13: Membrane Preparation and Characterization

Chemicals and Materials: Polyvinylidene fluoride (PVDF) [Kynar 761] was provided by Arkema (King of Prussia, Pa., USA). G0-NH2 and G1-NH2 PAMAM dendrimers were purchased as methanol solutions (˜34 wt %) from Dendritech Inc, USA. Table 15 lists selected physical-chemical properties of the PAMAM dendrimers. Epichlorohydrin (ECH) was purchased from Sigma-Aldrich. Triethyl phosphate (TEP), ethanol and nitric acid (60 wt % HNO3) were purchased from Daejung Chemicals (South Korea). Hydrochloric acid (12 M HCl) was purchased from Junsei (South Korea). Sodium hydroxide (NaOH pellets) and copper(II) nitrate trihydrate (ACS purus grade) were purchased from Sigma-Aldrich. A standard solution of copper (Cu) [10 mg/L in 5 wt % HNO3] (Multi-element calibration standard-2A) was purchased from Agilent Technologies. All chemicals were used as received. All aqueous solutions were prepared using Milli-Q deionized water (DIW) with a resistivity of 18.2MΩcm and total organic content <5 ppb.

TABLE 15
Selected physicochemical properties of the PAMAM dendrimers that were utilized as particle
precursors for the mixed matrix PVDF membranes with in situ synthesized PAMAM particles.

| | (Dalton) | | | | | | | | |
|---|---|---|---|---|---|---|---|---|---|
| | ^{a}M_{wth} | | | | (meq/g) | (meq/g) | (meq/g) | (meq/g) | (nm) |
| Dendrimer | (Da) | ^{b}N_{Pamine} | ^{c}N_{Tamine} | ^{d}N_{Amide} | ^{e}C_{Pamine} | ^{f}C_{Tamine} | ^{g}C_{Amide} | ^{h}C_{Ligand} | ⁱD_{H} |
| G0-NH₂ | 517 | 4 | 2 | 4 | 5.56 | 2.78 | 5.56 | 19.47 | 1.5 |
| G1-NH₂ | 1430 | 8 | 6 | 12 | 4 | 3 | 6 | 18.99 | 2.2 |

^{a}Mwth: theoretical molecular weight.
^{b}NPamine: number of primary groups.
^{c}NTamine: number of tertiary amine groups.
^{d}NAmide: number of amide groups.
Each amide group has 2 potential electron donors: 1 N donor and 1 O donor.
^{e}CPamine and ^{f}CTamine are, respectively, the concentrations of primary and tertiary amino groups per gram of PAMAM respectively.
^{g}CAmide and ^{h}CLigand are the concentration of amide and ligand functionalities per gram of PAMAM respectively.
ⁱDH: theoretical hydrodynamic diameter of dendrimer molecule.

Membranes were prepared using a combined thermally-induced phase separation (TIPS) and non-solvent induced phase separation (NIPS) process. Table 16 lists the compositions of the membrane casting solutions.

TABLE 16
Compositions of the casting solutions, neat PVDF membrane and
mixed matrix PVDF membranes with in situ synthesized crosslinked
PAMAM particles that were prepared in this example.

| | | | | | PVDF | |
|---|---|---|---|---|---|---|
| | MDP-G0 | | MDP-G1 | | (Neat) | |
| Membrane | M (g) | wt. % | M (g) | wt. % | M( g) | wt. % |

A. Compositions of Membrane Casting Solutions

| | | | | | | |
|---|---|---|---|---|---|---|
| ^{a)}PVDF | 18 | 11 | 18 | 10.99 | 18 | 15 |
| ^{b)}PAMAM + ^{c)}ECH | 19.46 | 11.9 | 19.46 | 11.88 | — | — |
| ^{d)}TEP | 120.1 | 73.46 | 120.1 | 73.31 | 102.0 | 85.0 |
| ^{e)}PAMAM Solution | 5.95 | 3.64 | 6.27 | 3.83 | — | — |
| (Methanol) | | | | | | |

B. Estimated Membrane Compositions (Dry mass wt. %)

| | | | | | | |
|---|---|---|---|---|---|---|
| PVDF | 18 | 52.29 | 18 | 52.29 | 18 | 100 |
| ¹Crosslinked PAMAM | 16.43 | 47.71 | 16.43 | 47.71 | — | — |
| particles | | | | | | |

C. Estimated degree of crosslinking of PAMAM particles based
on ECH concentration (Dry mass wt. %)

| | | | | | | |
|---|---|---|---|---|---|---|
| ²ECH | 7.71 | 39.62 | 7.71 | 39.62 | — | — |
| PAMAM | 11.75 | 60.38 | 11.75 | 60.38 | — | — |

^{a)}PVDF: Polyvinylidene fluoride;
^{b)}PAMAM: Polyamidoamine;
^{c)}ECH: Epichlorohydrin;
^{d)}TEP: Triethyl phosphate;
^{e)}Methanol solutions of G0-NH₂ PAMAM (33.6 wt. %) and G1-NH₂ PAMAM (34.79 wt. %).
¹The mass fraction of crosslinked PAMAM particles in each membrane was estimated based on the following assumptions: i) All ECH crosslinker molecules were reacted with the segregated PAMAM molecules by the reaction between epoxy & chloro groups of ECH and primary/secondary amino groups of PAMAM molecules in the dope solutions (FIG. 1B). ii) Each ECH molecule produces one molecule of hydrogen chloride (HCl) following the crosslinking reaction (FIG. 1B). iii) All unreacted PAMAM molecules were washed away in the coagulation bath and subsequent membrane washes with methanol and DIW.
²The weight fraction (dry mass wt. %) of ECH was taken as a surrogate for the degree of crosslinking of the PEI based on our previous work on the synthesis of perchlorate-selective resin beads (Chen, D.P.; Yu, C.J.; Chang, C-Y.; Wan, Y.; Frechet, J.M.J.; Goddard, W.A. III.; Diallo, M.S. Branched polymeric media: perchlorate-selective resins from hyperbranched polyethyleneimine. Environ. Sci. Technol. 2012, 46, 10718-10726.).

A control PVDF membrane and two mixed matrix PVDF membranes with in situ synthesized PAMAM particles (MDP-G0 and MDP-G1) were prepared using the three-step process given below. The recipe used to prepare the mixed matrix membranes (MMMs) was selected to achieve a high particle loading (˜50 wt %) based on the results of our previous work on mixed matrix PVDF membranes with in situ synthesized PEI particles. The MDP-G0 and MDP-G1 membranes were prepared using G0-NH2 and G1-NH2 PAMAM dendrimers as particle precursors, respectively.

1) Preparation of Membrane Casting Solutions. A typical membrane casting solution was prepared by mixing the required amounts of PVDF and TEP in a three neck round-bottom flask equipped with a condenser and an overhead stirrer. A homogeneous PVDF dope solution was obtained after mixing for 24 hours at 80° C. Following this, the prepared PVDF dope solution was transferred into a glass container and covered with aluminum foil.

2) In Situ Synthesis of Crosslinked PAMAM Dendrimer Particles. Prior to membrane casting, the PVDF dope solution was homogenized at 4000 rpm for 7 minutes using a Silverson L5M high shear mixer (HSM). During the homogenization, the temperature of the dope solution was raised to 80° C. and kept constant. A solution of PAMAM in TEP was then added drop wise to the PVDF dope solution for 5 minutes followed by high shear mixing for 15 minutes to obtain a homogeneous PVD+PAMAM dispersion in TEP. A solution of ECH in TEP was then added drop wise to the dispersion and homogenized for 5 minutes under similar HSM conditions to obtain a stable dispersion of PAMAM particles in the PVDF+TEP dope. Finally, the curing reaction was continued in a round bottom flask equipped with an overhead stirrer at 80° C. for 3 hours.

3) Membrane Casting. Following the completion of the curing reactions, the dispersion of PVDF+TEP+ECH crosslinked PAMAM particles dope was allowed to cool to ambient temperature to initiate the TIPS step of the membrane casting process. The membranes were prepared with and without a polyethylene terephthalate (PET) microporous support. To prepare a membrane without support, the cooled dispersion of PVDF+PAMAM particle in TEP was poured onto a clean glass plate. A casting knife (BYK Chemie) [with 300 μm air gap] was used to uniformly coat the casting solution onto the glass plate. The nascent membrane was kept for 30 seconds at ambient temperature (25±1° C., RH: 55%) followed by immersion into a DIW bath with a temperature of 23±1° C. After 1 hr, the nascent membrane was transferred to a fresh DIW bath and immersed for 24 h. Following this, the membrane was soaked in ethanol for 10 h. Finally, the membranes were air dried and stored in a desiccator. A similar procedure was used to prepare a membrane with microporous support by pouring the casting solution on a PET non-woven fabric. The supported membranes were stored in DIW with the water periodically replaced with fresh DIW until the metal binding experiments were initiated.

1) Membrane Morphology. The cross-sectional and top surface of each membrane was imaged with a field emission scanning electron microscope (FESEM, Magellan Series 400, FEI Corporation) at an acceleration voltage of 2.0 kV. Before imaging, all samples were first coated with platinum for 30 seconds followed by osmium for 30 seconds to minimize the charging effect. To obtain the membrane cross section morphology, the membranes were frozen and fractured following immersion in liquid nitrogen. The SEM images were subsequently analyzed to estimate membrane thickness and PAMAM particle size using the Image J Version 1.45m image processing/analysis software.

2) N₂ Adsorption Permporometry. The average pore diameter of each membrane top/skin layer was determined by N₂ adsorption permporometry at 77 K using a Micromeritics ASAP 2020 accelerated surface area and porosimetry analyzer. The Barrett-Joyner-Halenda (BJH) methodology was utilized to extract membrane pore diameters from the N₂ adsorption/desorption data.

3) Membrane Surface Composition. The surface chemical composition was characterized by Fourier transform infrared (FT-IR) spectroscopy. The mid IR spectra (500 cm⁻¹ to 4000 cm⁻¹) of the membranes were scanned in attenuated total reflectance (ATR) mode. The spectra were acquired by averaging 32 scans at a resolution of 2 cm-1 using a JASCO 4100 FT-IR spectrometer (Japan) and a zinc selenide ATR crystal plate with an aperture angle of 45°. In contrast, the near IR (NIR) spectrum of each membrane (4000 cm-1 to 10000 cm-1) was recorded by reflection using a Bruker MPA FT-NIR spectrometer equipped with a quartz beam splitter and an external RT-PbS detector. The NIR spectra were acquired by averaging 32 scans at a resolution of 8 cm⁻¹. The elemental composition of each membrane surface was analyzed by X-ray photoelectron spectroscopy (XPS) using an SSX-100 UHV spectrometer from Surface Science Instruments. The sample was irradiated with a beam of monochromatic Al Kα X-rays with energy of 1.486 keV.

4) Contact Angle Measurements. The hydrophobicity of each membrane was determined from contact angle measurements using a Phoenix 300 contact angle analyzer. A micro syringe was utilized to place a water droplet on the surface of each membrane. After 30 and 120 seconds, the image was captured and analyzed using the instrument's image processing software. Each reported contact angle is the average of five different measurements.

5) Particle Size Measurements by DLS. A 0.2 g of dry membrane was added to 20 g of TEP solvent in sample vial. It was allowed for dissolution for 15 hours at ambient temperature as a result fine dispersion was obtained. Then the dispersion was sonicated for 15 minutes. A 1.0 mL aliquot was sampled from the dispersion and diluted with 10 mL of TEP solution for the DLS measurements. These were conducted in duplicate at 25° C. using TEP solvent.

6) Zeta Potential Measurements. The zeta potentials of the membranes were determined using the electrophoresis method. An ELSZ-2 electrophoretic light scattering spectrophotometer from Otsuka Electronics, Japan [with a plate quartz cell as membrane holder] was employed to measure the electrophoretic mobility of the monitoring particles. The monitoring particles consisted of polystyrene (PS) latex particles (Otsuka Electronics, Japan) with an amide surface coating and S5 diameter of 520 nm. The PS particles were dispersed in 0.01 M NaCl solutions at pH 7.0. The measured electrophoretic mobilities (U) of the monitoring PS particles [cm²/(V·s)] were utilized to calculate membrane zeta potentials (ζ) [mV] using the Smoluchowski equation as given below:

$$ϛ = \frac{4 πη U}{{ɛ}_{r} {ɛ}_{0}} \qquad Eq 1$$

where η is the liquid viscosity (0.89×10⁻³ Pa·s), eᵣ is the relative permittivity of liquid (78.38) and e₀ is the vacuum permittivity (8.854×10⁻¹² s·m⁻¹).

### Example 14: Particle Size Determination

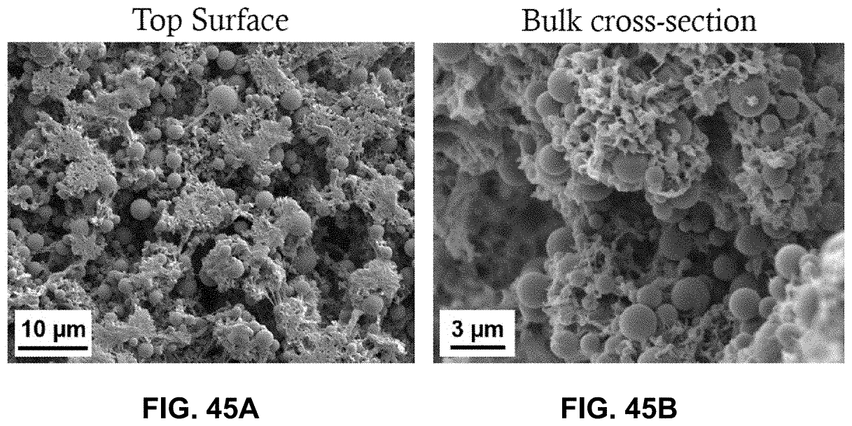

This example demonstrates how to calculate average particle size from an SEM micrograph, such as the SEM micrograph of FIGS. 45A-45B.

Generally, this method is applicable when the polymer particles are polymer micro gels (which collapse in volume when they dry). It also requires that the volume fraction of the scaffold pores that is occupied by the structural polymer is less than 10% and the volume fraction of the scaffold pores that is occupied by dry functional polymer is greater than that occupied by the structural polymer and less than 20%. These ranges provide sufficient ability to see and count a statistically significant number of dry functional polymer microgel particles.

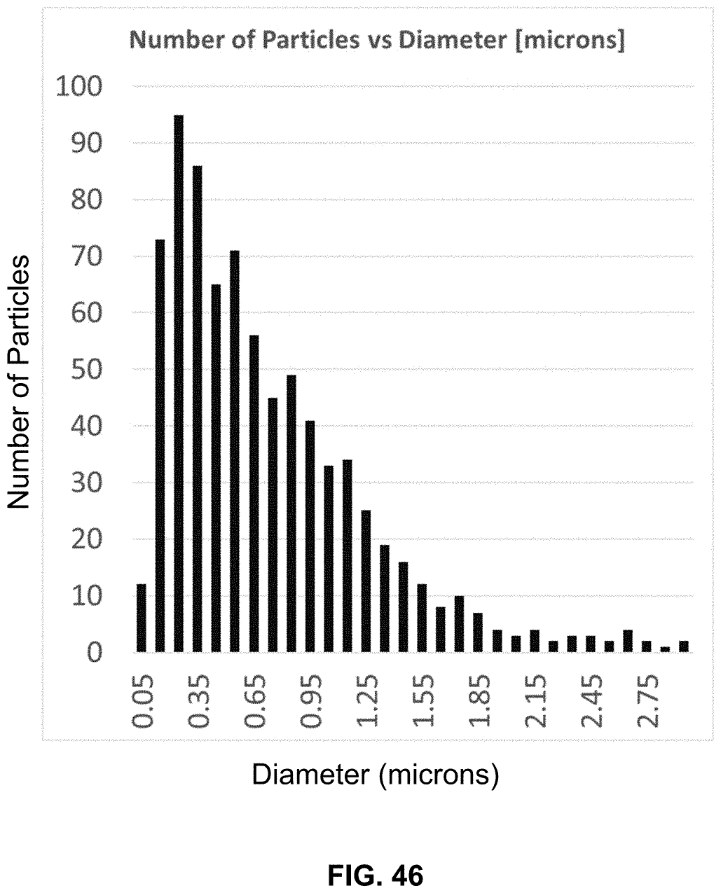

Based on a visual inspection of an SEM micrograph (FIGS. 45A-45B) for which detailed image analysis was not available, a particle size distribution (FIG. 46 and Table 17) was constructed that was similar to that shown in the SEM micrograph.

TABLE 17

| Diameter (um) | Number |
|---|---|
| 0 | |
| 0.05 | 12 |
| 0.15 | 73 |
| 0.25 | 95 |
| 0.35 | 86 |
| 0.45 | 65 |
| 0.55 | 71 |
| 0.65 | 56 |
| 0.75 | 45 |
| 0.85 | 49 |
| 0.95 | 41 |
| 1.05 | 33 |
| 1.15 | 34 |
| 1.25 | 25 |
| 1.35 | 19 |
| 1.45 | 16 |
| 1.55 | 12 |
| 1.65 | 8 |
| 1.75 | 10 |
| 1.85 | 7 |
| 1.95 | 4 |
| 2.05 | 3 |
| 2.15 | 4 |
| 2.25 | 2 |
| 2.35 | 3 |
| 2.45 | 3 |
| 2.55 | 2 |
| 2.65 | 4 |
| 2.75 | 2 |
| 2.85 | 1 |
| 2.95 | 2 |

For this distribution (FIG. 46 and Table 17), the number average volume and the volume average volume were calculated, as shown in Table 18.

TABLE 18

| Volume | | Volume * | Volume² * |
|---|---|---|---|
| (μm³) | Number | Number (μm³) | Number |
| 6.54498E−05 | 12 | 0.0007854 | 5.14042E−08 |
| 0.001767146 | 73 | 0.12900165 | 0.000227965 |
| 0.008181231 | 95 | 0.77721693 | 0.006358591 |
| 0.022449298 | 86 | 1.93063959 | 0.043341502 |
| 0.047712938 | 65 | 3.101341 | 0.147974092 |
| 0.087113746 | 71 | 6.18507599 | 0.53880514 |
| 0.143793314 | 56 | 8.05242557 | 1.157884956 |
| 0.220893233 | 45 | 9.94019551 | 2.195721926 |
| 0.321555098 | 49 | 15.7561998 | 5.066486373 |
| 0.4489205 | 41 | 18.4057405 | 8.262714237 |
| 0.606131033 | 33 | 20.0023241 | 12.12402935 |
| 0.796328288 | 37 | 29.4641467 | 23.46313345 |
| 1.022653859 | 25 | 25.5663465 | 26.14552286 |
| 1.288249338 | 21 | 27.0532361 | 34.85131347 |
| 1.596256317 | 16 | 25.5401011 | 40.76854769 |
| 1.94981639 | 12 | 23.3977967 | 45.62140748 |
| 2.35207115 | 8 | 18.8165692 | 44.25790955 |
| 2.806162188 | 5 | 14.0308109 | 39.37273113 |
| 3.315231098 | 7 | 23.2066177 | 76.93530061 |
| 3.882419471 | 8 | 31.0593558 | 120.5854476 |
| 4.510868902 | 9 | 40.5978201 | 183.1314442 |
| 5.203720981 | 10 | 52.0372098 | 270.7871205 |
| 5.964117303 | 11 | 65.6052903 | 391.2776473 |
| 6.79519946 | 12 | 81.5423935 | 554.0968284 |
| 7.700109044 | 13 | 100.101418 | 770.7918307 |
| 8.681987648 | 14 | 121.547827 | 1055.276733 |
| 9.743976864 | 15 | 146.159653 | 1424.176277 |
| 10.88921829 | 16 | 174.227493 | 1897.201198 |
| 12.12085351 | 17 | 206.05451 | 2497.556525 |
| 13.44202412 | 18 | 241.956434 | 3252.384222 |
| Sum | 900 | 1532.24597 | 12778.22469 |

The number average volume was calculated to be 1.7 by dividing 1532.2 by 900. The volume average volume was calculated to be 8.34 by dividing 12778.2 by 1532.2. PDI was calculated to be 4.9 by dividing the volume average volume (8.34) by the number average volume (1.7).

These values were then converted to number average molecular weight (Mₙ) and weight average molecular weight (M_{w}) using the density of PEI of 1.05 g/mol (literature ranges from 1.03 g/mol to 1.08 g/mol). Specifically, we multiply by density (density of PEI dry is 1.03 to 10.8 g/cc) to obtain number average mass per particle, which is 1×10⁻¹² g/(μm³). Then multiply by Avogadro's number to get molecular weight.

In some aspects, the Mₙ and M_{w} values shown in this example (e.g., Mₙ of 1×10³ g/mol to 1×10¹⁰ g/mol, and M_{w}/Mₙ of 2 to 20) are useful for producing membranes in which the functional polymer particle comprises a plurality of particles having an average diameter in a dry state of 0.3 μm to 3 μm, for use in a scaffold having pores with an average diameter of 30 μm to 60 μm.

### Example 15: Removal of Metals from a Fluid

This example demonstrates how removal of metals from a fluid, such as a fluid comprising water, can be achieved. As disclosed in Stebbins et al., “Cactus Mucilage as an Emergency Response Biomaterial to Provide Clean Drinking Water,” Monitoring Water Quality, 249-260 (2013), a naturally occurring polymer gel is disclosed to remove barium, zinc, boron, chromium, iron, selenium, arsenic, nickel, lead, or a combination thereof.

We propose employing the concept of Stebbins et al. with the functional polymer particles and composite membrane as disclosed herein. By employing functional polymer particles with at least one functional group capable of binding to species (metals) of interest, the resulting composite membrane is expected to remove barium, zinc, boron, chromium, iron, selenium, arsenic, nickel, lead, or any combination thereof from a fluid, such as a fluid comprising water.

### Example 16: Calculation of Crosslink Density

This example demonstrates examples of how to calculate crosslink density. A specific crosslinker is capable of forming n bonds with available functional groups on the precursor of a functional polymer particle (e.g., microgel) and the average number reactive groups on the precursor of the functional polymer particle (e.g., microgel) could form up to m bonds covalent bonds to the crosslinker; and if n moles of crosslinker are reacted with m moles of precursor of the functional polymer particle (e.g., microgel), then the crosslinking ratio (e.g., crosslinking density) is (n bonds*n moles)/(m bonds*m moles).

In this example, the average number of each type of functional group was rounded to the nearest integer; using the integrated intensity of distinctive peaks in the 1H NMR spectrum of a specific batch of PEI, a more precise value for the average number of primary amines and secondary amines could be evaluated.

For the case of ECH/PEI, each primary amine of a PEI can potentially form two bonds to functional groups of crosslinker molecules, whereas each secondary amine can only form one additional bond. If a PEI of 600 g/mol is used, it contains on average four (4) primary amines, seven (7) secondary amines and 3 tertiary amines. The tertiary amines provide an example of a functional group that may play role in the function of the microgel particles, but does not contribute to bond formation during crosslinking. The primary amines are an example of a functional group that enables more than one bond to form during cross linking: each primary amine has the potential to form two bonds in the presence of ECH. The secondary amines are an example of a functional group that can only form one bond upon reaction with ECH. ECH is capable of forming two chemical bonds to PEI, one via the epoxide and one via the alkylhalide present in each ECH molecule. It is noted that the number of bonds that can form for each of the crosslinking functional groups can be readily determined.

To illustrate how the formula is used in the case of ECH and PEI of 600 g/mol: for the crosslinker, n moles is the number of moles of ECH and n bonds is two; for the precursor of the functional gel, m moles is the number of moles of PEI and m bonds is 4*2+7=15; and the crosslink density is calculated in this case as (2 n moles)/(15 m moles).

For the case of ECH and G0 PAMAM, each primary amine of G0 PAMAM can potentially form two bond to functional groups of crosslinker molecules. A G0 PAMAM has four (4) primary amines, four amides, and two tertiary amines. The only ones that participate in chemical reactions with ECH are the primary amines. To illustrate how the formula is used in the case of ECH and G0 PAMAM: for the crosslinker, n moles is the number of moles of ECH and n bonds is two (2); for the precursor of the functional gel, m moles is the number of moles of G0 PAMAM and m bonds is 4*2=8; and the crosslink density is calculated in this case as (2 n moles)/(8 m moles).

For the case of ECH and G1 PAMAM, each primary amine of G1 PAMAM can potentially form two bond to functional groups of crosslinker molecules. A G1 PAMAM has eight (8) primary amines, 12 amides, and 6 tertiary amines. The only ones that participate in chemical reactions with ECH are the primary amines. To illustrate how the formula is used in the case of ECH and G1 PAMAM: for the crosslinker, n moles is the number of moles of ECH and n bonds is two (2); for the precursor of the functional gel, m moles is the number of moles of G1 PAMAM and m bonds is 4*2=8; and the crosslink density is calculated in this case as (2 n moles)/(16 m moles).

### Example 17: MOF as the Functional Polymer Particle

This example demonstrates a method of making a MOF as the functional polymer particles on a PVDF structural polymer by a two step infusion/phase-separation (MOFs via pH-IPS). This method involves a first infiltration of PVDF dope solution, a NIPS step, then a second infusion of an aqueous solution followed by a pH-induced phase separation.

First, a macroporous scaffold is prepared as described elsewhere herein. For the purposes of this example, the scaffold can be ceramic, such as silicon oxycarbide prepared through a freeze casting method.

Second, infiltration into the pores can be performed, in which PVDF structural polymer cab be introduced and grafted. Then NIPS can be performed, such that the PVDF forms a web of nanoscopic-diameter filaments that crisscross each pore.

Third, one can apply an adaptation of the procedure described in Huelsenbeck et al., which describes control of nucleation and growth of MOFs using pH (Huelsenbeck et al., “Generalized Approach for Rapid Aqueous MOF Synthesis by Controlling Solution pH,” Cryst. Growth Des. 2020, 20, 10, 6787-6795). The adaption for the present example would include using a second infusion performed with a low pH so the solution will not solidify prior to infiltration. Then, one would submerge in a solution that has a somewhat lower concentration of reagents and a high pH so that as the hydroxyl groups diffuse into the pores, they induce nucleation and growth of MOFs. This can be considered a phase separation process, neither TIPS nor NIPS, but rather a PIPS process, which is a pH-induced phase separation. As in the in situ formation of other polymer particles, the in situ formation of the MOFs is expected to produce some particles that encircle the PVDF fibers; these particles will be stably incorporated into the hybrid material, especially those that nucleate on the PVDF. Here, the MOF particle represents a functional polymer particle with extremely high porosity, in which the repeat units are the metal and the ligands of the framework.

### Example 18: MOF as a Second Set of Functional Polymer Particles

This example demonstrates a method of making a MOF as the second set of polymer particles in a PVDF/PEO (structural polymer/microgel) first infusion/NIPS followed by MOF preparation.

In this example, we propose that a very highly swollen PEO microgel might be used to favor a specific crystal morph of a MOF grown into a first polymer matrix comprising a structural polymer and PEO microgel polymer particles by a second infusion step of an aqueous solution of the MOF precursors and a second phase separation step to synthesize a second set of polymer particles that are highly porous MOF (with the metal and ligand that form the MOF as the repeat units of the polymer). This concept can be used when preparing a composite membrane using the methods, which disclosed elsewhere herein. It is believed such a concept is possible in view of Westendorff et al., which studies polymorphism in the ZIF-8/ZIF-L system as a function of metal:ligand ratio during synthesis and shows a significant shift in the phase transition point towards ZIF-8 with addition of dilute polyethylene oxide during synthesis. Computational results in Westendorff et al. suggest a simple pathway for controlling

MOF polymorphism where the choice of polymer can be guided via first-principles simulations. (Westendorff et al., “Polymer-induced polymorphism in a Zn-based metal organic framework,” Chem. Comm. (2021) 57, 88)).

### Example 19: Utility of MOFs as a Type of Polymer Particle

This example demonstrates the utility of MOFs as a type of polymer particle.

As disclosed in PCT/US2019/042586 “Methods of Making MOFs, Systems For Synthesizing MOFs, and Methods of Coating Textiles with MOFs,” MOFs are useful in a variety of applications, including a textile that has a MOF coating. Such MOF-coated textiles can be used for a variety of applications, including catalysis, separations, sensing, gas storage, and medicine.

As a result, MOFs prepared in a composite membrane as disclosed herein can also find utility in a variety of applications, including catalysis, separations, sensing, gas storage, and medicine.

### Example 20: Catalysis Using a Composite Membrane

This example demonstrates catalysis applications of the composite membrane disclosed herein.

A composite membrane can be prepared as described elsewhere herein, in which the composite membrane comprises at least one metal chelated to the polymer matrix (e.g., functional polymer particle and/or structural polymer). The at least one metal can be a transition metal, an alloy, or any combination thereof. In some aspects, the at least one metal can be a noble metal. In some aspects, the metal can be copper, palladium, platinum, iron, rhodium, ruthenium, or any combination thereof. In some aspects, suitable metals, mixed matrices, and catalyzed reactions include those disclosed in US 2016/0303517 (Diallo et al.), hereby incorporated by reference in its entirety for all purposes.

In some aspects, the composite membrane comprising the at least one metal chelated to the polymer matrix can be used in a catalysis method, the method comprising passing a mixture through the composite membrane, in which the composite membrane catalyzes a chemical reaction in the mixture.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects, exemplary aspects and optional features, modification and variation of the concepts herein disclosed may be resorted to by those skilled in the art, and that such modifications and variations are considered to be within the scope of this invention as defined by the appended claims. The specific aspects provided herein are examples of useful aspects of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some aspects is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules, polymers, and/or crosslinkers disclosed herein may contain one or more ionizable groups, groups from which a proton can be removed (e.g., —COOH) or added (e.g., amines), or which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific aspects that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

1. A composite, comprising:
a macroporous scaffold comprising pores; and
a polymer matrix positioned within the pores;
wherein the polymer matrix comprises:
a functional polymer particle; and
a structural polymer.

2. The composite of claim 1,
wherein the functional polymer particle comprises a functional gel.

3. The composite of claim 1,
wherein the functional polymer particle comprises at least one primary amine, at least one primary ammonium, at least one secondary amine, at least one secondary ammonium, at least one tertiary amine, at least one tertiary ammonium, or any combination thereof.

4.-5. (canceled)

6. The composite of claim 1, wherein:
the pores comprise through-pores;
the functional polymer particle is in a form of a plurality of particles; and
the plurality of particles has an average particle size that is from 0.01 D to 0.2 D when measured in a wet state, wherein D is an average diameter of the through-pores, and optionally the plurality of particles is in a swollen state in the wet state.

7. The composite of claim 1,
wherein the functional polymer particle comprises a functional gel comprising a hydrogel.

8. The composite of claim 1,
wherein the functional polymer particle comprises polyethylenimine (PEI), branched PEI, hyperbranched PEI, poly(ethylene oxide) (PEO), poly-N-isopropylacrylamide, polyamidoamine dendrimers (PAMAM), low generation PAMAM, chitosan, gelatin, a biopolymer, a functional biopolymer, carrageenan, or any combination thereof.

9.-11. (canceled)

12. The composite of claim 1,
wherein the functional polymer particle comprises at least one functional group capable of binding to a species of interest selected from a peptide, a protein, a glycoprotein, barium, zinc, boron, chromium, iron, selenium, arsenic, nickel, lead, platinum, or any combination thereof.

13.-19. (canceled)

20. The composite of claim 1,
(1) wherein the functional polymer particle is crosslinked and has at least one crosslinked structure comprising formula (2), (3), (4), (5), (6), or any combination thereof: 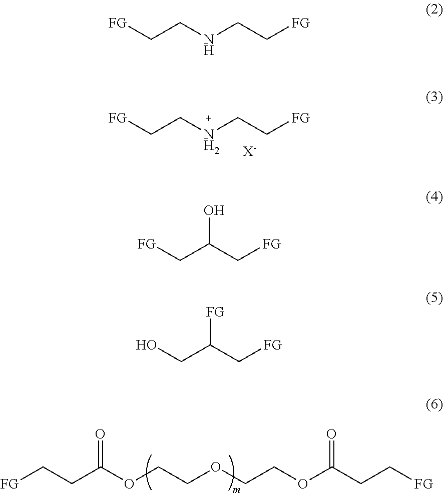
wherein:
FG is the functional polymer particle;
X is a counterion; and
m is an integer from 0 to 20; or
(2) wherein the functional polymer particle is crosslinked from a crosslinker comprising: 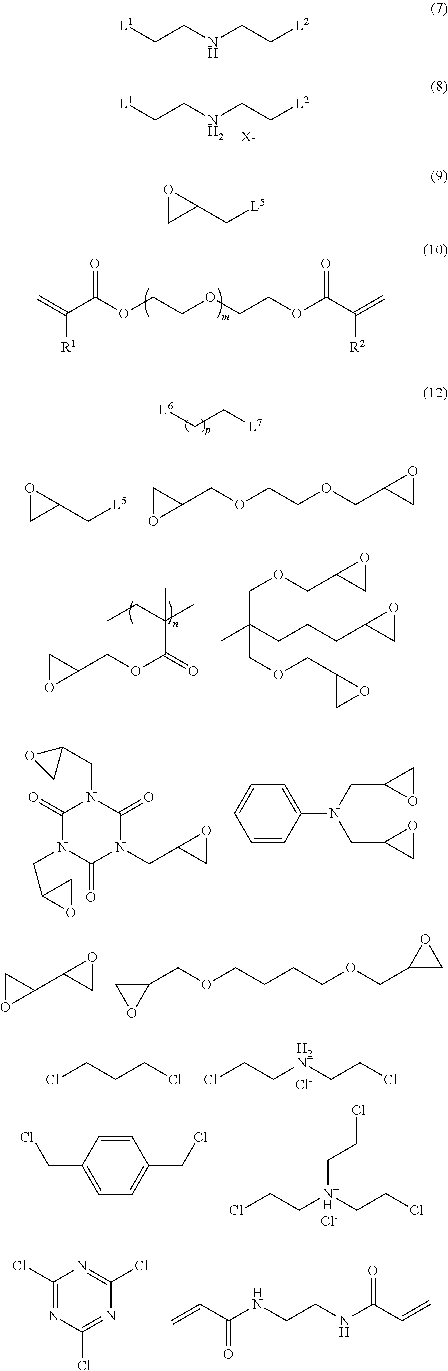 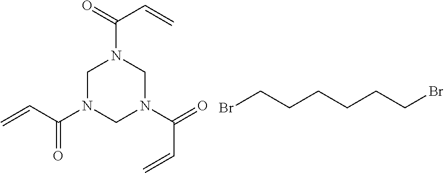
or any combination thereof,
wherein each of L¹, L², L³, L⁴, L⁵, L⁶, and L⁷, is a leaving group optionally selected from a halide, tosylate (OTs), mesylate (OMs), triflate (OTf), 2,2,2-trifluoroethanesulfonate, alkylsulfonate, benzenesulfonate, substituted benzenesulfonate, or phosphate;
X is a counterion optionally selected from chloride, bromide, or iodide;
each of R¹ and R² independently is hydrogen or C₁-C₆ alkyl;
n is an integer from 2 to 50;
m is an integer from 0 to 20; and
p is an integer from 1 to 9.

21.-25. (canceled)

26. The composite of claim 1,
wherein the functional polymer particle and/or structural polymer is covalently attached indirectly to a surface of the pores via an oligomer or polymer, wherein the oligomer or polymer comprises at least one primary amine, at least one primary ammonium, at least one secondary amine, at least one secondary ammonium, at least one tertiary amine, at least one tertiary ammonium, or any combination thereof.

27.-28. (canceled)

29. The composite of claim 1, wherein:
the functional polymer particle and/or structural polymer is indirectly attached to a surface of the pores;
the functional polymer particle and/or structural polymer is crosslinked to PEI; and
the PEI is crosslinked to a functional group on the surface of the pores.

30. The composite of claim 1, wherein:
the functional polymer particle comprises a metal-organic framework (MOF), a covalent organic framework (COF), a nanoporous polymer, a functional gel, or any combination thereof.

31. (canceled)

32. The composite of claim 1,
wherein the macroporous scaffold comprises ceramic, organic glass, inorganic glass, carbon, charcoal, graphene, graphite, metal, fused metal particles, polymer, crystalline polymer, semicrystalline polymer, fused polymer particles, other dispersed species, or any combination thereof.

33. The composite of claim 1,
wherein the macroporous scaffold comprises the ceramic or inorganic glass, and the ceramic or inorganic glass comprises silicon oxycarbide.

34.-35. (canceled)

36. The composite of claim 1,
wherein the macroporous scaffold comprises a pore volume fraction of 10% to 70% of the composite.

37.-38. (canceled)

39. The composite of claim 1,
wherein the pores are oriented along a primary axis.

40. The composite of claim 1, wherein:
the functional polymer particle comprises a functional gel, and
the structural polymer is insoluble or slightly soluble in a solvent capable of swelling the functional gel, optionally wherein the solvent comprises water.

41. The composite of claim 1,
wherein the structural polymer comprises polyvinylidene fluoride (PVDF), cellulose acetate, polysulfone, polyvinyl chloride, poly(acrylonitrile), polyethersulfone (PES), polypropylene, polytetrafluoroethylene, polyamide imide, natural rubber, or any combination thereof.

42.-44. (canceled)

45. The composite of claim 1, further comprising:
at least one metal chelated to the polymer matrix,
optionally wherein the metal comprises a transition metal optionally selected from copper, palladium, platinum, iron, rhodium, ruthenium, or any combination thereof.

46. (canceled)

47. A method for making the composite of claim 1, the method comprising:
infiltrating the pores with a liquid comprising the polymer matrix or a precursor thereof; and
performing nonsolvent induced phase separation (NIPS) on the macroporous scaffold infiltrated with the liquid.

48.-53. (canceled)

54. A method comprising:
passing a mixture through the composite of claim 1, wherein the composite (1) isolates a component from the mixture, (2) catalyzes a chemical reaction in the mixture, or (3) both.

55.-58. (canceled)
